(19) United States
(12) Patent Application Publication
(10) Pub. No.: US 2023/0165138 A1
(43) Pub. Date: May 25, 2023

(54) COMPOUND AND ORGANIC ELECTROLUMINESCENCE DEVICE

(71) Applicant: IDEMITSU KOSAN CO.,LTD., Tokyo (JP)

(72) Inventors: Yoshinao SHIRASAKI, Sodegaura-shi (JP); Ryota TAKAHASHI, Sodegaura-shi (JP); Yuki NAKANO, Sodegaura-shi (JP); Kazuma MASE, Sodegaura-shi (JP); Tetsuya MASUDA, Sodegaura-shi (JP)

(73) Assignee: IDEMITSU KOSAN CO.,LTD., Tokyo (JP)

(21) Appl. No.: 17/909,701

(22) PCT Filed: Feb. 24, 2021
(86) PCT No.: PCT/JP2021/006789

§ 371 Date: Sep. 6, 2022

(30) Foreign Application Priority Data
Mar. 9, 2020 (JP) 2020-040065

(51) Int. Cl. H10K 85/60; C07D 307/77; C09K 11/06

(52) U.S. Cl. CPC H10K 85/636; C07D 307/77; C09K 11/06; C09K 2211/1018; H10K 85/6574; H10K 50/11

(57) ABSTRACT

A compound represented by the following formula (1), wherein at least one of R₁₁ to R₂₈ and R₃₁ to R₄₀ is not a hydrogen atom.

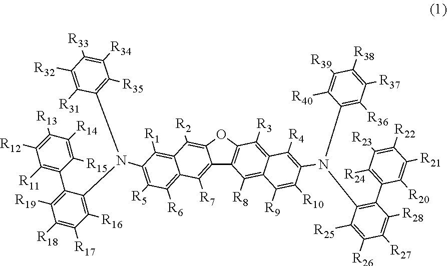

### TECHNICAL FIELD

The present invention relates to a novel compound and an organic electroluminescence device.

### BACKGROUND ART

When voltage is applied to an organic electroluminescence device (hereinafter, frequently referred to as an organic EL device), holes and electrons are injected into an emitting layer from an anode and a cathode, respectively. Then, thus injected holes and electrons are recombined in the emitting layer, and excitons are formed therein.

Conventional organic EL devices have not yet had sufficient device performance. Although materials used for the organic EL device are gradually improved to enhance the device performance, further performance enhancement is required. In particular, since the improvement of the lifetime of the organic EL device is an important problem leading to the lifetime of the commercialized product, a material capable of achieving an organic EL device having a long lifetime is required.

Patent Documents 1 and 2 disclose that a compound having the specific structure is used for a emitting layer of an organic EL device.

### RELATED ART DOCUMENTS

### Patent Documents

[Patent Document 1] WO 2018/235953 A1
[Patent Document 2] WO 2014/104144 A1

### SUMMARY OF THE INVENTION

It is an object of the present invention to provide a compound capable of fabricating an organic EL device with high efficiency and long lifetime.

As a result of intensive studies to achieve the above object, the present inventors have found that an organic EL device having high efficiency and long lifetime can be obtained by using a compound having the specific structure, and have completed the present invention.

According to the present invention, the following compound and the like are provided.

A compound represented by the following formula (1):

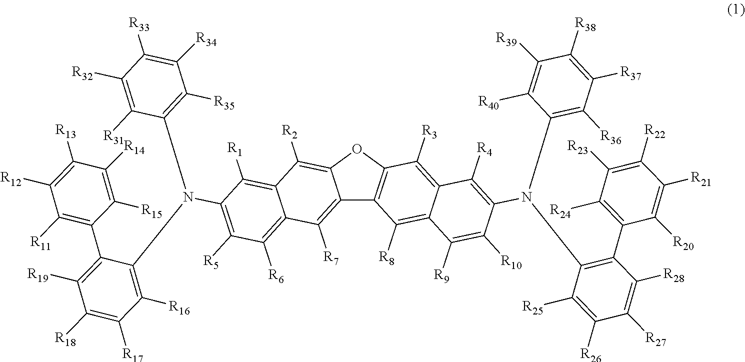

wherein in the formula (1),

R₁ to R₁₀ are independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a substituted or unsubstituted alkoxy group having 1 to 50 carbon atoms, a substituted or unsubstituted alkylthio group having 1 to 50 carbon atoms, a substituted or unsubstituted aryloxy group having 6 to 50 ring carbon atoms, a substituted or unsubstituted arylthio group having 6 to 50 ring carbon atoms, a substituted or unsubstituted aralkyl group having 7 to 50 carbon atoms, —Si(R₉₁)(R₉₂)(R₉₃), —C(═O)R₉₄, —COOR₉₅, —N(R₉₆)(R₉₇), a halogen atom, a cyano group, a nitro group, a substituted or unsubstituted aryl group having 6 to 50 ring carbon atoms, or a substituted or unsubstituted monovalent heterocyclic group having 5 to 50 ring atoms;

R₉₁ to R₉₇ are independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted monovalent heterocyclic group having 5 to 50 ring atoms;

when a plurality of each of R₉₁ to R₉₇ are present, the plurality of each of R₉₁ to R₉₇ may be the same as or different from each other; R₁₁ to R₂₈ are independently a hydrogen atom, a substituted or unsubstituted alkyl group having 1 to 20 carbon atoms, a substituted or unsubstituted cycloalkyl group having 3 to 20 ring carbon atoms, a silyl group substituted with an alkyl group having 1 to 20 carbon atoms, or a cyano group; R₃₁ to R₄₀ are independently a hydrogen atom, a substituted or unsubstituted alkyl group having 1 to 20 carbon atoms, a substituted or unsubstituted cycloalkyl group having 3 to 20 ring carbon atoms, a substituted or unsubstituted aryl group having 6 to 20 ring carbon atoms, a silyl group substituted with an alkyl group having 1 to 20 carbon atoms, or a cyano group;

at least one of R₁₁ to R₂₈ and R₃₁ to R₄₀ is not a hydrogen atom.

According to the present invention, there can be provided a compound capable of fabricating an organic EL device with high efficiency and long lifetime.

### BRIEF DESCRIPTION OF THE DRAWINGS

FIG. 1 is a diagram showing a schematic configuration of an organic EL device according to an aspect of the present invention.

### MODE FOR CARRYING OUT THE INVENTION

### Definition

In this specification, a hydrogen atom includes its isotopes different in the number of neutrons, namely, a protium, a deuterium and a tritium.

In this specification, at a bondable position in a chemical formula where a symbol such as “R”, or “D” representing a deuterium atom is not indicated, a hydrogen atom, that is, a protium atom, a deuterium atom or a tritium atom is bonded.

In this specification, the number of ring carbon atoms represents the number of carbon atoms forming a subject ring itself among the carbon atoms of a compound having a structure in which atoms are bonded in a ring form (for example, a monocyclic compound, a fused ring compound, a cross-linked compound, a carbocyclic compound, or a heterocyclic compound). When the subject ring is substituted by a substituent, the carbon contained in the substituent is not included in the number of ring carbon atoms. The same shall apply to “the number of ring carbon atoms” described below, unless otherwise specified. For example, a benzene ring has 6 ring carbon atoms, a naphthalene ring includes 10 ring carbon atoms, a pyridine ring includes 5 ring carbon atoms, and a furan ring includes 4 ring carbon atoms. Further, for example, a 9,9-diphenylfluorenyl group includes 13 ring carbon atoms, and a 9,9′-spirobifluorenyl group includes 25 ring carbon atoms.

When a benzene ring is substituted by, for example, an alkyl group as a substituent, the number of carbon atoms of the alkyl group is not included in the number of ring carbon atoms of the benzene ring. Therefore, the number of ring carbon atoms of the benzene ring substituted by the alkyl group is 6. When a naphthalene ring is substituted by, for example, an alkyl group as a substituent, the number of carbon atoms of the alkyl group is not included in the number of ring carbon atoms of the naphthalene ring. Therefore, the number of ring carbon atoms of the naphthalene ring substituted by the alkyl group is 10.

In this specification, the number of ring atoms represents the number of atoms forming a subject ring itself among the atoms of a compound having a structure in which atoms are bonded in a ring form (for example, the structure includes a monocyclic ring, a fused ring and a ring assembly) (for example, a monocyclic compound, a fused ring compound, a cross-linked compound, a carbocyclic compound and a heterocyclic compound). The number of ring atoms does not include atoms which do not form the ring (for example, a hydrogen atom which terminates a bond of the atoms forming the ring), or atoms contained in a substituent when the ring is substituted by the substituent. The same shall apply to “the number of ring atoms” described below, unless otherwise specified. For example, the number of atoms of a pyridine ring is 6, the number of atoms of a quinazoline ring is 10, and the number of a furan ring is 5. For example, hydrogen atoms bonded to a pyridine ring and atoms constituting a substituent substituted on the pyridine ring are not included in the number of ring atoms of the pyridine ring. Therefore, the number of ring atoms of a pyridine ring with which a hydrogen atom or a substituent is bonded is 6. For example, hydrogen atoms and atoms constituting a substituent which are bonded with a quinazoline ring is not included in the number of ring atoms of the quinazoline ring. Therefore, the number of ring atoms of a quinazoline ring with which a hydrogen atom or a substituent is bonded is 10.

In this specification, “XX to YY carbon atoms” in the expression “a substituted or unsubstituted ZZ group including XX to YY carbon atoms” represents the number of carbon atoms in the case where the ZZ group is unsubstituted by a substituent, and does not include the number of carbon atoms of a substituent in the case where the ZZ group is substituted by the substituent. Here, “YY” is larger than “XX”, and “XX” means an integer of 1 or more and “YY” means an integer of 2 or more.

In this specification, “XX to YY atoms” in the expression “a substituted or unsubstituted ZZ group including XX to YY atoms” represents the number of atoms in the case where the ZZ group is unsubstituted by a substituent, and does not include the number of atoms of a substituent in the case where the ZZ group is substituted by the substituent. Here, “YY” is larger than “XX”, and “XX” means an integer of 1 or more and “YY” means an integer of 2 or more.

In this specification, the unsubstituted ZZ group represents the case where the “substituted or unsubstituted ZZ group” is a “ZZ group unsubstituted by a substituent”, and the substituted ZZ group represents the case where the “substituted or unsubstituted ZZ group” is a “ZZ group substituted by a substituent”.

In this specification, a term “unsubstituted” in the case of “a substituted or unsubstituted ZZ group” means that hydrogen atoms in the ZZ group are not substituted by a substituent. Hydrogen atoms in a term “unsubstituted ZZ group” are a protium atom, a deuterium atom, or a tritium atom.

In this specification, a term “substituted” in the case of “a substituted or unsubstituted ZZ group” means that one or more hydrogen atoms in the ZZ group are substituted by a substituent. Similarly, a term “substituted” in the case of “a BB group substituted by an AA group” means that one or more hydrogen atoms in the BB group are substituted by the AA group.

“Substituent as Described in this Specification”

Hereinafter, the substituent described in this specification will be explained.

The number of ring carbon atoms of the “unsubstituted aryl group” described in this specification is 6 to 50, preferably 6 to 30, and more preferably 6 to 18, unless otherwise specified.

The number of ring atoms of the “unsubstituted heterocyclic group” described in this specification is 5 to 50, preferably 5 to 30, and more preferably 5 to 18, unless otherwise specified.

The number of carbon atoms of the “unsubstituted alkyl group” described in this specification is 1 to 50, preferably 1 to 20, and more preferably 1 to 6, unless otherwise specified.

The number of carbon atoms of the “unsubstituted alkenyl group” described in this specification is 2 to 50, preferably 2 to 20, and more preferably 2 to 6, unless otherwise specified.

The number of carbon atoms of the “unsubstituted alkynyl group” described in this specification is 2 to 50, preferably 2 to 20, and more preferably 2 to 6, unless otherwise specified.

The number of ring carbon atoms of the “unsubstituted cycloalkyl group” described in this specification is 3 to 50, preferably 3 to 20, and more preferably 3 to 6, unless otherwise specified.

The number of ring carbon atoms of the “unsubstituted arylene group” described in this specification is 6 to 50, preferably 6 to 30, and more preferably 6 to 18, unless otherwise specified.

The number of ring atoms of the “unsubstituted divalent heterocyclic group” described in this specification is 5 to 50, preferably 5 to 30, and more preferably 5 to 18, unless otherwise specified.

The number of carbon atoms of the “unsubstituted alkylene group” described in this specification is 1 to 50, preferably 1 to 20, and more preferably 1 to 6, unless otherwise specified.

“Substituted or Unsubstituted Aryl Group”

Specific examples of the “substituted or unsubstituted aryl group” described in this specification (specific example group G1) include the following unsubstituted aryl groups (specific example group G1A), substituted aryl groups (specific example group G1B), and the like. (Here, the unsubstituted aryl group refers to the case where the “substituted or unsubstituted aryl group” is an “aryl group unsubstituted by a substituent”, and the substituted aryl group refers to the case where the “substituted or unsubstituted aryl group” is an “aryl group substituted by a substituent”). In this specification, in the case where simply referred as an “aryl group”, it includes both a “unsubstituted aryl group” and a “substituted aryl group.”

The “substituted aryl group” means a group in which one or more hydrogen atoms of the “unsubstituted aryl group” are substituted by a substituent. Specific examples of the “substituted aryl group” include, for example, groups in which one or more hydrogen atoms of the “unsubstituted aryl group” of the following specific example group G1A are substituted by a substituent, the substituted aryl groups of the following specific example group G1B, and the like. It should be noted that the examples of the “unsubstituted aryl group” and the examples of the “substituted aryl group” enumerated in this specification are mere examples, and the “substituted aryl group” described in this specification also includes a group in which a hydrogen atom bonded with a carbon atom of the aryl group itself in the “substituted aryl group” of the following specific group G1B is further substituted by a substituent, and a group in which a hydrogen atom of a substituent in the “substituted aryl group” of the following specific group G1B is further substituted by a substituent.
Unsubstituted aryl group (specific example group G1A):
   a phenyl group,
   a p-biphenyl group,
   a m-biphenyl group,
   an o-biphenyl group,
   a p-terphenyl-4-yl group,
   a p-terphenyl-3-yl group,
   a p-terphenyl-2-yl group,
   a m-terphenyl-4-yl group,
   a m-terphenyl-3-yl group,
   a m-terphenyl-2-yl group,
   an o-terphenyl-4-yl group,
   an o-terphenyl-3-yl group,
   an o-terphenyl-2-yl group,
   a 1-naphthyl group,
   a 2-naphthyl group,
   an anthryl group,
   a benzanthryl group,
   a phenanthryl group,
   a benzophenanthryl group,
   a phenalenyl group,
   a pyrenyl group,
   a chrysenyl group,
   a benzochrysenyl group,
   a triphenylenyl group,
   a benzotriphenylenyl group,
   a tetracenyl group,
   a pentacenyl group,
   a fluorenyl group,
   a 9,9′-spirobifluorenyl group,
   a benzofluorenyl group,
   a dibenzofluorenyl group,
   a fluoranthenyl group,
   a benzofluoranthenyl group,
   a perylenyl group, and
   a monovalent aryl group derived by removing one hydrogen atom from the ring structures represented by any of the following general formulas (TEMP-1) to (TEMP-15).

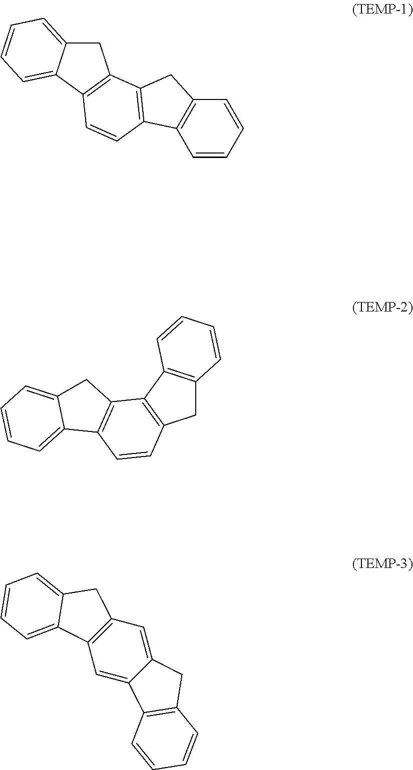

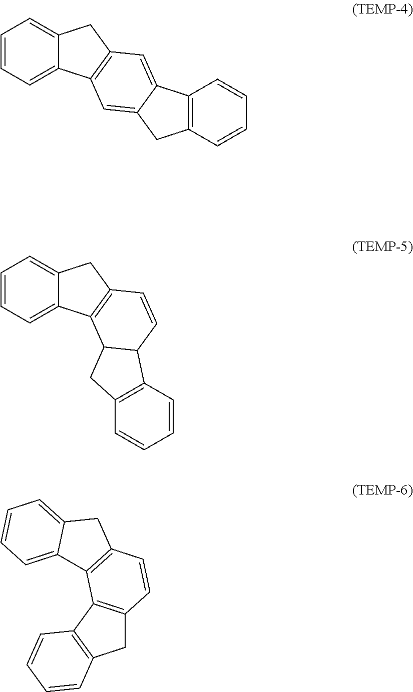

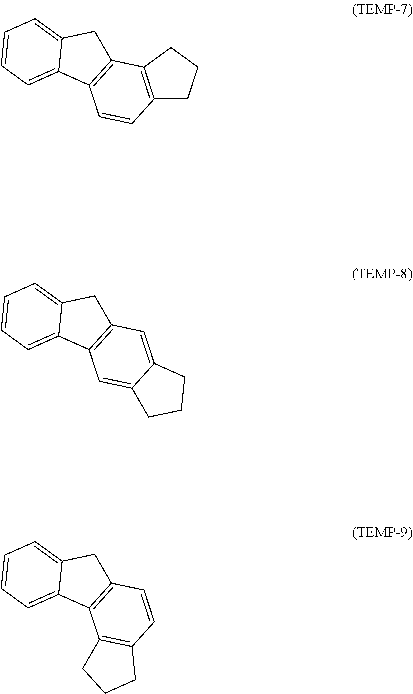

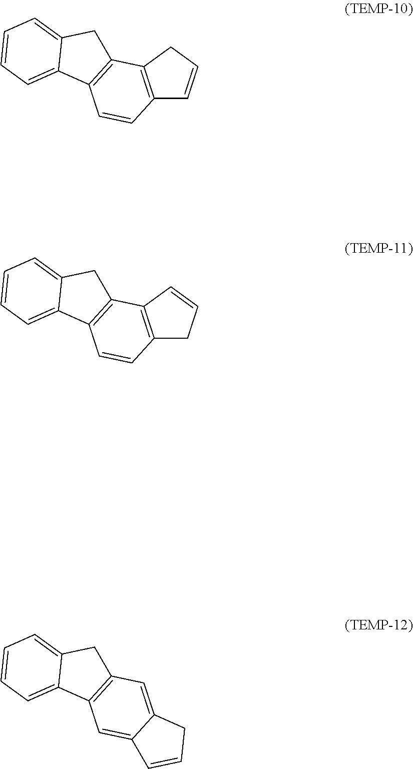

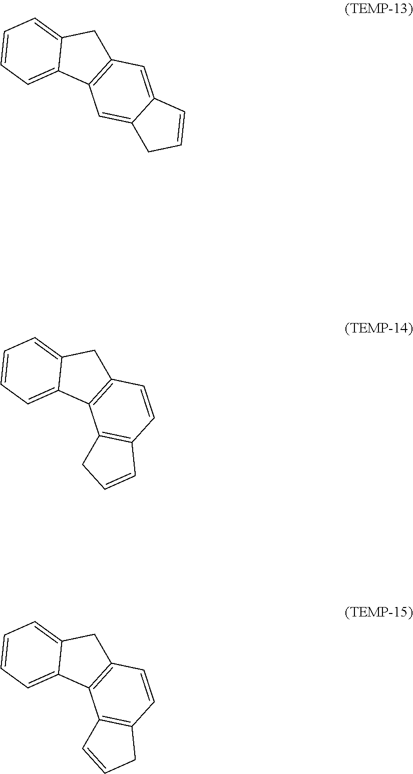

substituted aryl group (specific example group G1B):
   an o-tolyl group,
   a m-tolyl group,
   a p-tolyl group,
   a p-xylyl group,
   a m-xylyl group,
   an o-xylyl group,
   a p-isopropylphenyl group,
   a m-isopropylphenyl group,
   an o-isopropylphenyl group,
   a p-t-butylphenyl group,
   a m-t-butylphenyl group,
   an o-t-butylphenyl group,
   a 3,4,5-trimethylphenyl group,
   a 9,9-dimethylfluorenyl group,
   a 9,9-diphenylfluorenyl group,
   a 9,9-bis(4-methylphenyl)fluorenyl group,
   a 9,9-bis(4-isopropylphenyl)fluorenyl group,
   a 9,9-bis(4-t-butylphenyl)fluorenyl group,
   a cyanophenyl group,
   a triphenylsilylphenyl group,
   a trimethylsilylphenyl group,
   a phenylnaphthyl group,
   a naphthylphenyl group, and
   a group in which one or more hydrogen atoms of a monovalent group derived from the ring structures represented by any of the general formulas (TEMP-1) to (TEMP-15) are substituted by a substituent.

“Substituted or Unsubstituted Heterocyclic Group”

The “heterocyclic group” described in this specification is a ring group having at least one hetero atom in the ring atom. Specific examples of the hetero atom include a nitrogen atom, an oxygen atom, a sulfur atom, a silicon atom, a phosphorus atom, and a boron atom.

The “heterocyclic group” in this specification is a monocyclic group or a fused ring group.

The “heterocyclic group” in this specification is an aromatic heterocyclic group or a non-aromatic heterocyclic group.

Specific examples of the “substituted or unsubstituted heterocyclic group” (specific example group G2) described in this specification include the following unsubstituted heterocyclic group (specific example group G2A), the following substituted heterocyclic group (specific example group G2B), and the like. (Here, the unsubstituted heterocyclic group refers to the case where the “substituted or unsubstituted heterocyclic group” is a “heterocyclic group unsubstituted by a substituent”, and the substituted heterocyclic group refers to the case where the “substituted or unsubstituted heterocyclic group” is a “heterocyclic group substituted by a substituent”). In this specification, in the case where simply referred as a “heterocyclic group”, it includes both the “unsubstituted heterocyclic group” and the “substituted heterocyclic group.”

The “substituted heterocyclic group” means a group in which one or more hydrogen atom of the “unsubstituted heterocyclic group” are substituted by a substituent. Specific examples of the “substituted heterocyclic group” include a group in which a hydrogen atom of “unsubstituted heterocyclic group” of the following specific example group G2A is substituted by a substituent, the substituted heterocyclic groups of the following specific example group G2B, and the like. It should be noted that the examples of the “unsubstituted heterocyclic group” and the examples of the “substituted heterocyclic group” enumerated in this specification are mere examples, and the “substituted heterocyclic group” described in this specification includes groups in which hydrogen atom bonded with a ring atom of the heterocyclic group itself in the “substituted heterocyclic group” of the specific example group G2B is further substituted by a substituent, and a group in which hydrogen atom of a substituent in the “substituted heterocyclic group” of the specific example group G2B is further substituted by a substituent.

Specific example group G2A includes, for example, the following unsubstituted heterocyclic group containing a nitrogen atom (specific example group G2A1), the following unsubstituted heterocyclic group containing an oxygen atom (specific example group G2A2), the following unsubstituted heterocyclic group containing a sulfur atom (specific example group G2A3), and the monovalent heterocyclic group derived by removing one hydrogen atom from the ring structures represented by any of the following general formulas (TEMP-16) to (TEMP-33) (specific example group G2A4).

Specific example group G2B includes, for example, the following substituted heterocyclic group containing a nitrogen atom (specific example group G2B1), the following substituted heterocyclic group containing an oxygen atom (specific example group G2B2), the following substituted heterocyclic group containing a sulfur atom (specific example group G2B3), and the following group in which one or more hydrogen atoms of the monovalent heterocyclic group derived from the ring structures represented by any of the following general formulas (TEMP-16) to (TEMP-33) are substituted by a substituent (specific example group G2B4).
Unsubstituted heterocyclic group containing a nitrogen atom (specific example group G2A1):
   a pyrrolyl group,
   an imidazolyl group,
   a pyrazolyl group,
   a triazolyl group,
   a tetrazolyl group,
   an oxazolyl group,
   an isoxazolyl group,
   an oxadiazolyl group,
   a thiazolyl group,
   an isothiazolyl group,
   a thiadiazolyl group,
   a pyridyl group,
   a pyridazinyl group,
   a pyrimidinyl group,
   a pyrazinyl group,
   a triazinyl group,
   an indolyl group,
   an isoindolyl group,
   an indolizinyl group,
   a quinolizinyl group,
   a quinolyl group,
   an isoquinolyl group,
   a cinnolyl group,
   a phthalazinyl group,
   a quinazolinyl group,
   a quinoxalinyl group,
   a benzimidazolyl group,
   an indazolyl group,
   a phenanthrolinyl group,
   a phenanthridinyl group,
   an acridinyl group,
   a phenazinyl group,
   a carbazolyl group,
   a benzocarbazolyl group,
   a morpholino group,
   a phenoxazinyl group,
   a phenothiazinyl group,
   an azacarbazolyl group, and
   a diazacarbazolyl group.
Unsubstituted heterocyclic group containing an oxygen atom (specific example group G2A2):
   a furyl group,
   an oxazolyl group,
   an isoxazolyl group,
   an oxadiazolyl group,
   a xanthenyl group,
   a benzofuranyl group,
   an isobenzofuranyl group,
   a dibenzofuranyl group,
   a naphthobenzofuranyl group,
   a benzoxazolyl group,
   a benzisoxazolyl group,
   a phenoxazinyl group,
   a morpholino group,
   a dinaphthofuranyl group,
   an azadibenzofuranyl group,
   a diazadibenzofuranyl group,
   an azanaphthobenzofuranyl group, and
   a diazanaphthobenzofuranyl group.
Unsubstituted heterocyclic group containing a sulfur atom (specific example group G2A3):
   a thienyl group,
   a thiazolyl group,
   an isothiazolyl group,
   a thiadiazolyl group,
   a benzothiophenyl group (benzothienyl group),
   an isobenzothiophenyl group (isobenzothienyl group),
   a dibenzothiophenyl group (dibenzothienyl group),
   a naphthobenzothiophenyl group (naphthobenzothienyl group),
   a benzothiazolyl group,
   a benzisothiazolyl group,
   a phenothiazinyl group,
   a dinaphthothiophenyl group (dinaphthothienyl group),
   an azadibenzothiophenyl group (azadibenzothienyl group),
   a diazadibenzothiophenyl group (diazadibenzothienyl group),
   an azanaphthobenzothiophenyl group (azanaphthobenzothienyl group), and
   a diazanaphthobenzothiophenyl group (diazanaphthobenzothienyl group).
   Monovalent heterocyclic group derived by removing one hydrogen atom from the ring structures represented by any of the following general formulas (TEMP-16) to (TEMP-33) (specific example group G2A4):

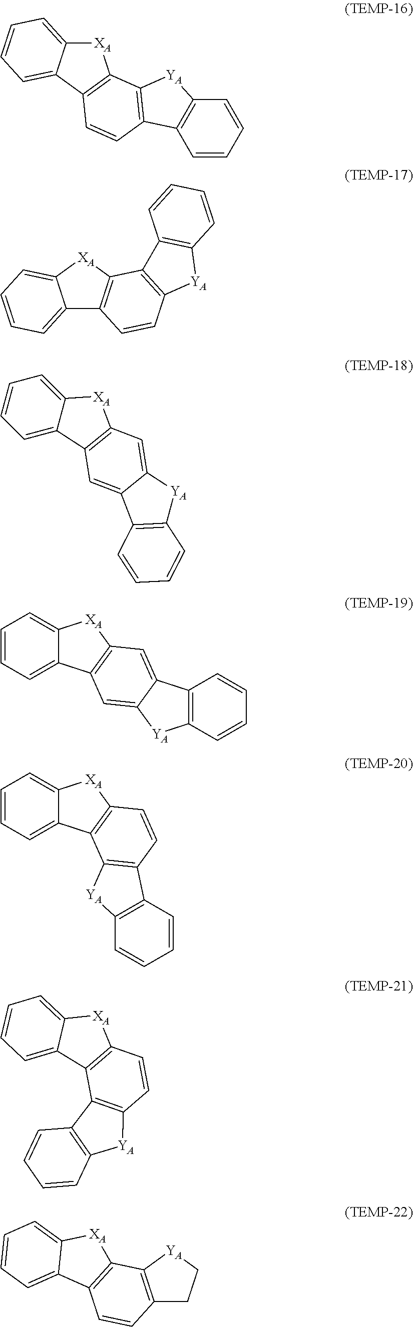

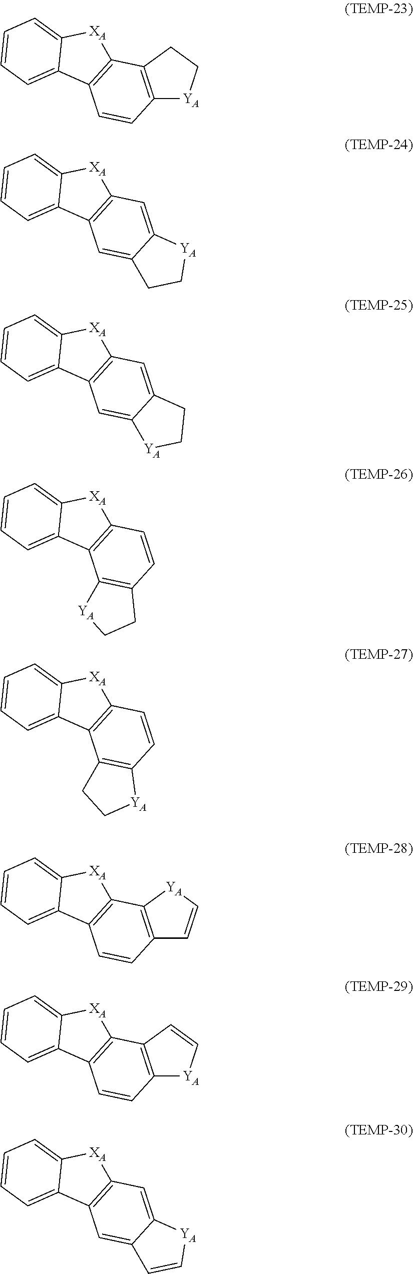

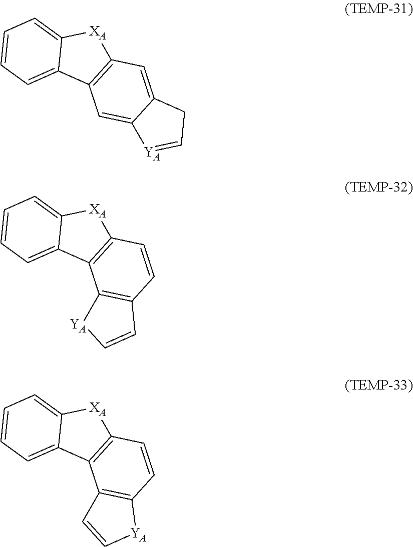

In the general formulas (TEMP-16) to (TEMP-33), X_{A} and Y_{A} are independently an oxygen atom, a sulfur atom, NH, or CH₂. Provided that at least one of X_{A} and Y_{A} is an oxygen atom, a sulfur atom, or NH.

In the general formulas (TEMP-16) to (TEMP-33), when at least one of X_{A} and Y_{A} is NH or CH₂, the monovalent heterocyclic group derived from the ring structures represented by any of the general formulas (TEMP-16) to (TEMP-33) includes a monovalent group derived by removing one hydrogen atom from these NH or CH₂.
Substituted heterocyclic group containing a nitrogen atom (specific example group G2B1):
   a (9-phenyl)carbazolyl group,
   a (9-biphenylyl)carbazolyl group,
   a (9-phenyl)phenylcarbazolyl group,
   a (9-naphthyl)carbazolyl group,
   a diphenylcarbazol-9-yl group,
   a phenylcarbazol-9-yl group,
   a methylbenzimidazolyl group,
   an ethylbenzimidazolyl group,
   a phenyltriazinyl group,
   a biphenylyltriazinyl group,
   a diphenyltriazinyl group,
   a phenylquinazolinyl group, and
   a biphenylylquinazolinyl group.
Substituted heterocyclic group containing an oxygen atom (specific example group G2B2):
   a phenyldibenzofuranyl group,
   a methyldibenzofuranyl group,
   a t-butyldibenzofuranyl group, and
   a monovalent residue of spiro[9H-xanthene-9,9′-[9H]fluorene].
Substituted heterocyclic group containing a sulfur atom (specific example group G2B3):
   a phenyldibenzothiophenyl group,
   a methyldibenzothiophenyl group,
   a t-butyldibenzothiophenyl group, and
   a monovalent residue of spiro[9H-thioxanthene-9,9′-[9H]fluorene].
Group in which one or more hydrogen atoms of the monovalent heterocyclic group derived from the ring structures represented by any of the following general formulas (TEMP-16) to (TEMP-33) are substituted by a substituent (specific example group G2B4):

The “one or more hydrogen atoms of the monovalent heterocyclic group” means one or more hydrogen atoms selected from hydrogen atoms bonded with ring carbon atoms of the monovalent heterocyclic group, a hydrogen atom bonded with a nitrogen atom when at least one of X_{A} and Y_{A} is NH, and hydrogen atoms of a methylene group when one of X_{A} and Y_{A} is CH₂.

“Substituted or Unsubstituted Alkyl Group”

Specific examples of the “substituted or unsubstituted alkyl group” (specific example group G3) described in this specification include the following unsubstituted alkyl groups (specific example group G3A) and the following substituted alkyl groups (specific example group G3B). (Here, the unsubstituted alkyl group refers to the case where the “substituted or unsubstituted alkyl group” is an “alkyl group unsubstituted by a substituent”, and the substituted alkyl group refers to the case where the “substituted or unsubstituted alkyl group” is an “alkyl group substituted by a substituent”). In this specification, in the case where simply referred as an “alkyl group” includes both the “unsubstituted alkyl group” and the “substituted alkyl group.”

The “substituted alkyl group” means a group in which one or more hydrogen atoms in the “unsubstituted alkyl group” are substituted by a substituent. Specific examples of the “substituted alkyl group” include groups in which one or more hydrogen atoms in the following “unsubstituted alkyl group” (specific example group G3A) are substituted by a substituent, the following substituted alkyl group (specific example group G3B), and the like. In this specification, the alkyl group in the “unsubstituted alkyl group” means a linear alkyl group. Thus, the “unsubstituted alkyl group” includes a straight-chain “unsubstituted alkyl group” and a branched-chain “unsubstituted alkyl group”. It should be noted that the examples of the “unsubstituted alkyl group” and the examples of the “substituted alkyl group” enumerated in this specification are mere examples, and the “substituted alkyl group” described in this specification includes a group in which hydrogen atom of the alkyl group itself in the “substituted alkyl group” of the specific example group G3B is further substituted by a substituent, and a group in which hydrogen atom of a substituent in the “substituted alkyl group” of the specific example group G3B is further substituted by a substituent.
Unsubstituted alkyl group (specific example group G3A):
   a methyl group,
   an ethyl group,
   a n-propyl group,
   an isopropyl group,
   a n-butyl group,
   an isobutyl group,
   a s-butyl group, and
   a t-butyl group.
Substituted alkyl group (specific example group G3B):
   a heptafluoropropyl group (including isomers),
   a pentafluoroethyl group,
   a 2,2,2-trifluoroethyl group, and
   a trifluoromethyl group.

“Substituted or Unsubstituted Alkenyl Group”

Specific examples of the “substituted or unsubstituted alkenyl group” described in this specification (specific example group G4) include the following unsubstituted alkenyl group (specific example group G4A), the following substituted alkenyl group (specific example group G4B), and the like. (Here, the unsubstituted alkenyl group refers to the case where the “substituted or unsubstituted alkenyl group” is a “alkenyl group unsubstituted by a substituent”, and the “substituted alkenyl group” refers to the case where the “substituted or unsubstituted alkenyl group” is a “alkenyl group substituted by a substituent.”). In this specification, in the case where simply referred as an “alkenyl group” includes both the “unsubstituted alkenyl group” and the “substituted alkenyl group.”

The “substituted alkenyl group” means a group in which one or more hydrogen atoms in the “unsubstituted alkenyl group” are substituted by a substituent. Specific examples of the “substituted alkenyl group” include a group in which the following “unsubstituted alkenyl group” (specific example group G4A) has a substituent, the following substituted alkenyl group (specific example group G4B), and the like. It should be noted that the examples of the “unsubstituted alkenyl group” and the examples of the “substituted alkenyl group” enumerated in this specification are mere examples, and the “substituted alkenyl group” described in this specification includes a group in which a hydrogen atom of the alkenyl group itself in the “substituted alkenyl group” of the specific example group G4B is further substituted by a substituent, and a group in which a hydrogen atom of a substituent in the “substituted alkenyl group” of the specific example group G4B is further substituted by a substituent.
Unsubstituted alkenyl group (specific example group G4A):
   a vinyl group,
   an allyl group,
   a 1-butenyl group,
   a 2-butenyl group, and
   a 3-butenyl group.
Substituted alkenyl group (specific example group G4B):
   a 1,3-butanedienyl group,
   a 1-methylvinyl group,
   a 1-methylallyl group,
   a 1,1-dimethylallyl group,
   a 2-methylally group, and
   a 1,2-dimethylallyl group.

“Substituted or Unsubstituted Alkynyl Group”

Specific examples of the “substituted or unsubstituted alkynyl group” described in this specification (specific example group G5) include the following unsubstituted alkynyl group (specific example group G5A) and the like. (Here, the unsubstituted alkynyl group refers to the case where the “substituted or unsubstituted alkynyl group” is an “alkynyl group unsubstituted by a substituent”). In this specification, in the case where simply referred as an “alkynyl group” includes both the “unsubstituted alkynyl group” and the “substituted alkynyl group.”

The “substituted alkynyl group” means a group in which one or more hydrogen atoms in the “unsubstituted alkynyl group” are substituted by a substituent. Specific examples of the “substituted alkynyl group” include a group in which one or more hydrogen atoms in the following “unsubstituted alkynyl group” (specific example group G5A) are substituted by a substituent, and the like.
Unsubstituted alkynyl group (specific example group G5A):
   an ethynyl group.

“Substituted or Unsubstituted Cycloalkyl Group”

Specific examples of the “substituted or unsubstituted cycloalkyl group” described in this specification (specific example group G6) include the following unsubstituted cycloalkyl group (specific example group G6A), the following substituted cycloalkyl group (specific example group G6B), and the like. (Here, the unsubstituted cycloalkyl group refers to the case where the “substituted or unsubstituted cycloalkyl group” is a “cycloalkyl group unsubstituted by a substituent”, and the substituted cycloalkyl group refers to the case where the “substituted or unsubstituted cycloalkyl group” is a “cycloalkyl group substituted by a substituent”). In this specification, in the case where simply referred as a “cycloalkyl group” includes both the “unsubstituted cycloalkyl group” and the “substituted cycloalkyl group.”

The “substituted cycloalkyl group” means a group in which one or more hydrogen atoms in the “unsubstituted cycloalkyl group” are substituted by a substituent. Specific examples of the “substituted cycloalkyl group” include a group in which one or more hydrogen atoms in the following “unsubstituted cycloalkyl group” (specific example group G6A) are substituted by a substituent, and examples of the following substituted cycloalkyl group (specific example group G6B), and the like. It should be noted that the examples of the “unsubstituted cycloalkyl group” and the examples of the “substituted cycloalkyl group” enumerated in this specification are mere examples, and the “substituted cycloalkyl group” in this specification includes a group in which one or more hydrogen atoms bonded with the carbon atom of the cycloalkyl group itself in the “substituted cycloalkyl group” of the specific example group G6B are substituted by a substituent, and a group in which a hydrogen atom of a substituent in the “substituted cycloalkyl group” of specific example group G6B is further substituted by a substituent.
Unsubstituted cycloalkyl group (specific example group G6A):
   a cyclopropyl group,
   a cyclobutyl group,
   a cyclopentyl group,
   a cyclohexyl group,
   a 1-adamantyl group,
   a 2-adamantyl group,
   a 1-norbornyl group, and
   a 2-norbornyl group.
Substituted cycloalkyl group (specific example group G6B):
   a 4-methylcyclohexyl group.

“Group Represented by —Si(R₉₀₁)(R₉₀₂)(R₉₀₃)”

Specific examples of the group represented by —Si(R₉₀₁)(R₉₀₂)(R₉₀₃) described in this specification (specific example group G7) include:
Si(G1)(G1)(G1),
Si(G1)(G2)(G2),
Si(G1)(G1)(G2),
Si(G2)(G2)(G2),
Si(G3)(G3)(G3), and
Si(G6)(G6)(G6).

G1 is the “substituted or unsubstituted aryl group” described in the specific example group G1.

G2 is the “substituted or unsubstituted heterocyclic group” described in the specific example group G2.

G3 is the “substituted or unsubstituted alkyl group” described in the specific example group G3.

G6 is the “substituted or unsubstituted cycloalkyl group” described in the specific example group G6.

Plural G1's in —Si(G1)(G1)(G1) are the same or different.

Plural G2's in —Si(G1)(G2)(G2) are the same or different.

Plural G1's in —Si(G1)(G1)(G2) are the same or different.

Plural G2's in —Si(G2)(G2)(G2) are be the same or different.

Plural G3's in —Si(G3)(G3)(G3) are the same or different.

Plural G6's in —Si(G6)(G6)(G6) are be the same or different.

“Group Represented by —O—(R₉₀₄)”

Specific examples of the group represented by —O—(R₉₀₄) in this specification (specific example group G8) include:
—O(G1),
—O(G2),
—O(G3), and
—O(G6).

G1 is the “substituted or unsubstituted aryl group” described in the specific example group G1.

G2 is the “substituted or unsubstituted heterocyclic group” described in the specific example group G2.

G3 is the “substituted or unsubstituted alkyl group” described in the specific example group G3.

G6 is the “substituted or unsubstituted cycloalkyl group” described in the specific example group G6.

“Group Represented by —S—(R₉₀₅)”

Specific examples of the group represented by —S—(R₉₀₅) in this specification (specific example group G9) include:
—S(G1),
—S(G2),
—S(G3), and
—S(G6).

G1 is the “substituted or unsubstituted aryl group” described in the specific example group G1.

G2 is the “substituted or unsubstituted heterocyclic group” described in the specific example group G2.

G3 is the “substituted or unsubstituted alkyl group” described in the specific example group G3.

G6 is the “substituted or unsubstituted cycloalkyl group” described in the specific example group G6.

“Group Represented by —N(R₉₀₆)(R₉₀₇)”

Specific examples of the group represented by —N(R₉₀₆)(R₉₀₇) in this specification (specific example group G10) include:
—N(G1)(G1),
—N(G2)(G2),
—N(G1)(G2),
—N(G3)(G3), and
—N(G6)(G6).

G1 is the “substituted or unsubstituted aryl group” described in the specific example group G1.

G2 is the “substituted or unsubstituted heterocyclic group” described in the specific example group G2.

G3 is the “substituted or unsubstituted alkyl group” described in the specific example group G3.

G6 is the “substituted or unsubstituted cycloalkyl group” described in the specific example group G6.

Plural G1's in —N(G1)(G1) are the same or different.

Plural G2's in —N(G2)(G2) are the same or different.

Plural G3's in —N(G3)(G3) are the same or different.

Plural G6's in —N(G6)(G6) are the same or different.

“Halogen Atom”

Specific examples of the “halogen atom” described in this specification (specific example group G11) include a fluorine atom, a chlorine atom, a bromine atom, an iodine atom, and the like.

“Substituted or Unsubstituted Fluoroalkyl Group”

The “substituted or unsubstituted fluoroalkyl group” described in this specification is a group in which at least one hydrogen atom bonded with a carbon atom constituting the alkyl group in the “substituted or unsubstituted alkyl group” is substituted by a fluorine atom, and includes a group in which all hydrogen atoms bonded with a carbon atom constituting the alkyl group in the “substituted or unsubstituted alkyl group” are substituted by a fluorine atom (a perfluoro group). The number of carbon atoms of the “unsubstituted fluoroalkyl group” is 1 to 50, preferably 1 to 30, more preferably 1 to 18, unless otherwise specified in this specification. The “substituted fluoroalkyl group” means a group in which one or more hydrogen atoms of the “fluoroalkyl group” are substituted by a substituent. The “substituted fluoroalkyl group” described in this specification also includes a group in which one or more hydrogen atoms bonded with a carbon atom of the alkyl chains in the “substituted fluoroalkyl group” are further substituted by a substituent, and a group in which one or more hydrogen atom of a substituent in the “substituted fluoroalkyl group” are further substituted by a substituent. Specific examples of the “unsubstituted fluoroalkyl group” include a group in which one or more hydrogen atoms in the “alkyl group” (specific group G3) are substituted by a fluorine atom, and the like.

“Substituted or Unsubstituted Haloalkyl Group”

The “substituted or unsubstituted haloalkyl group” described in this specification is a group in which at least one hydrogen atom bonded with a carbon atom constituting the alkyl group in the “substituted or unsubstituted alkyl group” is substituted by a halogen atom, and also includes a group in which all hydrogen atoms bonded with a carbon atom constituting the alkyl group in the “substituted or unsubstituted alkyl group” are substituted by a halogen atom. The number of carbon atoms of the “unsubstituted haloalkyl group” is 1 to 50, preferably 1 to 30, more preferably 1 to 18, unless otherwise specified in this specification. The “substituted haloalkyl group” means a group in which one or more hydrogen atoms of the “haloalkyl group” are substituted by a substituent. The “substituted haloalkyl group” described in this specification also includes a group in which one or more hydrogen atoms bonded with a carbon atom of the alkyl chain in the “substituted haloalkyl group” are further substituted by a substituent, and a group in which one or more hydrogen atoms of a substituent in the “substituted haloalkyl group” are further substituted by a substituent. Specific examples of the “unsubstituted haloalkyl group” include a group in which one or more hydrogen atoms in the “alkyl group” (specific example group G3) are substituted by a halogen atom, and the like. A haloalkyl group is sometimes referred to as an alkyl halide group.

“Substituted or Unsubstituted Alkoxy Group”

Specific examples of the “substituted or unsubstituted alkoxy group” described in this specification include a group represented by —O(G3), wherein G3 is the “substituted or unsubstituted alkyl group” described in the specific example group G3. The number of carbon atoms of the “unsubstituted alkoxy group” is 1 to 50, preferably 1 to 30, more preferably 1 to 18, unless otherwise specified in this specification.

“Substituted or Unsubstituted Alkylthio Group”

Specific examples of the “substituted or unsubstituted alkylthio group” described in this specification include a group represented by —S(G3), wherein G3 is the “substituted or unsubstituted alkyl group” described in the specific example group G3. The number of carbon atoms of the “unsubstituted alkylthio group” is 1 to 50, preferably 1 to 30, more preferably 1 to 18, unless otherwise specified in this specification.

“Substituted or Unsubstituted Aryloxy Group”

Specific examples of the “substituted or unsubstituted aryloxy group” described in this specification include a group represented by —O(G1), wherein G1 is the “substituted or unsubstituted aryl group” described in the specific example group G1. The number of ring carbon atoms of the “unsubstituted aryloxy group” is 6 to 50, preferably 6 to 30, more preferably 6 to 18, unless otherwise specified in this specification.

“Substituted or Unsubstituted Arylthio Group”

Specific examples of the “substituted or unsubstituted arylthio group” described in this specification include a group represented by —S(G1), wherein G1 is a “substituted or unsubstituted aryl group” described in the specific example group G1. The number of ring carbon atoms of the “unsubstituted arylthio group” is 6 to 50, preferably 6 to 30, more preferably 6 to 18, unless otherwise specified in this specification.

“Substituted or Unsubstituted Trialkylsilyl Group”

Specific examples of the “trialkylsilyl group” described in this specification include a group represented by —Si(G3)(G3)(G3), where G3 is the “substituted or unsubstituted alkyl group” described in the specific example group G3. Plural G3's in —Si(G3)(G3)(G3) are the same or different. The number of carbon atoms in each alkyl group of the “trialkylsilyl group” is 1 to 50, preferably 1 to 20, more preferably 1 to 6, unless otherwise specified in this specification.

“Substituted or Unsubstituted Aralkyl Group”

Specific examples of the “substituted or unsubstituted aralkyl group” described in this specification is a group represented by -(G3)-(G1), wherein G3 is the “substituted or unsubstituted alkyl group” described in the specific example group G3, and G1 is the “substituted or unsubstituted aryl group” described in the specific example group G1. Therefore, the “aralkyl group” is a group in which a hydrogen atom of the “alkyl group” is substituted by an “aryl group” as a substituent, and is one form of the “substituted alkyl group.” The “unsubstituted aralkyl group” is the “unsubstituted alkyl group” substituted by the “unsubstituted aryl group”, and the number of carbon atoms of the “unsubstituted aralkyl group” is 7 to 50, preferably 7 to 30, more preferably 7 to 18, unless otherwise specified in this specification.

Specific examples of the “substituted or unsubstituted aralkyl group” include a benzyl group, a 1-phenylethyl group, a 2-phenylethyl group, a 1-phenylisopropyl group, a 2-phenylisopropyl group, a phenyl-t-butyl group, an α-naphthylmethyl group, a 1-α-naphthylethyl group, a 2-α-naphthylethyl group, a 1-α-naphthylisopropyl group, a 2-α-naphthylisopropyl group, a β-naphthylmethyl group, a 1-p-naphthylethyl group, a 2-β-naphthylethyl group, a 1-β-naphthylisopropyl group, a 2-β-naphthylisopropyl group, and the like.

Unless otherwise specified in this specification, examples of the substituted or unsubstituted aryl group described in this specification preferably include a phenyl group, a p-biphenyl group, a m-biphenyl group, an o-biphenyl group, a p-terphenyl-4-yl group, a p-terphenyl-3-yl group, a p-terphenyl-2-yl group, a m-terphenyl-4-yl group, a m-terphenyl-3-yl group, a m-terphenyl-2-yl group, an o-terphenyl-4-yl group, an o-terphenyl-3-yl group, an o-terphenyl-2-yl group, a 1-naphthyl group, a 2-naphthyl group, an anthryl group, a phenanthryl group, a pyrenyl group, a chrysenyl group, a triphenylenyl group, a fluorenyl group, a 9,9′-spirobifluorenyl group, 9,9-dimethylfluorenyl group, 9,9-diphenylfluorenyl group, and the like.

Unless otherwise specified in this specification, examples of the substituted or unsubstituted heterocyclic groups described in this specification preferably include a pyridyl group, a pyrimidinyl group, a triazinyl group, a quinolyl group, an isoquinolyl group, a quinazolinyl group, a benzimidazolyl group, a phenanthrolinyl group, a carbazolyl group (a 1-carbazolyl group, a 2-carbazolyl group, a 3-carbazolyl group, a 4-carbazolyl group, or a 9-carbazolyl group), a benzocarbazolyl group, an azacarbazolyl group, a diazacarbazolyl group, a dibenzofuranyl group, a naphthobenzofuranyl group, an azadibenzofuranyl group, a diazadibenzofuranyl group, a dibenzothiophenyl group, a naphthobenzothiophenyl group, an azadibenzothiophenyl group, a diazadibenzothiophenyl group, a (9-phenyl)carbazolyl group (a (9-phenyl)carbazol-1-yl group, a (9-phenyl)carbazol-2-yl group, a (9-phenyl)carbazol-3-yl group, or a (9-phenyl)carbazol-4-yl group), a (9-biphenylyl)carbazolyl group, a (9-phenyl)phenylcarbazolyl group, a diphenylcarbazol-9-yl group, a phenylcarbazol-9-yl group, a phenyltriazinyl group, a biphenylyltriazinyl group, a diphenyltriazinyl group, a phenyldibenzofuranyl group, a phenyldibenzothiophenyl group, and the like.

In this specification, the carbazolyl group is specifically any of the following groups, unless otherwise specified in this specification.

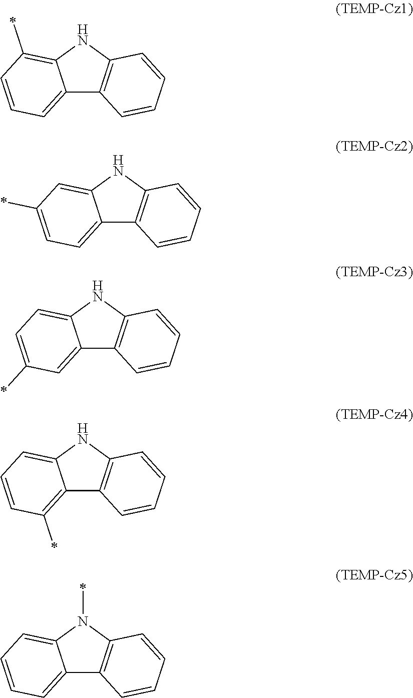 [SMILES: *c1cccc2c1[nH]c1ccccc12 ; *c1ccc2c(c1)[nH]c1ccccc12 ; *c1ccc2[nH]c3ccccc3c2c1 ; *c1cccc2[nH]c3ccccc3c12 ; *n1c2ccccc2c2ccccc21]

In this specification, the (9-phenyl)carbazolyl group is specifically any of the following groups, unless otherwise specified in this specification.

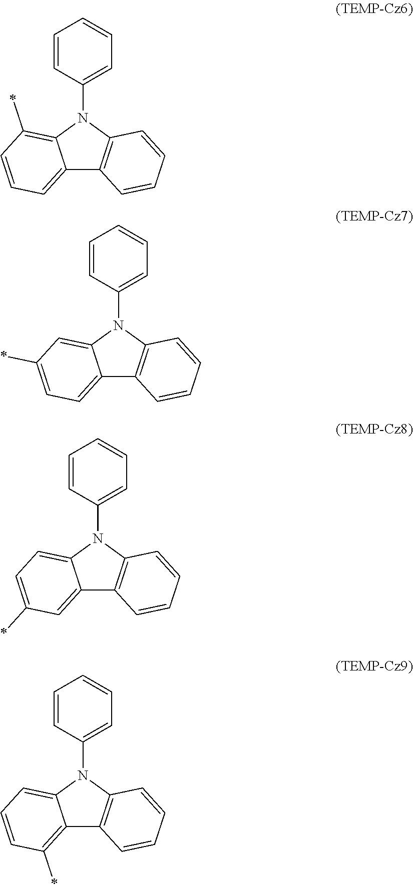

In the general formulas (TEMP-Cz1) to (TEMP-Cz9), * represents a bonding position.

In this specification, the dibenzofuranyl group and the dibenzothiophenyl group are specifically any of the following groups, unless otherwise specified in this specification.

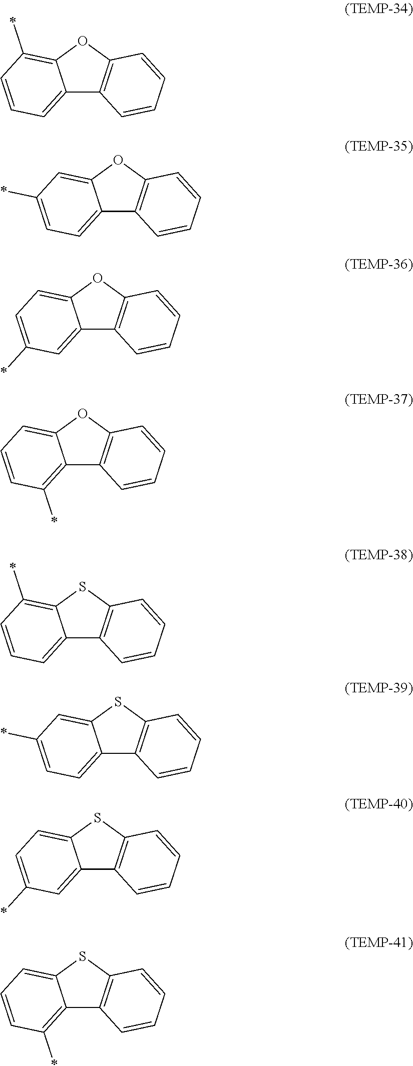

In the general formulas (TEMP-34) to (TEMP-41), * represents a bonding position.

The substituted or unsubstituted alkyl group described in this specification is preferably a methyl group, an ethyl group, a propyl group, an isopropyl group, a n-butyl group, an isobutyl group, a t-butyl group, or the like, unless otherwise specified in this specification.

“Substituted or Unsubstituted Arylene Group”

The “substituted or unsubstituted arylene group” described in this specification is a divalent group derived by removing one hydrogen atom on the aryl ring of the “substituted or unsubstituted aryl group”, unless otherwise specified. Specific examples of the “substituted or unsubstituted arylene group” (specific example group G12) include a divalent group derived by removing one hydrogen atom on the aryl ring of the “substituted or unsubstituted aryl group” described in the specific example group G1, and the like.

“Substituted or Unsubstituted Divalent Heterocyclic Group”

The “substituted or unsubstituted divalent heterocyclic group” described in this specification is a divalent group derived by removing one hydrogen atom on the heterocyclic ring of the “substituted or unsubstituted heterocyclic group”, unless otherwise specified. Specific examples of the “substituted or unsubstituted divalent heterocyclic group” (specific example group G13) include a divalent group derived by removing one hydrogen atom on the heterocyclic ring of the “substituted or unsubstituted heterocyclic group” described in the specific example group G2, and the like.

“Substituted or Unsubstituted Alkylene Group”

The “substituted or unsubstituted alkylene group” described in this specification is a divalent group derived by removing one hydrogen atom on the alkyl chain of the “substituted or unsubstituted alkyl group”, unless otherwise specified. Specific examples of the “substituted or unsubstituted alkylene group” (specific example group G14) include a divalent group derived by removing one hydrogen atom on the alkyl chain of the “substituted or unsubstituted alkyl group” described in the specific example group G3, and the like.

The substituted or unsubstituted arylene group described in this specification is preferably any group of the following general formulas (TEMP-42) to (TEMP-68), unless otherwise specified in this specification.

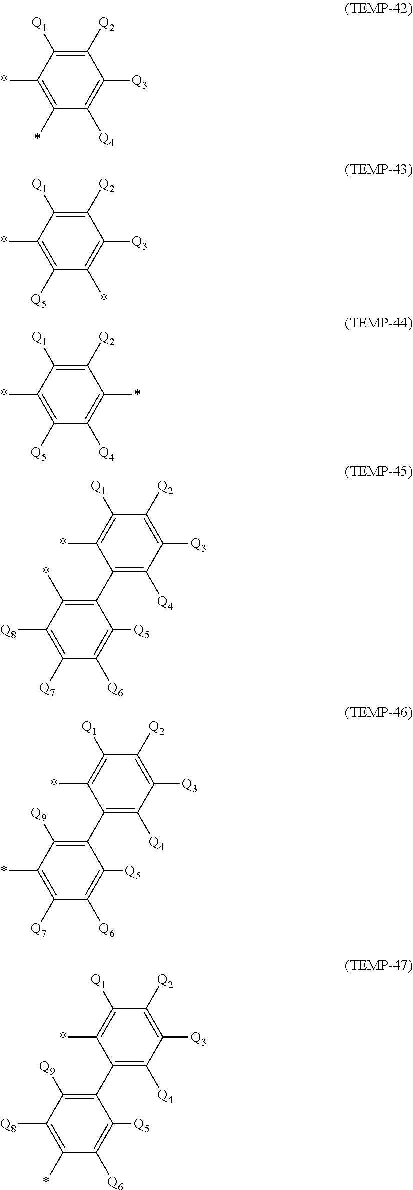

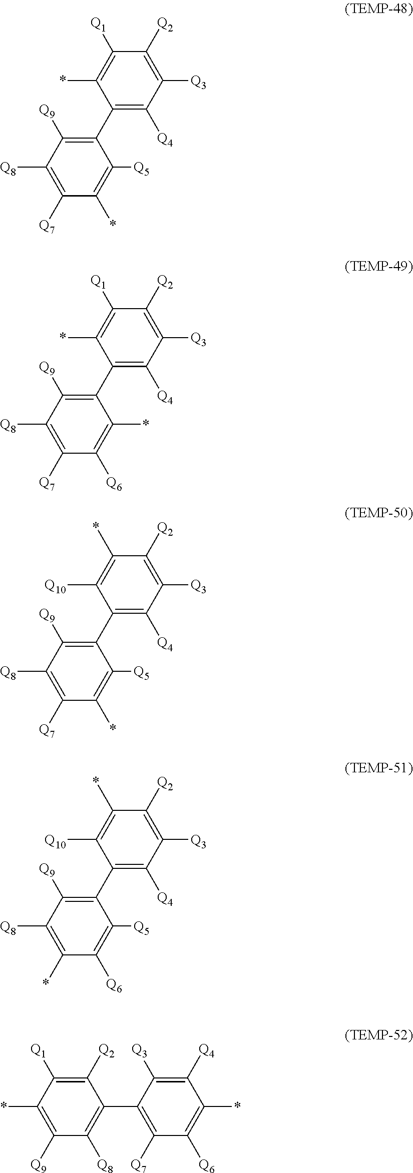

In the general formulas (TEMP-42) to (TEMP-52), Q₁ to Q₁₀ are independently a hydrogen atom or a substituent.

In the general formulas (TEMP-42) to (TEMP-52), * represents a bonding position.

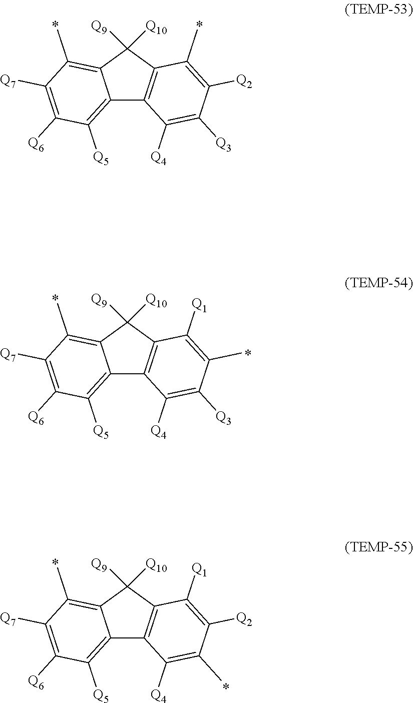

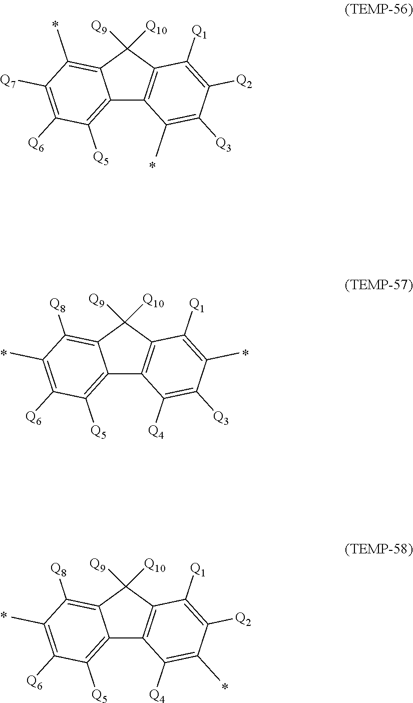

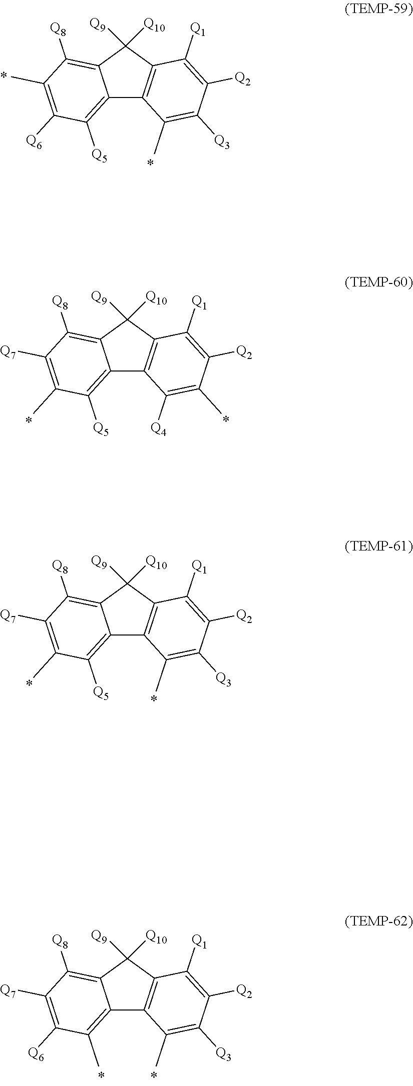

In the general formulas (TEMP-53) to (TEMP-62), Q₁ to Q₁₀ are independently a hydrogen atom or a substituent.

Q₉ and Q₁₀ may be bonded with each other via a single bond to form a ring.

In the general formulas (TEMP-53) to (TEMP-62), * represents a bonding position.

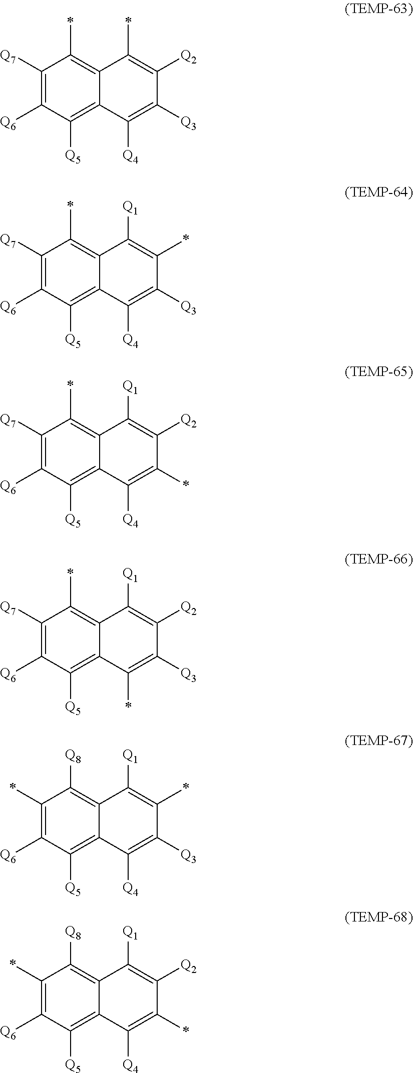

In the general formulas (TEMP-63) to (TEMP-68), Q₁ to Q₈ are independently a hydrogen atom or a substituent.

In the general formulas (TEMP-63) to (TEMP-68), * represents a bonding position.

The substituted or unsubstituted divalent heterocyclic group described in this specification is preferably any group of the following general formulas (TEMP-69) to (TEMP-102), unless otherwise specified in this specification.

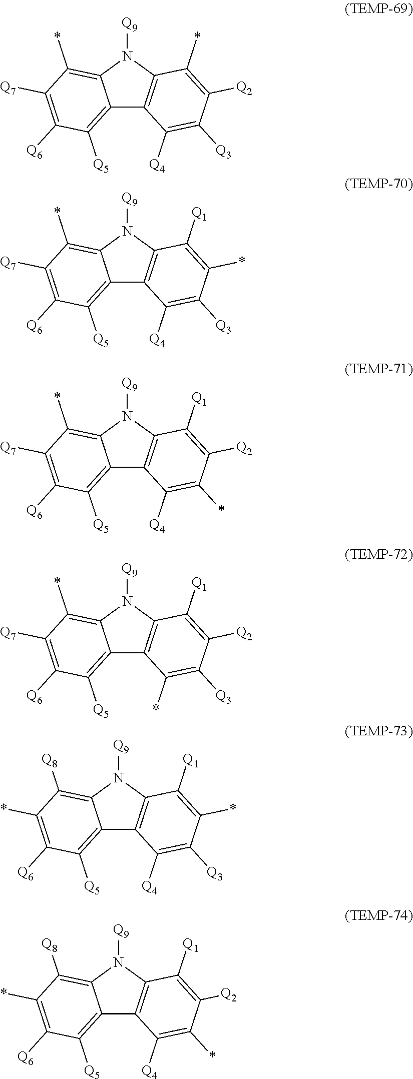

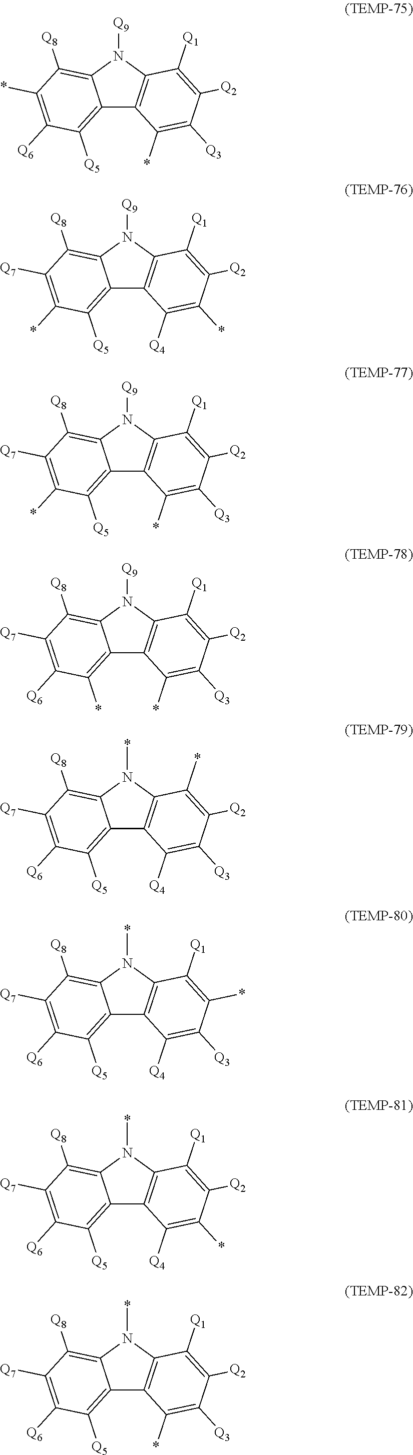

In the general formulas (TEMP-69) to (TEMP-82), Q₁ to Qs are independently a hydrogen atom or a substituent.

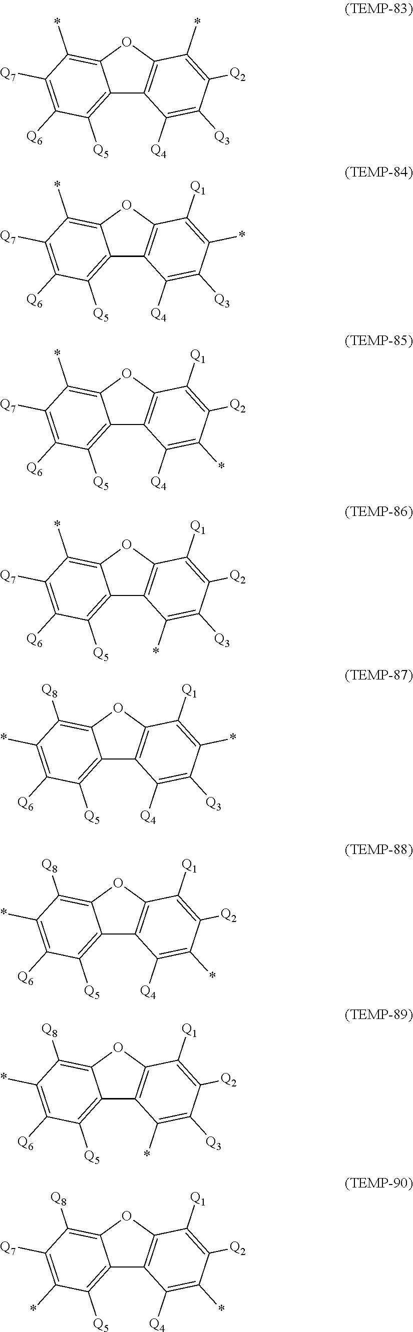

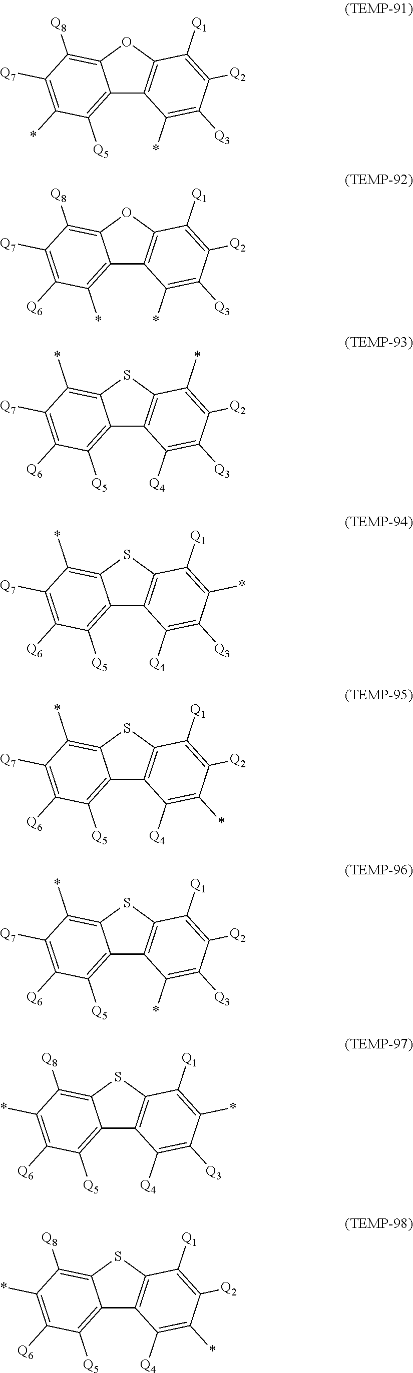

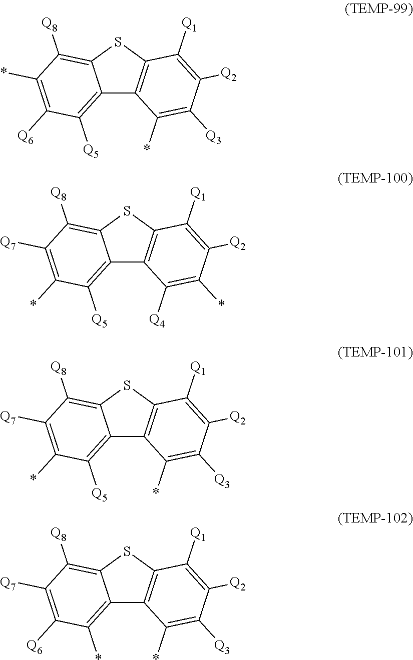

In the general formulas (TEMP-83) to (TEMP-102), Q₁ to Q₈ are independently a hydrogen atom or a substituent.

The above is the explanation of the “Substituent described in this specification.”

“The Case where Bonded with Each Other to Form a Ring”

In this specification, the case where “one or more sets of adjacent two or more form a substituted or unsubstituted monocycle by bonding with each other, form a substituted or unsubstituted fused ring by bonding with each other, or do not bond with each other” means the case where “one or more sets of adjacent two or more form a substituted or unsubstituted monocycle by bonding with each other”; the case where “one or more sets of adjacent two or more form a substituted or unsubstituted fused ring by bonding with each other”; and the case where “one or more sets of adjacent two or more do not bond with each other.”

The case where “one or more sets of adjacent two or more form a substituted or unsubstituted monocycle by bonding with each other” and the case where “one or more sets of adjacent two or more form a substituted or unsubstituted fused ring by bonding with each other” in this specification (these cases may be collectively referred to as “the case where forming a ring by bonding with each other”) will be described below. The case of an anthracene compound represented by the following general formula (TEMP-103) in which the mother skeleton is an anthracene ring will be described as an example.

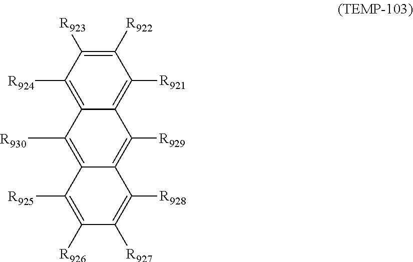

For example, in the case where “one or more sets of adjacent two or more among R₉₂₁ to R₉₃₀ form a ring by bonding with each other”, the one set of adjacent two includes a pair of R₉₂₁ and R₉₂₂, a pair of R₉₂₂ and R₉₂₃, a pair of R₉₂₃ and R₉₂₄, a pair of R₉₂₄ and R₉₃₀, a pair of R₉₃₀ and R₉₂₅, a pair of R₉₂₅ and R₉₂₆, a pair of R₉₂₆ and R₉₂₇, a pair of R₉₂₇ and R₉₂₈, a pair of R₉₂₈ and R₉₂₉, and a pair of R₉₂₉ and R₉₂₁.

The “one or more sets” means that two or more sets of the adjacent two or more sets may form a ring at the same time. For example, R₉₂₁ and R₉₂₂ form a ring Q_{A} by bonding with each other, and at the same, time R₉₂₅ and R₉₂₆ form a ring Q_{B} by bonding with each other, the anthracene compound represented by the general formula (TEMP-103) is represented by the following general formula (TEMP-104).

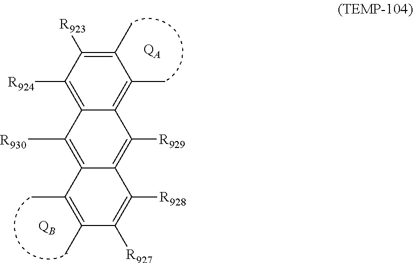

The case where the “set of adjacent two or more” form a ring includes not only the case where the set (pair) of adjacent “two” is bonded with as in the above-mentioned examples, but also the case where the set of adjacent “three or more” are bonded with each other. For example, it means the case where R₉₂₁ and R₉₂₂ form a ring Q_{A} by bonding with each other, and R₉₂₂ and R₉₂₃ form a ring Qc by bonding with each other, and adjacent three (R₉₂₁, R₉₂₂ and R₉₂₃) form rings by bonding with each other and together fused to the anthracene mother skeleton. In this case, the anthracene compound represented by the general formula (TEMP—103) is represented by the following general formula (TEMP—105). In the following general formula (TEMP—105), the ring Q_{A} and the ring Qc share R₉₂₂.

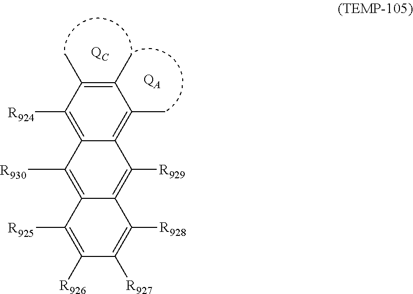

The “monocycle” or “fused ring” formed may be a saturated ring or an unsaturated ring, as a structure of the formed ring alone. Even when the “one pair of adjacent two” forms a “monocycle” or a “fused ring”, the “monocycle” or the “fused ring” may form a saturated ring or an unsaturated ring. For example, the ring Q_{A} and the ring Q_{B} formed in the general formula (TEMP—104) are independently a “monocycle” or a “fused ring.” The ring Q_{A} and the ring Qc formed in the general formula (TEMP-105) are “fused ring.” The ring Q_{A} and ring Qc of the general formula (TEMP—105) are fused ring by fusing the ring Q_{A} and the ring Qc together. When the ring Q_{A} of the general formula (TMEP—104) is a benzene ring, the ring Q_{A} is a monocycle. When the ring Q_{A} of the general formula (TMEP—104) is a naphthalene ring, the ring Q_{A} is a fused ring.

The “unsaturated ring” means an aromatic hydrocarbon ring or an aromatic heterocyclic ring. The “saturated ring” means an aliphatic hydrocarbon ring, or a non-aromatic heterocyclic ring.

Specific examples of the aromatic hydrocarbon ring include a structure in which the group listed as a specific example in the specific example group G1 is terminated by a hydrogen atom.

Specific examples of the aromatic heterocyclic ring include a structure in which the aromatic heterocyclic group listed as a specific example in the example group G2 is terminated by a hydrogen atom.

Specific examples of the aliphatic hydrocarbon ring include a structure in which the group listed as a specific example in the specific example group G6 is terminated by a hydrogen atom.

The term “to form a ring” means forming a ring only with plural atoms of the mother skeleton, or with plural atoms of the mother skeleton and one or more arbitrary elements in addition. For example, the ring Q_{A} shown in the general formula (TEMP—104), which is formed by bonding R₉₂₁ and R₉₂₂ with each other, is a ring formed from the carbon atom of the anthracene skeleton with which R₉₂₁ is bonded, the carbon atom of the anthracene skeleton with which R₉₂₂ is bonded, and one or more arbitrary elements. For example, in the case where the ring Q_{A} is formed with R₉₂₁ and R₉₂₂, when a monocyclic unsaturated ring is formed with the carbon atom of the anthracene skeleton with which R₉₂₁ is bonded, the carbon atom of the anthracene skeleton with which R₉₂₂ is bonded, and four carbon atoms, the ring formed with R₉₂₁ and R₉₂₂ is a benzene ring.

Here, the “arbitrary element” is preferably at least one element selected from the group consisting of a carbon element, a nitrogen element, an oxygen element, and a sulfur element, unless otherwise specified in this specification. In the arbitrary element (for example, a carbon element or a nitrogen element), a bond which does not form a ring may be terminated with a hydrogen atom or the like, or may be substituted with “arbitrary substituent” described below. When an arbitrary element other than a carbon element is contained, the ring formed is a heterocyclic ring.

The number of “one or more arbitrary element(s)” constituting a monocycle or a fused ring is preferably 2 or more and 15 or less, more preferably 3 or more and 12 or less, and still more preferably 3 or more and 5 or less, unless otherwise specified in this specification.

The “monocycle” is preferable among the “monocycle” and the “fused ring”, unless otherwise specified in this specification.

The “unsaturated ring” is preferable among the “saturated ring” and the “unsaturated ring”, unless otherwise specified in this specification.

Unless otherwise specified in this specification, the “monocycle” is preferably a benzene ring.

Unless otherwise specified in this specification, the “unsaturated ring” is preferably a benzene ring.

Unless otherwise specified in this specification, when “one or more sets of adjacent two or more” are “bonded with each other to form a substituted or unsubstituted monocycle” or “bonded with each other to form a substituted or unsubstituted fused ring”, this specification, one or more sets of adjacent two or more are preferably bonded with each other to form a substituted or unsubstituted “unsaturated ring” from plural atoms of the mother skeleton and one or more and 15 or less elements which is at least one kind selected from a carbon elements, a nitrogen element, an oxygen element, and a sulfur element.

The substituent in the case where the above-mentioned “monocycle” or “fused ring” has a substituent is, for example, an “arbitrary substituent” described below. Specific examples of the substituent which the above-mentioned “monocycle” or “fused ring” has include the substituent described above in the “Substituent described in this specification” section.

The substituent in the case where the above-mentioned “saturated ring” or “unsaturated ring” has a substituent is, for example, an “arbitrary substituent” described below. Specific examples of the substituent which the above-mentioned “monocycle” or “fused ring” has include the substituent described above in the “Substituent described in this specification” section.

The foregoing describes the case where “one or more sets of adjacent two or more form a substituted or unsubstituted monocycle by bonding with each other” and the case where “one or more sets of adjacent two or more form a substituted or unsubstituted fused ring by bonding with each other” (the case where “forming a ring by bonding with each other”).

Substituent in the Case of “Substituted or Unsubstituted”

In one embodiment in this specification, the substituent (in this specification, sometimes referred to as an “arbitrary substituent”) in the case of “substituted or unsubstituted” is, for example, a group selected from the group consisting of:

an unsubstituted alkyl group including 1 to 50 carbon atoms,

an unsubstituted alkenyl group including 2 to 50 carbon atoms,

an unsubstituted alkynyl group including 2 to 50 carbon atoms,

an unsubstituted cycloalkyl group including 3 to 50 ring carbon atoms,

—Si(R₉₀₁)(R₉₀₂)(R₉₀₃),

—O—(R₉₀₄),

—S—(R₉₀₅),

—N(R₉₀₆)(R₉₀₇),

a halogen atom, a cyano group, a nitro group,

an unsubstituted aryl group including 6 to 50 ring carbon atoms, and

an unsubstituted heterocyclic group including 5 to 50 ring atoms,

wherein, R₉₀₁ to R₉₀₇ are independently

a hydrogen atom,

a substituted or unsubstituted alkyl group including 1 to 50 carbon atoms,

a substituted or unsubstituted cycloalkyl group including 3 to 50 ring carbon atoms,

a substituted or unsubstituted aryl group including 6 to 50 ring carbon atoms, or

a substituted or unsubstituted heterocyclic group including 5 to 50 ring atoms.

When two or more R₉₀₁'s are present, the two or more R₉₀₁'s may be the same or different.

When two or more R₉₀₂'s are present, the two or more R₉₀₂'s may be the same or different.

When two or more R₉₀₃'s are present, the two or more R₉₀₃'s may be the same or different.

When two or more R₉₀₄'s are present, the two or more R₉₀₄'s may be the same or different.

When two or more R₉₀₅'s are present, the two or more R₉₀₅'s may be the same or different.

When two or more R₉₀₆'s are present, the two or more R₉₀₆'s may be the same or different.

When two or more R₉₀₇'s are present, the two or more R₉₀₇'s may be the same or different.

In one embodiment, the substituent in the case of “substituted or unsubstituted” is a group selected from the group consisting of:

an alkyl group including 1 to 50 carbon atoms,

an aryl group including 6 to 50 ring carbon atoms, and

a heterocyclic group including 5 to 50 ring atoms.

In one embodiment, the substituent in the case of “substituted or unsubstituted” is a group selected from the group consisting of:

an alkyl group including 1 to 18 carbon atoms,

an aryl group including 6 to 18 ring carbon atoms, and

a heterocyclic group including 5 to 18 ring atoms.

Specific examples of each of the arbitrary substituents include specific examples of substituent described in the section “Substituent described in this specification” above.

Unless otherwise specified in this specification, adjacent arbitrary substituents may form a “saturated ring” or an “unsaturated ring”, preferably form a substituted or unsubstituted saturated 5-membered ring, a substituted or unsubstituted saturated 6-membered ring, a substituted or unsubstituted unsaturated 5-membered ring, or a substituted or unsubstituted unsaturated 6-membered ring, more preferably form a benzene ring.

Unless otherwise specified in this specification, the arbitrary substituent may further have a substituent. The substituent which the arbitrary substituent further has is the same as that of the above-mentioned arbitrary substituent.

In this specification, the numerical range represented by “AA to BB” means the range including the numerical value AA described on the front side of “AA to BB” as the lower limit and the numerical value BB described on the rear side of “AA to BB” as the upper limit.

### [Novel Compound]

A compound according to an aspect of the present invention is represented by the following formula (1):

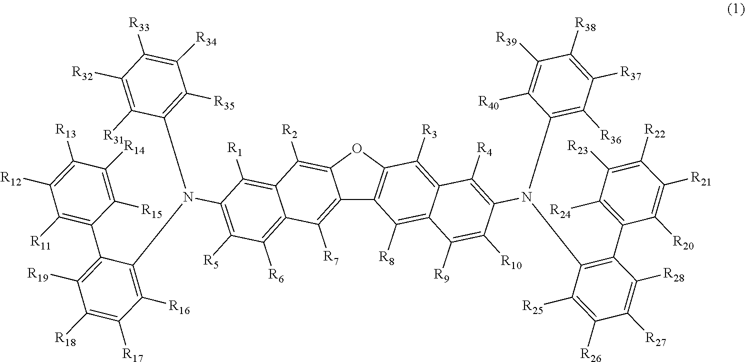

wherein in the formula (1),

R₁ to R₁₀ are independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a substituted or unsubstituted alkoxy group having 1 to 50 carbon atoms, a substituted or unsubstituted alkylthio group having 1 to 50 carbon atoms, a substituted or unsubstituted aryloxy group having 6 to 50 ring carbon atoms, a substituted or unsubstituted arylthio group having 6 to 50 ring carbon atoms, a substituted or unsubstituted aralkyl group having 7 to 50 carbon atoms, —Si(R₉₁)(R₉₂)(R₉₃), —C(═O)R₉₄, —COOR₉₅, —N(R₉₆)(R₉₇), a halogen atom, a cyano group, a nitro group, a substituted or unsubstituted aryl group having 6 to 50 ring carbon atoms, or a substituted or unsubstituted monovalent heterocyclic group having 5 to 50 ring atoms;

R₉₁ to R₉₇ are independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted monovalent heterocyclic group having 5 to 50 ring atoms;

when a plurality of each of R₉₁ to R₉₇ are present, the plurality of each of R₉₁ to R₉₇ may be the same as or different from each other; R₁₁ to R₂₈ are independently a hydrogen atom, a substituted or unsubstituted alkyl group having 1 to 20 carbon atoms, a substituted or unsubstituted cycloalkyl group having 3 to 20 ring carbon atoms, a silyl group substituted with an alkyl group having 1 to 20 carbon atoms, or a cyano group;

R₃₁ to R₄₀ are independently a hydrogen atom, a substituted or unsubstituted alkyl group having 1 to 20 carbon atoms, a substituted or unsubstituted cycloalkyl group having 3 to 20 ring carbon atoms, a substituted or unsubstituted aryl group having 6 to 20 ring carbon atoms, a silyl group substituted with an alkyl group having 1 to 20 carbon atoms, or a cyano group; at least one of R₁₁ to R₂₈ and R₃₁ to R₄₀ is not a hydrogen atom.

When the compound according to an aspect of the present invention is used, an organic EL device with high luminous efficiency and long lifetime can be fabricated.

In the compound represented by the formula (1), two biphenyl-2-yl groups and two phenyl groups, which are bonded to two nitrogen atoms, have one or more groups other than a hydrogen atom. For example, the formula (1) satisfies one or both of the following conditions A and B.

Condition A: At least one of R₁₁ to R₂₈ is a substituted or unsubstituted alkyl group having 1 to 20 carbon atoms, a substituted or unsubstituted cycloalkyl group having 3 to 20 ring carbon atoms, a silyl group substituted with an alkyl group having 1 to 20 carbon atoms, or a cyano group.

Condition B: At least one of R₃₁ to R₄₀ is a substituted or unsubstituted alkyl group having 1 to 20 carbon atoms, a substituted or unsubstituted cycloalkyl group having 3 to 20 ring carbon atoms, a substituted or unsubstituted aryl group having 6 to 20 ring carbon atoms, a silyl group substituted with an alkyl group having 1 to 20 carbon atoms, or a cyano group.

The silyl group substituted with an alkyl group having 1 to 20 carbon atoms is represented by “—Si(Rₓ)(R_{y})(R_{z})”, Rₓ, R_{y} and R_{z} are independently a hydrogen atom or an unsubstituted alkyl group having 1 to 20 carbon atoms, and at least one of Rₓ, R_{y} and R_{z} is the alkyl group.

In one embodiment, at least one of R₁₈, R₂₇, R₃₃ and R₃₈ of the formula (1) is a group other than hydrogen. For example, one or both of the following conditions A and B are satisfied.

Condition A: At least one of R₁₈ and R₂₇ is a substituted or unsubstituted alkyl group having 1 to 20 carbon atoms, a substituted or unsubstituted cycloalkyl group having 3 to 20 ring carbon atoms, a silyl group substituted with an alkyl group having 1 to 20 carbon atoms, or a cyano group.

Condition B: At least one of R₃₃ and R₃₈ is a substituted or unsubstituted alkyl group having 1 to 20 carbon atoms, a substituted or unsubstituted cycloalkyl group having 3 to 20 ring carbon atoms, a substituted or unsubstituted aryl group having 6 to 20 ring carbon atoms, a silyl group substituted with an alkyl group having 1 to 20 carbon atoms, or a cyano group.

In one embodiment, R₁₈, R₂₇, R₃₃ and R₃₈ are a substituted or unsubstituted alkyl group having 1 to 20 carbon atoms, a substituted or unsubstituted cycloalkyl group having 3 to 20 ring carbon atoms, a silyl group substituted with an alkyl group having 1 to 20 carbon atoms. When the compound of the present embodiment is used, an organic EL device having a longer lifetime can be fabricated.

In one embodiment, R₁₈, R₂₇, R₃₃ and R₃₈ are a substituted or unsubstituted alkyl group having 1 to 20 carbon atoms.

In one embodiment, the compound represented by the formula (1) is a compound represented by the following formula (1-1):

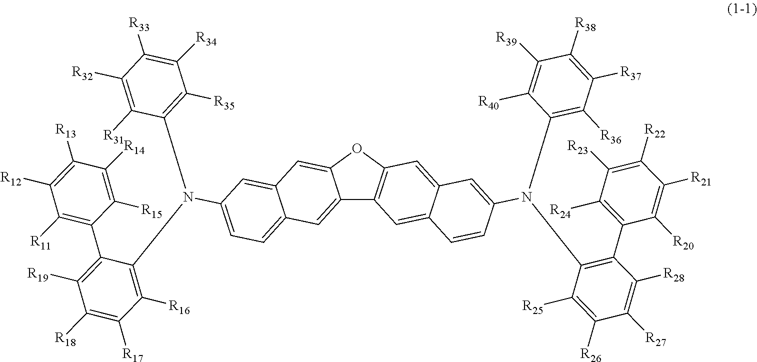

wherein in the formula (1-1), R₁₁ to R₂₈ and R₃₁ to R₄₀ are the same as defined in the formula (1).

In one embodiment, the compound represented by the formula (1) is a compound represented by the following formula (1-2):

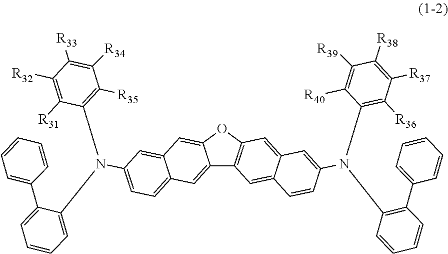

wherein in the formula (1-2), R₃₁ to R₄₀ are the same as defined in the formula (1); at least one of R₃₁ to R₄₀ is not a hydrogen atom.

In the compound represented by the formula (1-2), two phenyl groups bonded to two nitrogen atoms have one or more groups other than a hydrogen atom. That is, at least one of R₃₁ to R₄₀ is a substituted or unsubstituted alkyl group having 1 to 20 carbon atoms, a substituted or unsubstituted cycloalkyl group having 3 to 20 ring carbon atoms, a substituted or unsubstituted aryl group having 6 to 20 ring carbon atoms, a silyl group substituted with an alkyl group having 1 to 20 carbon atoms, or a cyano group.

In one embodiment, at least one of R₃₁ to R₄₀ is a substituted or unsubstituted alkyl group having 1 to 20 carbon atoms, or a substituted or unsubstituted aryl group having 6 to 20 ring carbon atoms.

In one embodiment, at least one of R₃₁ to R₃₅ is a substituted or unsubstituted alkyl group having 1 to 20 carbon atoms, or a substituted or unsubstituted aryl group having 6 to 20 ring carbon atoms, and at least one of R₃₆ to R₄₀ is a substituted or unsubstituted alkyl group having 1 to 20 carbon atoms, or a substituted or unsubstituted aryl group having 6 to 20 ring carbon atoms.

In one embodiment, the compound represented by the formula (1) is a compound represented by the following formula (1-3):

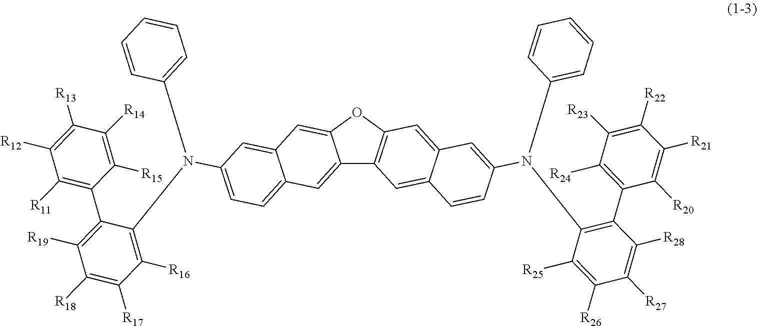

wherein in the formula (1-3), R₁₁ to R₂₈ are the same as defined in the formula (1); at least one of R₁₁ to R₂₈ is not a hydrogen atom.

In the compound represented by the formula (1-3), two biphenyl-2-yl groups bonded to two nitrogen atoms have one or more groups other than a hydrogen atom. That is, at least one of R₁₁ to R₂₈ is a substituted or unsubstituted alkyl group having 1 to 20 carbon atoms, a substituted or unsubstituted cycloalkyl group having 3 to 20 ring carbon atoms, a silyl group substituted with an alkyl group having 1 to 20 carbon atoms, or a cyano group.

In the compound represented by the formula (1), a substituent in the case of “substituted or unsubstituted” is selected from the group consisting of an alkyl group having 1 to 50 carbon atoms, a haloalkyl group having 1 to 50 carbon atoms, an alkenyl group having 2 to 50 carbon atoms, an alkynyl group having 2 to 50 carbon atoms, a cycloalkyl group having 3 to 50 ring carbon atoms, an alkoxy group having 1 to 50 carbon atoms, an alkylthio group having 1 to 50 carbon atoms, an aryloxy group having 6 to 50 ring carbon atoms, an arylthio group having 6 to 50 ring carbon atoms, an aralkyl group having 7 to 50 carbon atoms, —Si(R₄₁)(R₄₂)(R₄₃), —C(═O)R₄₄, —COOR₄₅, —S(═O)₂R₄₆, —P(═O)(R₄₇)(R₄₈), —Ge(R₄₉)(R₅₀)(R₅₁), —N(R₅₂)(R₅₃) (wherein, R₄₁ to R₅₃ are independently a hydrogen atom, an alkyl group having 1 to 50 carbon atoms, an aryl group having 6 to 50 ring carbon atoms, or a monovalent heterocyclic group having 5 to 50 ring atoms; when two or more of each of R₄₁ to R₅₃ are present, the two or more of each of R₄₁ to R₅₃ may be the same as or different from each other), a hydroxy group, a halogen atom, a cyano group, a nitro group, an aryl group having 6 to 50 ring carbon atoms, and a monovalent heterocyclic group having 5 to 50 ring atoms.

In one embodiment, the substituent in the case of “substituted or unsubstituted” in the compound represented by the formula (1) is an alkyl group having 1 to 50 carbon atoms, an aryl group having 6 to 50 ring carbon atoms, and a monovalent heterocyclic group having 5 to 50 ring atoms.

In one embodiment, the substituent in the case of “substituted or unsubstituted” in the compound represented by the formula (1) is selected from the group consisting of an alkyl group having 1 to 30 carbon atoms, an aryl group having 6 to 30 ring carbon atoms, and a monovalent heterocyclic group having 5 to 30 ring atoms.

In one embodiment, the substituent in the case of “substituted or unsubstituted” in the compound represented by the formula (1) is selected from the group consisting of an alkyl group having 1 to 18 carbon atoms, an aryl group having 6 to 18 ring carbon atoms, and a monovalent heterocyclic group having 5 to 18 ring atoms.

Specific examples of each substituent of the compound represented by the formula (1), the substituent in the case of “substituted or unsubstituted” and the halogen atom are the same as those described above.

The compound represented by the formula (1) can be synthesized in accordance with Examples by using known alternative reactions or raw materials adapted to the target compound.

Specific examples of the compound represented by the formula (1) will be described below, but these are merely examples, and the compound represented by the formula (1) is not limited to the following specific examples.

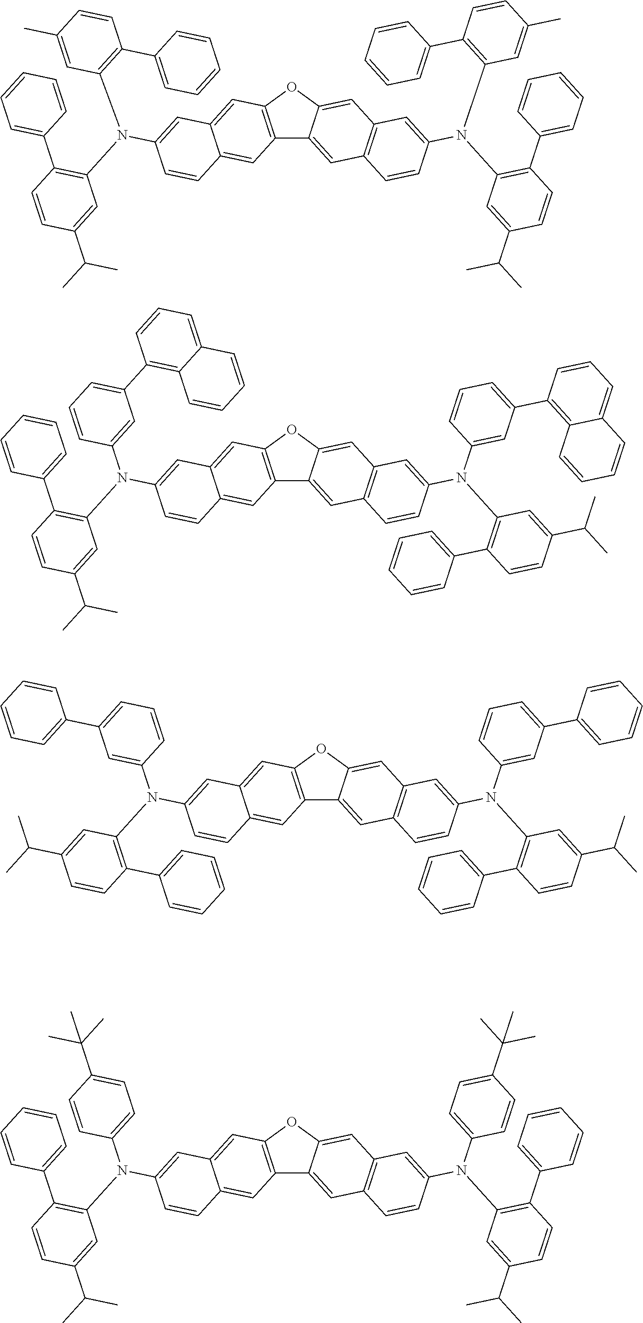

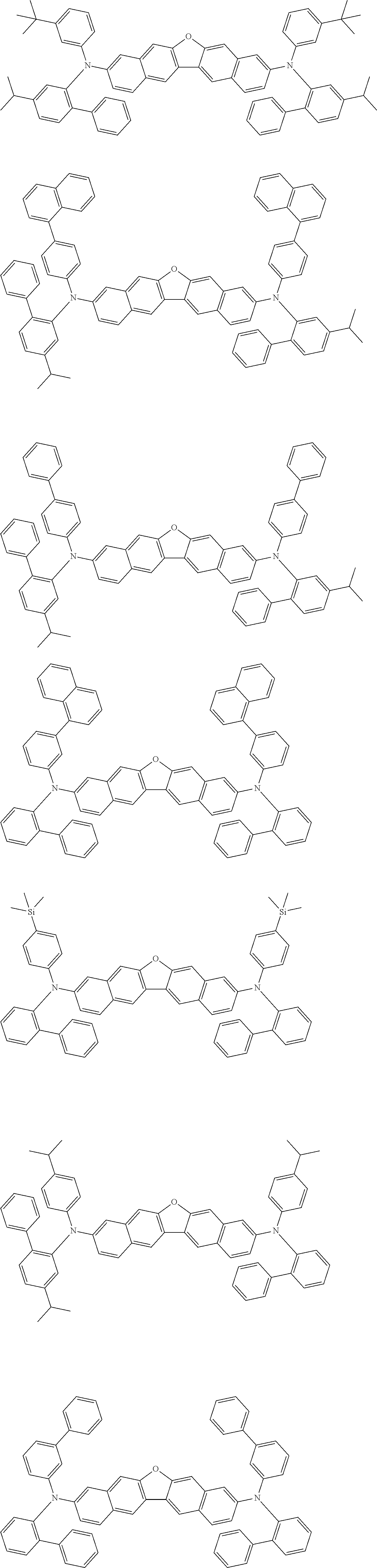

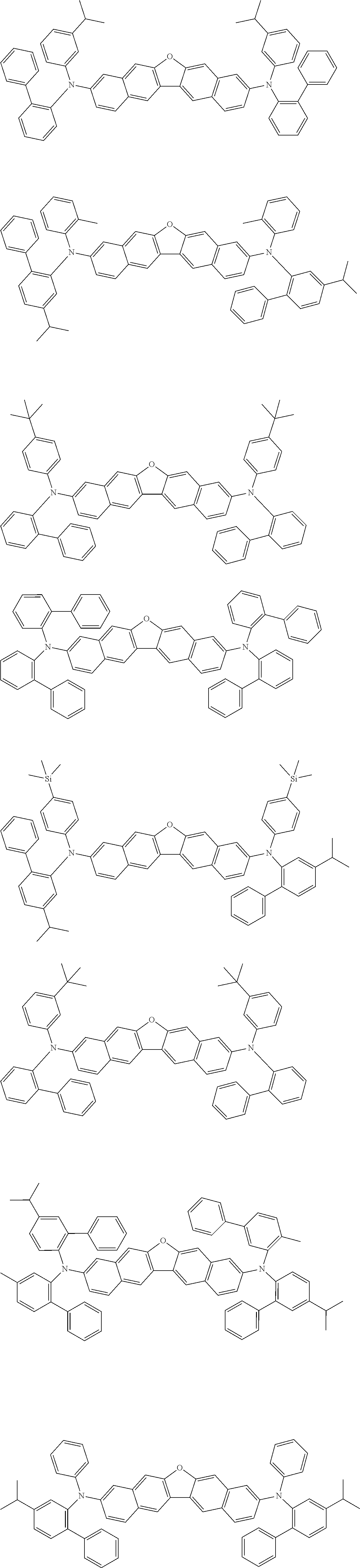

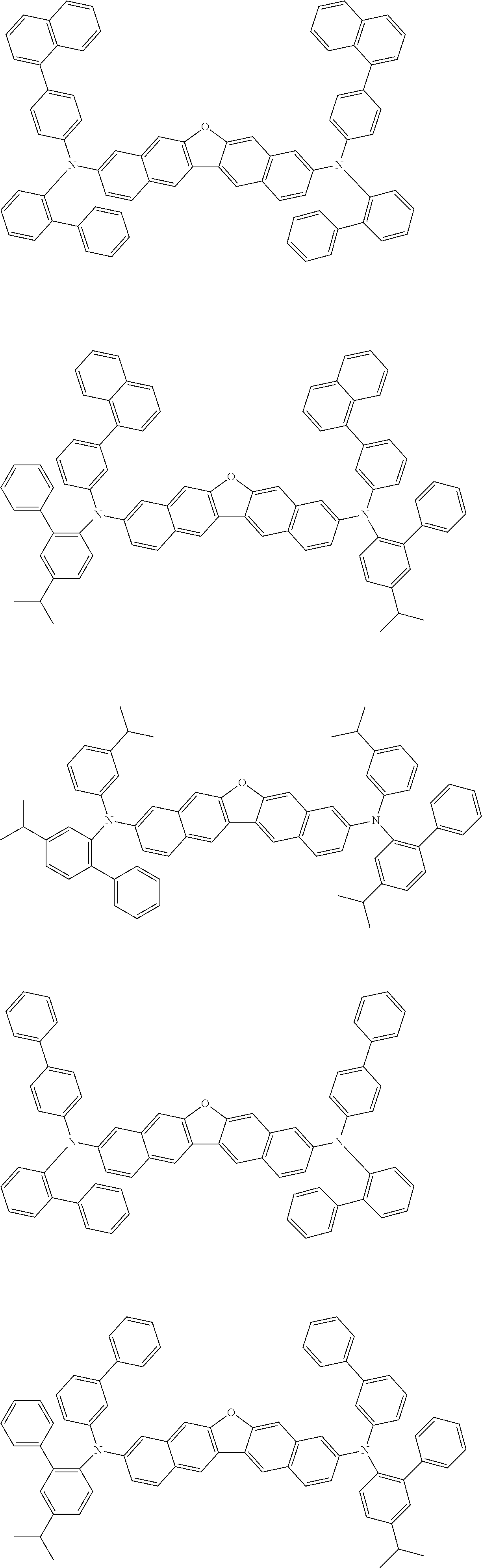

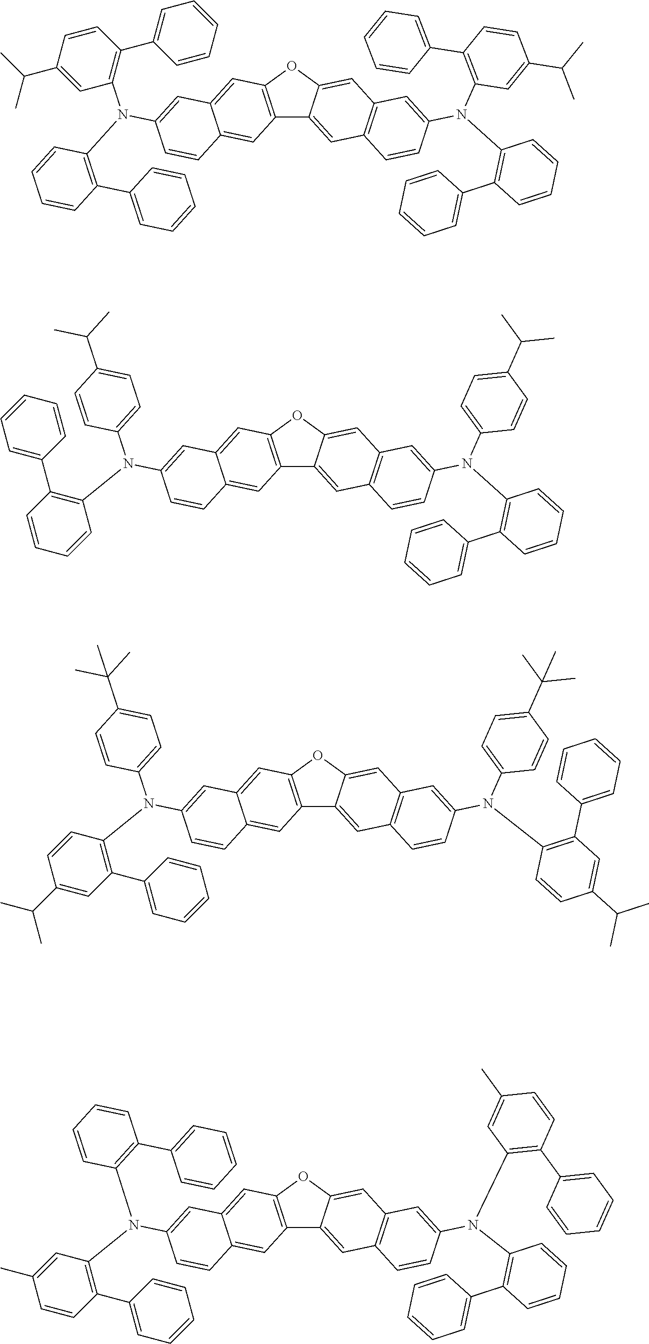

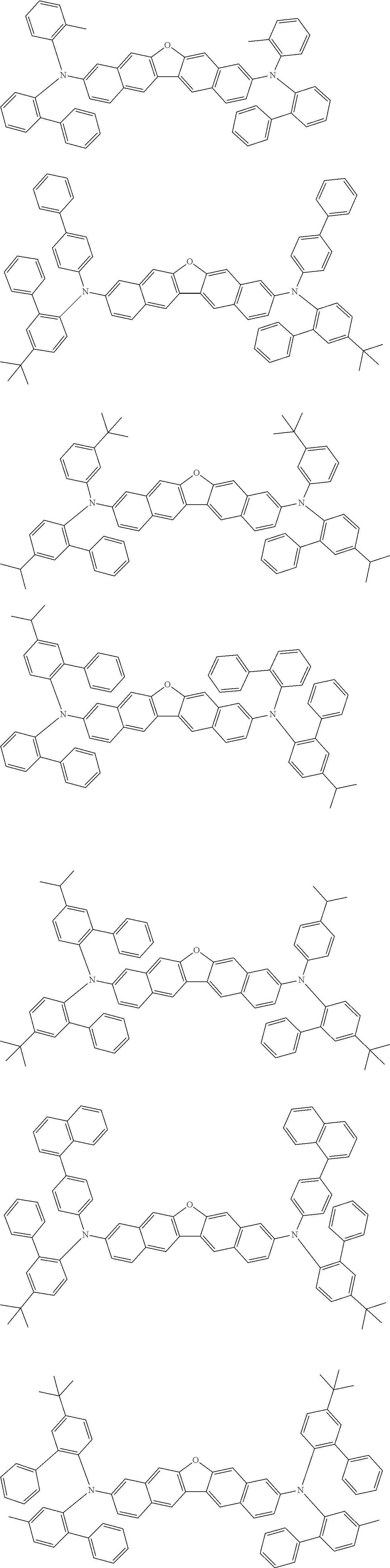

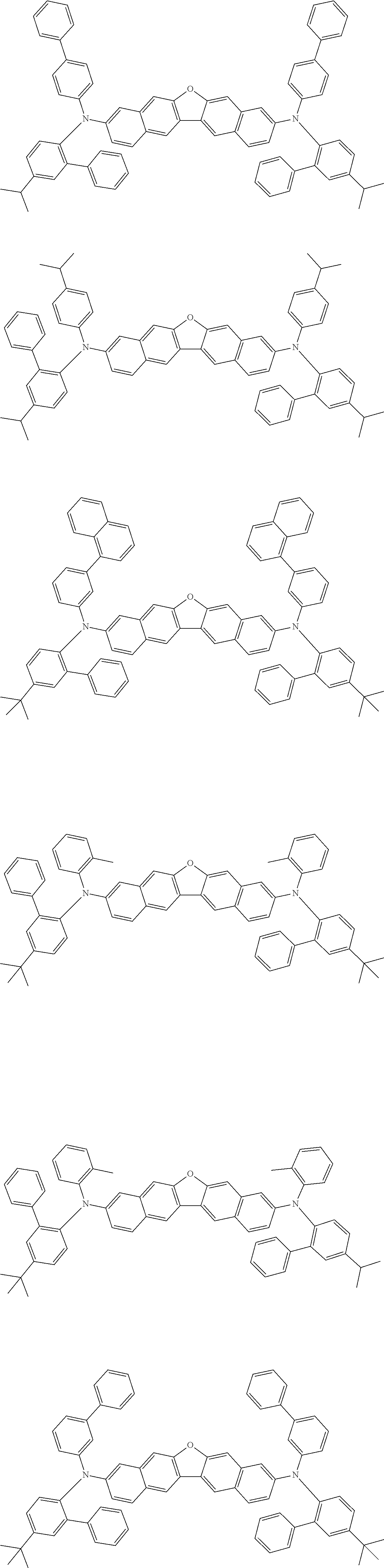

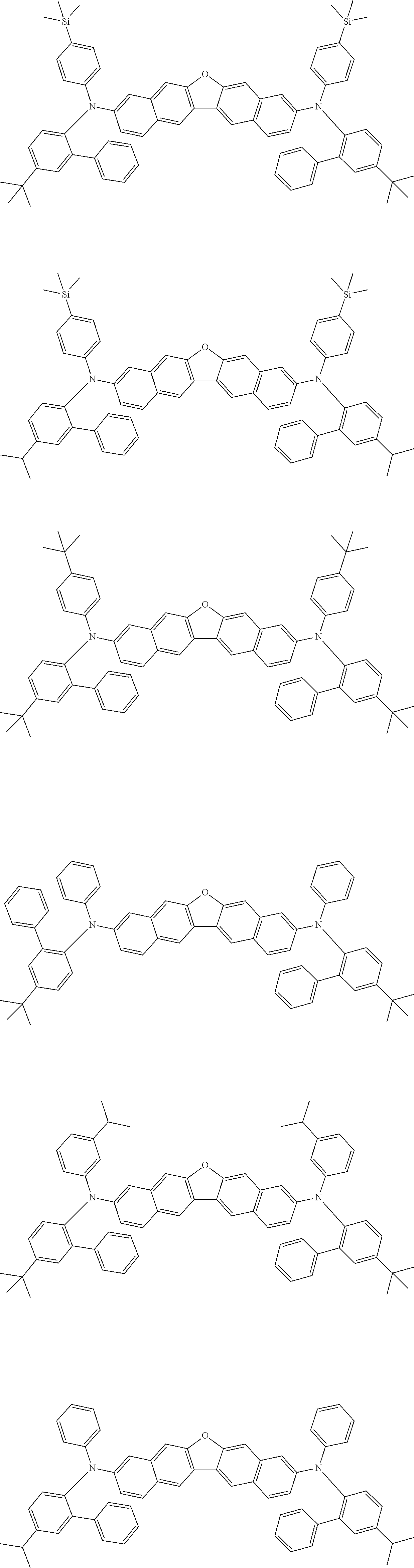

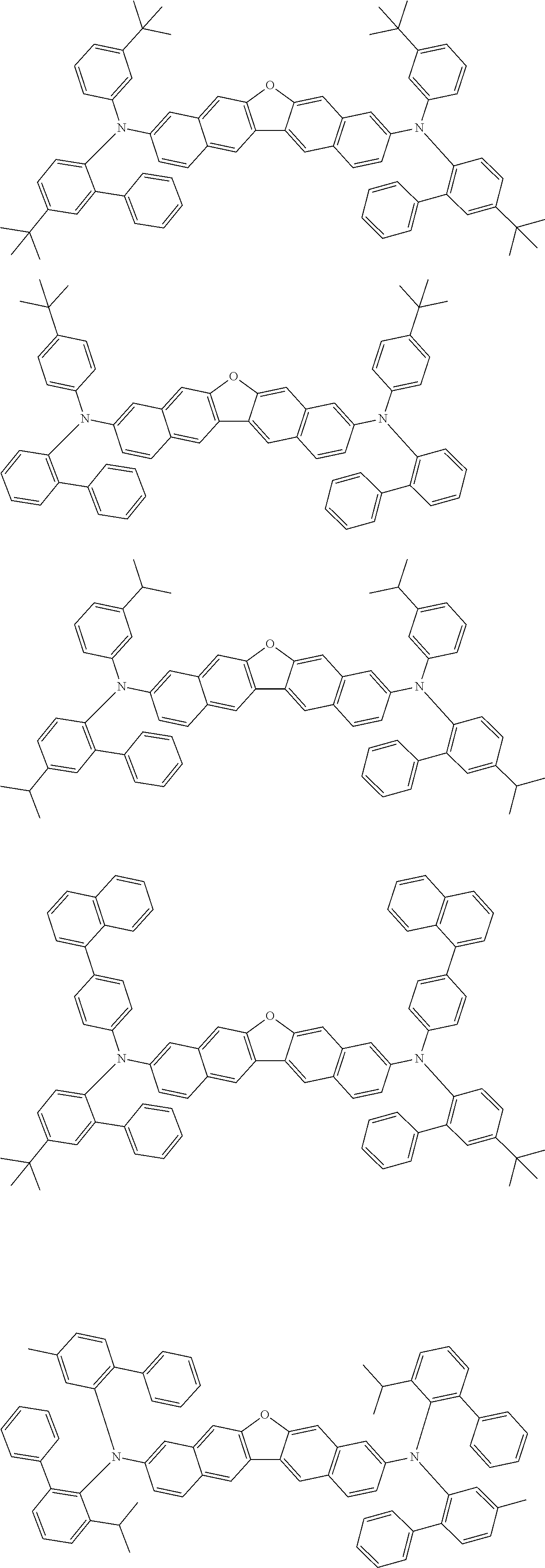

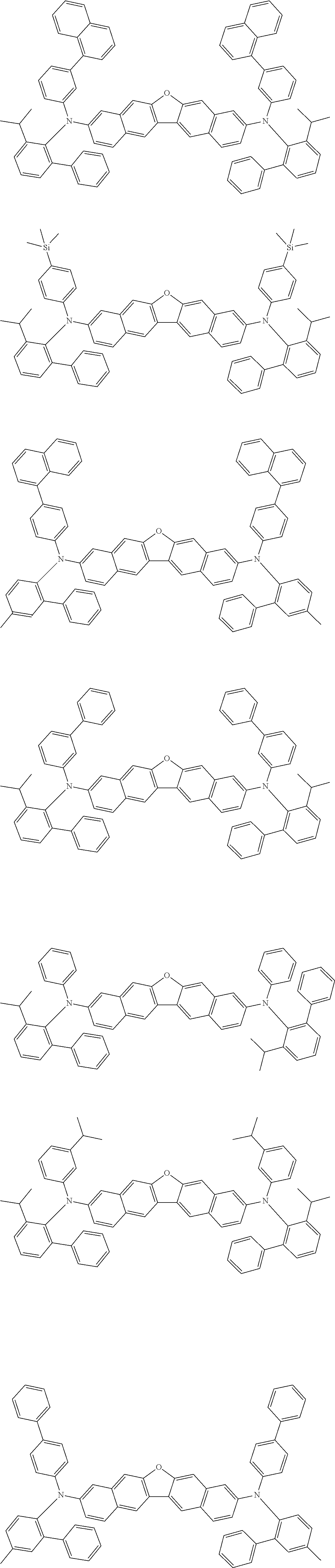

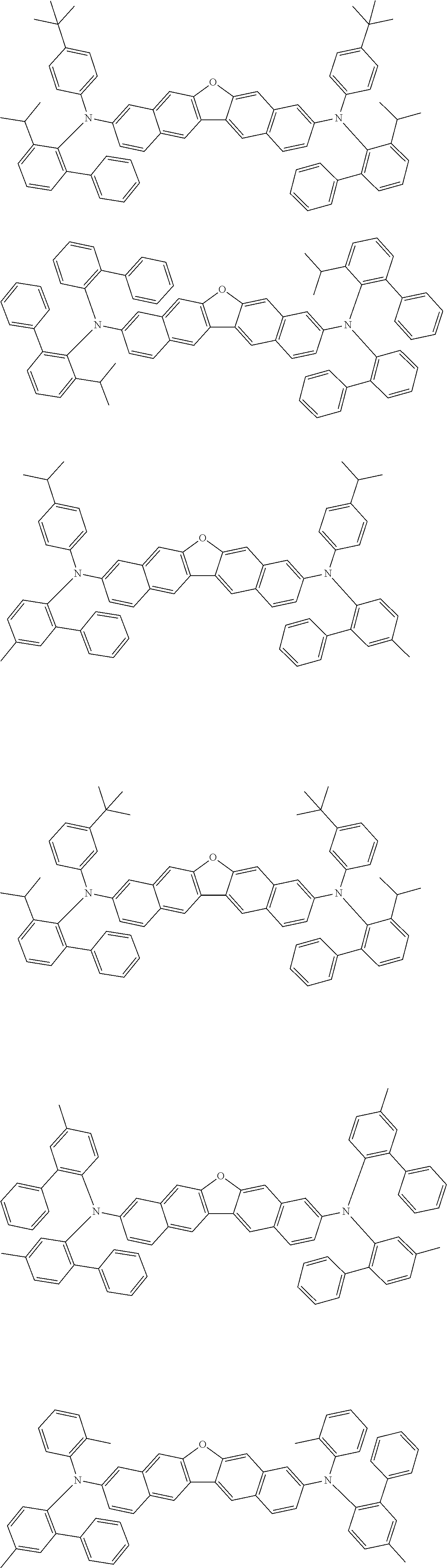

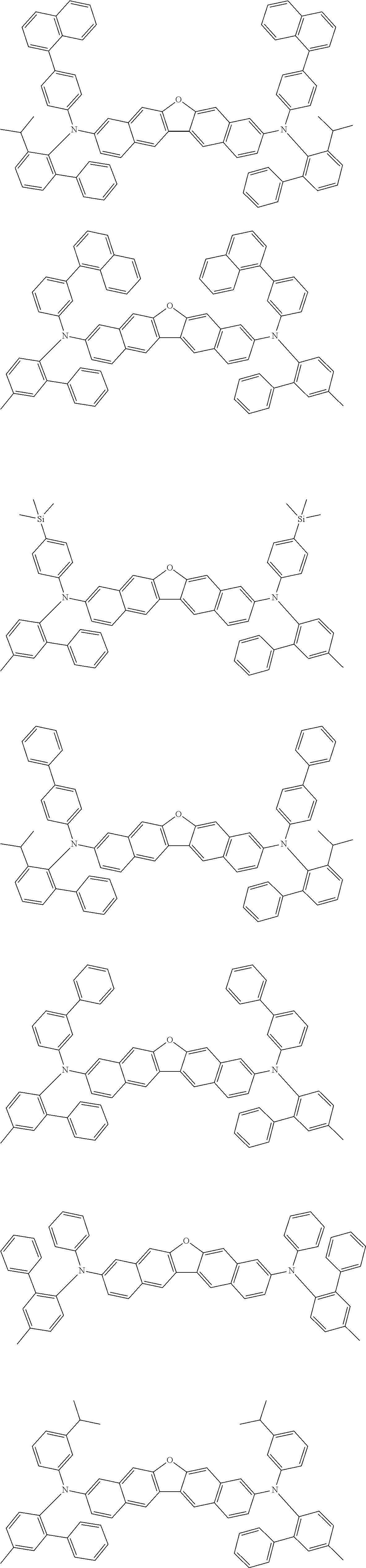

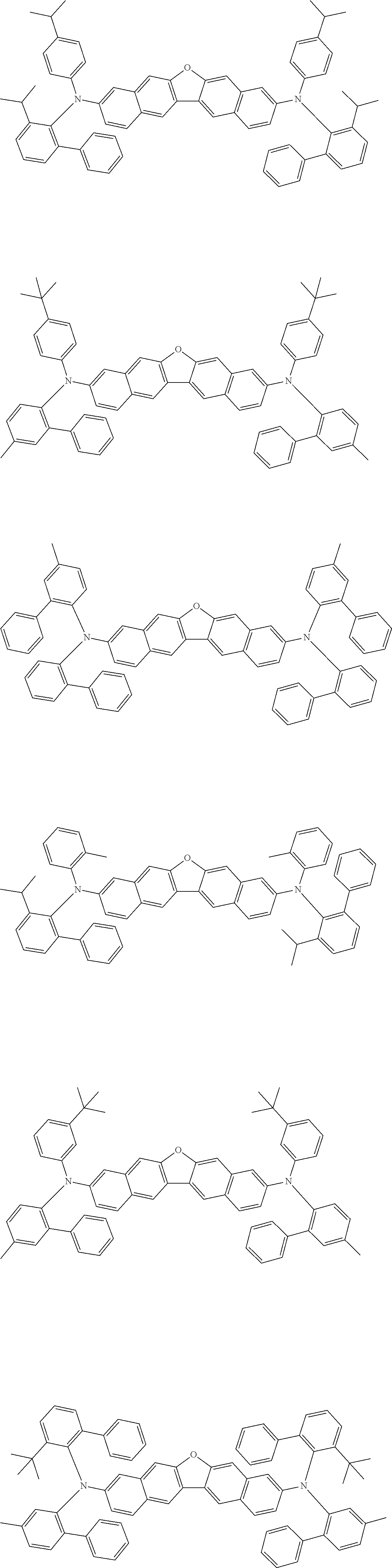

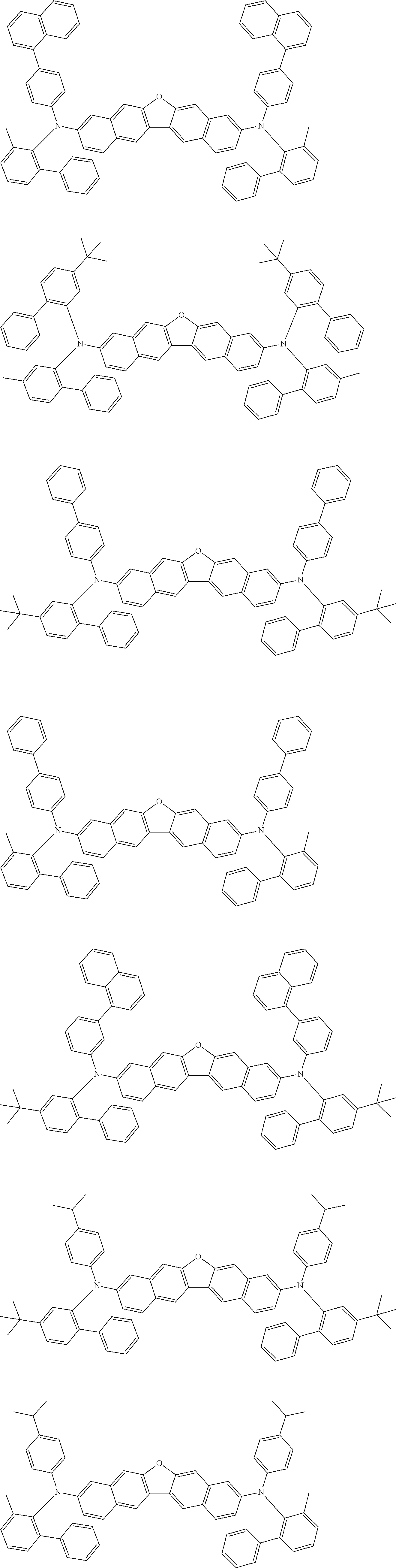

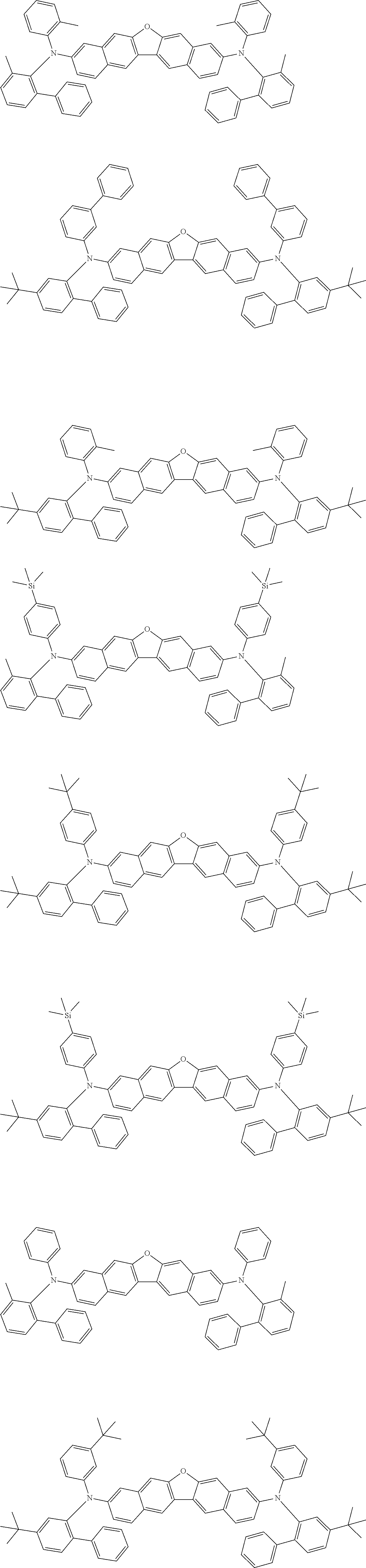

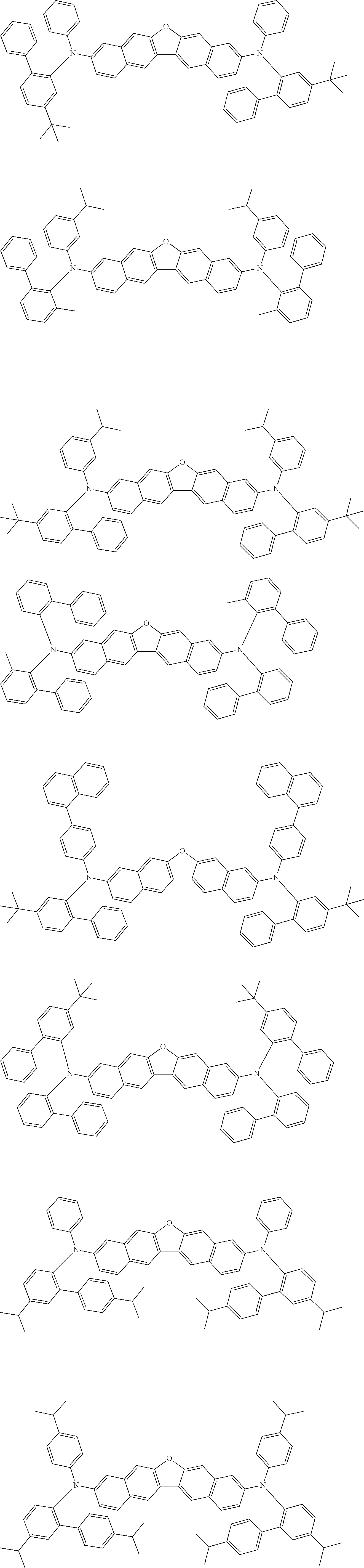

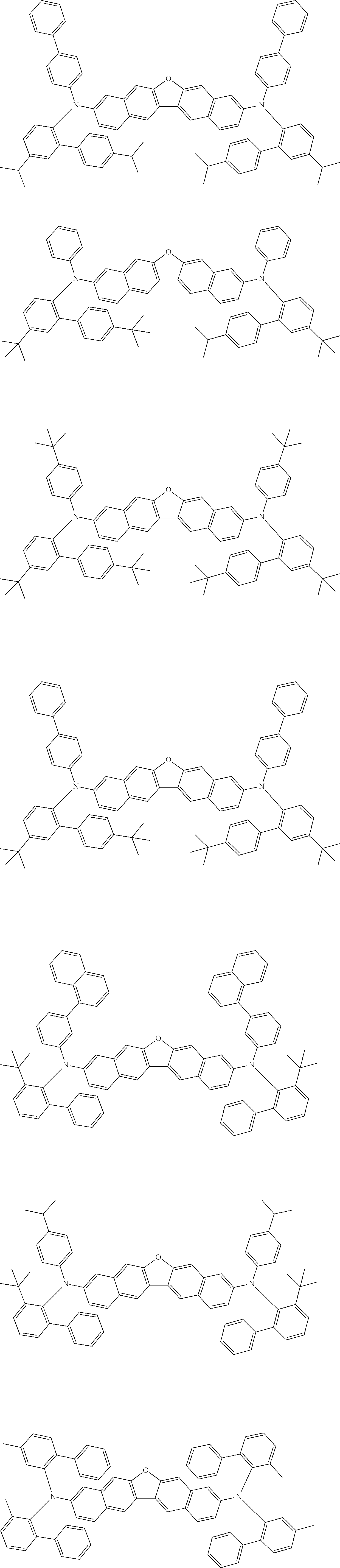

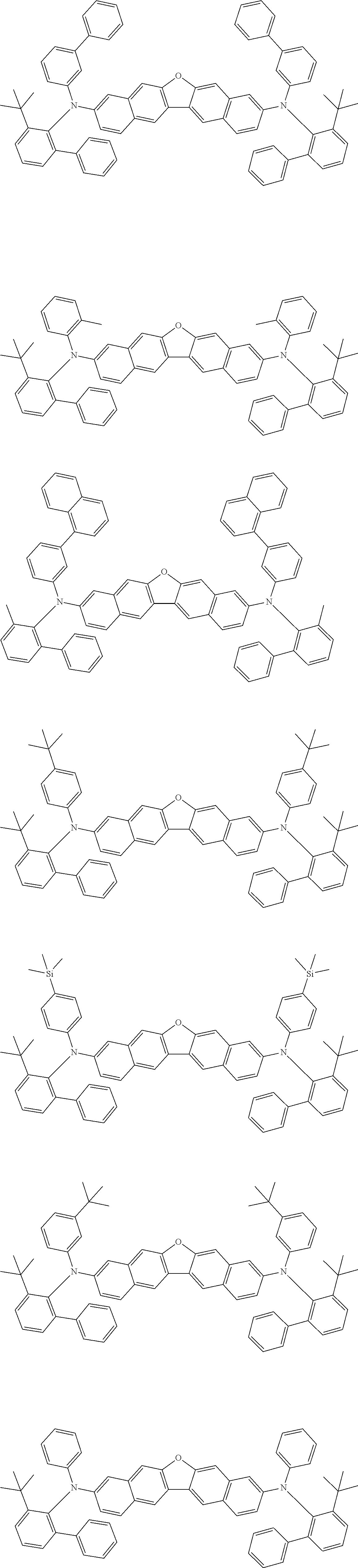

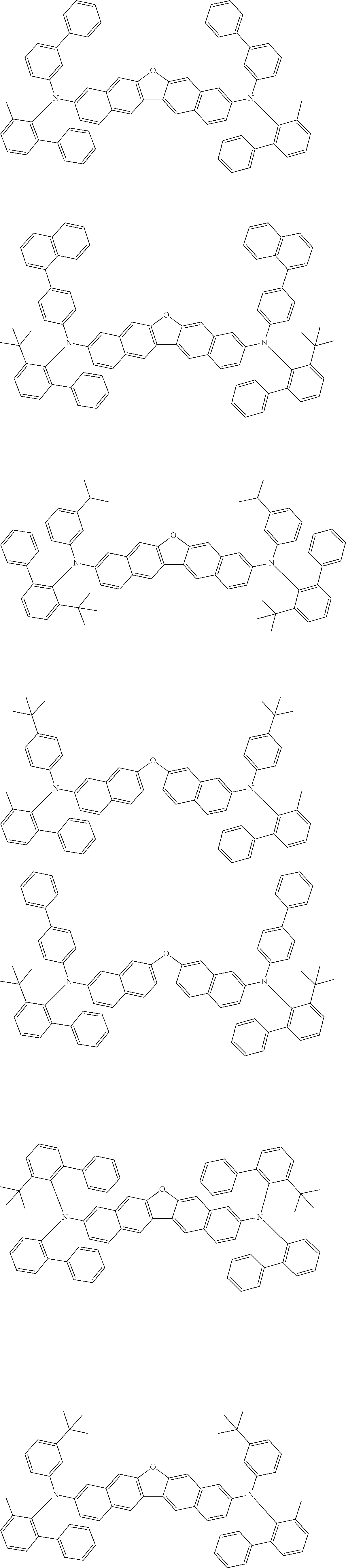

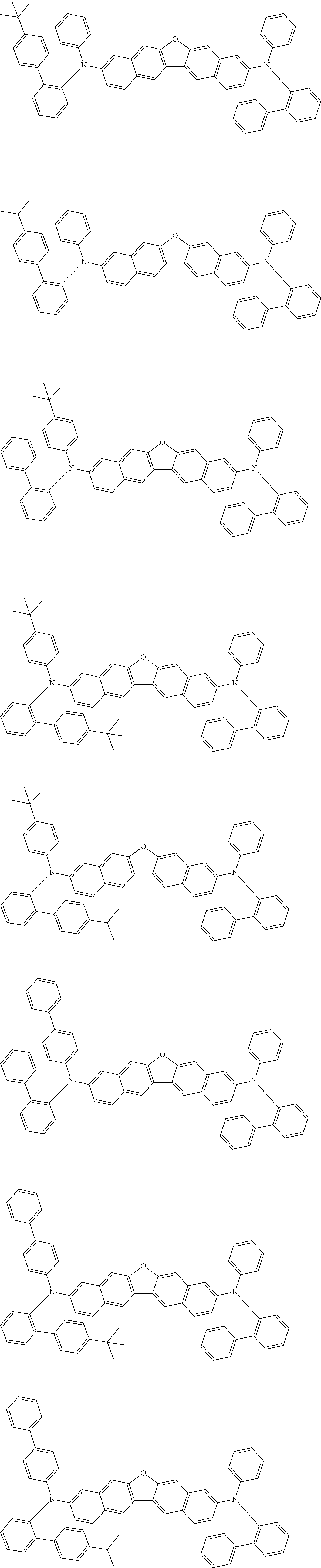

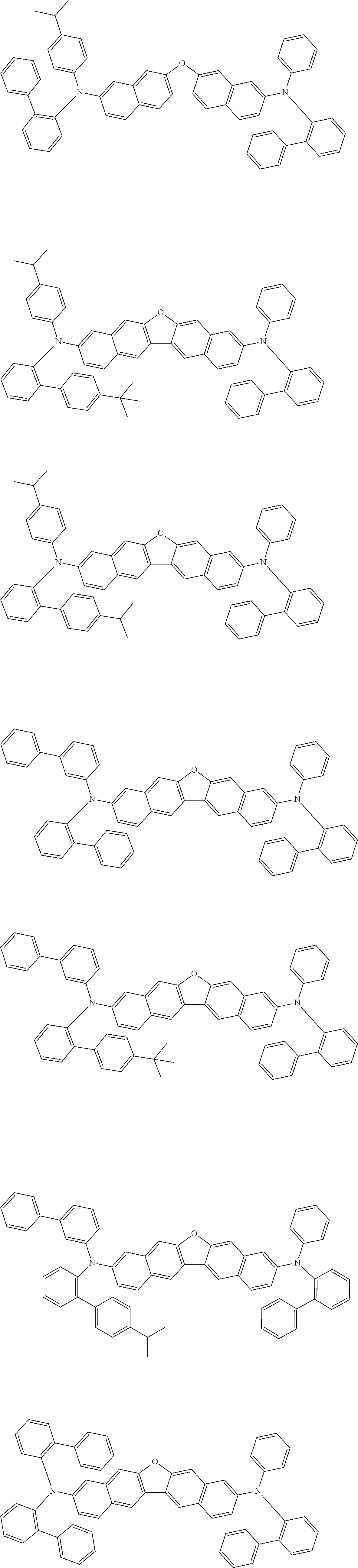

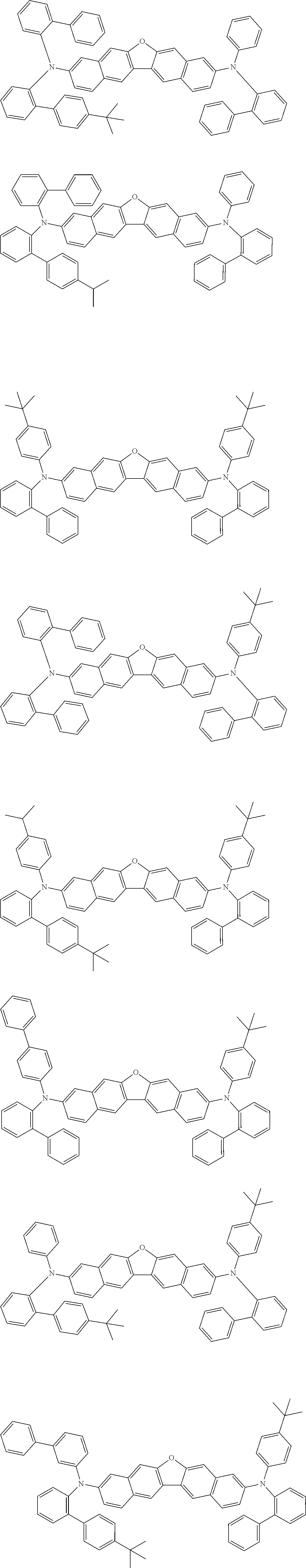

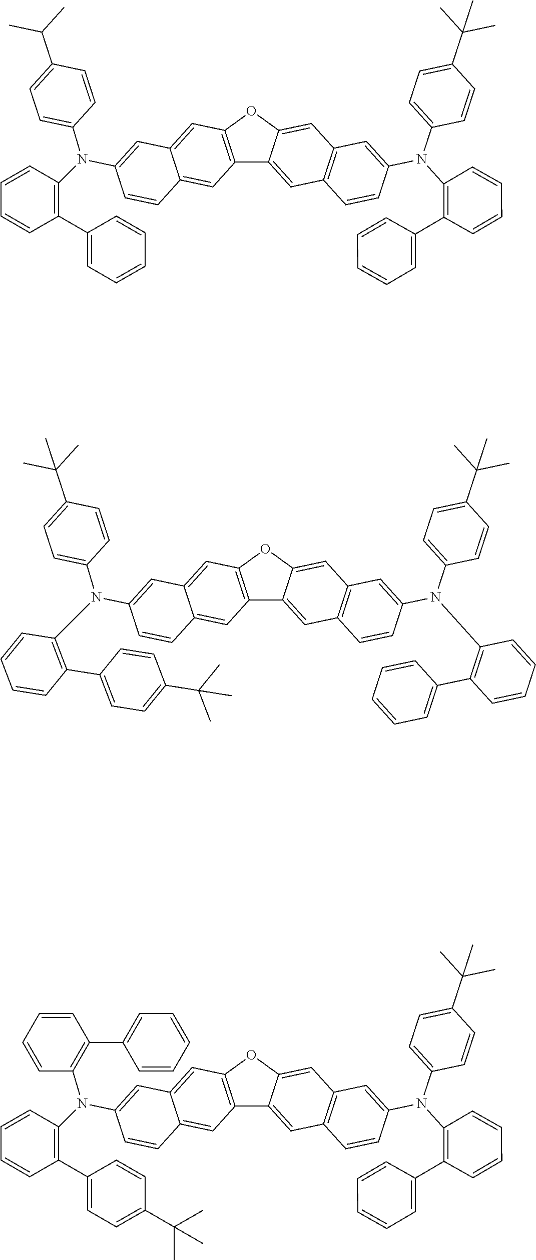 [SMILES: CC(C)c1ccc(N(c2ccc3cc4c(cc3c2)oc2cc3cc(N(c5ccc(C(C)(C)C)cc5)c5ccccc5-c5ccccc5)ccc3cc24)c2ccccc2-c2ccccc2)cc1 ; CC(C)(C)c1ccc(-c2ccccc2N(c2ccc(C(C)(C)C)cc2)c2ccc3cc4c(cc3c2)oc2cc3cc(N(c5ccc(C(C)(C)C)cc5)c5ccccc5-c5ccccc5)ccc3cc24)cc1 ; CC(C)(C)c1ccc(-c2ccccc2N(c2ccc3cc4c(cc3c2)oc2cc3cc(N(c5ccc(C(C)(C)C)cc5)c5ccccc5-c5ccccc5)ccc3cc24)c2ccccc2-c2ccccc2)cc1]

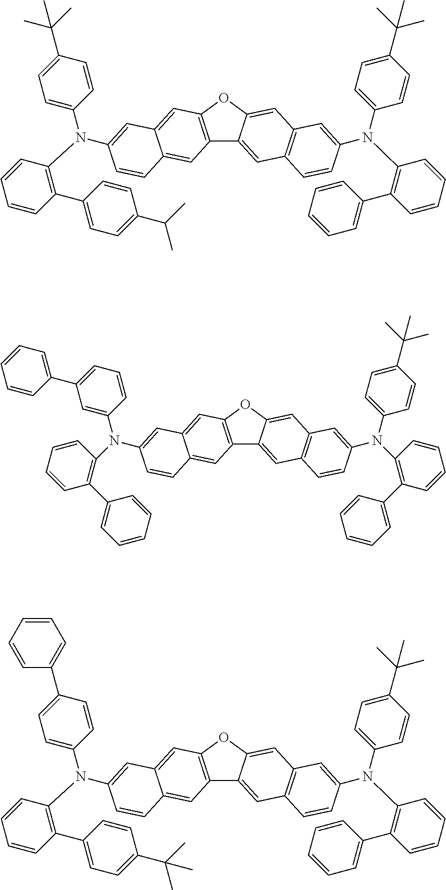 [SMILES: CC(C)c1ccc(-c2ccccc2N(c2ccc(C(C)(C)C)cc2)c2ccc3cc4c(cc3c2)oc2cc3cc(N(c5ccc(C(C)(C)C)cc5)c5ccccc5-c5ccccc5)ccc3cc24)cc1 ; CC(C)(C)c1ccc(N(c2ccc3cc4c(cc3c2)oc2cc3cc(N(c5cccc(-c6ccccc6)c5)c5ccccc5-c5ccccc5)ccc3cc24)c2ccccc2-c2ccccc2)cc1 ; CC(C)(C)c1ccc(-c2ccccc2N(c2ccc(-c3ccccc3)cc2)c2ccc3cc4c(cc3c2)oc2cc3cc(N(c5ccc(C(C)(C)C)cc5)c5ccccc5-c5ccccc5)ccc3cc24)cc1]

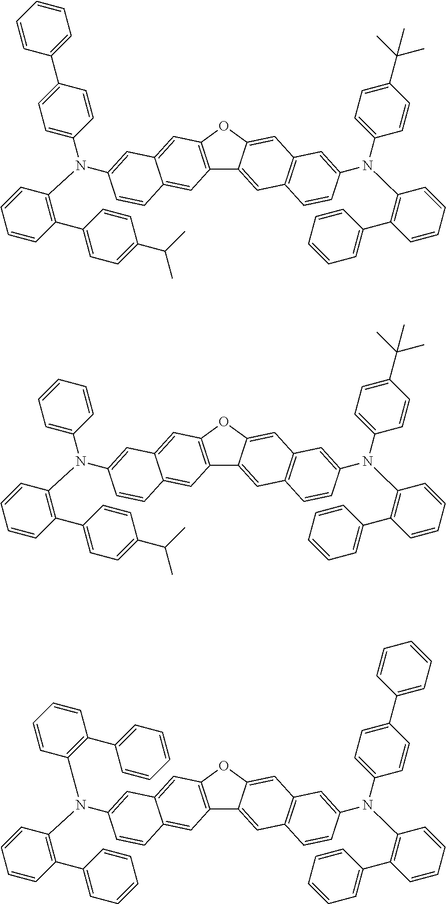 [SMILES: CC(C)c1ccc(-c2ccccc2N(c2ccc(-c3ccccc3)cc2)c2ccc3cc4c(cc3c2)oc2cc3cc(N(c5ccc(C(C)(C)C)cc5)c5ccccc5-c5ccccc5)ccc3cc24)cc1 ; CC(C)c1ccc(-c2ccccc2N(c2ccccc2)c2ccc3cc4c(cc3c2)oc2cc3cc(N(c5ccc(C(C)(C)C)cc5)c5ccccc5-c5ccccc5)ccc3cc24)cc1 ; c1ccc(-c2ccc(N(c3ccc4cc5c(cc4c3)oc3cc4cc(N(c6ccccc6-c6ccccc6)c6ccccc6-c6ccccc6)ccc4cc35)c3ccccc3-c3ccccc3)cc2)cc1]

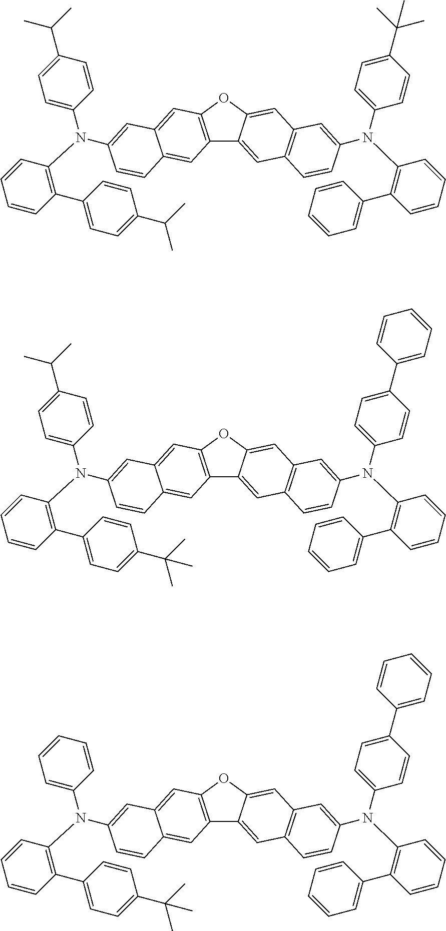 [SMILES: CC(C)c1ccc(-c2ccccc2N(c2ccc(C(C)C)cc2)c2ccc3cc4c(cc3c2)oc2cc3cc(N(c5ccc(C(C)(C)C)cc5)c5ccccc5-c5ccccc5)ccc3cc24)cc1 ; CC(C)c1ccc(N(c2ccc3cc4c(cc3c2)oc2cc3cc(N(c5ccc(-c6ccccc6)cc5)c5ccccc5-c5ccccc5)ccc3cc24)c2ccccc2-c2ccc(C(C)(C)C)cc2)cc1 ; CC(C)(C)c1ccc(-c2ccccc2N(c2ccccc2)c2ccc3cc4c(cc3c2)oc2cc3cc(N(c5ccc(-c6ccccc6)cc5)c5ccccc5-c5ccccc5)ccc3cc24)cc1]

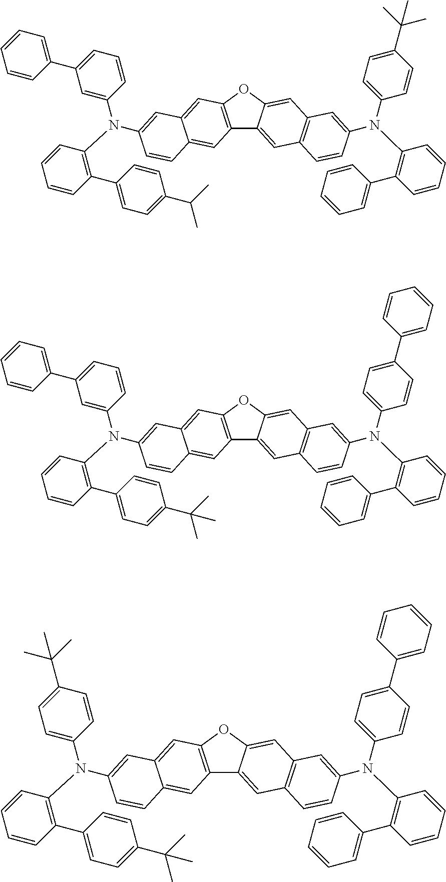 [SMILES: CC(C)c1ccc(-c2ccccc2N(c2cccc(-c3ccccc3)c2)c2ccc3cc4c(cc3c2)oc2cc3cc(N(c5ccc(C(C)(C)C)cc5)c5ccccc5-c5ccccc5)ccc3cc24)cc1 ; CC(C)(C)c1ccc(-c2ccccc2N(c2cccc(-c3ccccc3)c2)c2ccc3cc4c(cc3c2)oc2cc3cc(N(c5ccc(-c6ccccc6)cc5)c5ccccc5-c5ccccc5)ccc3cc24)cc1 ; CC(C)(C)c1ccc(-c2ccccc2N(c2ccc(C(C)(C)C)cc2)c2ccc3cc4c(cc3c2)oc2cc3cc(N(c5ccc(-c6ccccc6)cc5)c5ccccc5-c5ccccc5)ccc3cc24)cc1]

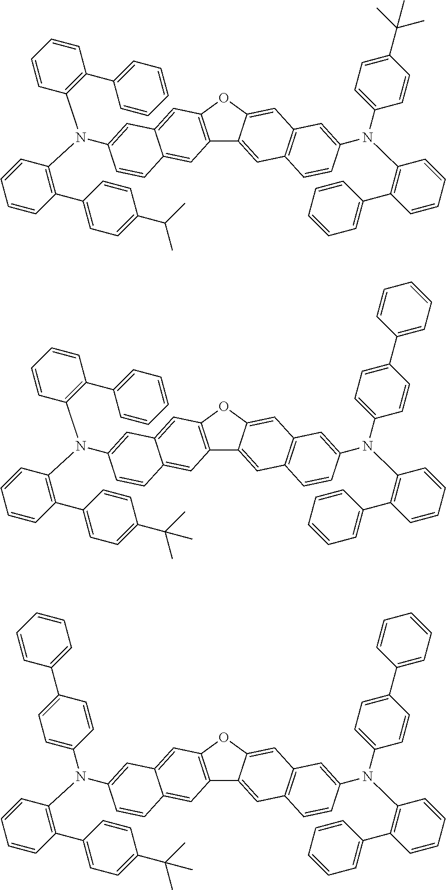 [SMILES: CC(C)c1ccc(-c2ccccc2N(c2ccc3cc4c(cc3c2)oc2cc3cc(N(c5ccc(C(C)(C)C)cc5)c5ccccc5-c5ccccc5)ccc3cc24)c2ccccc2-c2ccccc2)cc1 ; CC(C)(C)c1ccc(-c2ccccc2N(c2ccc3cc4c(cc3c2)oc2cc3cc(N(c5ccc(-c6ccccc6)cc5)c5ccccc5-c5ccccc5)ccc3cc24)c2ccccc2-c2ccccc2)cc1 ; CC(C)(C)c1ccc(-c2ccccc2N(c2ccc(-c3ccccc3)cc2)c2ccc3cc4c(cc3c2)oc2cc3cc(N(c5ccc(-c6ccccc6)cc5)c5ccccc5-c5ccccc5)ccc3cc24)cc1]

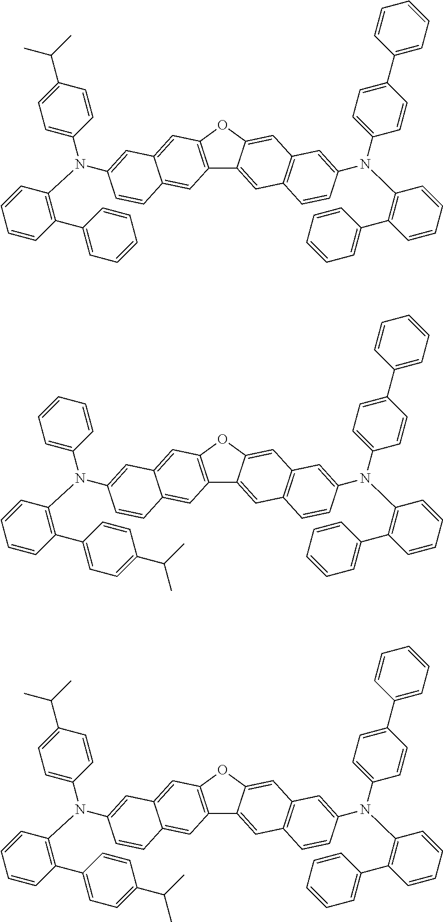 [SMILES: CC(C)c1ccc(N(c2ccc3cc4c(cc3c2)oc2cc3cc(N(c5ccc(-c6ccccc6)cc5)c5ccccc5-c5ccccc5)ccc3cc24)c2ccccc2-c2ccccc2)cc1 ; CC(C)c1ccc(-c2ccccc2N(c2ccccc2)c2ccc3cc4c(cc3c2)oc2cc3cc(N(c5ccc(-c6ccccc6)cc5)c5ccccc5-c5ccccc5)ccc3cc24)cc1 ; CC(C)c1ccc(-c2ccccc2N(c2ccc(C(C)C)cc2)c2ccc3cc4c(cc3c2)oc2cc3cc(N(c5ccc(-c6ccccc6)cc5)c5ccccc5-c5ccccc5)ccc3cc24)cc1]

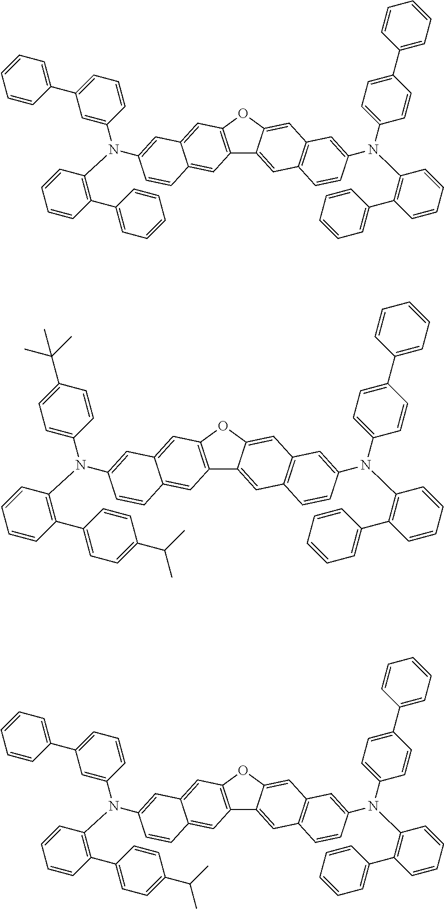 [SMILES: c1ccc(-c2ccc(N(c3ccc4cc5c(cc4c3)oc3cc4cc(N(c6cccc(-c7ccccc7)c6)c6ccccc6-c6ccccc6)ccc4cc35)c3ccccc3-c3ccccc3)cc2)cc1 ; CC(C)c1ccc(-c2ccccc2N(c2ccc(C(C)(C)C)cc2)c2ccc3cc4c(cc3c2)oc2cc3cc(N(c5ccc(-c6ccccc6)cc5)c5ccccc5-c5ccccc5)ccc3cc24)cc1 ; CC(C)c1ccc(-c2ccccc2N(c2cccc(-c3ccccc3)c2)c2ccc3cc4c(cc3c2)oc2cc3cc(N(c5ccc(-c6ccccc6)cc5)c5ccccc5-c5ccccc5)ccc3cc24)cc1]

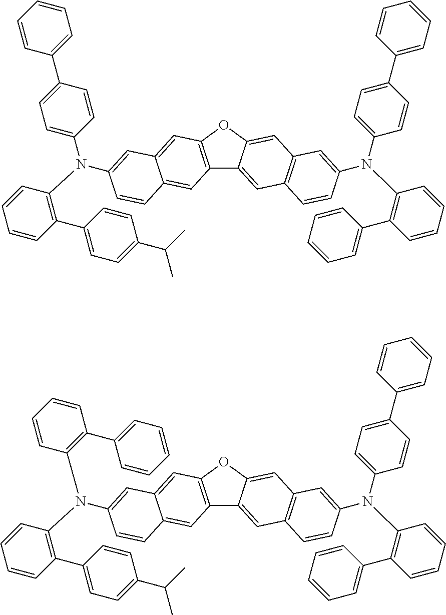 [SMILES: CC(C)c1ccc(-c2ccccc2N(c2ccc(-c3ccccc3)cc2)c2ccc3cc4c(cc3c2)oc2cc3cc(N(c5ccc(-c6ccccc6)cc5)c5ccccc5-c5ccccc5)ccc3cc24)cc1 ; CC(C)c1ccc(-c2ccccc2N(c2ccc3cc4c(cc3c2)oc2cc3cc(N(c5ccc(-c6ccccc6)cc5)c5ccccc5-c5ccccc5)ccc3cc24)c2ccccc2-c2ccccc2)cc1]

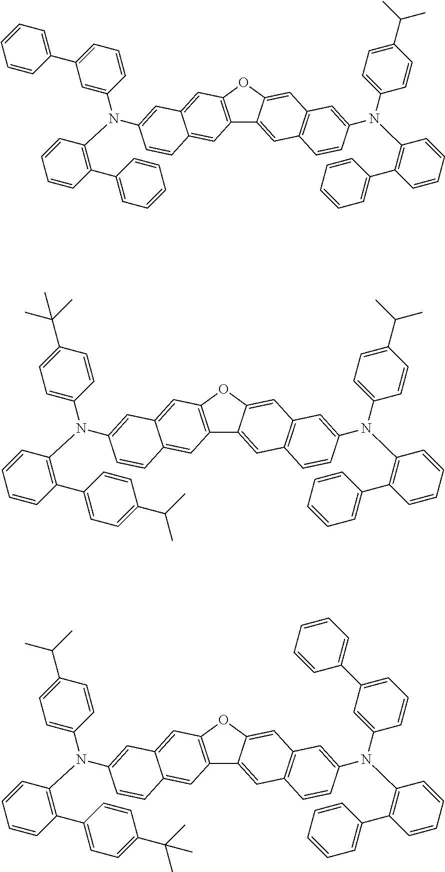 [SMILES: CC(C)c1ccc(N(c2ccc3cc4c(cc3c2)oc2cc3cc(N(c5cccc(-c6ccccc6)c5)c5ccccc5-c5ccccc5)ccc3cc24)c2ccccc2-c2ccccc2)cc1 ; CC(C)c1ccc(-c2ccccc2N(c2ccc(C(C)(C)C)cc2)c2ccc3cc4c(cc3c2)oc2cc3cc(N(c5ccc(C(C)C)cc5)c5ccccc5-c5ccccc5)ccc3cc24)cc1 ; CC(C)c1ccc(N(c2ccc3cc4c(cc3c2)oc2cc3cc(N(c5cccc(-c6ccccc6)c5)c5ccccc5-c5ccccc5)ccc3cc24)c2ccccc2-c2ccc(C(C)(C)C)cc2)cc1]

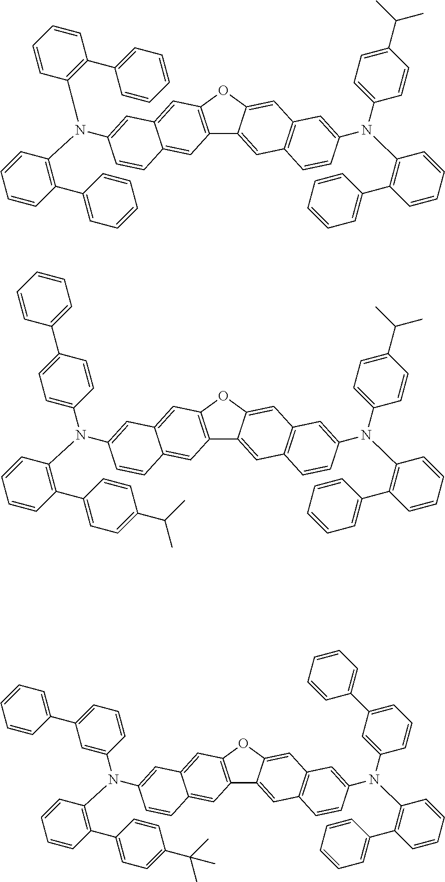 [SMILES: CC(C)c1ccc(N(c2ccc3cc4c(cc3c2)oc2cc3cc(N(c5ccccc5-c5ccccc5)c5ccccc5-c5ccccc5)ccc3cc24)c2ccccc2-c2ccccc2)cc1 ; CC(C)c1ccc(-c2ccccc2N(c2ccc(-c3ccccc3)cc2)c2ccc3cc4c(cc3c2)oc2cc3cc(N(c5ccc(C(C)C)cc5)c5ccccc5-c5ccccc5)ccc3cc24)cc1 ; CC(C)(C)c1ccc(-c2ccccc2N(c2cccc(-c3ccccc3)c2)c2ccc3cc4c(cc3c2)oc2cc3cc(N(c5cccc(-c6ccccc6)c5)c5ccccc5-c5ccccc5)ccc3cc24)cc1]

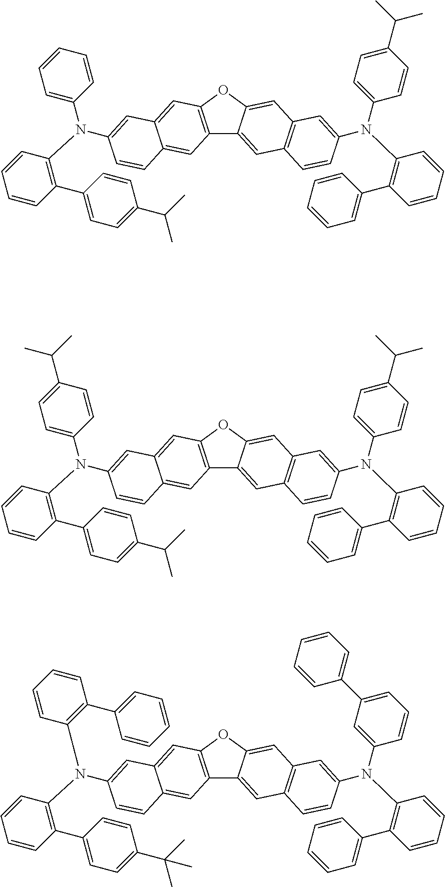 [SMILES: CC(C)c1ccc(-c2ccccc2N(c2ccccc2)c2ccc3cc4c(cc3c2)oc2cc3cc(N(c5ccc(C(C)C)cc5)c5ccccc5-c5ccccc5)ccc3cc24)cc1 ; CC(C)c1ccc(-c2ccccc2N(c2ccc(C(C)C)cc2)c2ccc3cc4c(cc3c2)oc2cc3cc(N(c5ccc(C(C)C)cc5)c5ccccc5-c5ccccc5)ccc3cc24)cc1 ; CC(C)(C)c1ccc(-c2ccccc2N(c2ccc3cc4c(cc3c2)oc2cc3cc(N(c5cccc(-c6ccccc6)c5)c5ccccc5-c5ccccc5)ccc3cc24)c2ccccc2-c2ccccc2)cc1]

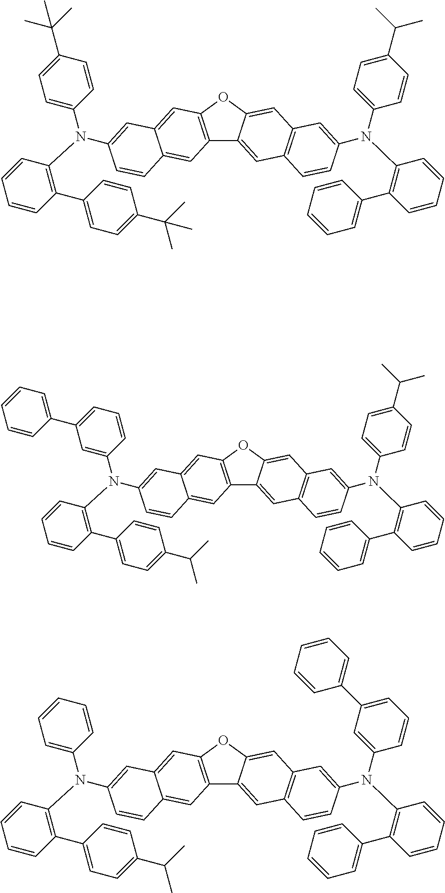 [SMILES: CC(C)c1ccc(N(c2ccc3cc4c(cc3c2)oc2cc3cc(N(c5ccc(C(C)(C)C)cc5)c5ccccc5-c5ccc(C(C)(C)C)cc5)ccc3cc24)c2ccccc2-c2ccccc2)cc1 ; CC(C)c1ccc(-c2ccccc2N(c2cccc(-c3ccccc3)c2)c2ccc3cc4c(cc3c2)oc2cc3cc(N(c5ccc(C(C)C)cc5)c5ccccc5-c5ccccc5)ccc3cc24)cc1 ; CC(C)c1ccc(-c2ccccc2N(c2ccccc2)c2ccc3cc4c(cc3c2)oc2cc3cc(N(c5cccc(-c6ccccc6)c5)c5ccccc5-c5ccccc5)ccc3cc24)cc1]

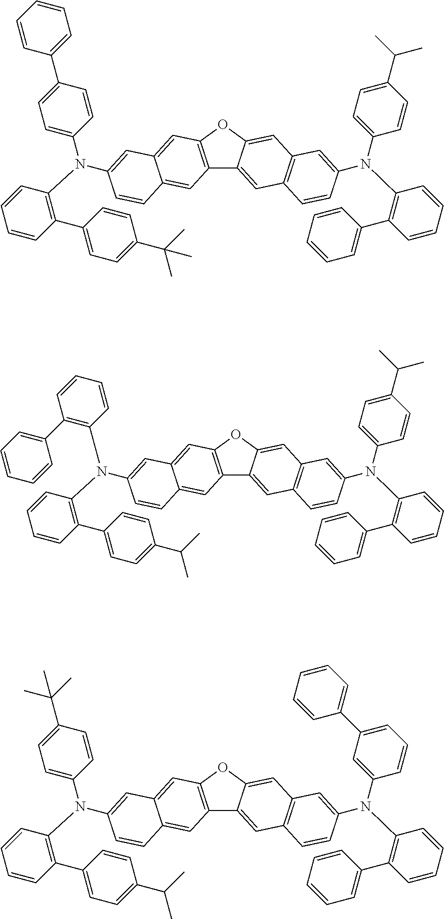 [SMILES: CC(C)c1ccc(N(c2ccc3cc4c(cc3c2)oc2cc3cc(N(c5ccc(-c6ccccc6)cc5)c5ccccc5-c5ccc(C(C)(C)C)cc5)ccc3cc24)c2ccccc2-c2ccccc2)cc1 ; CC(C)c1ccc(-c2ccccc2N(c2ccc3cc4c(cc3c2)oc2cc3cc(N(c5ccc(C(C)C)cc5)c5ccccc5-c5ccccc5)ccc3cc24)c2ccccc2-c2ccccc2)cc1 ; CC(C)c1ccc(-c2ccccc2N(c2ccc(C(C)(C)C)cc2)c2ccc3cc4c(cc3c2)oc2cc3cc(N(c5cccc(-c6ccccc6)c5)c5ccccc5-c5ccccc5)ccc3cc24)cc1]

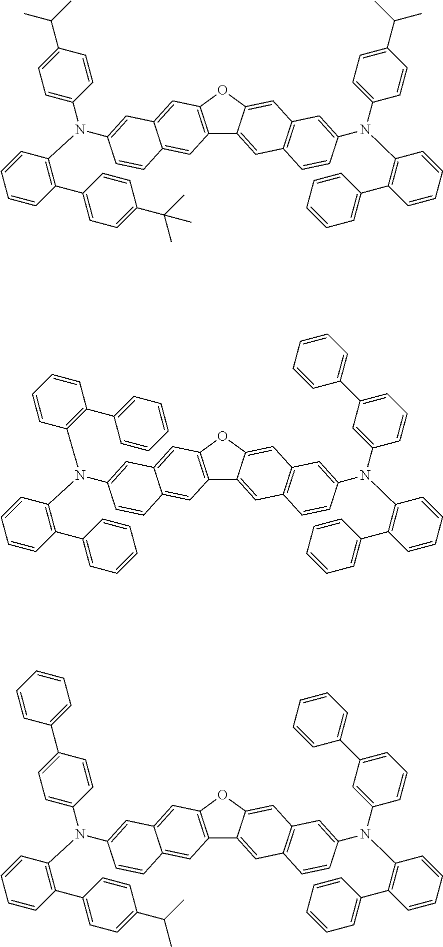 [SMILES: CC(C)c1ccc(N(c2ccc3cc4c(cc3c2)oc2cc3cc(N(c5ccc(C(C)C)cc5)c5ccccc5-c5ccc(C(C)(C)C)cc5)ccc3cc24)c2ccccc2-c2ccccc2)cc1 ; c1ccc(-c2cccc(N(c3ccc4cc5c(cc4c3)oc3cc4cc(N(c6ccccc6-c6ccccc6)c6ccccc6-c6ccccc6)ccc4cc35)c3ccccc3-c3ccccc3)c2)cc1 ; CC(C)c1ccc(-c2ccccc2N(c2ccc(-c3ccccc3)cc2)c2ccc3cc4c(cc3c2)oc2cc3cc(N(c5cccc(-c6ccccc6)c5)c5ccccc5-c5ccccc5)ccc3cc24)cc1]

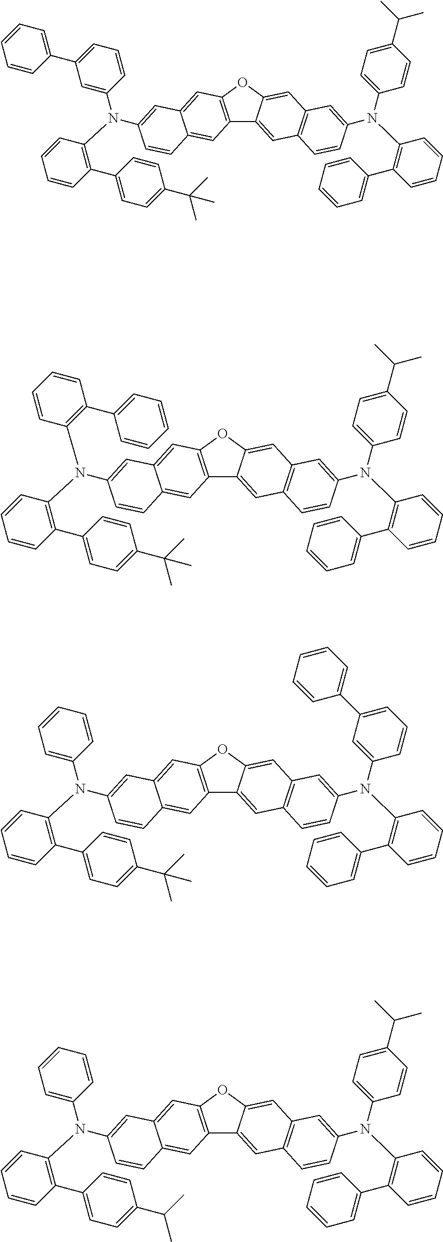 [SMILES: CC(C)c1ccc(N(c2ccc3cc4c(cc3c2)oc2cc3cc(N(c5cccc(-c6ccccc6)c5)c5ccccc5-c5ccc(C(C)(C)C)cc5)ccc3cc24)c2ccccc2-c2ccccc2)cc1 ; CC(C)c1ccc(N(c2ccc3cc4c(cc3c2)oc2cc3cc(N(c5ccccc5-c5ccccc5)c5ccccc5-c5ccc(C(C)(C)C)cc5)ccc3cc24)c2ccccc2-c2ccccc2)cc1 ; CC(C)(C)c1ccc(-c2ccccc2N(c2ccccc2)c2ccc3cc4c(cc3c2)oc2cc3cc(N(c5cccc(-c6ccccc6)c5)c5ccccc5-c5ccccc5)ccc3cc24)cc1 ; CC(C)c1ccc(-c2ccccc2N(c2ccccc2)c2ccc3cc4c(cc3c2)oc2cc3cc(N(c5ccc(C(C)C)cc5)c5ccccc5-c5ccccc5)ccc3cc24)cc1]

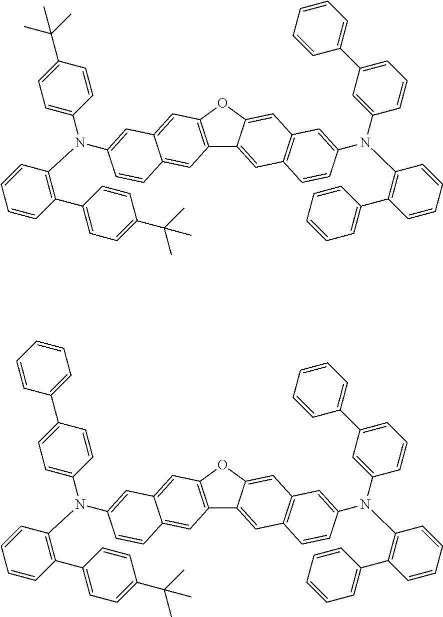 [SMILES: CC(C)(C)c1ccc(-c2ccccc2N(c2ccc(C(C)(C)C)cc2)c2ccc3cc4c(cc3c2)oc2cc3cc(N(c5cccc(-c6ccccc6)c5)c5ccccc5-c5ccccc5)ccc3cc24)cc1 ; CC(C)(C)c1ccc(-c2ccccc2N(c2ccc(-c3ccccc3)cc2)c2ccc3cc4c(cc3c2)oc2cc3cc(N(c5cccc(-c6ccccc6)c5)c5ccccc5-c5ccccc5)ccc3cc24)cc1]

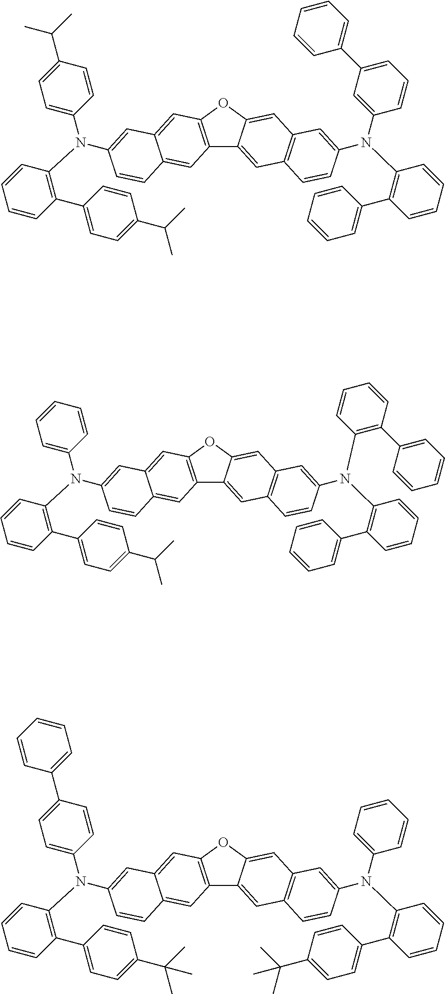 [SMILES: CC(C)c1ccc(-c2ccccc2N(c2ccc(C(C)C)cc2)c2ccc3cc4c(cc3c2)oc2cc3cc(N(c5cccc(-c6ccccc6)c5)c5ccccc5-c5ccccc5)ccc3cc24)cc1 ; CC(C)c1ccc(-c2ccccc2N(c2ccccc2)c2ccc3cc4c(cc3c2)oc2cc3cc(N(c5ccccc5-c5ccccc5)c5ccccc5-c5ccccc5)ccc3cc24)cc1 ; CC(C)(C)c1ccc(-c2ccccc2N(c2ccccc2)c2ccc3cc4c(cc3c2)oc2cc3cc(N(c5ccc(-c6ccccc6)cc5)c5ccccc5-c5ccc(C(C)(C)C)cc5)ccc3cc24)cc1]

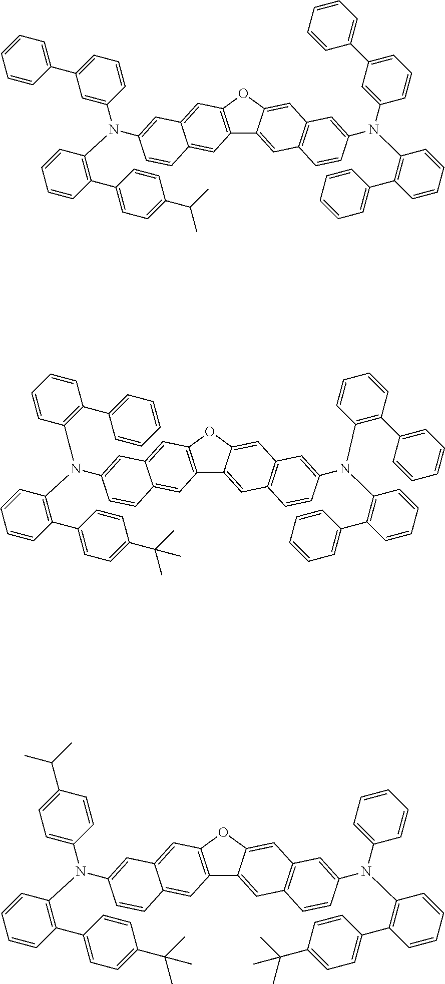 [SMILES: CC(C)c1ccc(-c2ccccc2N(c2cccc(-c3ccccc3)c2)c2ccc3cc4c(cc3c2)oc2cc3cc(N(c5cccc(-c6ccccc6)c5)c5ccccc5-c5ccccc5)ccc3cc24)cc1 ; CC(C)(C)c1ccc(-c2ccccc2N(c2ccc3cc4c(cc3c2)oc2cc3cc(N(c5ccccc5-c5ccccc5)c5ccccc5-c5ccccc5)ccc3cc24)c2ccccc2-c2ccccc2)cc1 ; CC(C)c1ccc(N(c2ccc3cc4c(cc3c2)oc2cc3cc(N(c5ccccc5)c5ccccc5-c5ccc(C(C)(C)C)cc5)ccc3cc24)c2ccccc2-c2ccc(C(C)(C)C)cc2)cc1]

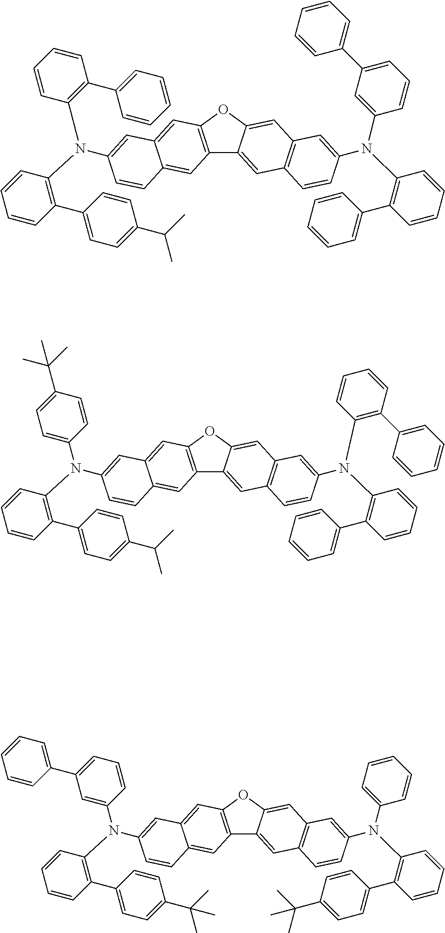 [SMILES: CC(C)c1ccc(-c2ccccc2N(c2ccc3cc4c(cc3c2)oc2cc3cc(N(c5cccc(-c6ccccc6)c5)c5ccccc5-c5ccccc5)ccc3cc24)c2ccccc2-c2ccccc2)cc1 ; CC(C)c1ccc(-c2ccccc2N(c2ccc(C(C)(C)C)cc2)c2ccc3cc4c(cc3c2)oc2cc3cc(N(c5ccccc5-c5ccccc5)c5ccccc5-c5ccccc5)ccc3cc24)cc1 ; CC(C)(C)c1ccc(-c2ccccc2N(c2ccccc2)c2ccc3cc4c(cc3c2)oc2cc3cc(N(c5cccc(-c6ccccc6)c5)c5ccccc5-c5ccc(C(C)(C)C)cc5)ccc3cc24)cc1]

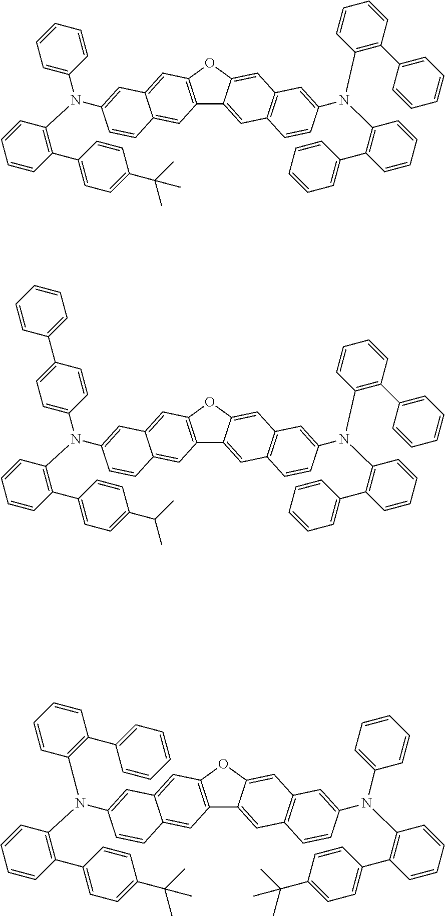 [SMILES: CC(C)(C)c1ccc(-c2ccccc2N(c2ccccc2)c2ccc3cc4c(cc3c2)oc2cc3cc(N(c5ccccc5-c5ccccc5)c5ccccc5-c5ccccc5)ccc3cc24)cc1 ; CC(C)c1ccc(-c2ccccc2N(c2ccc(-c3ccccc3)cc2)c2ccc3cc4c(cc3c2)oc2cc3cc(N(c5ccccc5-c5ccccc5)c5ccccc5-c5ccccc5)ccc3cc24)cc1 ; CC(C)(C)c1ccc(-c2ccccc2N(c2ccccc2)c2ccc3cc4c(cc3c2)oc2cc3cc(N(c5ccccc5-c5ccccc5)c5ccccc5-c5ccc(C(C)(C)C)cc5)ccc3cc24)cc1]

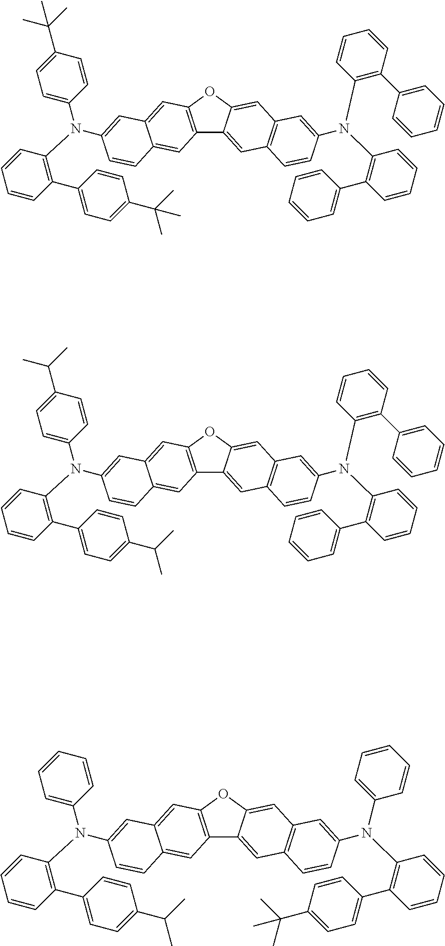 [SMILES: CC(C)(C)c1ccc(-c2ccccc2N(c2ccc(C(C)(C)C)cc2)c2ccc3cc4c(cc3c2)oc2cc3cc(N(c5ccccc5-c5ccccc5)c5ccccc5-c5ccccc5)ccc3cc24)cc1 ; CC(C)c1ccc(-c2ccccc2N(c2ccc(C(C)C)cc2)c2ccc3cc4c(cc3c2)oc2cc3cc(N(c5ccccc5-c5ccccc5)c5ccccc5-c5ccccc5)ccc3cc24)cc1 ; CC(C)c1ccc(-c2ccccc2N(c2ccccc2)c2ccc3cc4c(cc3c2)oc2cc3cc(N(c5ccccc5)c5ccccc5-c5ccc(C(C)(C)C)cc5)ccc3cc24)cc1]

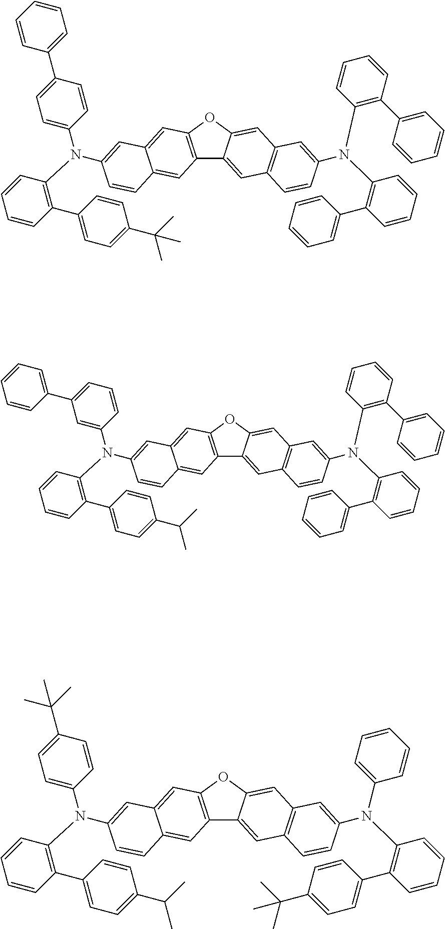 [SMILES: CC(C)(C)c1ccc(-c2ccccc2N(c2ccc(-c3ccccc3)cc2)c2ccc3cc4c(cc3c2)oc2cc3cc(N(c5ccccc5-c5ccccc5)c5ccccc5-c5ccccc5)ccc3cc24)cc1 ; CC(C)c1ccc(-c2ccccc2N(c2cccc(-c3ccccc3)c2)c2ccc3cc4c(cc3c2)oc2cc3cc(N(c5ccccc5-c5ccccc5)c5ccccc5-c5ccccc5)ccc3cc24)cc1 ; CC(C)c1ccc(-c2ccccc2N(c2ccc(C(C)(C)C)cc2)c2ccc3cc4c(cc3c2)oc2cc3cc(N(c5ccccc5)c5ccccc5-c5ccc(C(C)(C)C)cc5)ccc3cc24)cc1]

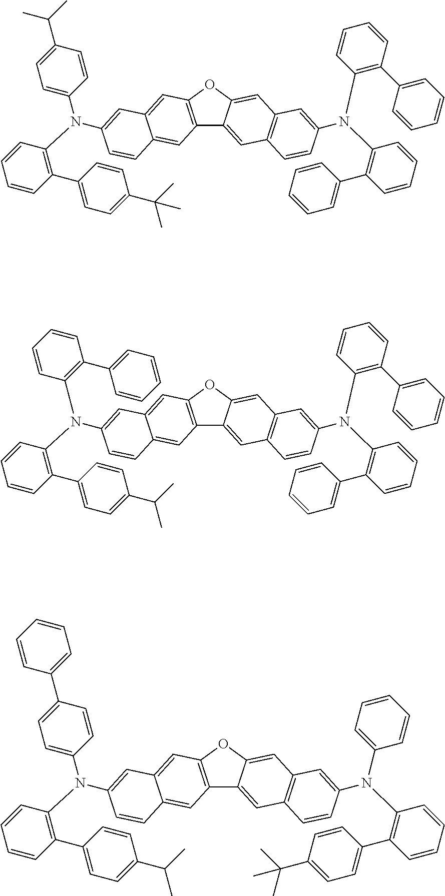 [SMILES: CC(C)c1ccc(N(c2ccc3cc4c(cc3c2)oc2cc3cc(N(c5ccccc5-c5ccccc5)c5ccccc5-c5ccccc5)ccc3cc24)c2ccccc2-c2ccc(C(C)(C)C)cc2)cc1 ; CC(C)c1ccc(-c2ccccc2N(c2ccc3cc4c(cc3c2)oc2cc3cc(N(c5ccccc5-c5ccccc5)c5ccccc5-c5ccccc5)ccc3cc24)c2ccccc2-c2ccccc2)cc1 ; CC(C)c1ccc(-c2ccccc2N(c2ccc(-c3ccccc3)cc2)c2ccc3cc4c(cc3c2)oc2cc3cc(N(c5ccccc5)c5ccccc5-c5ccc(C(C)(C)C)cc5)ccc3cc24)cc1]

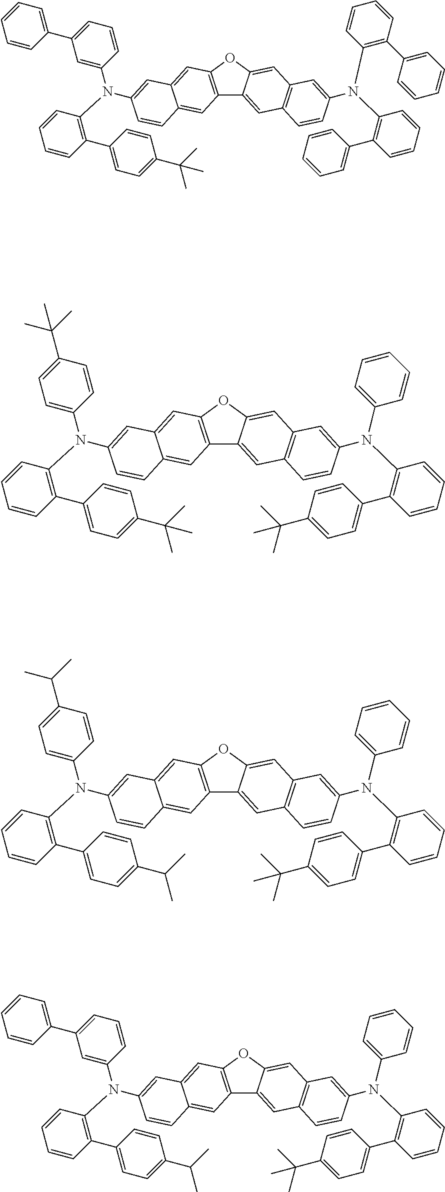 [SMILES: CC(C)(C)c1ccc(-c2ccccc2N(c2cccc(-c3ccccc3)c2)c2ccc3cc4c(cc3c2)oc2cc3cc(N(c5ccccc5-c5ccccc5)c5ccccc5-c5ccccc5)ccc3cc24)cc1 ; CC(C)(C)c1ccc(-c2ccccc2N(c2ccccc2)c2ccc3cc4c(cc3c2)oc2cc3cc(N(c5ccc(C(C)(C)C)cc5)c5ccccc5-c5ccc(C(C)(C)C)cc5)ccc3cc24)cc1 ; CC(C)c1ccc(-c2ccccc2N(c2ccc(C(C)C)cc2)c2ccc3cc4c(cc3c2)oc2cc3cc(N(c5ccccc5)c5ccccc5-c5ccc(C(C)(C)C)cc5)ccc3cc24)cc1 ; CC(C)c1ccc(-c2ccccc2N(c2cccc(-c3ccccc3)c2)c2ccc3cc4c(cc3c2)oc2cc3cc(N(c5ccccc5)c5ccccc5-c5ccc(C(C)(C)C)cc5)ccc3cc24)cc1]

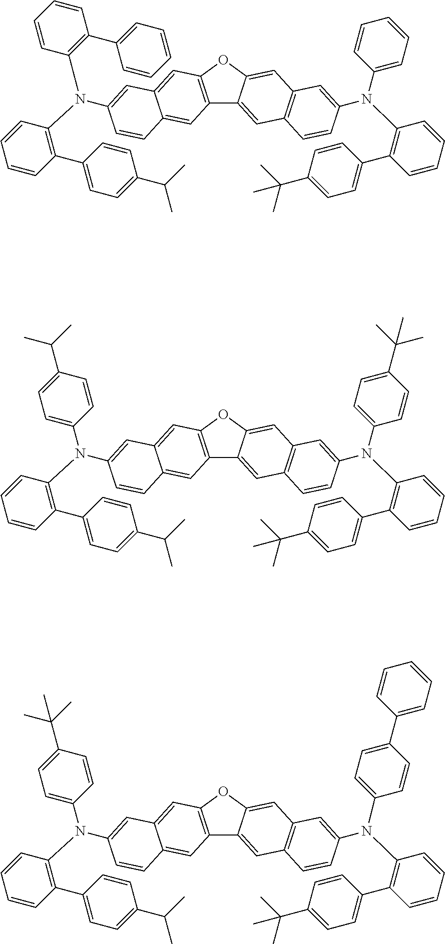 [SMILES: CC(C)c1ccc(-c2ccccc2N(c2ccc3cc4c(cc3c2)oc2cc3cc(N(c5ccccc5)c5ccccc5-c5ccc(C(C)(C)C)cc5)ccc3cc24)c2ccccc2-c2ccccc2)cc1 ; CC(C)c1ccc(-c2ccccc2N(c2ccc(C(C)C)cc2)c2ccc3cc4c(cc3c2)oc2cc3cc(N(c5ccc(C(C)(C)C)cc5)c5ccccc5-c5ccc(C(C)(C)C)cc5)ccc3cc24)cc1 ; CC(C)c1ccc(-c2ccccc2N(c2ccc(C(C)(C)C)cc2)c2ccc3cc4c(cc3c2)oc2cc3cc(N(c5ccc(-c6ccccc6)cc5)c5ccccc5-c5ccc(C(C)(C)C)cc5)ccc3cc24)cc1]

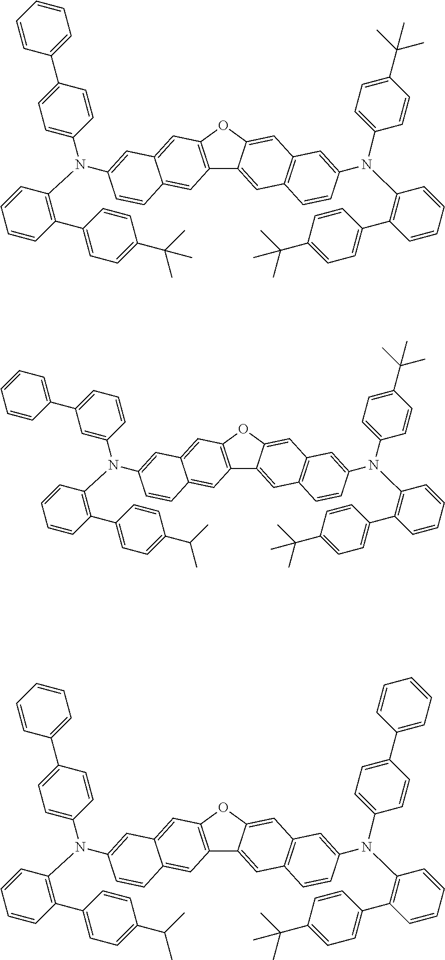 [SMILES: CC(C)(C)c1ccc(-c2ccccc2N(c2ccc(-c3ccccc3)cc2)c2ccc3cc4c(cc3c2)oc2cc3cc(N(c5ccc(C(C)(C)C)cc5)c5ccccc5-c5ccc(C(C)(C)C)cc5)ccc3cc24)cc1 ; CC(C)c1ccc(-c2ccccc2N(c2cccc(-c3ccccc3)c2)c2ccc3cc4c(cc3c2)oc2cc3cc(N(c5ccc(C(C)(C)C)cc5)c5ccccc5-c5ccc(C(C)(C)C)cc5)ccc3cc24)cc1 ; CC(C)c1ccc(-c2ccccc2N(c2ccc(-c3ccccc3)cc2)c2ccc3cc4c(cc3c2)oc2cc3cc(N(c5ccc(-c6ccccc6)cc5)c5ccccc5-c5ccc(C(C)(C)C)cc5)ccc3cc24)cc1]

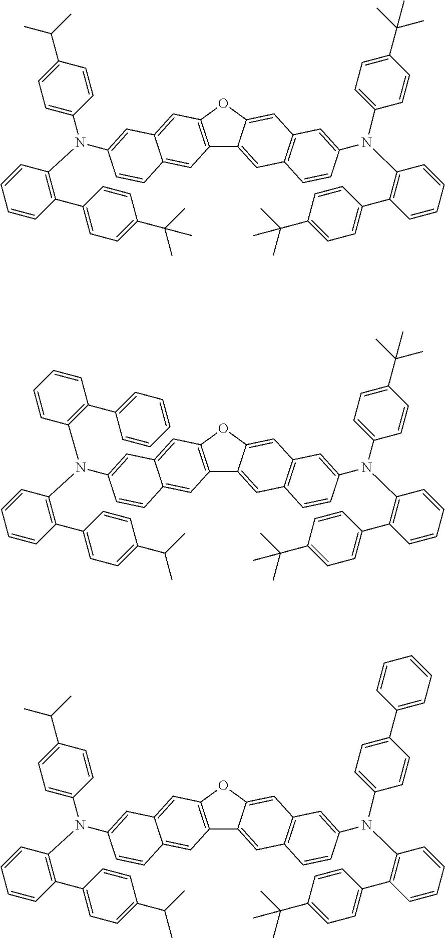 [SMILES: CC(C)c1ccc(N(c2ccc3cc4c(cc3c2)oc2cc3cc(N(c5ccc(C(C)(C)C)cc5)c5ccccc5-c5ccc(C(C)(C)C)cc5)ccc3cc24)c2ccccc2-c2ccc(C(C)(C)C)cc2)cc1 ; CC(C)c1ccc(-c2ccccc2N(c2ccc3cc4c(cc3c2)oc2cc3cc(N(c5ccc(C(C)(C)C)cc5)c5ccccc5-c5ccc(C(C)(C)C)cc5)ccc3cc24)c2ccccc2-c2ccccc2)cc1 ; CC(C)c1ccc(-c2ccccc2N(c2ccc(C(C)C)cc2)c2ccc3cc4c(cc3c2)oc2cc3cc(N(c5ccc(-c6ccccc6)cc5)c5ccccc5-c5ccc(C(C)(C)C)cc5)ccc3cc24)cc1]

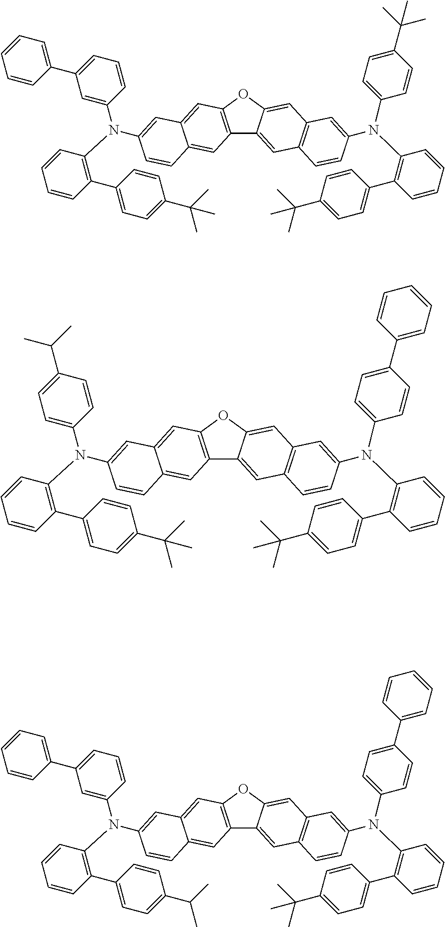 [SMILES: CC(C)(C)c1ccc(-c2ccccc2N(c2ccc(C(C)(C)C)cc2)c2ccc3cc4c(cc3c2)oc2cc3cc(N(c5cccc(-c6ccccc6)c5)c5ccccc5-c5ccc(C(C)(C)C)cc5)ccc3cc24)cc1 ; CC(C)c1ccc(N(c2ccc3cc4c(cc3c2)oc2cc3cc(N(c5ccc(-c6ccccc6)cc5)c5ccccc5-c5ccc(C(C)(C)C)cc5)ccc3cc24)c2ccccc2-c2ccc(C(C)(C)C)cc2)cc1 ; CC(C)c1ccc(-c2ccccc2N(c2cccc(-c3ccccc3)c2)c2ccc3cc4c(cc3c2)oc2cc3cc(N(c5ccc(-c6ccccc6)cc5)c5ccccc5-c5ccc(C(C)(C)C)cc5)ccc3cc24)cc1]

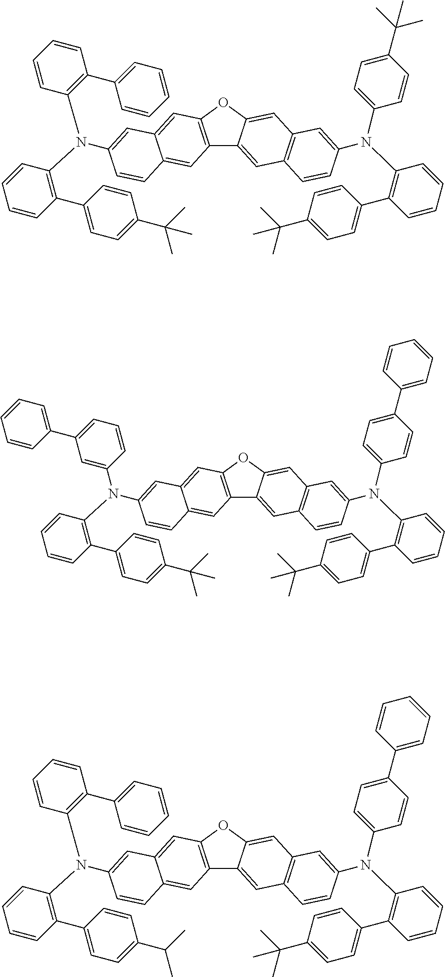 [SMILES: CC(C)(C)c1ccc(-c2ccccc2N(c2ccc(C(C)(C)C)cc2)c2ccc3cc4c(cc3c2)oc2cc3cc(N(c5ccccc5-c5ccccc5)c5ccccc5-c5ccc(C(C)(C)C)cc5)ccc3cc24)cc1 ; CC(C)(C)c1ccc(-c2ccccc2N(c2ccc(-c3ccccc3)cc2)c2ccc3cc4c(cc3c2)oc2cc3cc(N(c5cccc(-c6ccccc6)c5)c5ccccc5-c5ccc(C(C)(C)C)cc5)ccc3cc24)cc1 ; CC(C)c1ccc(-c2ccccc2N(c2ccc3cc4c(cc3c2)oc2cc3cc(N(c5ccc(-c6ccccc6)cc5)c5ccccc5-c5ccc(C(C)(C)C)cc5)ccc3cc24)c2ccccc2-c2ccccc2)cc1]

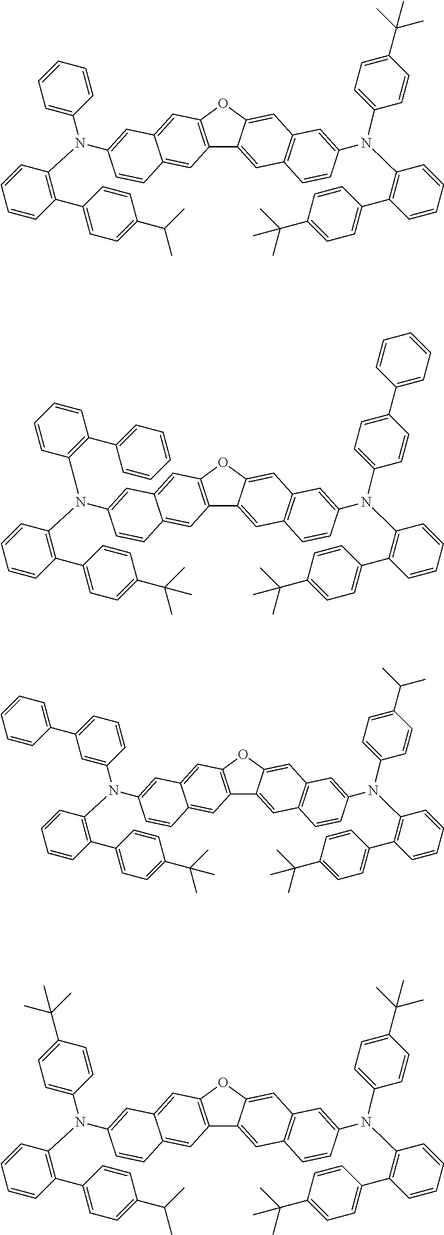

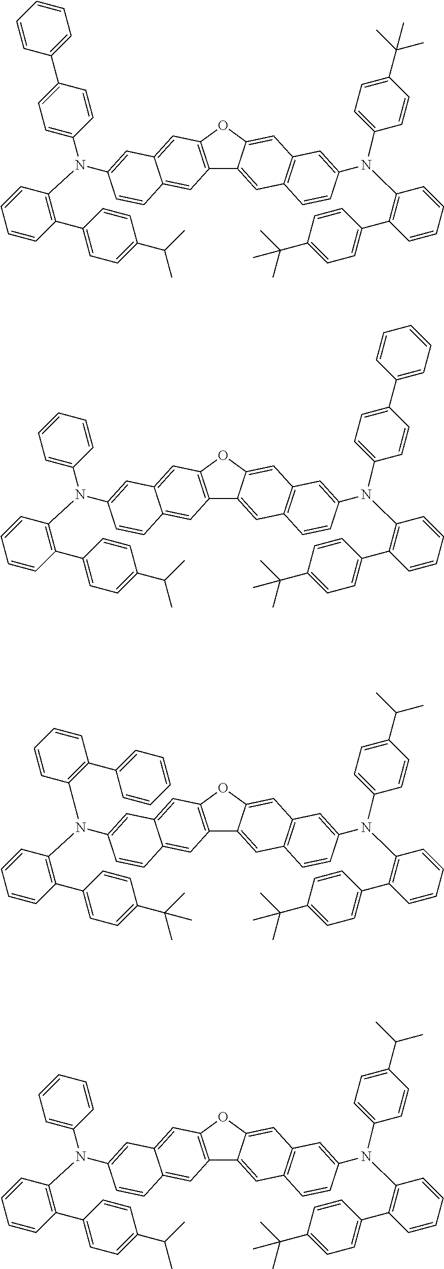 [SMILES: CC(C)c1ccc(-c2ccccc2N(c2ccc(-c3ccccc3)cc2)c2ccc3cc4c(cc3c2)oc2cc3cc(N(c5ccc(C(C)(C)C)cc5)c5ccccc5-c5ccc(C(C)(C)C)cc5)ccc3cc24)cc1 ; CC(C)c1ccc(-c2ccccc2N(c2ccccc2)c2ccc3cc4c(cc3c2)oc2cc3cc(N(c5ccc(-c6ccccc6)cc5)c5ccccc5-c5ccc(C(C)(C)C)cc5)ccc3cc24)cc1 ; CC(C)c1ccc(N(c2ccc3cc4c(cc3c2)oc2cc3cc(N(c5ccccc5-c5ccccc5)c5ccccc5-c5ccc(C(C)(C)C)cc5)ccc3cc24)c2ccccc2-c2ccc(C(C)(C)C)cc2)cc1 ; CC(C)c1ccc(-c2ccccc2N(c2ccccc2)c2ccc3cc4c(cc3c2)oc2cc3cc(N(c5ccc(C(C)C)cc5)c5ccccc5-c5ccc(C(C)(C)C)cc5)ccc3cc24)cc1]

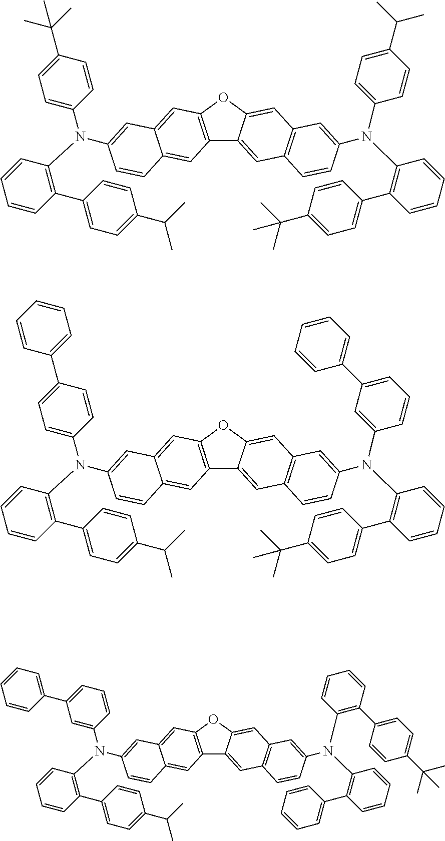 [SMILES: CC(C)c1ccc(-c2ccccc2N(c2ccc(C(C)(C)C)cc2)c2ccc3cc4c(cc3c2)oc2cc3cc(N(c5ccc(C(C)C)cc5)c5ccccc5-c5ccc(C(C)(C)C)cc5)ccc3cc24)cc1 ; CC(C)c1ccc(-c2ccccc2N(c2ccc(-c3ccccc3)cc2)c2ccc3cc4c(cc3c2)oc2cc3cc(N(c5cccc(-c6ccccc6)c5)c5ccccc5-c5ccc(C(C)(C)C)cc5)ccc3cc24)cc1 ; CC(C)c1ccc(-c2ccccc2N(c2cccc(-c3ccccc3)c2)c2ccc3cc4c(cc3c2)oc2cc3cc(N(c5ccccc5-c5ccccc5)c5ccccc5-c5ccc(C(C)(C)C)cc5)ccc3cc24)cc1]

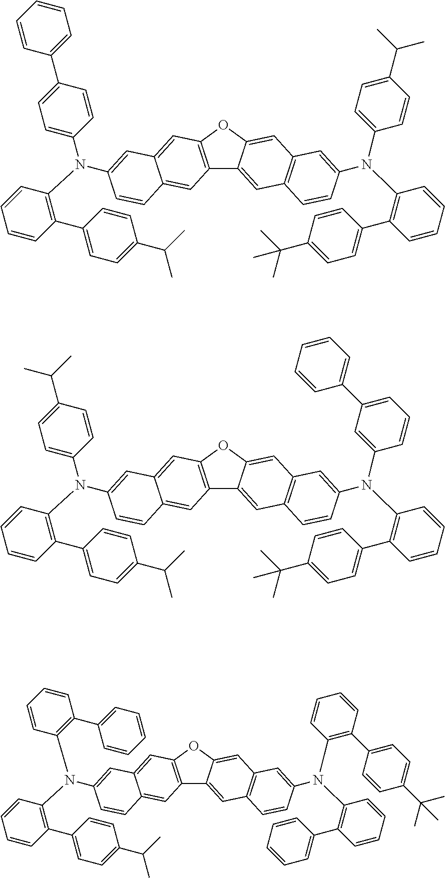 [SMILES: CC(C)c1ccc(-c2ccccc2N(c2ccc(-c3ccccc3)cc2)c2ccc3cc4c(cc3c2)oc2cc3cc(N(c5ccc(C(C)C)cc5)c5ccccc5-c5ccc(C(C)(C)C)cc5)ccc3cc24)cc1 ; CC(C)c1ccc(-c2ccccc2N(c2ccc(C(C)C)cc2)c2ccc3cc4c(cc3c2)oc2cc3cc(N(c5cccc(-c6ccccc6)c5)c5ccccc5-c5ccc(C(C)(C)C)cc5)ccc3cc24)cc1 ; CC(C)c1ccc(-c2ccccc2N(c2ccc3cc4c(cc3c2)oc2cc3cc(N(c5ccccc5-c5ccccc5)c5ccccc5-c5ccc(C(C)(C)C)cc5)ccc3cc24)c2ccccc2-c2ccccc2)cc1]

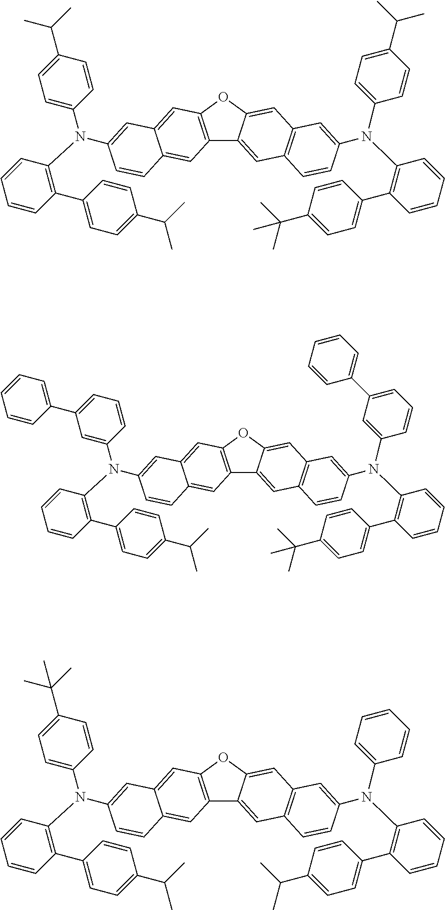 [SMILES: CC(C)c1ccc(-c2ccccc2N(c2ccc(C(C)C)cc2)c2ccc3cc4c(cc3c2)oc2cc3cc(N(c5ccc(C(C)C)cc5)c5ccccc5-c5ccc(C(C)(C)C)cc5)ccc3cc24)cc1 ; CC(C)c1ccc(-c2ccccc2N(c2cccc(-c3ccccc3)c2)c2ccc3cc4c(cc3c2)oc2cc3cc(N(c5cccc(-c6ccccc6)c5)c5ccccc5-c5ccc(C(C)(C)C)cc5)ccc3cc24)cc1 ; CC(C)c1ccc(-c2ccccc2N(c2ccccc2)c2ccc3cc4c(cc3c2)oc2cc3cc(N(c5ccc(C(C)(C)C)cc5)c5ccccc5-c5ccc(C(C)C)cc5)ccc3cc24)cc1]

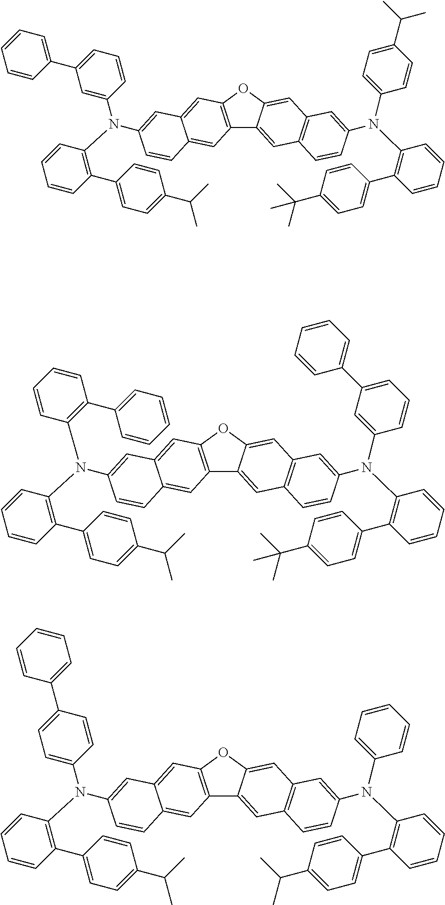 [SMILES: CC(C)c1ccc(-c2ccccc2N(c2cccc(-c3ccccc3)c2)c2ccc3cc4c(cc3c2)oc2cc3cc(N(c5ccc(C(C)C)cc5)c5ccccc5-c5ccc(C(C)(C)C)cc5)ccc3cc24)cc1 ; CC(C)c1ccc(-c2ccccc2N(c2ccc3cc4c(cc3c2)oc2cc3cc(N(c5cccc(-c6ccccc6)c5)c5ccccc5-c5ccc(C(C)(C)C)cc5)ccc3cc24)c2ccccc2-c2ccccc2)cc1 ; CC(C)c1ccc(-c2ccccc2N(c2ccccc2)c2ccc3cc4c(cc3c2)oc2cc3cc(N(c5ccc(-c6ccccc6)cc5)c5ccccc5-c5ccc(C(C)C)cc5)ccc3cc24)cc1]

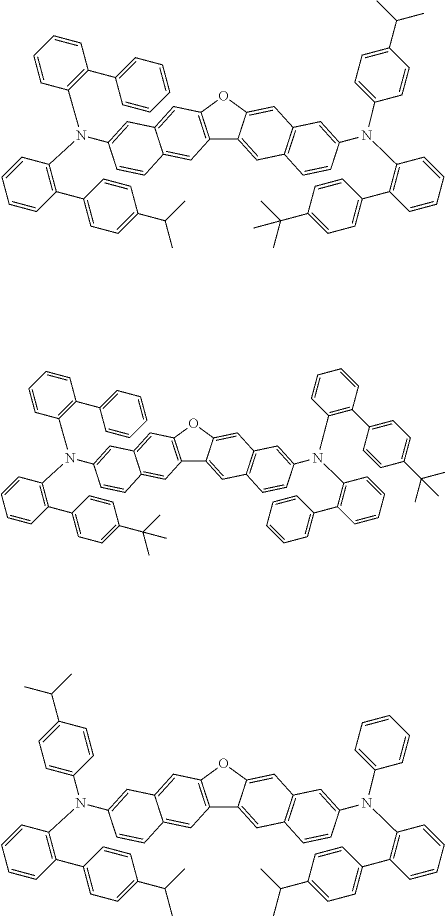 [SMILES: CC(C)c1ccc(-c2ccccc2N(c2ccc3cc4c(cc3c2)oc2cc3cc(N(c5ccc(C(C)C)cc5)c5ccccc5-c5ccc(C(C)(C)C)cc5)ccc3cc24)c2ccccc2-c2ccccc2)cc1 ; CC(C)(C)c1ccc(-c2ccccc2N(c2ccc3cc4c(cc3c2)oc2cc3cc(N(c5ccccc5-c5ccccc5)c5ccccc5-c5ccc(C(C)(C)C)cc5)ccc3cc24)c2ccccc2-c2ccccc2)cc1 ; CC(C)c1ccc(-c2ccccc2N(c2ccccc2)c2ccc3cc4c(cc3c2)oc2cc3cc(N(c5ccc(C(C)C)cc5)c5ccccc5-c5ccc(C(C)C)cc5)ccc3cc24)cc1]

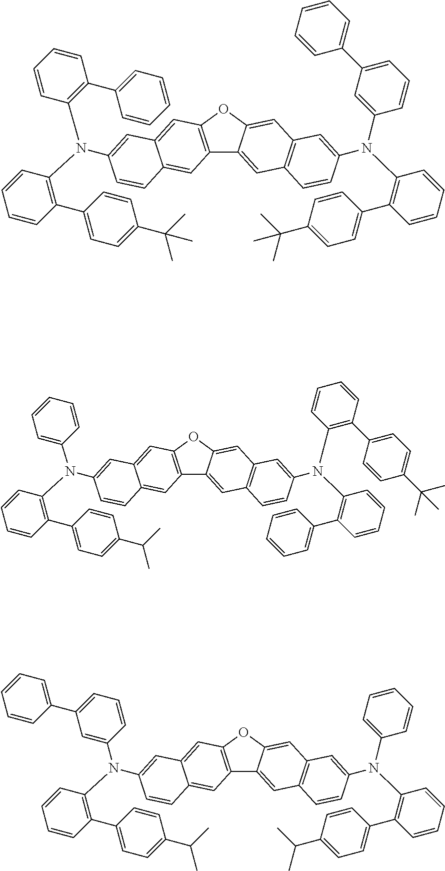 [SMILES: CC(C)(C)c1ccc(-c2ccccc2N(c2cccc(-c3ccccc3)c2)c2ccc3cc4c(cc3c2)oc2cc3cc(N(c5ccccc5-c5ccccc5)c5ccccc5-c5ccc(C(C)(C)C)cc5)ccc3cc24)cc1 ; CC(C)c1ccc(-c2ccccc2N(c2ccccc2)c2ccc3cc4c(cc3c2)oc2cc3cc(N(c5ccccc5-c5ccccc5)c5ccccc5-c5ccc(C(C)(C)C)cc5)ccc3cc24)cc1 ; CC(C)c1ccc(-c2ccccc2N(c2ccccc2)c2ccc3cc4c(cc3c2)oc2cc3cc(N(c5cccc(-c6ccccc6)c5)c5ccccc5-c5ccc(C(C)C)cc5)ccc3cc24)cc1]

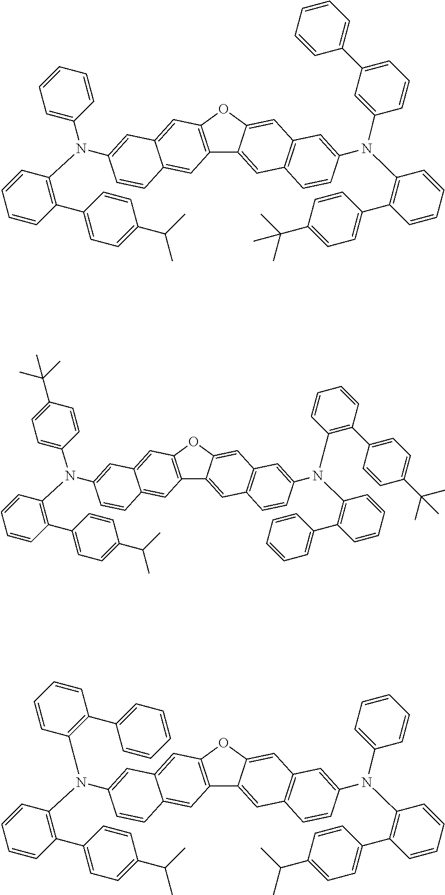 [SMILES: CC(C)c1ccc(-c2ccccc2N(c2ccccc2)c2ccc3cc4c(cc3c2)oc2cc3cc(N(c5cccc(-c6ccccc6)c5)c5ccccc5-c5ccc(C(C)(C)C)cc5)ccc3cc24)cc1 ; CC(C)c1ccc(-c2ccccc2N(c2ccc(C(C)(C)C)cc2)c2ccc3cc4c(cc3c2)oc2cc3cc(N(c5ccccc5-c5ccccc5)c5ccccc5-c5ccc(C(C)(C)C)cc5)ccc3cc24)cc1 ; CC(C)c1ccc(-c2ccccc2N(c2ccccc2)c2ccc3cc4c(cc3c2)oc2cc3cc(N(c5ccccc5-c5ccccc5)c5ccccc5-c5ccc(C(C)C)cc5)ccc3cc24)cc1]

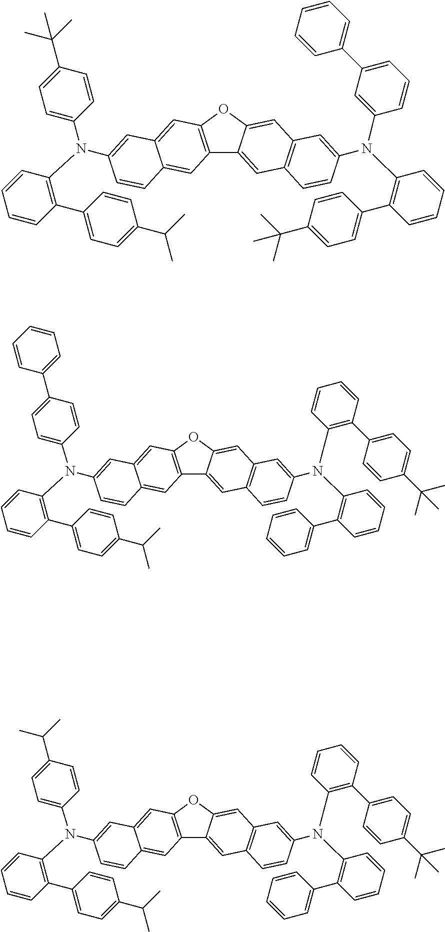 [SMILES: CC(C)c1ccc(-c2ccccc2N(c2ccc(C(C)(C)C)cc2)c2ccc3cc4c(cc3c2)oc2cc3cc(N(c5cccc(-c6ccccc6)c5)c5ccccc5-c5ccc(C(C)(C)C)cc5)ccc3cc24)cc1 ; CC(C)c1ccc(-c2ccccc2N(c2ccc(-c3ccccc3)cc2)c2ccc3cc4c(cc3c2)oc2cc3cc(N(c5ccccc5-c5ccccc5)c5ccccc5-c5ccc(C(C)(C)C)cc5)ccc3cc24)cc1 ; CC(C)c1ccc(-c2ccccc2N(c2ccc(C(C)C)cc2)c2ccc3cc4c(cc3c2)oc2cc3cc(N(c5ccccc5-c5ccccc5)c5ccccc5-c5ccc(C(C)(C)C)cc5)ccc3cc24)cc1]

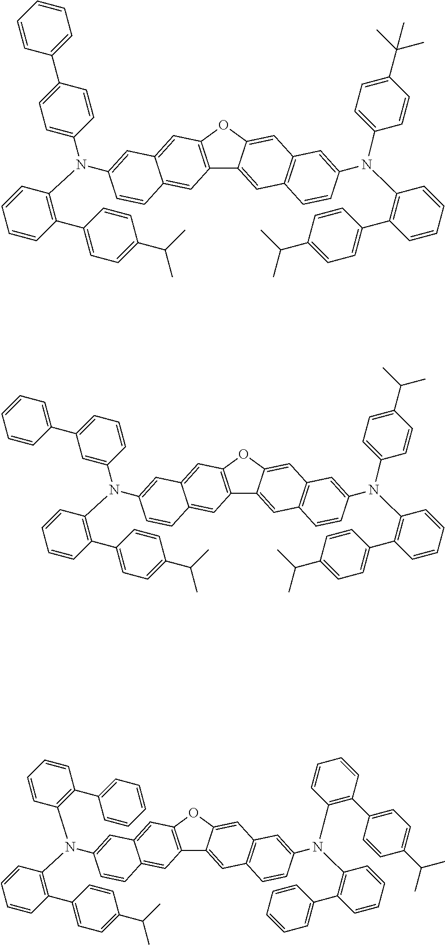 [SMILES: CC(C)c1ccc(-c2ccccc2N(c2ccc(-c3ccccc3)cc2)c2ccc3cc4c(cc3c2)oc2cc3cc(N(c5ccc(C(C)(C)C)cc5)c5ccccc5-c5ccc(C(C)C)cc5)ccc3cc24)cc1 ; CC(C)c1ccc(-c2ccccc2N(c2ccc(C(C)C)cc2)c2ccc3cc4c(cc3c2)oc2cc3cc(N(c5cccc(-c6ccccc6)c5)c5ccccc5-c5ccc(C(C)C)cc5)ccc3cc24)cc1 ; CC(C)c1ccc(-c2ccccc2N(c2ccc3cc4c(cc3c2)oc2cc3cc(N(c5ccccc5-c5ccccc5)c5ccccc5-c5ccc(C(C)C)cc5)ccc3cc24)c2ccccc2-c2ccccc2)cc1]

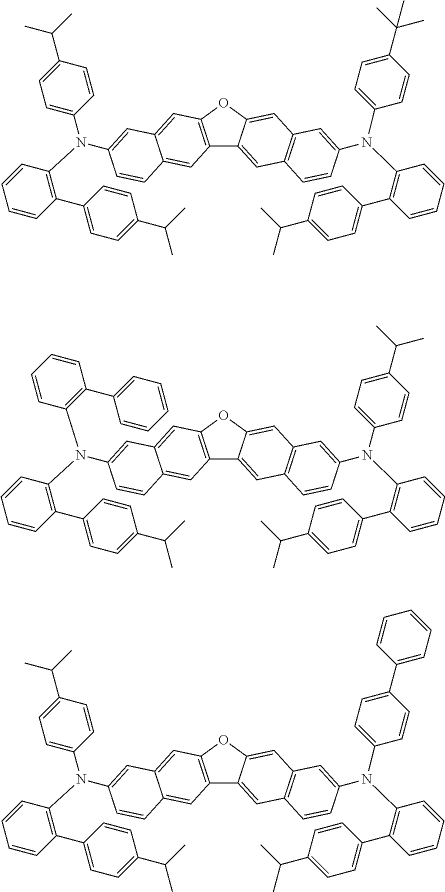 [SMILES: CC(C)c1ccc(-c2ccccc2N(c2ccc(C(C)C)cc2)c2ccc3cc4c(cc3c2)oc2cc3cc(N(c5ccc(C(C)(C)C)cc5)c5ccccc5-c5ccc(C(C)C)cc5)ccc3cc24)cc1 ; CC(C)c1ccc(-c2ccccc2N(c2ccc(C(C)C)cc2)c2ccc3cc4c(cc3c2)oc2cc3cc(N(c5ccccc5-c5ccccc5)c5ccccc5-c5ccc(C(C)C)cc5)ccc3cc24)cc1 ; CC(C)c1ccc(-c2ccccc2N(c2ccc(-c3ccccc3)cc2)c2ccc3cc4c(cc3c2)oc2cc3cc(N(c5ccc(C(C)C)cc5)c5ccccc5-c5ccc(C(C)C)cc5)ccc3cc24)cc1]

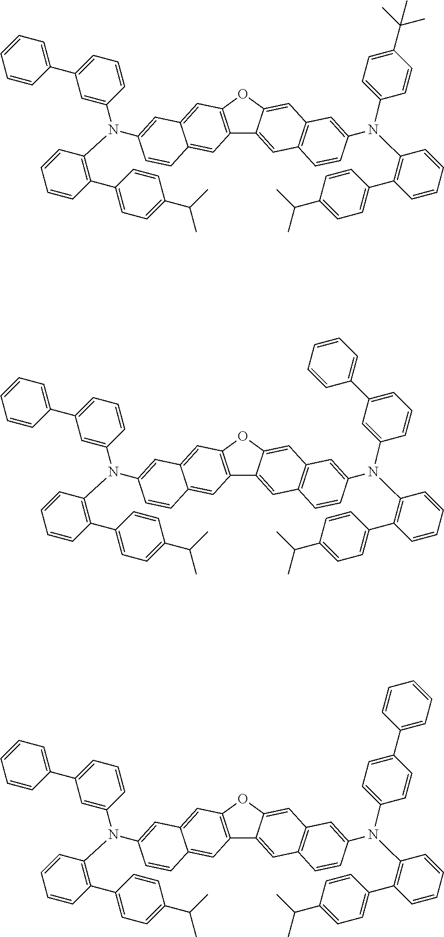 [SMILES: CC(C)c1ccc(-c2ccccc2N(c2ccc(C(C)(C)C)cc2)c2ccc3cc4c(cc3c2)oc2cc3cc(N(c5cccc(-c6ccccc6)c5)c5ccccc5-c5ccc(C(C)C)cc5)ccc3cc24)cc1 ; CC(C)c1ccc(-c2ccccc2N(c2cccc(-c3ccccc3)c2)c2ccc3cc4c(cc3c2)oc2cc3cc(N(c5cccc(-c6ccccc6)c5)c5ccccc5-c5ccc(C(C)C)cc5)ccc3cc24)cc1 ; CC(C)c1ccc(-c2ccccc2N(c2ccc(-c3ccccc3)cc2)c2ccc3cc4c(cc3c2)oc2cc3cc(N(c5cccc(-c6ccccc6)c5)c5ccccc5-c5ccc(C(C)C)cc5)ccc3cc24)cc1]

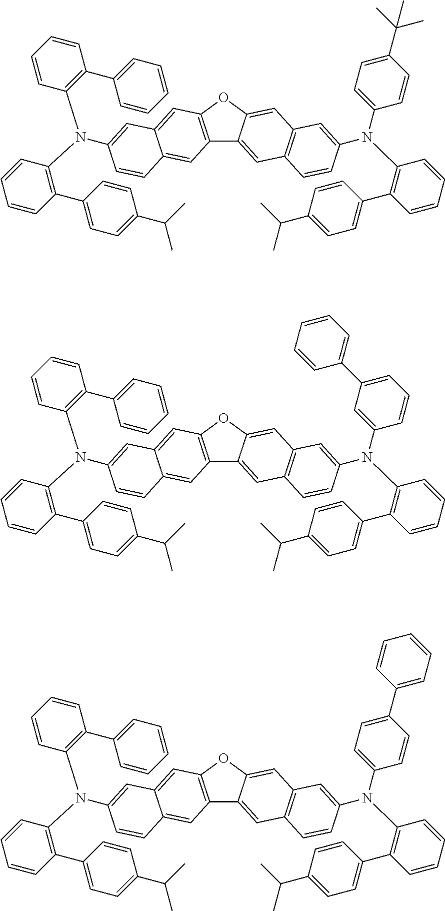 [SMILES: CC(C)c1ccc(-c2ccccc2N(c2ccc(C(C)(C)C)cc2)c2ccc3cc4c(cc3c2)oc2cc3cc(N(c5ccccc5-c5ccccc5)c5ccccc5-c5ccc(C(C)C)cc5)ccc3cc24)cc1 ; CC(C)c1ccc(-c2ccccc2N(c2cccc(-c3ccccc3)c2)c2ccc3cc4c(cc3c2)oc2cc3cc(N(c5ccccc5-c5ccccc5)c5ccccc5-c5ccc(C(C)C)cc5)ccc3cc24)cc1 ; CC(C)c1ccc(-c2ccccc2N(c2ccc(-c3ccccc3)cc2)c2ccc3cc4c(cc3c2)oc2cc3cc(N(c5ccccc5-c5ccccc5)c5ccccc5-c5ccc(C(C)C)cc5)ccc3cc24)cc1]

### [Material for Organic EL Device]

The compound according to an aspect of the present invention is useful as a material as an organic EL device, is useful as a material for a emitting layer of an organic EL device, and is particularly useful as a dopant material for a emitting layer.

When the compound according to an aspect of the present invention is used for a emitting layer of an organic EL device, an organic EL device having a long lifetime can be obtained.

### [Organic EL Device]

An organic EL device according to an aspect of the present invention includes a cathode; an anode; and at least one organic layer arranged between the cathode and the anode, wherein at least one layer of the at least one organic layer includes the compound represented by the formula (1).

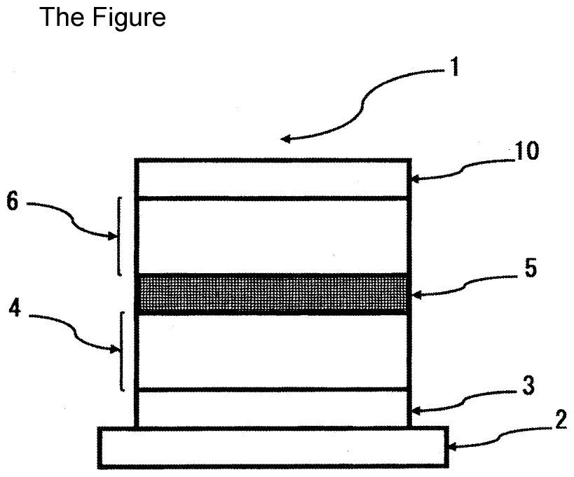

A schematic configuration of the organic EL device according to an aspect of the present invention will be described with reference to FIG. 1.

The organic EL device 1 according to an aspect of the present invention includes a substrate 2, an anode 3, an emitting layer 5 as an organic layer, a cathode 10, an organic layer 4 between the anode 3 and the emitting layer 5, and an organic layer 6 between the emitting layer 5 and the cathode 10.

Each of the organic layer 4 and the organic layer 6 may be a single layer or may composed of a plurality of layers.

Further, the organic layer 4 may include a hole-transporting region. The hole-transporting region may include a hole-injecting layer, a hole-transporting layer, an electron-barrier layer and the like. The organic layer 6 may include an electron-transporting region. The electron-transporting region may include an electron-injecting layer, an electron-transporting layer, a hole-barrier layer and the like.

The compound represented by the formula (1) is contained in the organic layer 4, the emitting layer 5 or the organic layer 6. In one embodiment, the compound represented by the formula (1) is included in the emitting layer 5. The compound represented by the formula (1) can function as a dopant material in the emitting layer 5.

In the organic EL device according to an aspect of the present invention, the at least one layer of the at least one organic layer includes a first compound and a second compound, and the first compound is the compound represented by the formula (1).

In the organic EL device according to an aspect of the present invention, the second compound is a heterocyclic compound or a fused aromatic compound.

In the organic EL device according to an aspect of the present invention, the second compound is an anthracene derivative.

In the organic EL device according to an aspect of the present invention, the second compound is a compound represented by the following formula (10).

### <Compound Represented by the Formula (10)>

A compound represented by formula (10) will be described.

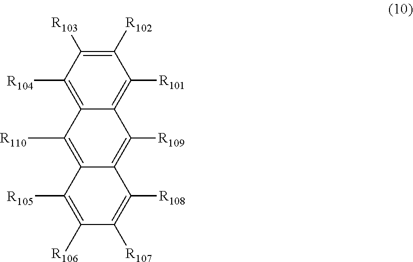

In the formula (10),

one or more sets of the adjacent two or more of R₁₀₁ to R₁₁₀ form a substituted or unsubstituted, saturated or unsaturated ring, or do not form the substituted or unsubstituted, saturated or unsaturated ring;

R₁₀₁ to R₁₁₀ which do not form the substituted or unsubstituted, saturated or unsaturated ring are independently

a hydrogen atom,

a substituent R, or

a group represented by the following formula (11):

-L₁₀₁-Ar₁₀₁ (11).

In the formula (11),

L₁₀₁ is

a single bond,

a substituted or unsubstituted arylene group having 6 to 50 ring carbon atoms, or

a substituted or unsubstituted divalent heterocyclic group having 5 to 50 ring atoms;

Ar₁₀₁ is

a substituted or unsubstituted aryl group having 6 to 50 ring carbon atoms, or

a substituted or unsubstituted monovalent heterocyclic group having 5 to 50 ring atoms;

the substituent R is

a substituted or unsubstituted alkyl group having 1 to 50 carbon atoms,

a substituted or unsubstituted alkenyl group having 2 to 50 carbon atoms,

a substituted or unsubstituted alkynyl group having 2 to 50 carbon atoms,

a substituted or unsubstituted cycloalkyl group having 3 to 50 ring carbon atoms,

—Si(R₉₀₁)(R₉₀₂)(R₉₀₃),

—O—(R₉₀₄),

—S—(R₉₀₅),

—N(R₉₀₆)(R₉₀₇),

a halogen atom, a cyano group, a nitro group,

a substituted or unsubstituted aryl group having 6 to 50 ring carbon atoms, or

a substituted or unsubstituted monovalent heterocyclic group having 5 to 50 ring atoms;

when two or more substituents R are present, the two or more substituents R may be the same as or different from each other;

R₉₀₁ to R₉₀₇ are independently

a hydrogen atom,

a substituted or unsubstituted alkyl group having 1 to 50 carbon atoms,

a substituted or unsubstituted cycloalkyl group having 3 to 50 ring carbon atoms,

a substituted or unsubstituted aryl group having 6 to 50 ring carbon atoms, or

a substituted or unsubstituted monovalent heterocyclic group having 5 to 50 ring atoms.

when two or more of each of R₉₀₁ to R₉₀₇ are present, the two or more of each of R₉₀₁ to R₉₀₇ may be the same as or different from each other;

here, at least one of R₁₀₁ to R₁₁₀ which does not form the substituted or unsubstituted, saturated or unsaturated ring is the group represented by the formula (11); when two or more groups represented by the formula (11) are present, each of the two or more groups represented by the formula (11) may be the same as or different from each other.

The compound represented by the formula (10) may have a deuterium atom as a hydrogen atom.

In one embodiment, at least one of Ar₁₀₁ in the formula (10) is a substituted or unsubstituted aryl group having 6 to 50 ring carbon atoms.

In one embodiment, at least one of Ar₁₀₁ in the formula (10) is a substituted or unsubstituted monovalent heterocyclic group having 5 to 50 ring atoms.

In one embodiment, all of Ar₁₀₁ in the formula (10) are a substituted or unsubstituted aryl group having 6 to 50 ring carbon atoms. The plurality of Ar₁₀₁'s may be the same as or different from each other.

In one embodiment, one of Ar₁₀₁ in the formula (10) is a substituted or unsubstituted monovalent heterocyclic group having 5 to 50 ring atoms, and the remaining Ar₁₀₁ is a substituted or unsubstituted aryl group having 6 to 50 ring carbon atoms. The plurality of Ar₁₀₁'s may be the same as or different from each other.

In one embodiment, at least one of L₁₀₁ in the formula 10 is a single bond.

In one embodiment, all of L₁₀₁ in the formula (10) are single bonds.

In one embodiment, at least one of L₁₀₁ in the formula (10) is a substituted or unsubstituted arylene group having 6 to 50 ring carbon atoms.

In one embodiment, at least one of L₁₀₁ in the formula (10) is a substituted or unsubstituted phenylene group, or a substituted or unsubstituted naphthyl group.

In one embodiment, the group represented by -L₁₀₁-Ar₁₀₁ in the formula (10) is selected from the group consisting of

a substituted or unsubstituted phenyl group,

a substituted or unsubstituted naphthyl group,

a substituted or unsubstituted biphenyl group,

a substituted or unsubstituted phenanthrenyl group,

a substituted or unsubstituted benzophenanthrenyl group,

a substituted or unsubstituted fluorenyl group,

a substituted or unsubstituted benzofluorenyl group,

a substituted or unsubstituted dibenzofuranyl group,

a substituted or unsubstituted naphthobenzofuranyl group,

a substituted or unsubstituted dibenzothiophenyl group, and

a substituted or unsubstituted carbazolyl group.

In one embodiment, the substituent R in the formula (10) are independently a substituted or unsubstituted alkyl group having 1 to 50 carbon atoms, a substituted or unsubstituted cycloalkyl group having 3 to 50 ring carbon atoms,

—Si(R₉₀₁)(R₉₀₂)(R₉₀₃),

—O—(R₉₀₄),

—S—(R₉₀₅),

—N(R₉₀₆)(R₉₀₇),

a halogen atom, a cyano group, a nitro group, or

a substituted or unsubstituted aryl group having 6 to 50 ring carbon atoms.

R₉₀₁ to R₉₀₇ are the same as defined in the formula (10).

In one embodiment, the substituent of “substituted or unsubstituted” in the formula (10) is independently

a substituted or unsubstituted alkyl group having 1 to 50 carbon atoms,

a substituted or unsubstituted alkenyl group having 2 to 50 carbon atoms,

a substituted or unsubstituted alkynyl group having 2 to 50 carbon atoms,

a substituted or unsubstituted cycloalkyl group having 3 to 50 ring carbon atoms,

—Si(R₉₀₁)(R₉₀₂)(R₉₀₃),

—O—(R₉₀₄),

—S—(R₉₀₅),

—N(R₉₀₆)(R₉₀₇),

a halogen atom, a cyano group, a nitro group,

a substituted or unsubstituted aryl group having 6 to 50 ring carbon atoms, or

a substituted or unsubstituted monovalent heterocyclic group having 5 to 50 ring atoms.

R₉₀₁ to R₉₀₇ are the same as defined in the formula (10).

In one embodiment, the substituent of “substituted or unsubstituted” in the formula (10) is independently

a substituted or unsubstituted alkyl group having 1 to 50 carbon atoms,

a substituted or unsubstituted cycloalkyl group having 3 to 50 ring carbon atoms,

—Si(R₉₀₁)(R₉₀₂)(R₉₀₃),

—O—(R₉₀₄),

—S—(R₉₀₅),

—N(R₉₀₆)(R₉₀₇),

a halogen atom, a cyano group, a nitro group, or

a substituted or unsubstituted aryl group having 6 to 50 ring carbon atoms.

R₉₀₁ to R₉₀₇ are the same as defined in the formula (10).

In one embodiment, the substituent in the case of “substituted or unsubstituted” in the formula (10) is selected from the group consisting of

an alkyl group having 1 to 18 carbon atoms,

an aryl group having 6 to 18 ring carbon atoms, and

a monovalent heterocyclic group having 5 to 18 ring atoms.

In one embodiment, the substituent in the case of “substituted or unsubstituted” in the formula (10) is an alkyl group having 1 to 5 carbon atoms.

In one embodiment, the compound represented by the formula (10) is a compound represented by the following formula (20).

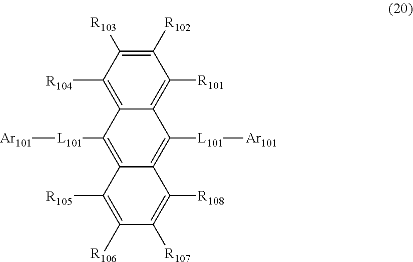

In the formula (20), R₁₀₁ to R₁₀₈, L₁₀₁'s and Ar₁₀₁'s are the same as defined in the formula (10).

The compound represented by the formula (20) may have a deuterium atom as a hydrogen atom.

That is, in one embodiment, the compound represented by the formula (10) or the formula (20) has at least two groups represented by the formula (11).

In one embodiment, the compound represented by the formula (10) or the formula (20) has two or three groups represented by the formula (11).

In one embodiment, R₁₀₁ to R₁₁₀ in formulas (10) and (20) do not form the substituted or unsubstituted, saturated or unsaturated ring.

In one embodiment, R₁₀₁ to R₁₁₀ in the formulas (10) and (20) is a hydrogen atom.

In one embodiment, the compound represented by the formula (20) is a compound represented by the following formula (30).

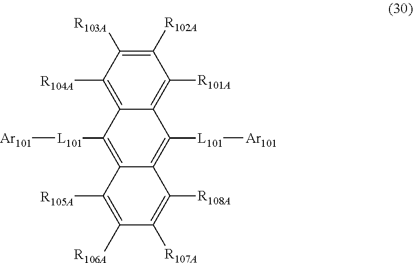

In the formula (30), L₁₀₁'s and Ar₁₀₁'s are the same as defined in the formula (10).

The adjacent two of R_{101A} to R_{108A} do not form any substituted or unsubstituted, saturated or unsaturated ring.

R_{101A} to R_{108A} are independently

a hydrogen atom, or

a substituent R.

The substituent R is the same as defined in the formula (10).

That is, the compound represented by the formula (30) is a compound having two groups represented by the formula (11).

The compound represented by the formula (30) has substantially only protium atoms as hydrogen atoms.

The expression “having substantially only protium atoms” means the case where the proportion of protium compound based on the total amount of a compound having only protium atoms as hydrogen atoms (protium compound) and a compound having a deuterium atom (deuterium compound), which have the same structure, is 90 mol % or more, 95 mol % or more, or 99 mol % or more.

In one embodiment, the compound represented by the formula (30) is a compound represented by the following formula (31).

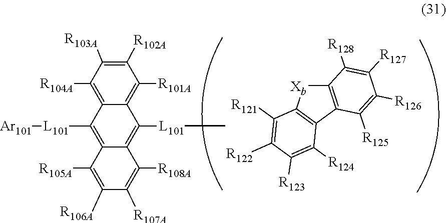

In the formula (31), L₁₀₁'s and Ar₁₀₁ are the same as defined in the formula (10).

R_{101A} to R₁₀₈ are the same as defined in the formula (30).

X_{b} is O, S, N(R₁₃₁), or C(R₁₃₂)(R₁₃₃).

One of R₁₂₁ to R₁₂₈, and R₁₃₁ to R₁₃₃ is a single bond bonding with L₁₀₁.

One or more sets of the adjacent two or more of R₁₂₁ to R₁₂₈ which are not single bonds bonding with L₁₀₁ form a substituted or unsubstituted, saturated or unsaturated ring, or do not form the substituted or unsubstituted, saturated or unsaturated ring.

R₁₂₁ to R₁₂₈ which are not single bonds bonding with L₁₀₁ and which do not form the substituted or unsubstituted, saturated or unsaturated ring are independently

a hydrogen atom, or

a substituent R.

The substituent R is the same as defined in the formula (10).

R₁₃₁ to R₁₃₃ which are not single bonds bonding with L₁₀₁ are independently a hydrogen atom,

a substituted or unsubstituted alkyl group having 1 to 50 carbon atoms,

a substituted or unsubstituted cycloalkyl group having 3 to 50 ring carbon atoms,

a substituted or unsubstituted aryl group having 6 to 50 ring carbon atoms, or

a substituted or unsubstituted monovalent heterocyclic group having 5 to 50 ring atoms.

When two or more R₁₃₁ to R₁₃₃ are present, each of the two or more R₁₃₁ to R₁₃₃ may be the same as or different from each other.

In one embodiment, the compound represented by the formula (31) is a compound represented by the following formula (32).

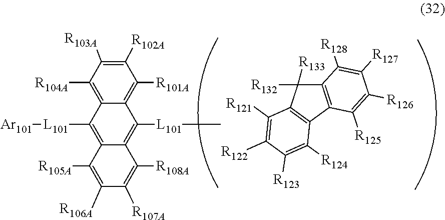

In the formula (32), R_{101A} to R_{108A}, L₁₀₁'s, Ar₁₀₁, R₁₂₁ to R₁₂₈, R₁₃₂ and R₁₃₃ are the same as defined in the formula (31).

In one embodiment, the compound represented by the formula (31) is a compound represented by the following formula (33).

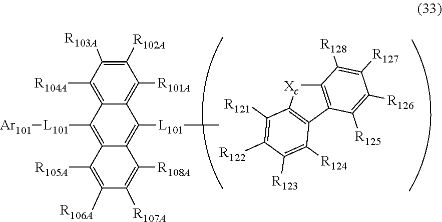

In the formula (33), R_{101A} to R_{108A}, L₁₀₁'s, Ar₁₀₁, and R₁₂₁ to R₁₂₈ are the same as defined in the formula (31).

X_{c} can be O, S, or NR₁₃₁.

R₁₃₁ is the same as defined in the formula (31).

In one embodiment, the compound represented by the formula (31) is a compound represented by the following formula (34).

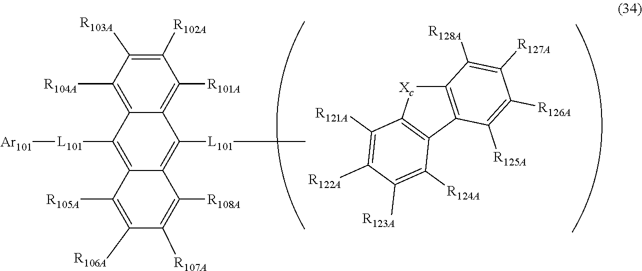

In the formula (34), R_{101A} to R_{108A}, L₁₀₁'s and Ar₁₀₁ are the same as defined in the formula (31).

X_{c} is O, S or NR₁₃₁.

R₁₃₁ is the same as defined in the formula (31).

One of R_{121A} to R_{128A} is a single bond bonding with L₁₀₁.

One or more sets of the adjacent two or more of R_{121A} to R_{128A} which are not single bonds bonding with L₁₀₁ do not form the substituted or unsubstituted, saturated or unsaturated ring.

R_{121A} to R_{128A} which are not single bonds bonding with L₁₀₁ are independently a hydrogen atom, or

a substituent R.

The substituent R is the same as defined in the formula (10).

In one embodiment, the compound represented by the formula (31) is a compound represented by the following formula (35).

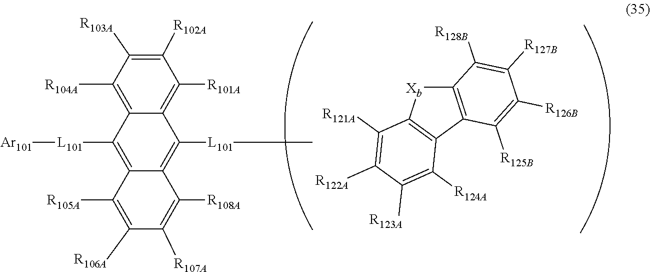

In the formula (35), R_{101A} to R_{108A}, L₁₀₁'s, Ar₁₀₁ and X_{b} are the same as defined in the formula (31).

One or more sets of the adjacent two or more of R_{121A} to R_{124A} do not form a substituted or unsubstituted, saturated or unsaturated ring by bonding with each other.

Any one set of R_{125B} and R_{126B}, R_{126B} and R_{127B}, and R_{127B} and R_{128B} forms a ring represented by the following formula (35a) or (35b) by bonding with each other.

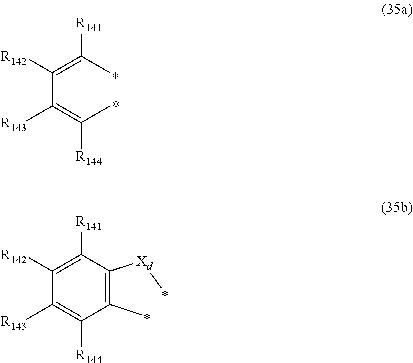

In the formulas (35a) and (35b), each of two *'s is bonded with each of any one set of R_{125B} and R_{126B}, R_{126B} and R_{127B}, and R_{127B} and R_{128B}.

R₁₄₁ to R₁₄₄ are independently

a hydrogen atom, or

a substituent R.

The substituent R is the same as defined in the formula (10).

X_{d} is O or S.

One of R_{121A} to R_{124A}, R_{125B} to R_{128B} which do not form the ring represented by the formula (35a) or (35b), and R₁₄₁ to R₁₄₄ is a single bond bonding with L₁₀₁.

R_{121A} to R_{124A} which are not single bonds bonding with L₁₀₁, and R_{125B} to R_{128B} which are not single bonds bonding with L₁₀₁ and which do not form the ring represented by the formula (35a) or (35b)

are independently

a hydrogen atom, or

a substituent R.

The substituent R is the same as defined in the formula (10).

In one embodiment, the compound represented by the formula (35) is a compound represented by the following formula (36).

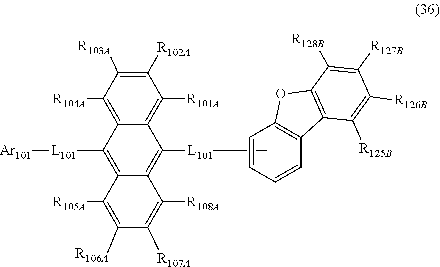

In the formula (36), R_{101A} to R_{108A}, L₁₀₁'s, Ar₁₀₁, and R_{125B} to R_{128B} are the same as defined in the formula (35).

In one embodiment, the compound represented by the formula (34) is a compound represented by the following formula (37).

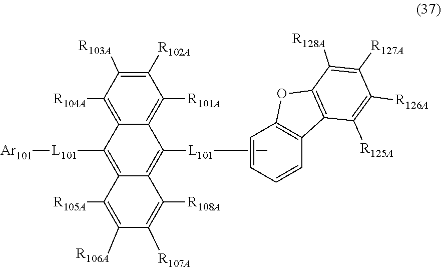

In the formula (37), R_{101A} to R_{108A}, R_{125A} to R_{128A}, L₁₀₁'s and Ar₁₀₁ are the same as defined in the formula (34).

In one embodiment, R_{101A} to R_{108A} in the formulas (30) to (37) is a hydrogen atom.

In one embodiment, the compound represented by the formula (10) is a compound represented by the following formula (40).

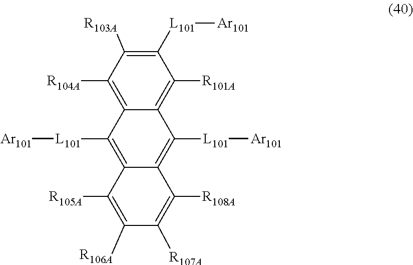

In the formula (40), L₁₀₁'s and Ar₁₀₁'s are the same as defined in the formula (10).

One or more sets of the adjacent two or more of R_{101A}, and R_{103A} to R_{108A} form a substituted or unsubstituted, saturated or unsaturated ring, or do not form the substituted or unsubstituted, saturated or unsaturated ring.

R_{101A}, and R_{103A} to R_{108A} which do not form the substituted or unsubstituted, saturated or unsaturated ring are independently a hydrogen atom, or a substituent R.

The substituent R is the same as defined in the formula (10).

That is, the compound represented by the formula (40) is a compound having three groups represented by the formula (11). Furthermore, the compound represented by the formula (40) has substantially only protium atoms as hydrogen atoms.

In one embodiment, the compound represented by the formula (40) is represented by the following formula (41).

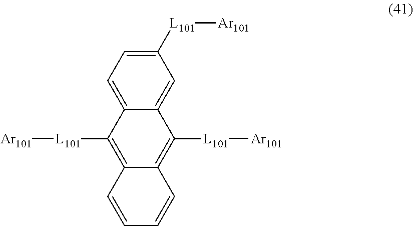

In the formula (41), L₁₀₁'s and Ar₁₀₁'s are the same as defined in the formula (40).

In one embodiment, the compound represented by the formula (40) is a compound represented by any one of the following formulas (42-1) to (42-3).

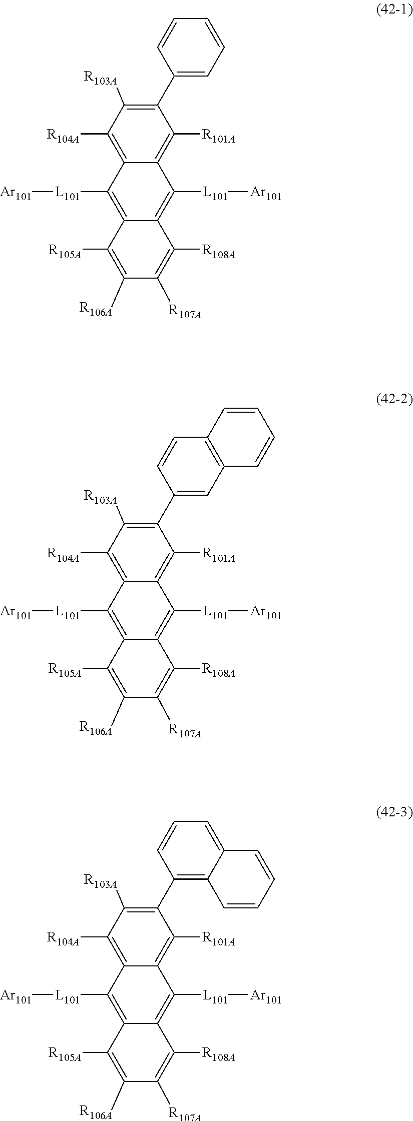

In the formulas (42-1) to (42-3), R_{101A} to R_{108A}, L₁₀₁'s and Ar₁₀₁'s are the same as defined in the formula (40).

In one embodiment, the compounds represented by the formulas (42-1) to (42-3) are a compound represented by any one of the following formulas (43-1) to (43-3).

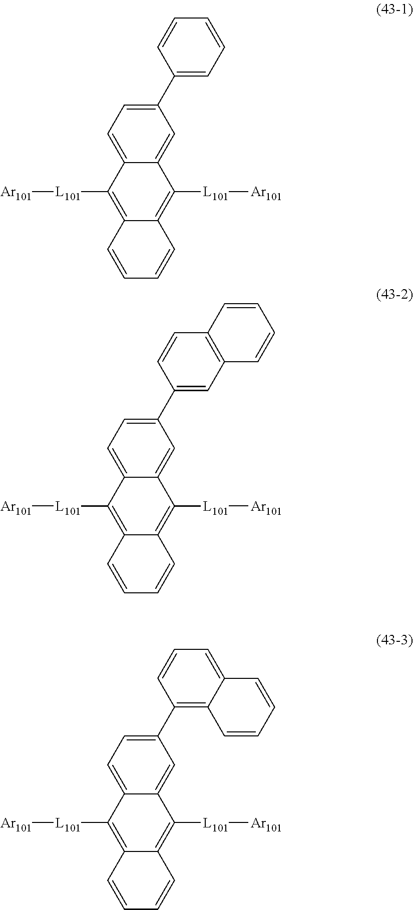

In the formulas (43-1) to (43-3), L₁₀₁'s and Ar₁₀₁'s are the same as defined in the formula (40).

In one embodiment, the group represented by -L₁₀₁-Ar₁₀₁ in the formulas (40), (41), (42-1) to (42-3), and (43-1) to (43-3) is selected from the group consisting of

a substituted or unsubstituted phenyl group,

a substituted or unsubstituted naphthyl group,

a substituted or unsubstituted biphenyl group,

a substituted or unsubstituted phenanthrenyl group,

a substituted or unsubstituted benzophenanthrenyl group,

a substituted or unsubstituted fluorenyl group,

a substituted or unsubstituted benzofluorenyl group,

a substituted or unsubstituted dibenzofuranyl group,

a substituted or unsubstituted naphthobenzofuranyl group,

a substituted or unsubstituted dibenzothiophenyl group, and

a substituted or unsubstituted carbazolyl group.

In one embodiment, the compound represented by the formula (10) or the formula (20) includes a compound in which at least one of the hydrogen atoms possessed by these compounds is a deuterium atom.

In one embodiment, in the formula (20), at least one of,

R₁₀₁ to R₁₀₈ which are hydrogen atoms,

hydrogen atoms possessed by R₁₀₁ to R₁₀₈ which are the substituents R,

hydrogen atoms possessed by L₁₀₁,

hydrogen atoms possessed by the substituent of L₁₀₁,

hydrogen atoms possessed by Ar₁₀₁, and

hydrogen atoms possessed by the substituent of Ar₁₀₁

is a deuterium atom.

The compounds represented by the formulas (30) to (37) include compounds in which at least one of the hydrogen atoms possessed by these compounds is a deuterium atom.

In one embodiment, at least one of the hydrogen atoms bonding to the carbon atoms constituting the anthracene skeletons in the compounds represented by the formulas (30) to (37) is a deuterium atom.

In one embodiment, the compound represented by the formula (30) is a compound represented by the following formula (30D).

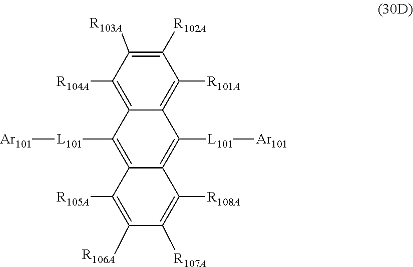

In the formula (30D), R_{101A} to R_{108A}, L₁₀₁'s and Ar₁₀₁'s are the same as defined in the formula (30).

Here, at least one of, R_{101A} to R_{110A} which are hydrogen atoms,

hydrogen atoms possessed by R_{101A} to R_{110A} which are the substituents R,

hydrogen atoms possessed by L₁₀₁,

hydrogen atoms possessed by the substituent of L₁₀₁,

hydrogen atoms possessed by Ar₁₀₁, and

hydrogen atoms possessed by the substituent of Ar₁₀₁

is a deuterium atom.

That is, the compound represented by the formula (30D) is a compound in which at least one of the hydrogen atoms possessed by the compound represented by the formula (30) is a deuterium atom.

In one embodiment, at least one of R_{101A} to R_{108A} which is a hydrogen atom in the formula (30D) is a deuterium atom.

In one embodiment, the compound represented by the formula (30D) is a compound represented by the following formula (31D).

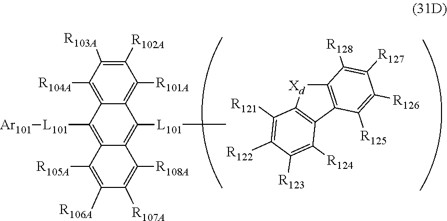

In the formula (31D), R_{101A} to R_{108A}, L₁₀₁'s and Ar₁₀₁ are the same as defined in the formula (30D).

X_{d} is OorS.

One of R₁₂₁ to R₁₂₈ is a single bond bonding with L₁₀₁.

One or more sets of the adjacent two or more of R₁₂₁ to R₁₂₈ which are not single bonds bonding with L₁₀₁ form a substituted or unsubstituted, saturated or unsaturated ring, or do not form the substituted or unsubstituted, saturated or unsaturated ring.

R₁₂₁ to R₁₂₈ which are not a single bond bonding with L₁₀₁ and which do not form the substituted or unsubstituted, saturated or unsaturated ring are independently

a hydrogen atom, or

a substituent R.

The substituent R is the same as defined in the formula (10).

Here, at least one of, R_{101A} to R_{110A} which are hydrogen atoms,

hydrogen atoms possessed by R_{101A} to R_{110A} which are the substituents R,

hydrogen atoms possessed by L₁₀₁,

hydrogen atoms possessed by the substituent of L₁₀₁,

hydrogen atoms possessed by Ar₁₀₁

hydrogen atoms possessed by the substituent of Ar₁₀₁,

R₁₂₁ to R₁₂₈ which are hydrogen atoms, and

hydrogen atoms possessed by R₁₂₁ to R₁₂₈ which are the substituents R

is a deuterium atom.

In one embodiment, the compound represented by the formula (31D) is a compound represented by the following formula (32D).

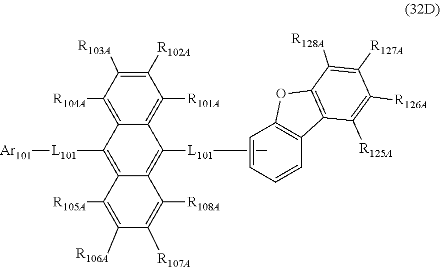

In the formula (32D), R_{101A} to R_{108A}, R_{125A} to R_{128A}, L₁₀₁'s and Ar₁₀₁ are the same as defined in the formula (31D).

Here, at least one of,

R_{101A} to R_{108A} which are hydrogen atoms,

hydrogen atoms possessed by R_{101A} to R₁₀₈ which are the substituents R,

R_{125A} to R_{128A} which are hydrogen atoms,

hydrogen atoms possessed by R_{125A} to R_{128A} which are the substituents R,

hydrogen atoms bonding to the carbon atoms of the dibenzofuran skeleton in the formula (32D),

hydrogen atoms possessed by L₁₀₁,

hydrogen atoms possessed by the substituent of L₁₀₁,

hydrogen atoms possessed by Ar₁₀₁, and

hydrogen atoms possessed by the substituent of Ar₁₀₁

is a deuterium atom.

In one embodiment, the compound represented by the formula (32D) is a compound represented by the following formula (32D-1) or (32D-2).

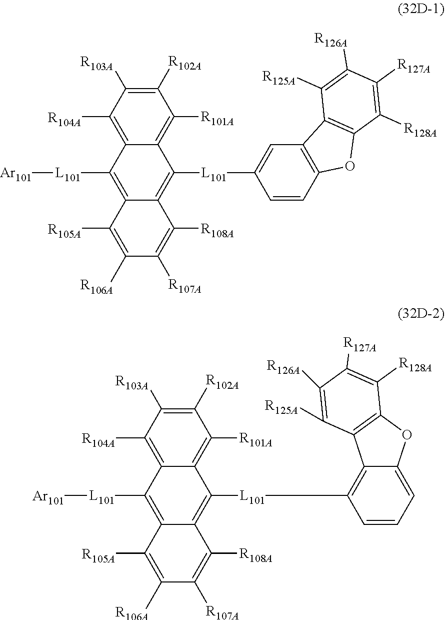

In the formulas (32D-1) and (32D-2), R_{101A} to R_{108A}, R_{125A} to R_{128A}, L₁₀₁'s and Ar₁₀₁ are the same as defined in the formula (32D).

Here, at least one of,

R_{101A} to R_{108A} which are hydrogen atoms,

hydrogen atoms possessed by R_{101A} to R₁₀₈ which are the substituents R,

R_{125A} to R_{128A} which are hydrogen atoms,

hydrogen atoms possessed by R_{125A} to R_{128A} which are the substituents R,

hydrogen atoms bonding to the carbon atoms of the dibenzofuran skeleton in the formulas (32D-1) and (32D-2),

hydrogen atoms possessed by L₁₀₁,

hydrogen atoms possessed by the substituent of L₁₀₁,

hydrogen atoms possessed by Ar₁₀₁, and

hydrogen atoms possessed by the substituent of Ar₁₀₁

is a deuterium atom.

In one embodiment, at least one of the hydrogen atoms possessed by the compounds represented by the formulas (40), (41), (42-1) to (42-3) and (43-1) to (43-3) is a deuterium atom.

In one embodiment, at least one of the hydrogen atoms (R_{101A} to R₁₀₈ which are hydrogen atoms) bonding to the carbon atoms constituting the anthracene skeletons in the compound represented by the formula (41) is a deuterium atom.

In one embodiment, the compound represented by the formula (40) is a compound represented by the following formula (40D).

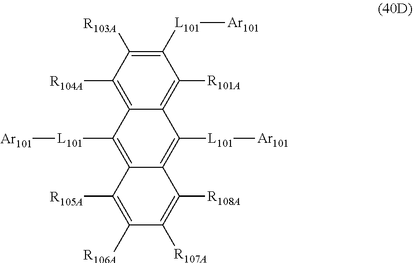

In the formula (40D), L₁₀₁'s and Ar₁₀₁'s are the same as defined in the formula (10).

One or more sets of the adjacent two or more of R_{101A}, and R_{103A} to R₁₀₈ do not form the substituted or unsubstituted, saturated or unsaturated ring.

R_{101A}, and R_{103A} to R_{108A} are independent

a hydrogen atom, or

a substituent R.

The substituent R is the same as defined in the formula (10).

Here, at least one of, R_{101A}, and R_{103A} to R_{108A} which are hydrogen atoms,

hydrogen atoms possessed by R_{101A}, and R_{103A} to R_{108A} which are the substituents R,

hydrogen atoms possessed by L₁₀₁,

hydrogen atoms possessed by the substituent of L₁₀₁,

hydrogen atoms possessed by Ar₁₀₁, and

hydrogen atoms possessed by the substituent of Ar₁₀₁

is a deuterium atom.

In one embodiment, at least one of R_{101A}, and R_{103A} to R₁₀₈ in the formula (40D) is a deuterium atom.

In one embodiment, the compound represented by the formula (40D) is a compound represented by the following formula (41 D).

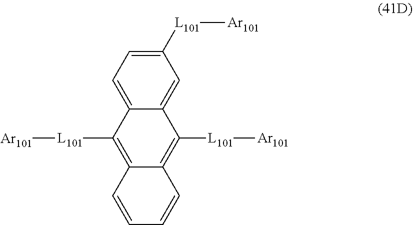

In the formula (41 D), L₁₀₁'s and Ar₁₀₁'s are the same as defined in the formula (40D).

Here, in the formula (41D), at least one of,

hydrogen atoms bonding to the carbon atoms constituting the anthracene skeleton,

hydrogen atoms possessed by L₁₀₁,

hydrogen atoms possessed by the substituent of L₁₀₁,

hydrogen atoms possessed by Ar₁₀₁, and

hydrogen atoms possessed by the substituent of Ar₁₀₁

is a deuterium atom.

In one embodiment, the compound represented by the formula (40D) is a compound represented by any one of the following formulas (42D-1) to (42D-3).

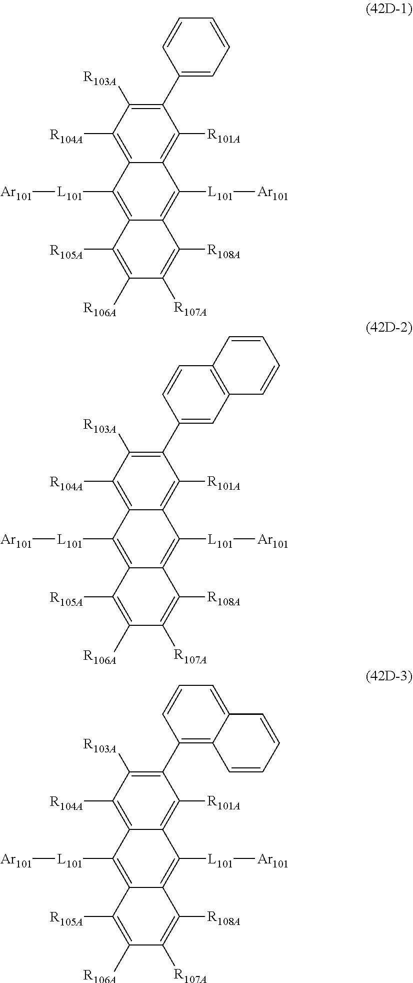

In the formula (42D-1) to (42D-3), R_{101A} to R_{108A}, L₁₀₁'s and Ar₁₀₁'s are the same as defined in the formula (40D).

Here, in the formula (42D-1), at least one of,

R_{101A}, and R_{103A} to R_{108A} which are hydrogen atoms,

hydrogen atoms possessed by R_{101A}, and R_{103A} to R_{108A} which are the substituents R,

hydrogen atoms possessed by L₁₀₁,

hydrogen atoms possessed by the substituent of L₁₀₁,

hydrogen atoms possessed by Ar₁₀₁,

hydrogen atoms possessed by the substituent of Ar₁₀₁, and

hydrogen atoms bonding to the carbon atoms constituting the phenyl group in the formula (42D-1) is a deuterium atom.

At least one of, R_{101A}, and R_{103A} to R_{108A} which are hydrogen atoms in the formula (42D-2),

hydrogen atoms possessed by R_{101A}, and R_{103A} to R_{108A} which are the substituents R,

hydrogen atoms possessed by L₁₀₁,

hydrogen atoms possessed by the substituent of L₁₀₁,

hydrogen atoms possessed by Ar₁₀₁,

hydrogen atoms possessed by the substituent of Ar₁₀₁, and

hydrogen atoms bonding to the carbon atoms constituting the naphthyl group in the formula (42D-2) is a deuterium atom.

At least one of, R_{101A}, and R_{103A} to R_{108A} which are hydrogen atoms in the formula (42D-3),

hydrogen atoms possessed by R_{101A}, and R_{103A} to R_{108A} which are the substituents R,

hydrogen atoms possessed by L₁₀₁,

hydrogen atoms possessed by the substituent of L₁₀₁,

hydrogen atoms possessed by Ar₁₀₁,

hydrogen atoms possessed by the substituent of Ar₁₀₁, and

hydrogen atoms bonding to the carbon atoms constituting the naphthyl group in the formula (42D-3)

is a deuterium atom.

In one embodiment, the compounds represented by the formulas (42D-1) to (42D-3) are a compound represented by any one of the following formulas (43D-1) to (43D-3).

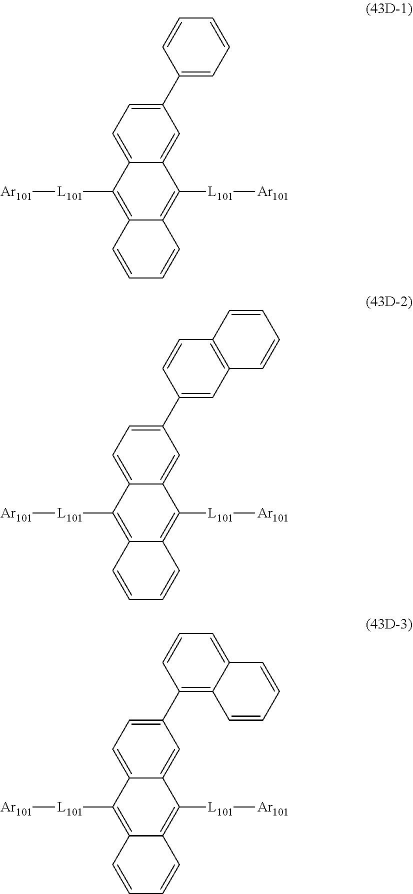

In the formula (43D-1) to (43D-3), L₁₀₁'s and Ar₁₀₁'s are the same as defined in the formula (40D).

Here, at least one of, hydrogen atoms bonding to the carbon atoms constituting the anthracene skeleton in the formula (43D-1),

hydrogen atoms possessed by L₁₀₁,

hydrogen atoms possessed by the substituent of L₁₀₁,

hydrogen atoms possessed by Ar₁₀₁,

hydrogen atoms possessed by the substituent of Ar₁₀₁, and

hydrogen atoms bonding to the carbon atoms constituting the phenyl group in the formula (43D-1) is a deuterium atom.

At least one of, hydrogen atoms bonding to the carbon atoms constituting the anthracene skeleton in the formula (43D-2),

hydrogen atoms possessed by L₁₀₁,

hydrogen atoms possessed by the substituent of L₁₀₁,

hydrogen atoms possessed by Ar₁₀₁,

hydrogen atoms possessed by the substituent of Ar₁₀₁, and

hydrogen atoms bonding to the carbon atoms constituting the naphthyl group in the formula (43D-2) is a deuterium atom.

At least one of, hydrogen atoms bonding to the carbon atoms constituting the anthracene skeleton in the formula (43D-3),

hydrogen atoms possessed by L₁₀₁,

hydrogen atoms possessed by the substituent of L₁₀₁,

hydrogen atoms possessed by Ar₁₀₁

hydrogen atoms possessed by the substituent of Ar₁₀₁, and

hydrogen atoms bonding to the carbon atoms constituting the naphthyl group in the formula (43D-3)

is a deuterium atom.

In one embodiment, in the compound represented by the formula (20), at least one of Ar₁₀₁'s is a monovalent group having a structure represented by the following formula (50).

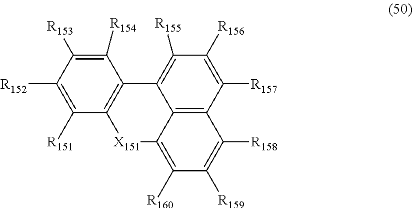

In the formula (50),

X₁₅₁ is O, S or C(R₁₆₁)(R₁₆₂).

One of R₁₅₁ to R₁₆₀ is a single bond bonding with L₁₀₁.

One or more sets of, the adjacent two or more of R₁₅₁ to R₁₅₄ and the adjacent two or more of R₁₅₅ to R₁₆₀, which are not single bonds bonding with L₁₀₁, form a substituted or unsubstituted, saturated or unsaturated ring by bonding with each other, or do not form the substituted or unsubstituted, saturated or unsaturated ring.

R₁₆₁ and R₁₆₂ form a substituted or unsubstituted, saturated or unsaturated ring by bonding with each other, or do not form the substituted or unsubstituted, saturated or unsaturated ring.

R₁₆₁ and R₁₆₂ which do not form the substituted or unsubstituted, saturated or unsaturated ring, and R₁₅₁ to R₁₆₀ which are not single bonds bonding with L₁₀₁ and which do not form the substituted or unsubstituted, saturated or unsaturated ring are independently hydrogen atoms or substituents R.

The substituent R is the same as defined in the formula (10).

Ar₁₀₁ which is not the monovalent group having the structure represented by the formula (50) is

a substituted or unsubstituted aryl group having 6 to 50 ring carbon atoms, or

a substituted or unsubstituted divalent heterocyclic group having 5 to 50 ring atoms.

The position of the single bond to L₁₀₁ in the formula (50) is not particularly limited.

In one embodiment, one of R₁₅₁ to R₁₅₄ or one of R₁₅₅ to R₁₆₀ in the formula (50) is a single bond that binds to an L₁₀₁.

In one embodiment, Ar₁₀₁ is a monovalent group represented by the formula (50-R₁₅₂), (50-R₁₅₃), (50-R₁₅₄), (50-R₁₅₇) or (50-R₁₅₈).

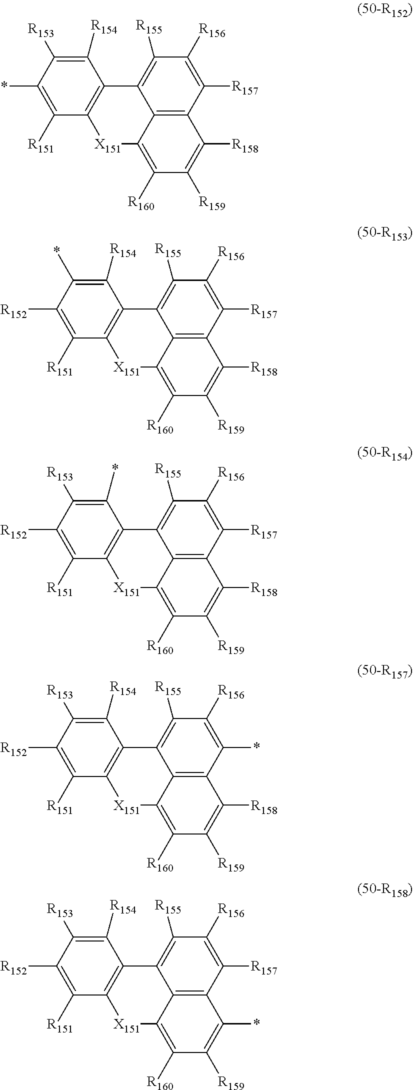

In the formulas (50-R₁₅₂), (50-R₁₅₃), (50-R₁₅₄), (50-R₁₅₇) and (50-R₁₅₈), X₁₅₁, and R₁₅₁ to R₁₆₀ are the same as defined in the formula (50).

* is bonded with L₁₀₁.

Specific examples of the compound represented by the formula (10) include the following compounds. The compound represented by the formula (10) is not limited to these specific examples. In the following specific examples, “D” represents a deuterium atom.

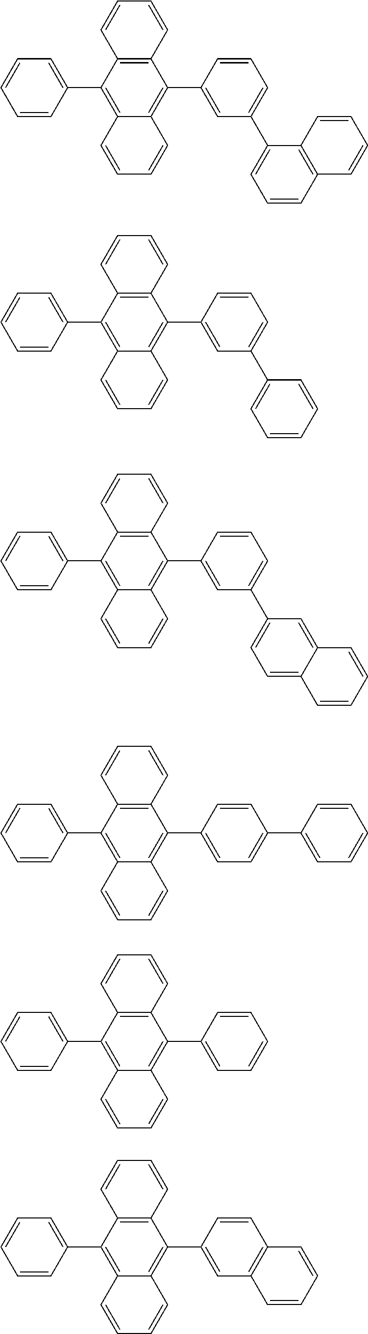

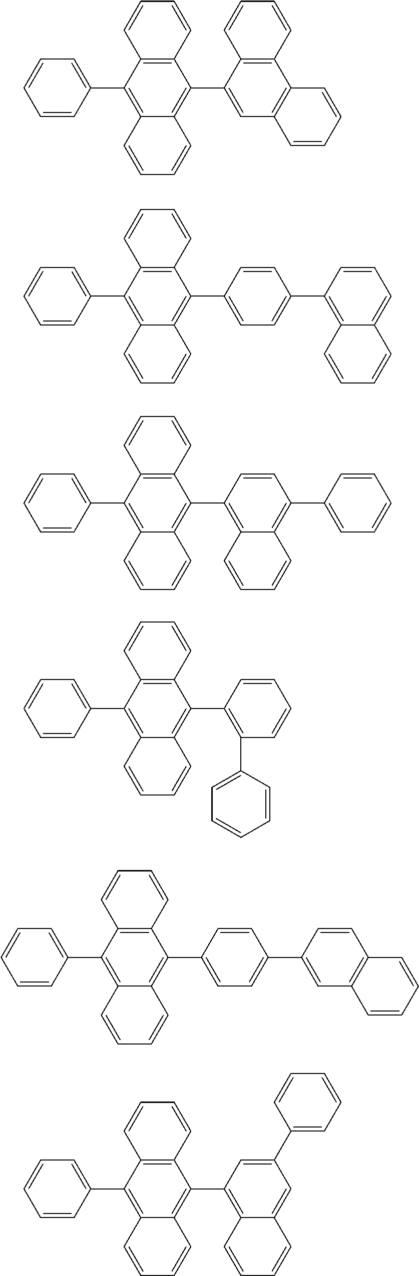

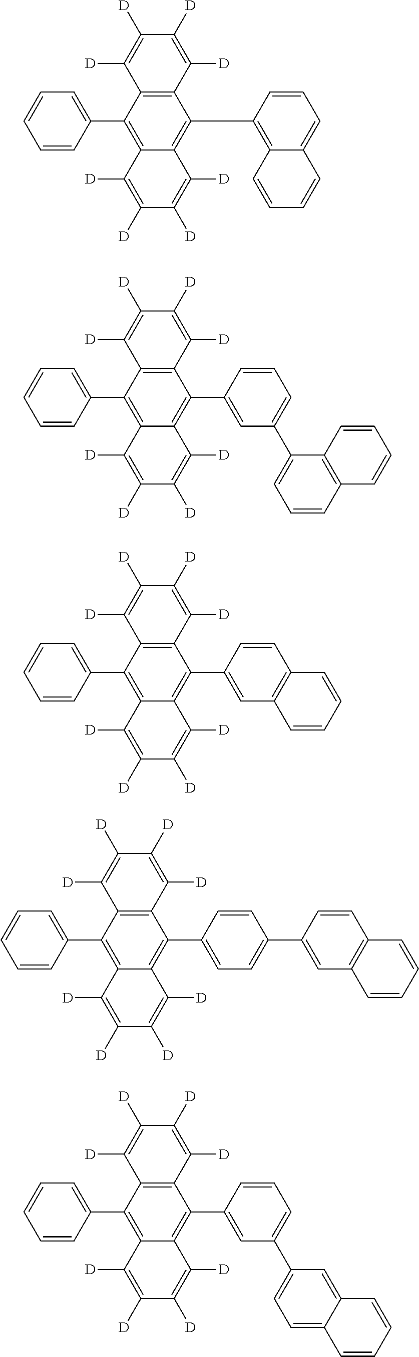

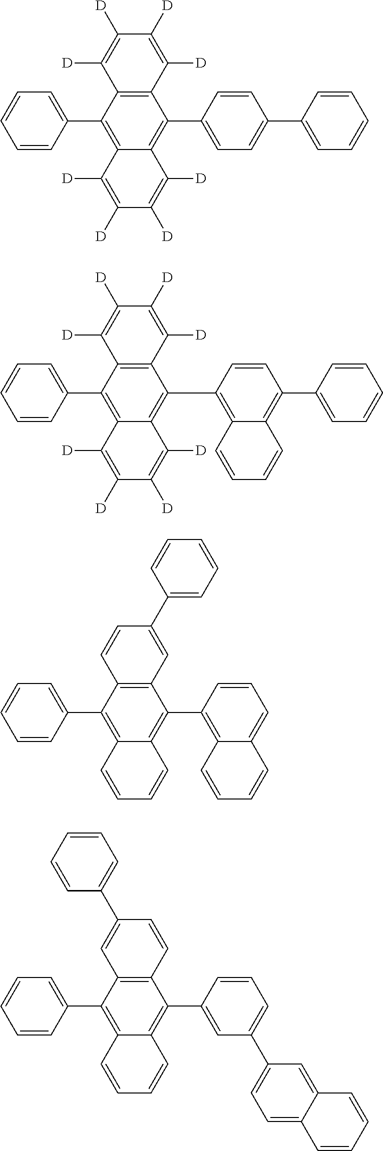

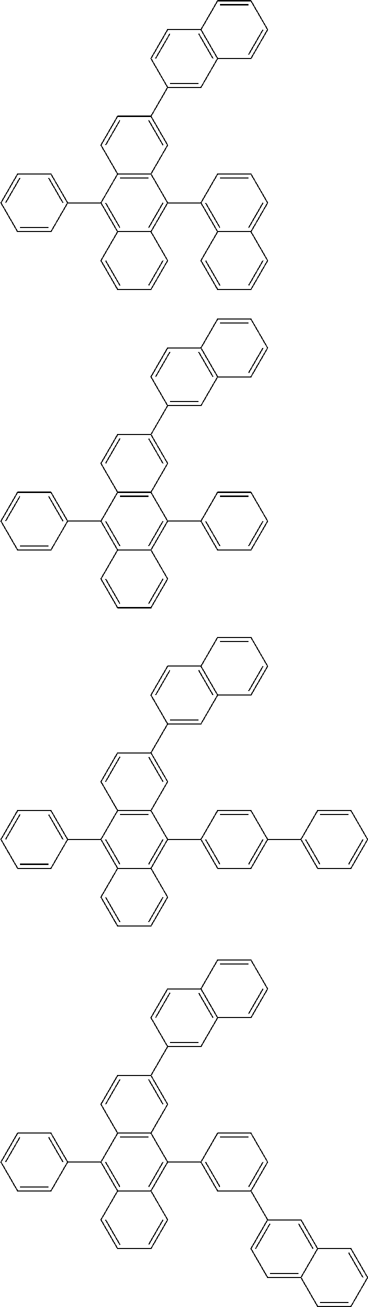

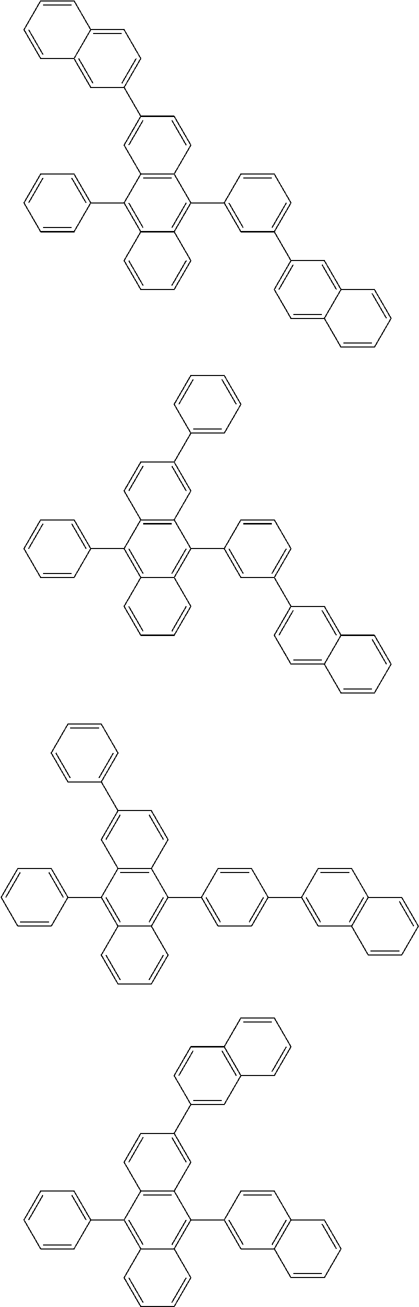

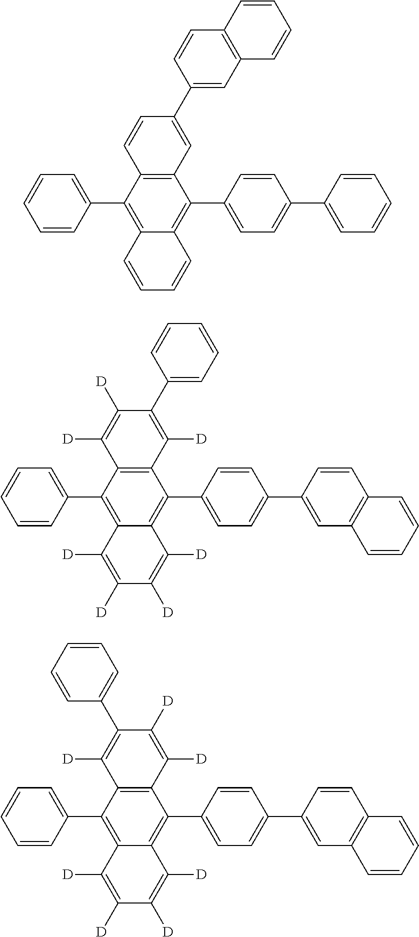

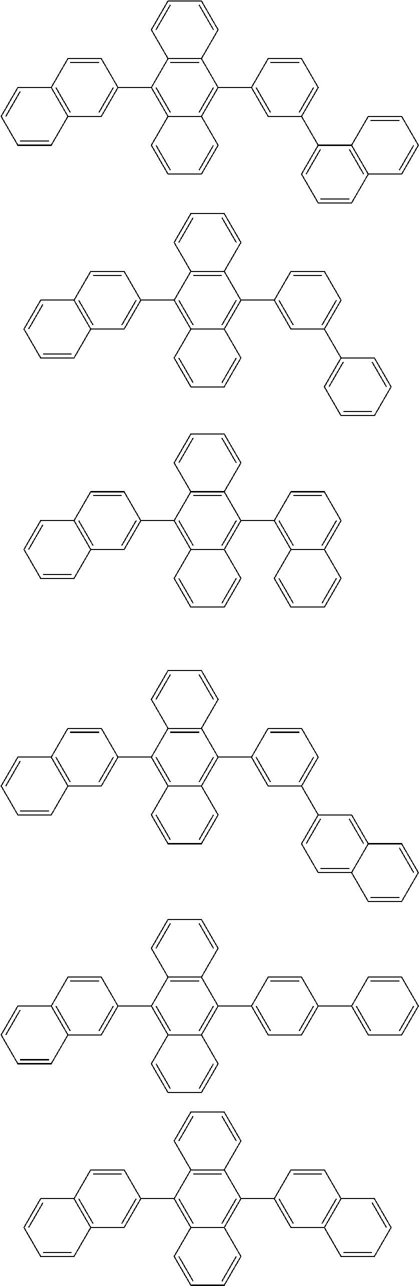

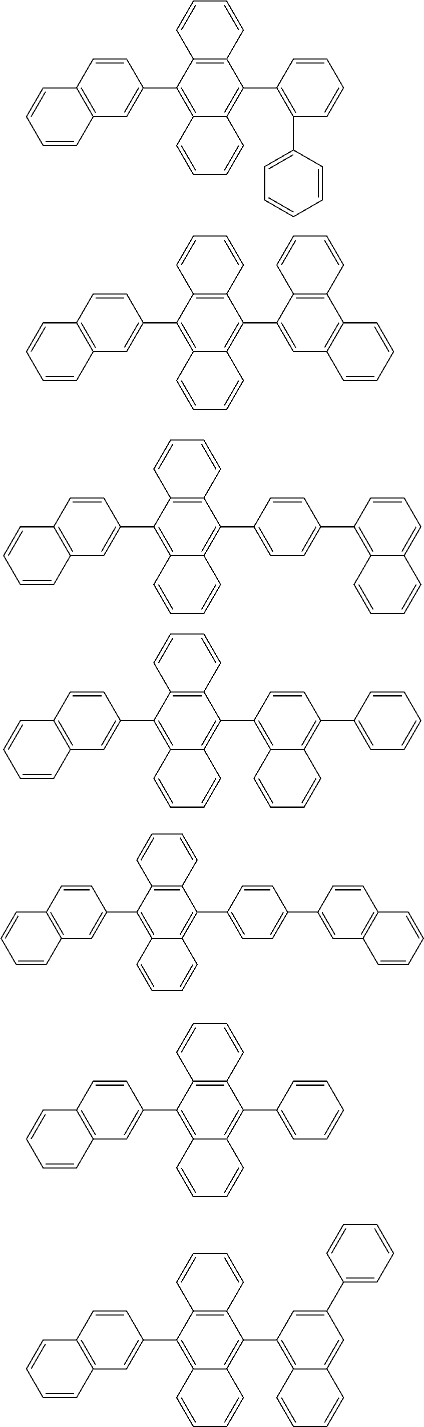

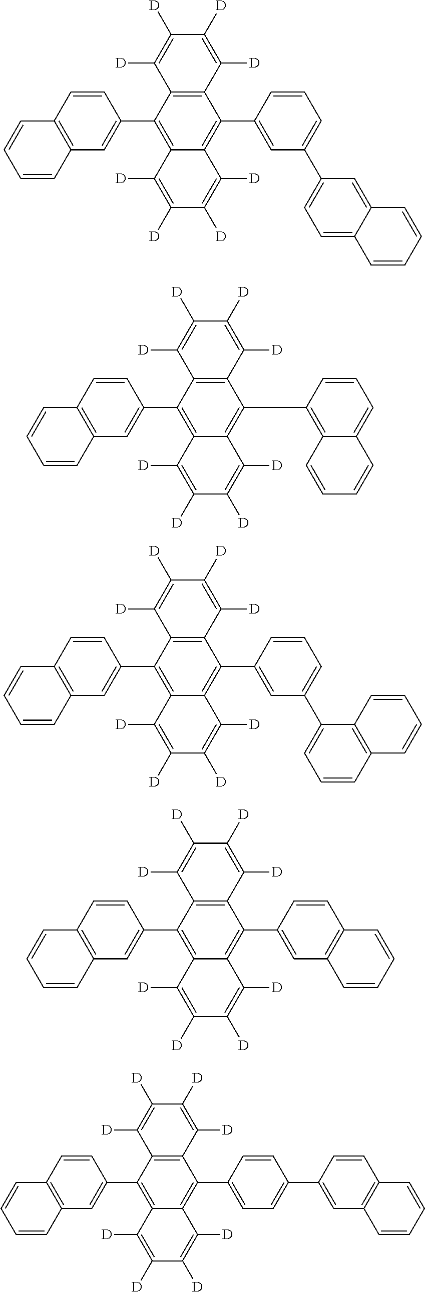

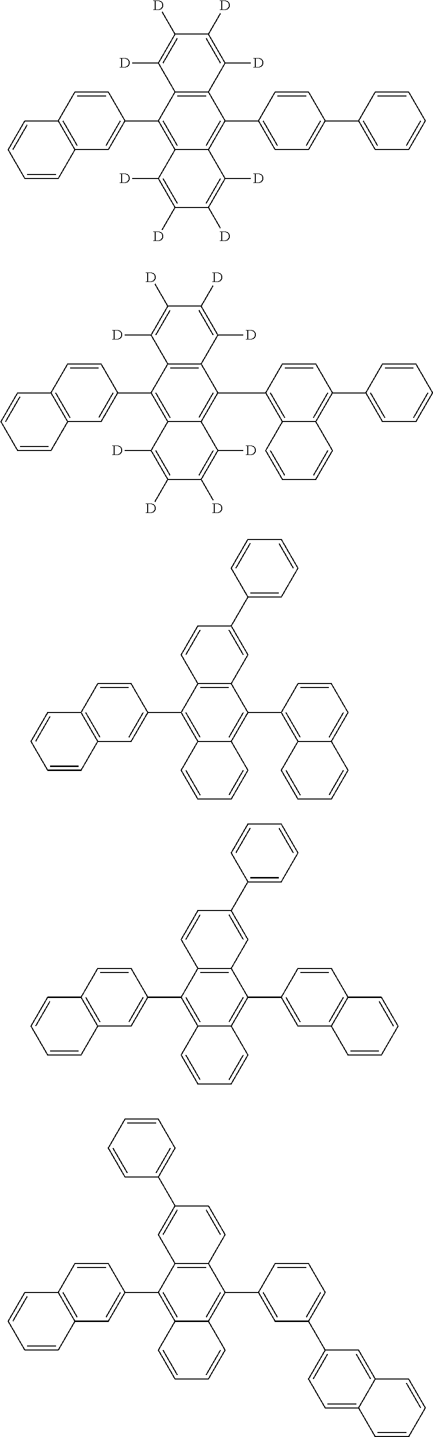

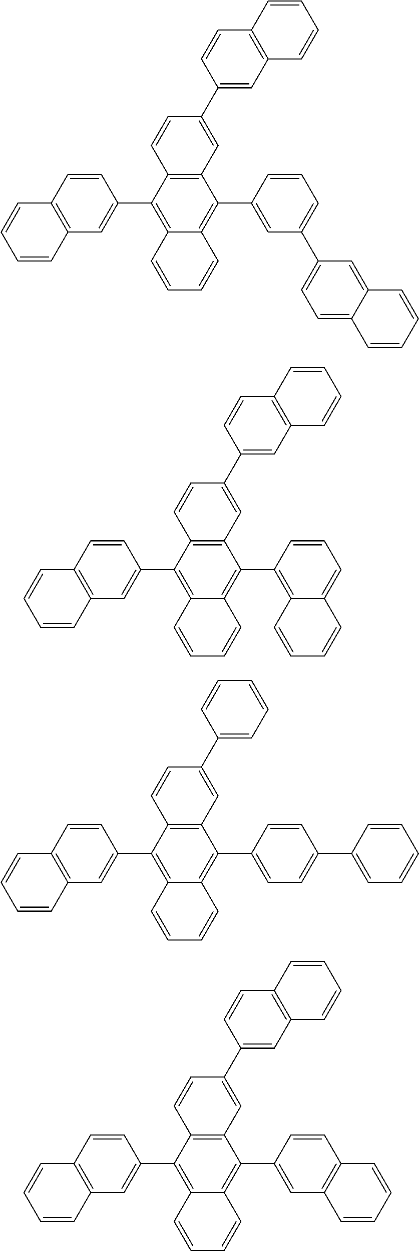

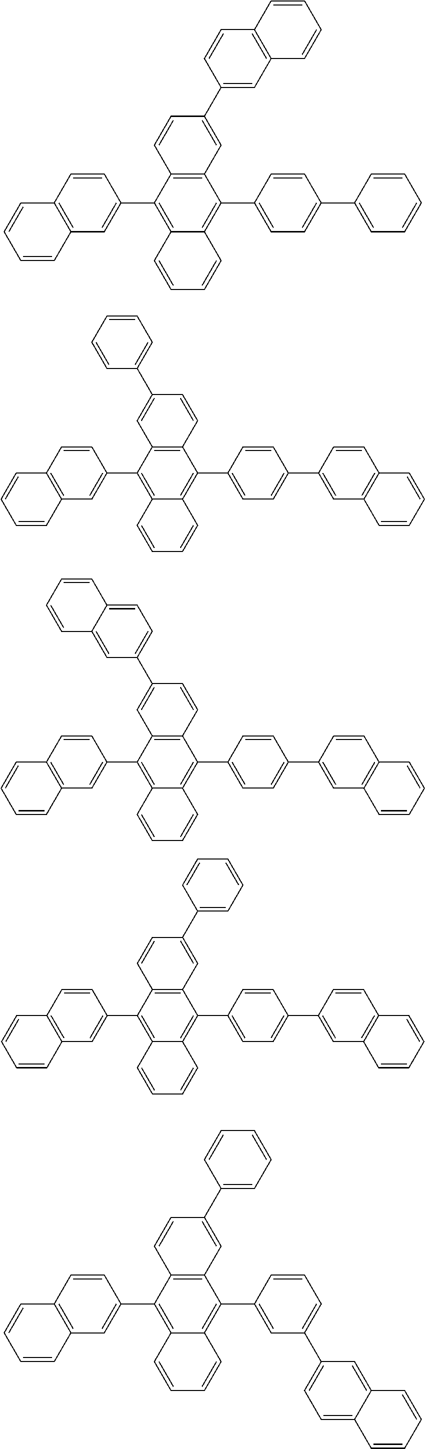

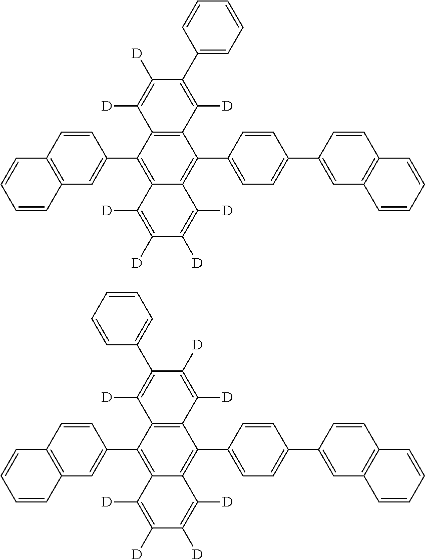

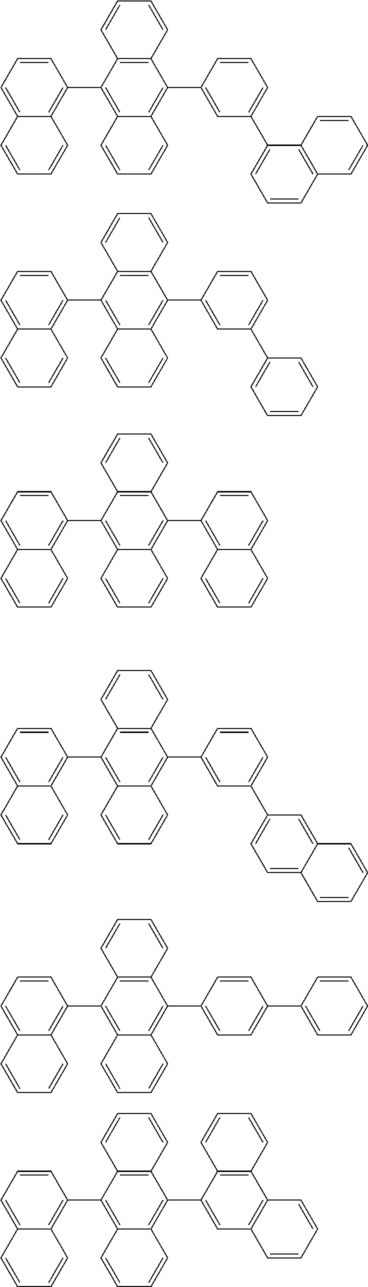

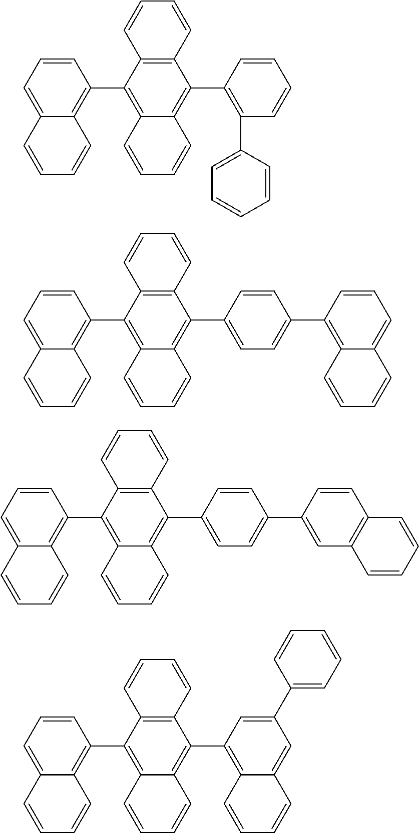

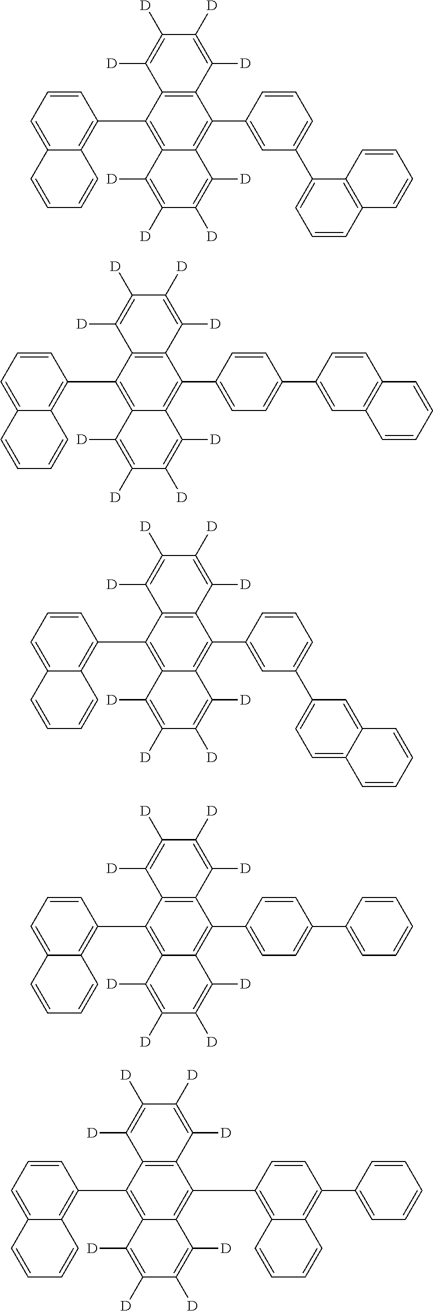

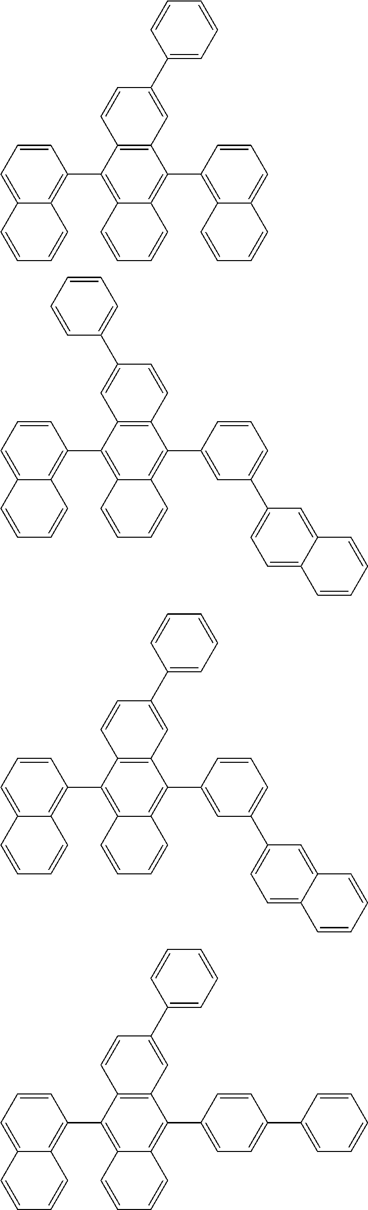

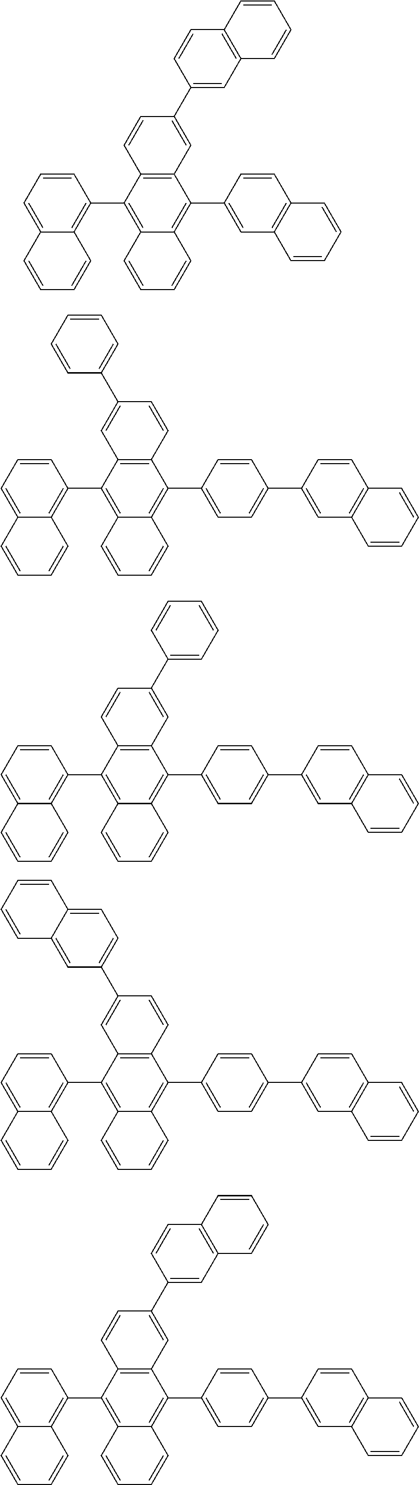

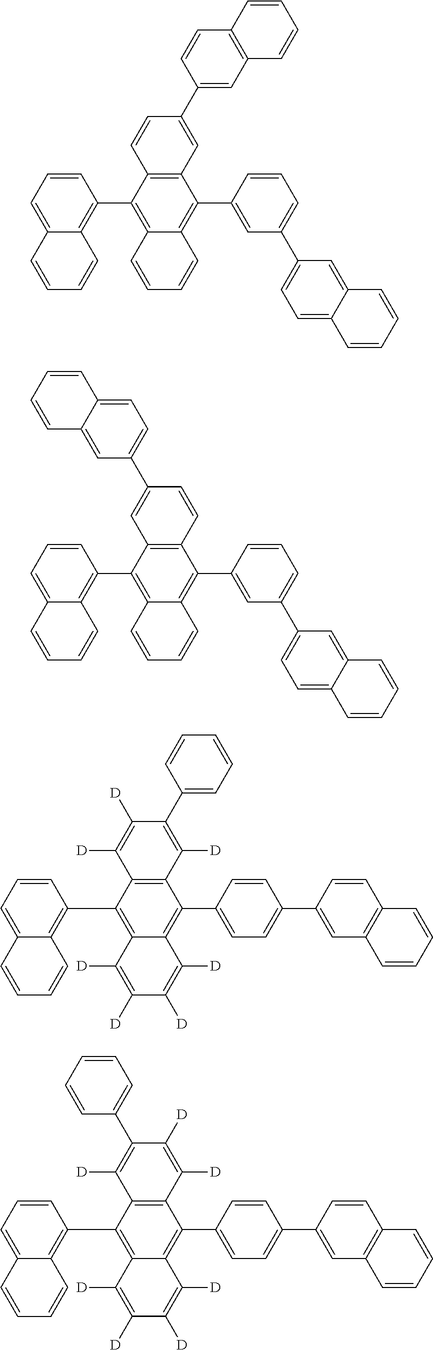

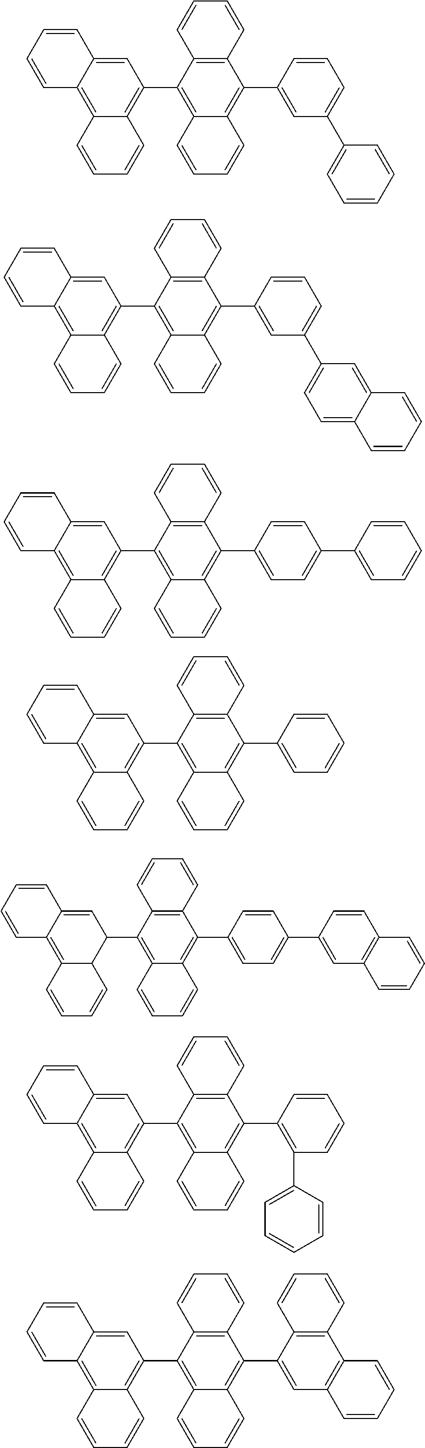

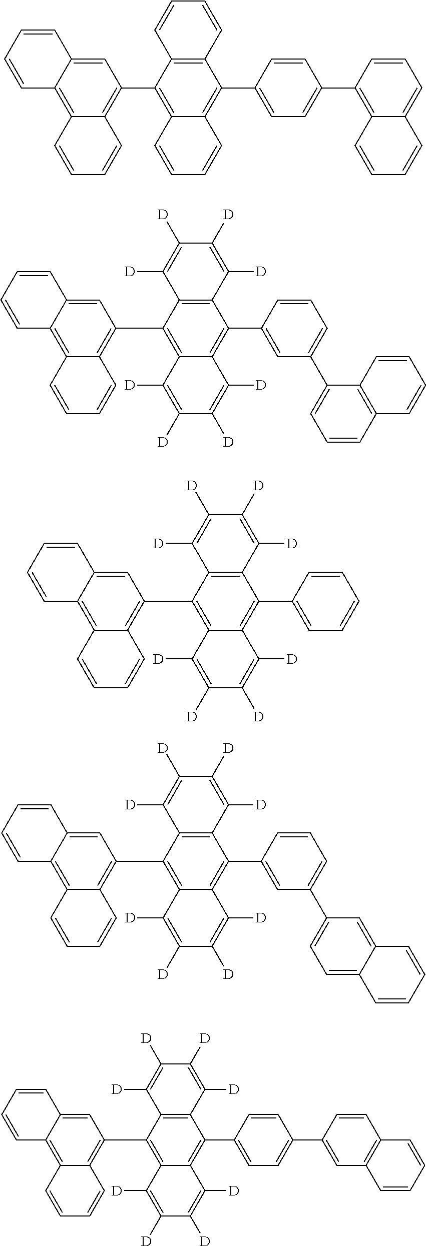

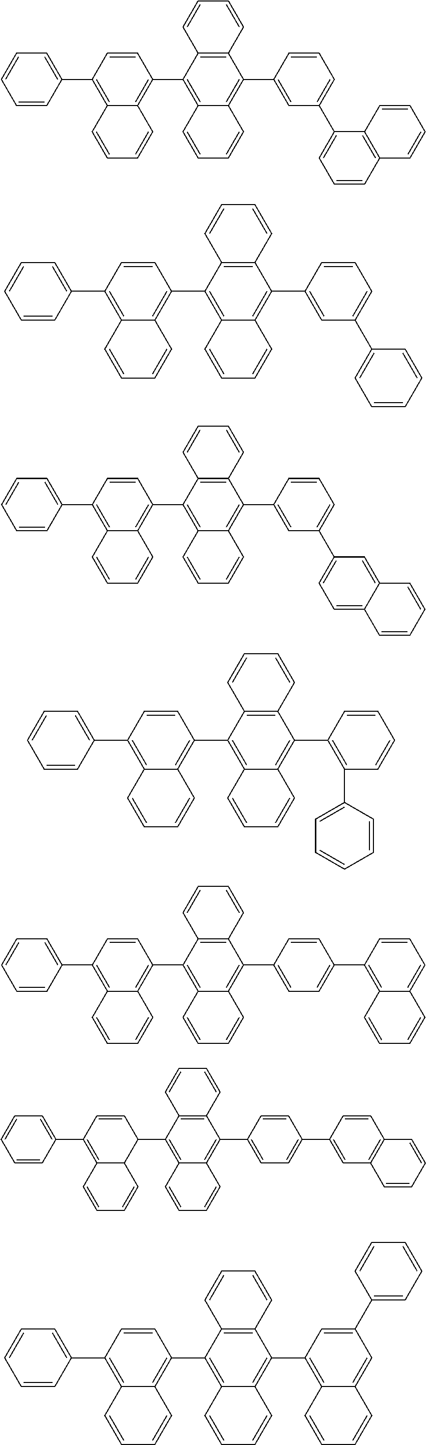

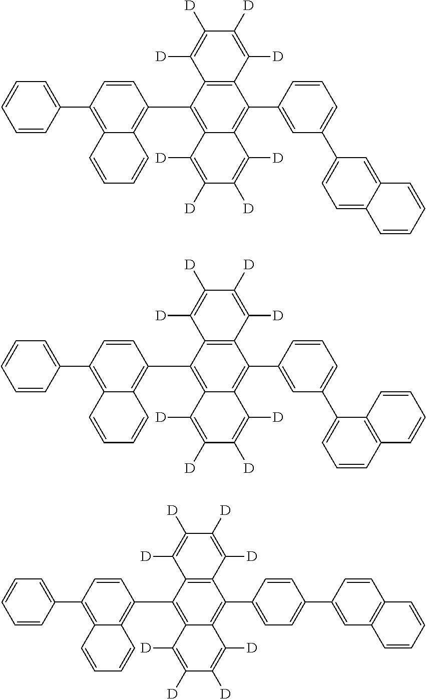 [SMILES: [2H]c1c([2H])c([2H])c2c(-c3ccc(-c4ccccc4)c4ccccc34)c3c([2H])c([2H])c([2H])c([2H])c3c(-c3cccc(-c4ccc5ccccc5c4)c3)c2c1[2H] ; [2H]c1c([2H])c([2H])c2c(-c3ccc(-c4ccccc4)c4ccccc34)c3c([2H])c([2H])c([2H])c([2H])c3c(-c3cccc(-c4cccc5ccccc45)c3)c2c1[2H] ; [2H]c1c([2H])c([2H])c2c(-c3ccc(-c4ccccc4)c4ccccc34)c3c([2H])c([2H])c([2H])c([2H])c3c(-c3ccc(-c4ccc5ccccc5c4)cc3)c2c1[2H]]

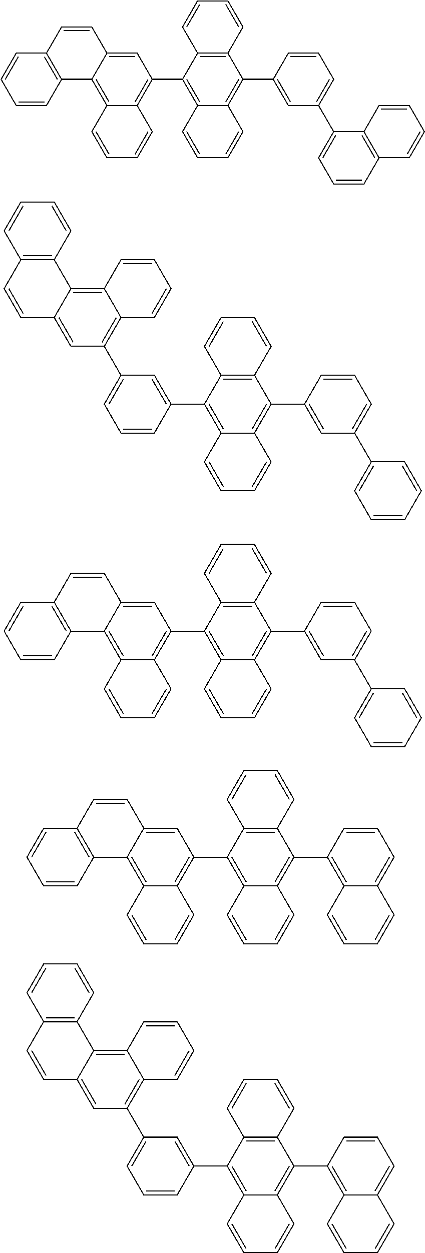

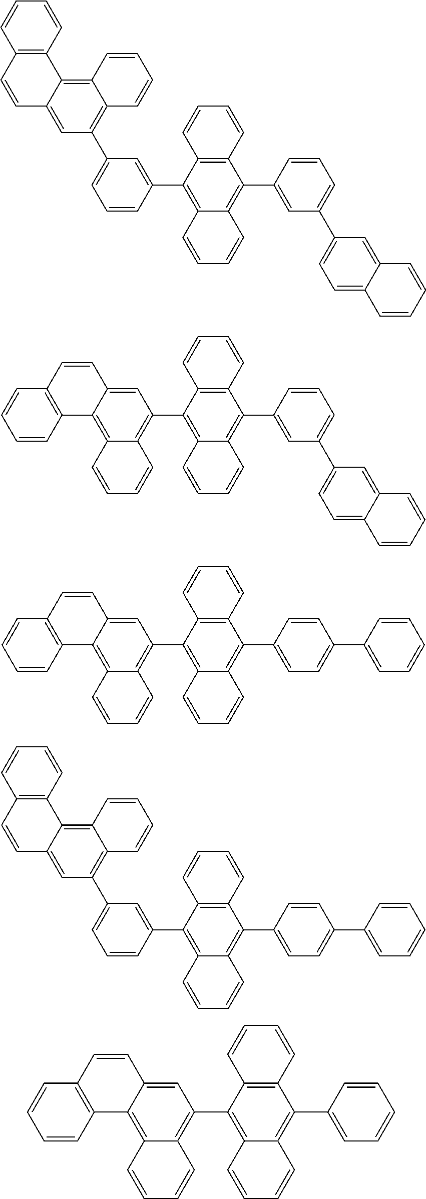

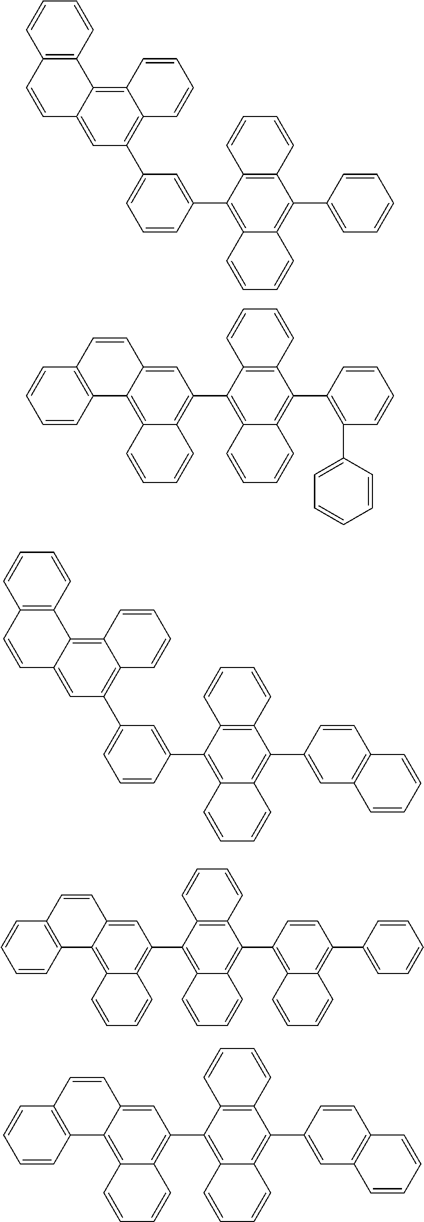

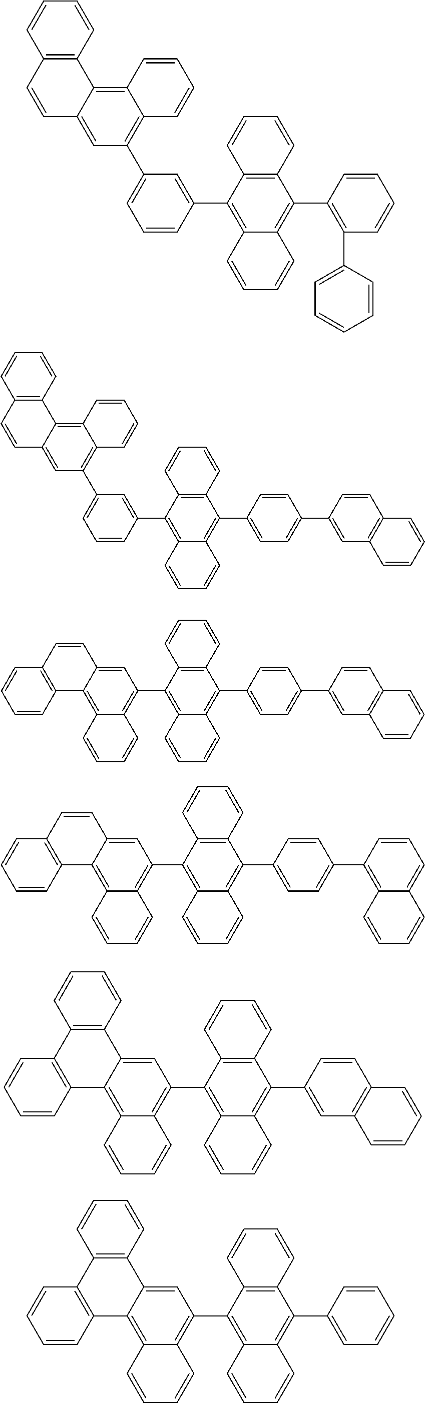

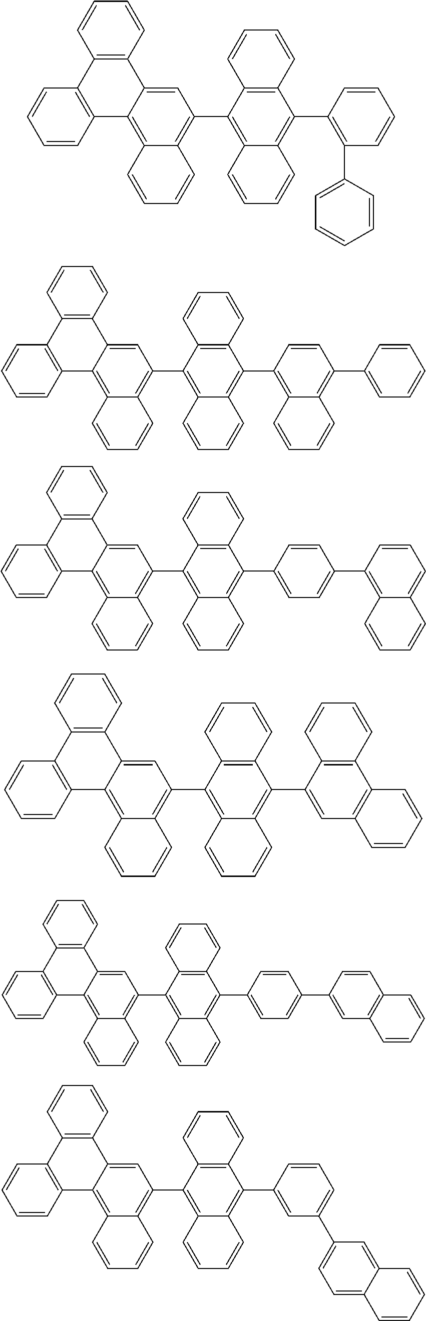

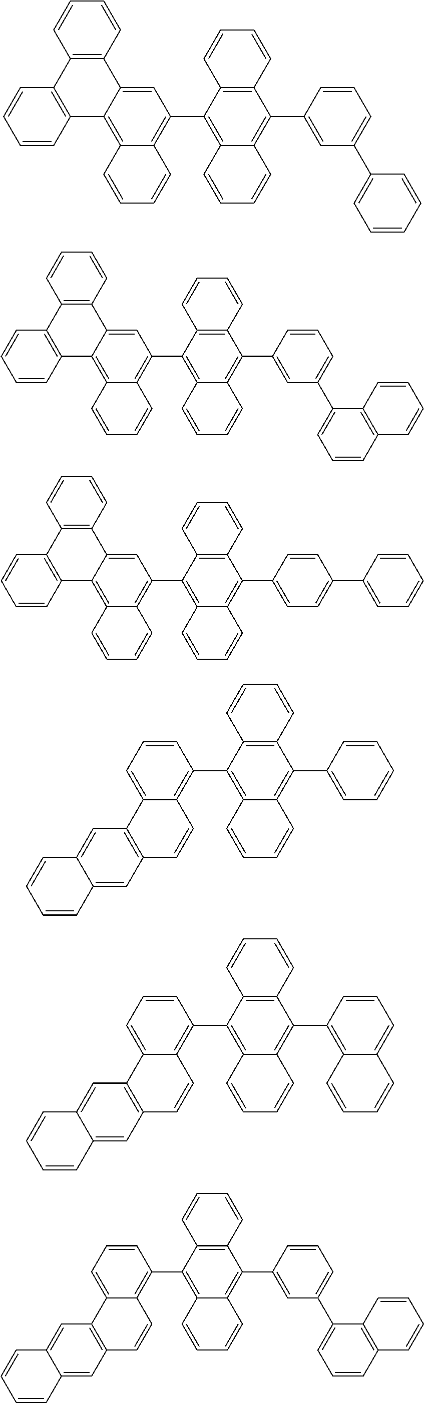

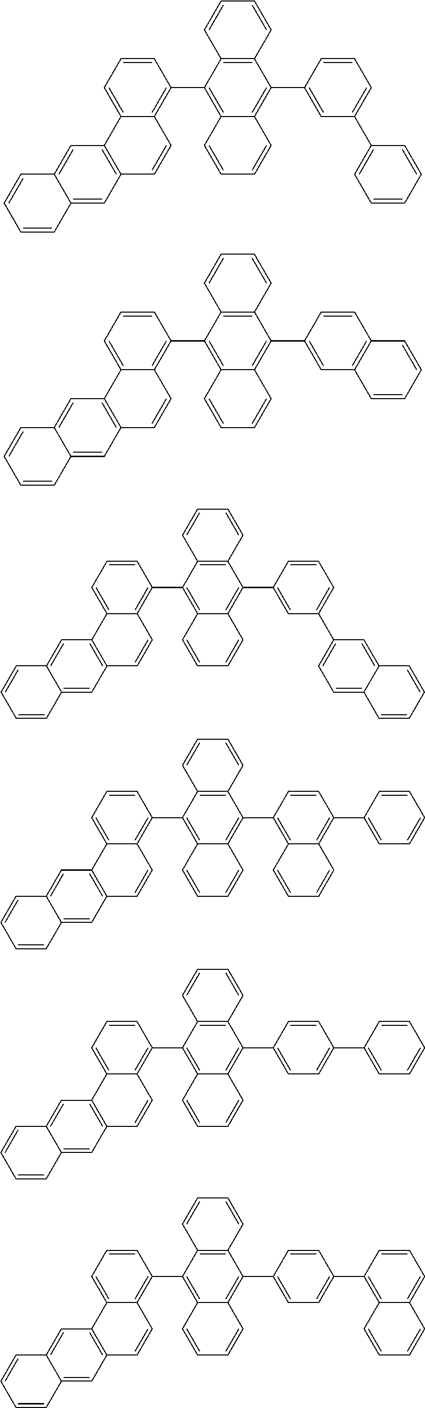

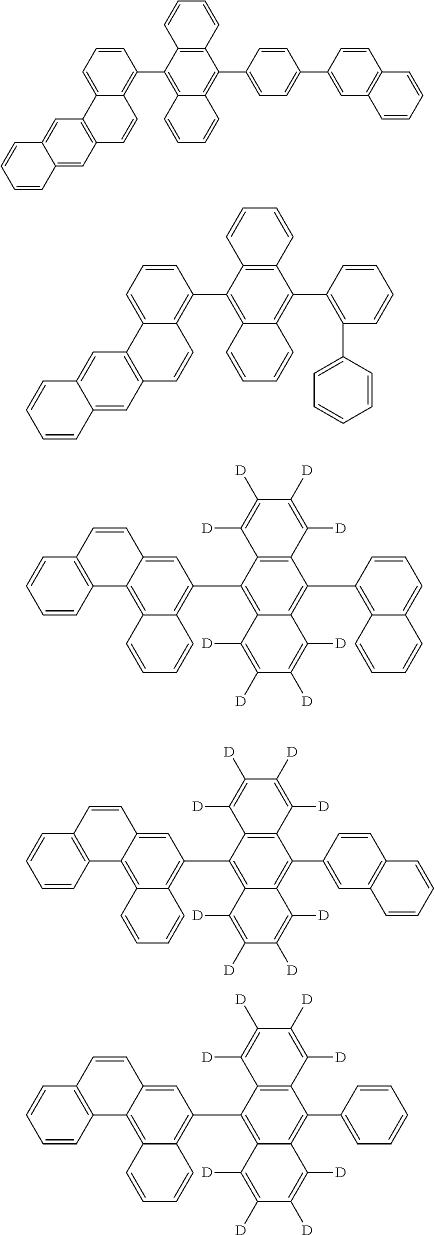

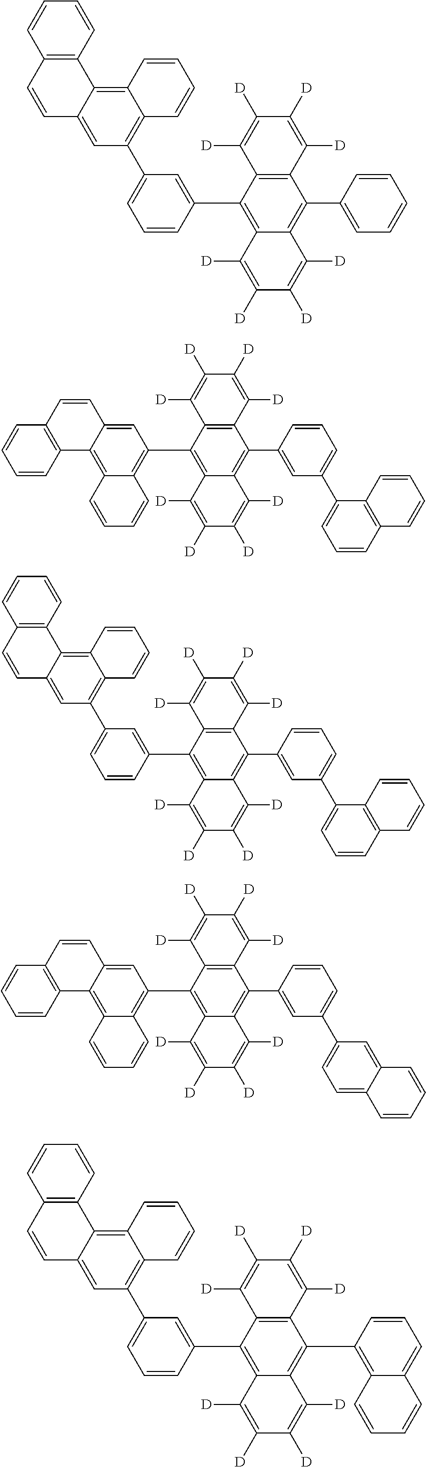

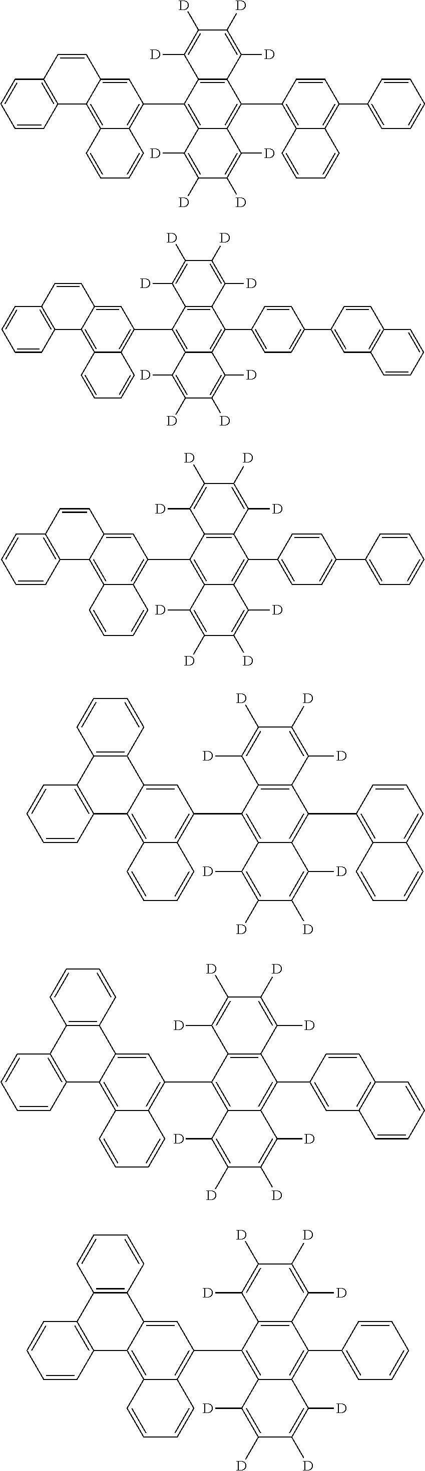

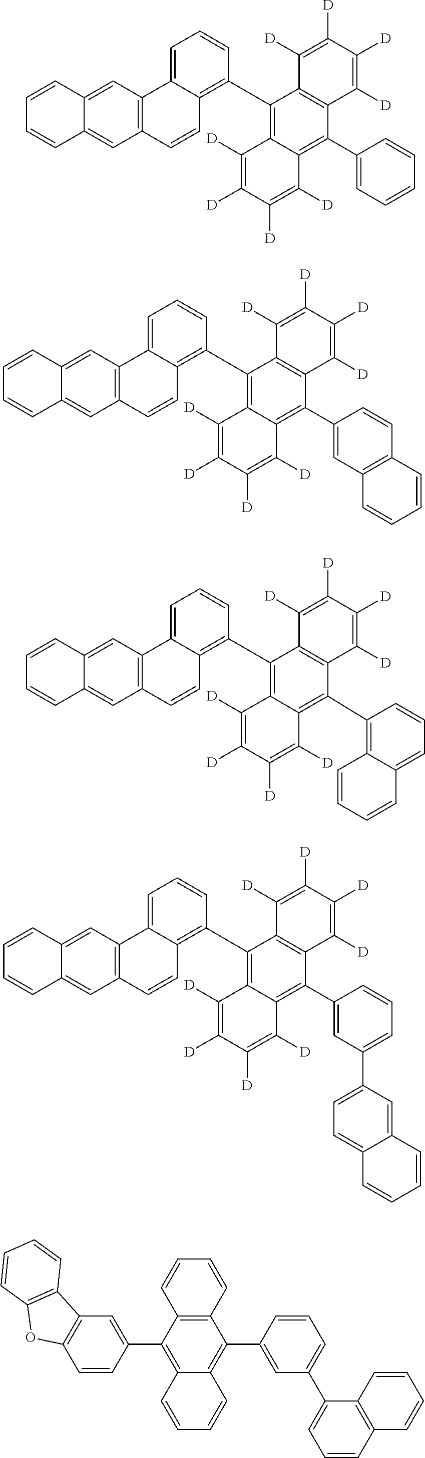

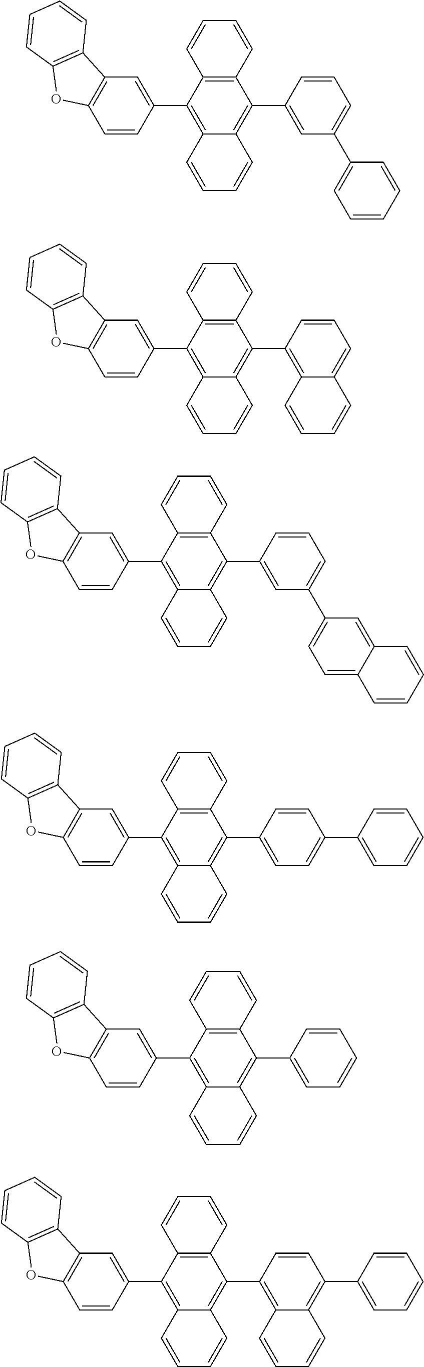

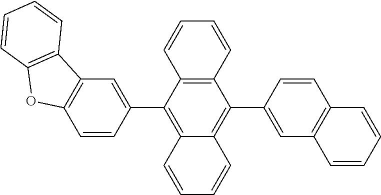 [SMILES: c1ccc2cc(-c3c4ccccc4c(-c4ccc5oc6ccccc6c5c4)c4ccccc34)ccc2c1]

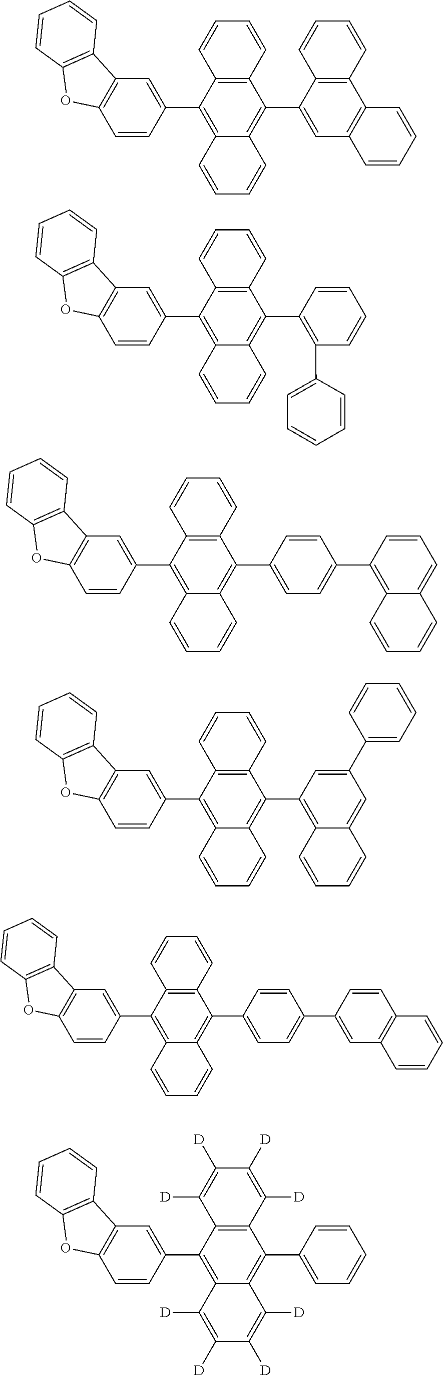

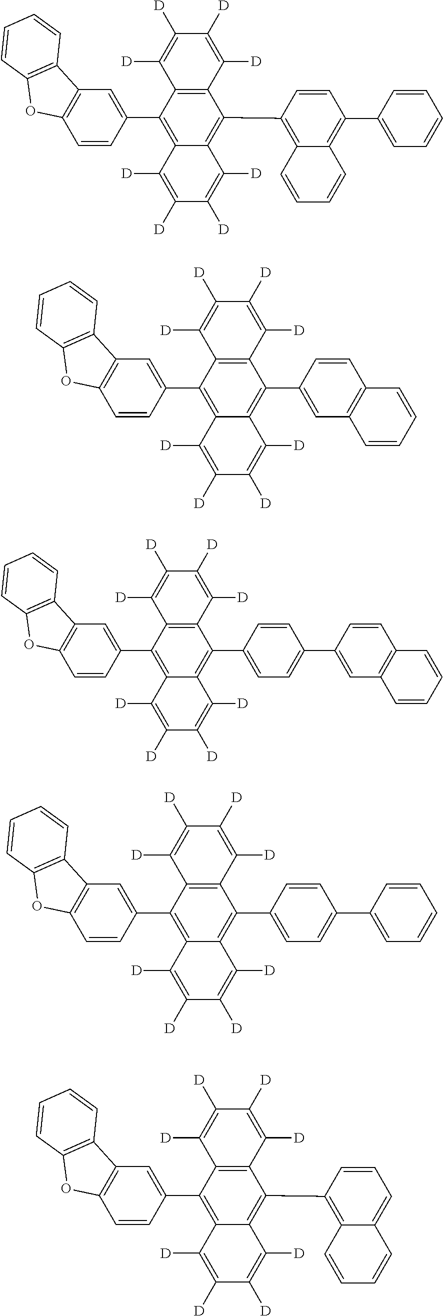

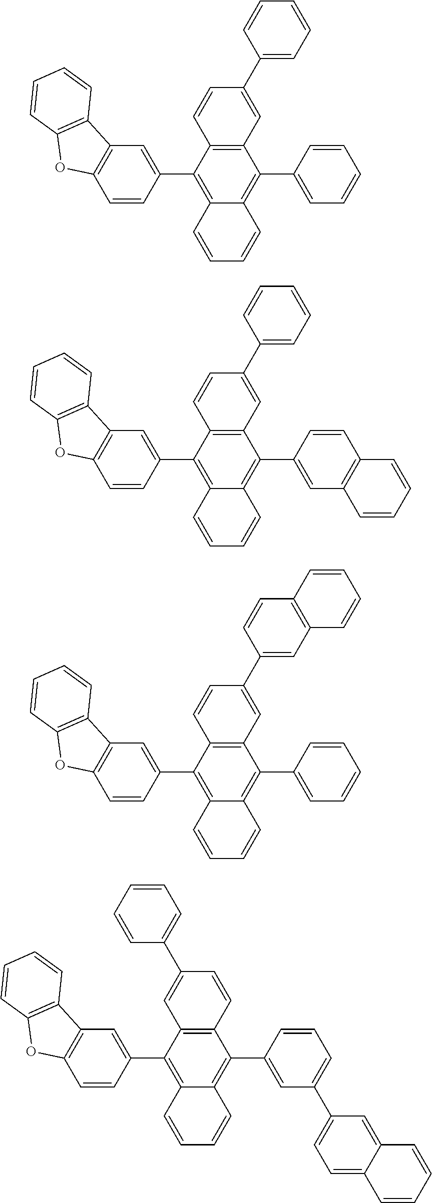

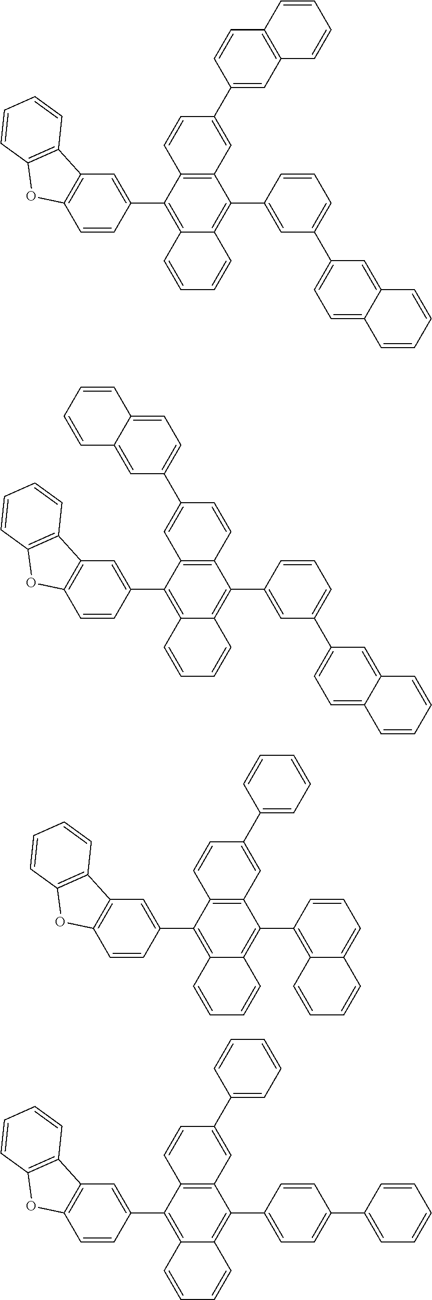

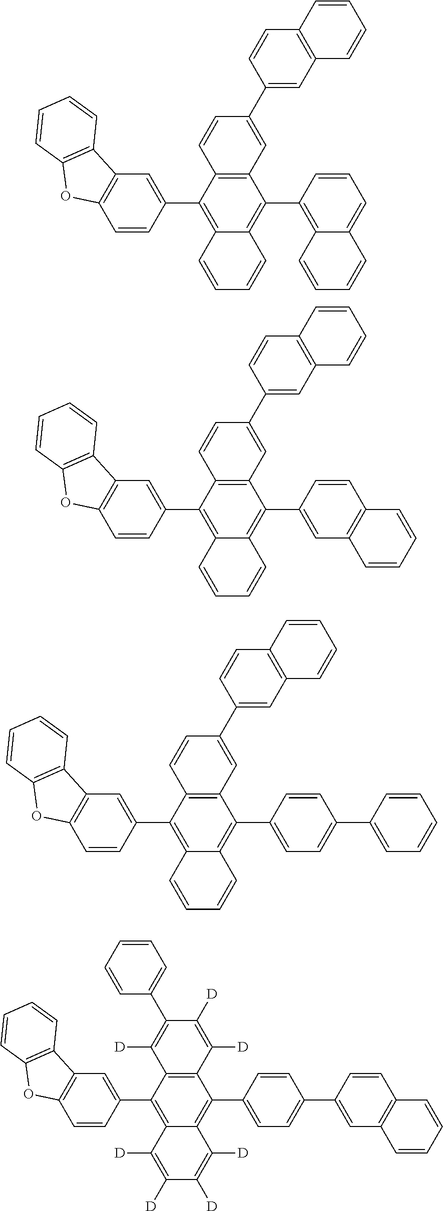

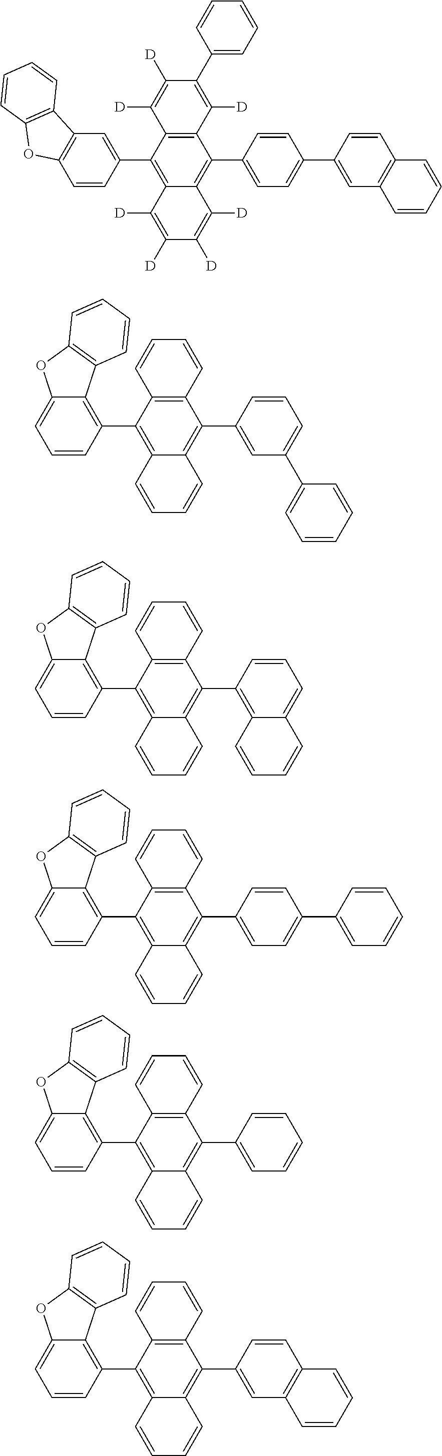

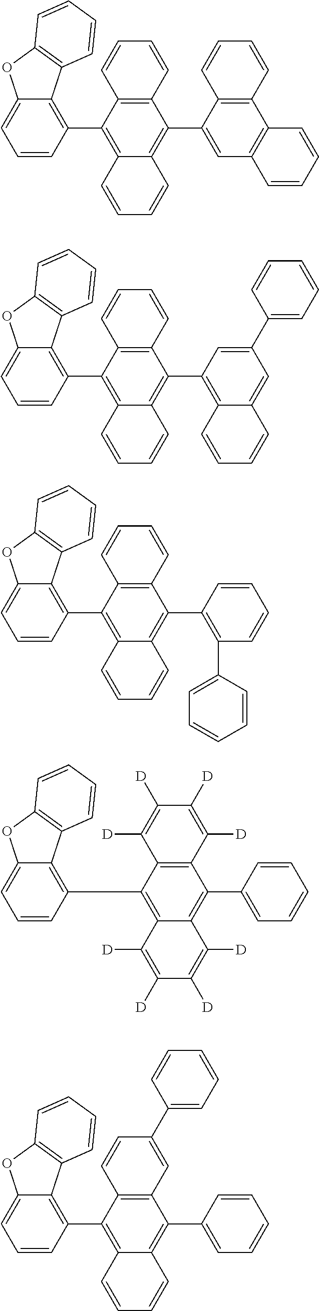

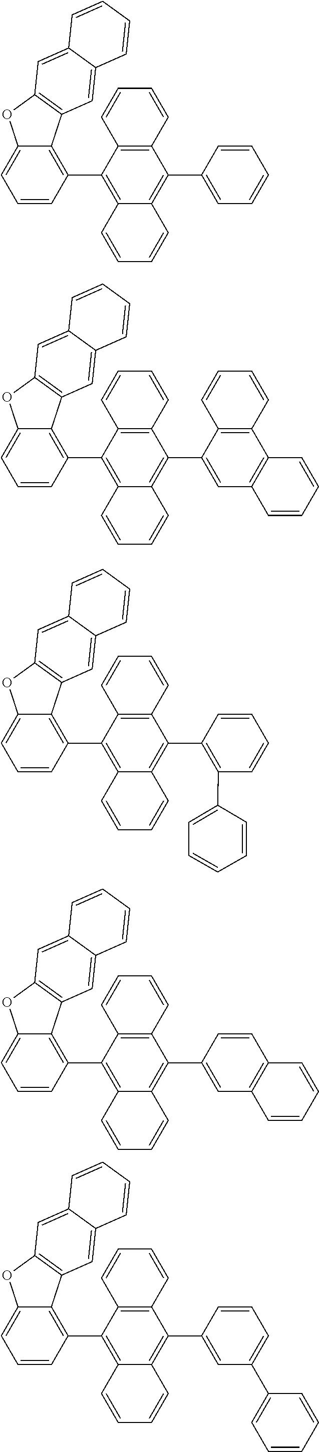

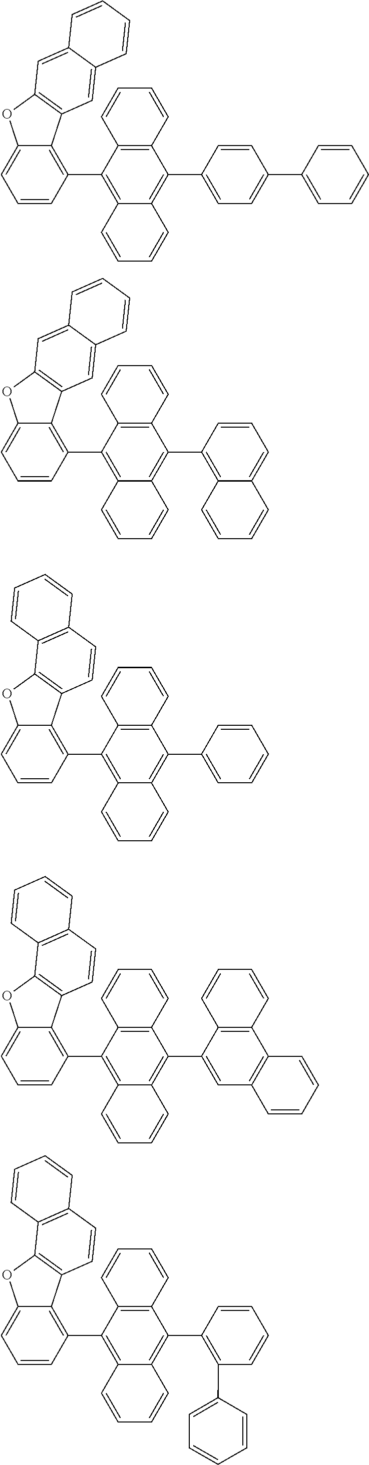

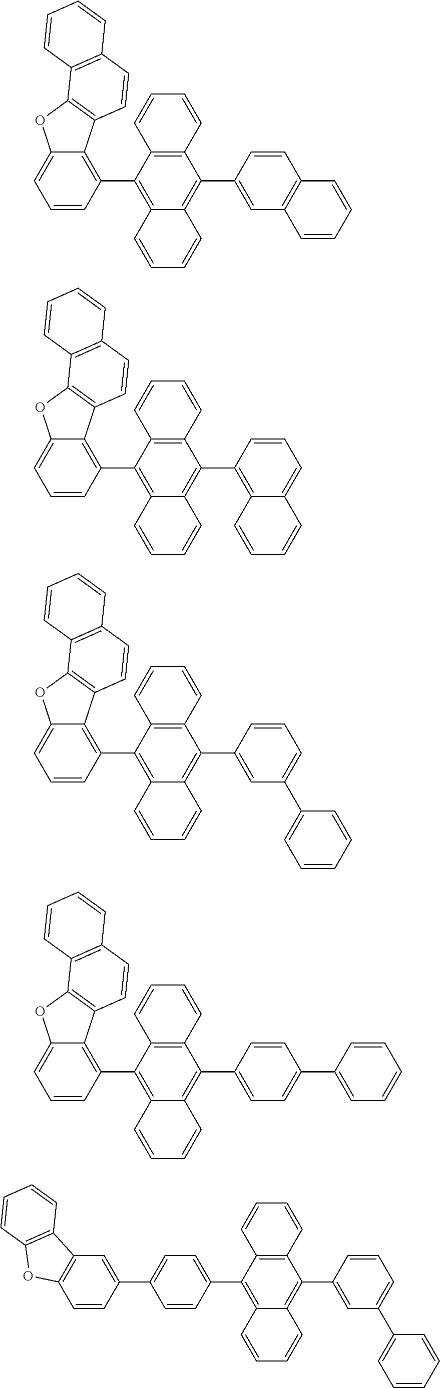 [SMILES: c1ccc2cc(-c3c4ccccc4c(-c4cccc5oc6c7ccccc7ccc6c45)c4ccccc34)ccc2c1 ; c1ccc2c(-c3c4ccccc4c(-c4cccc5oc6c7ccccc7ccc6c45)c4ccccc34)cccc2c1 ; c1ccc(-c2cccc(-c3c4ccccc4c(-c4cccc5oc6c7ccccc7ccc6c45)c4ccccc34)c2)cc1 ; c1ccc(-c2ccc(-c3c4ccccc4c(-c4cccc5oc6c7ccccc7ccc6c45)c4ccccc34)cc2)cc1 ; c1ccc(-c2cccc(-c3c4ccccc4c(-c4ccc(-c5ccc6oc7ccccc7c6c5)cc4)c4ccccc34)c2)cc1]

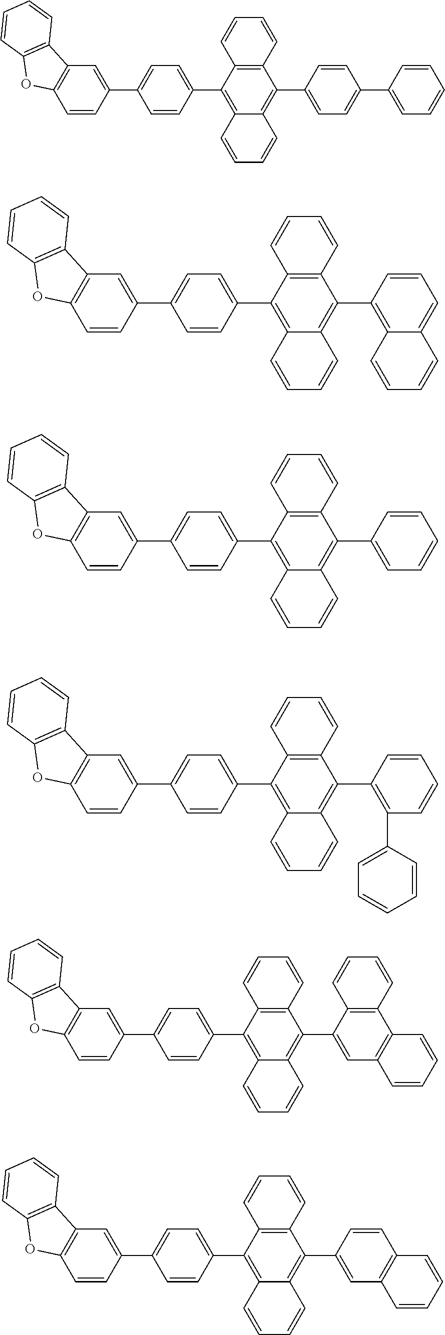 [SMILES: c1ccc(-c2ccc(-c3c4ccccc4c(-c4ccc(-c5ccc6oc7ccccc7c6c5)cc4)c4ccccc34)cc2)cc1 ; c1ccc2c(-c3c4ccccc4c(-c4ccc(-c5ccc6oc7ccccc7c6c5)cc4)c4ccccc34)cccc2c1 ; c1ccc(-c2c3ccccc3c(-c3ccc(-c4ccc5oc6ccccc6c5c4)cc3)c3ccccc23)cc1 ; c1ccc(-c2ccccc2-c2c3ccccc3c(-c3ccc(-c4ccc5oc6ccccc6c5c4)cc3)c3ccccc23)cc1 ; c1ccc2c(c1)cc(-c1c3ccccc3c(-c3ccc(-c4ccc5oc6ccccc6c5c4)cc3)c3ccccc13)c1ccccc12 ; c1ccc2cc(-c3c4ccccc4c(-c4ccc(-c5ccc6oc7ccccc7c6c5)cc4)c4ccccc34)ccc2c1]

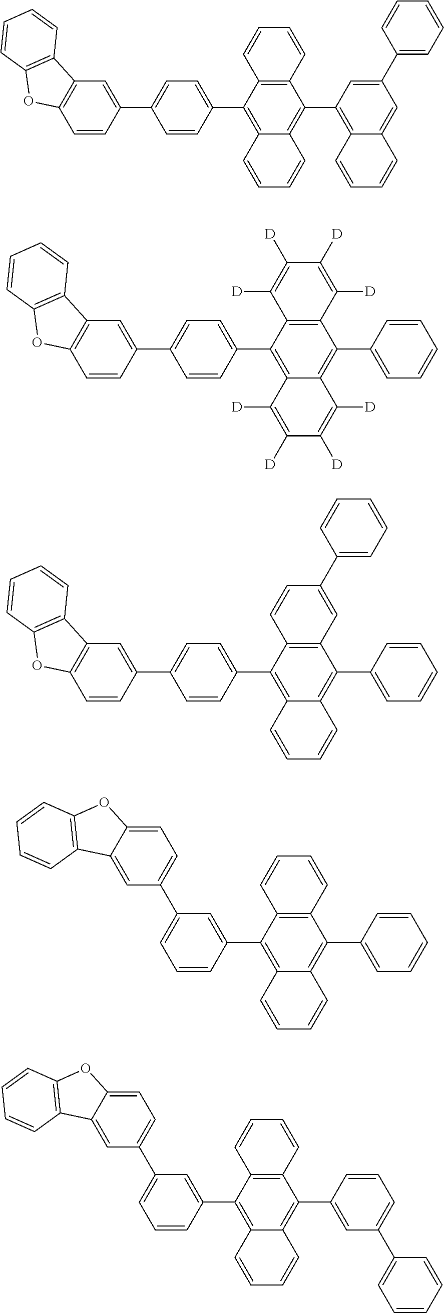

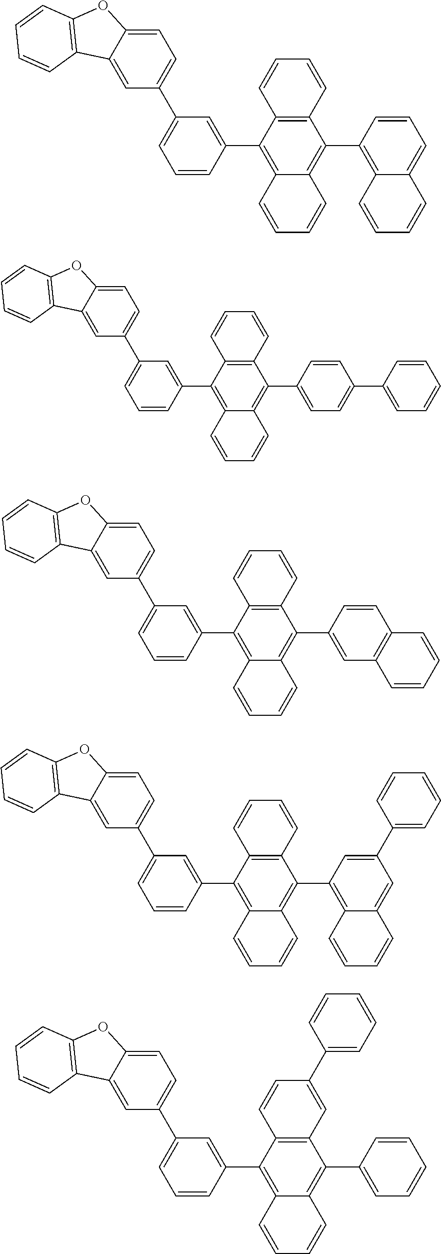

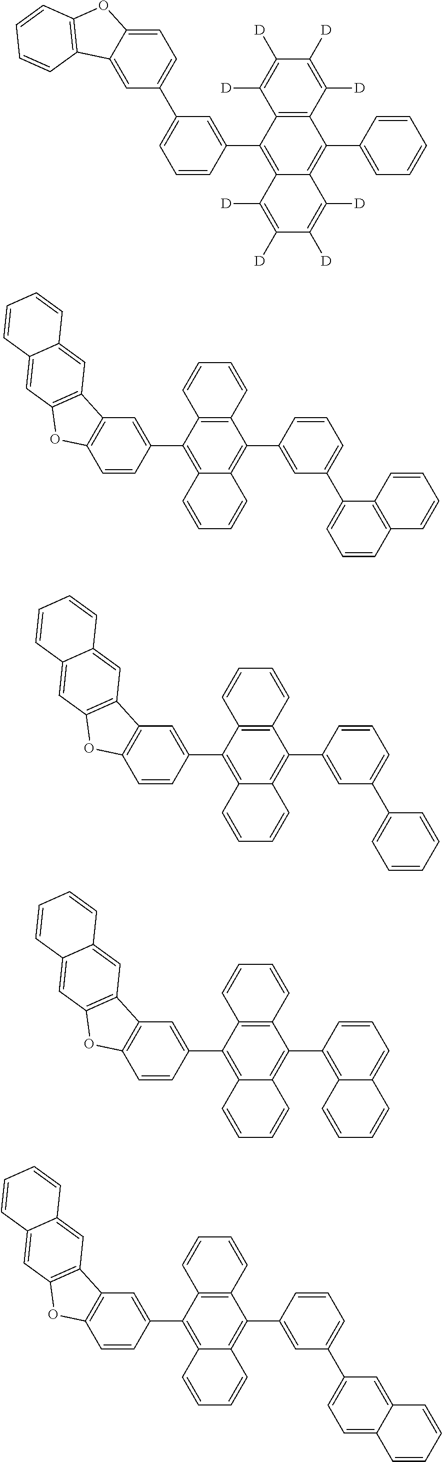 [SMILES: [2H]c1c([2H])c([2H])c2c(-c3cccc(-c4ccc5oc6ccccc6c5c4)c3)c3c([2H])c([2H])c([2H])c([2H])c3c(-c3ccccc3)c2c1[2H] ; c1cc(-c2cccc3ccccc23)cc(-c2c3ccccc3c(-c3ccc4oc5cc6ccccc6cc5c4c3)c3ccccc23)c1 ; c1ccc(-c2cccc(-c3c4ccccc4c(-c4ccc5oc6cc7ccccc7cc6c5c4)c4ccccc34)c2)cc1 ; c1ccc2cc3c(cc2c1)oc1ccc(-c2c4ccccc4c(-c4cccc5ccccc45)c4ccccc24)cc13 ; c1cc(-c2ccc3ccccc3c2)cc(-c2c3ccccc3c(-c3ccc4oc5cc6ccccc6cc5c4c3)c3ccccc23)c1]

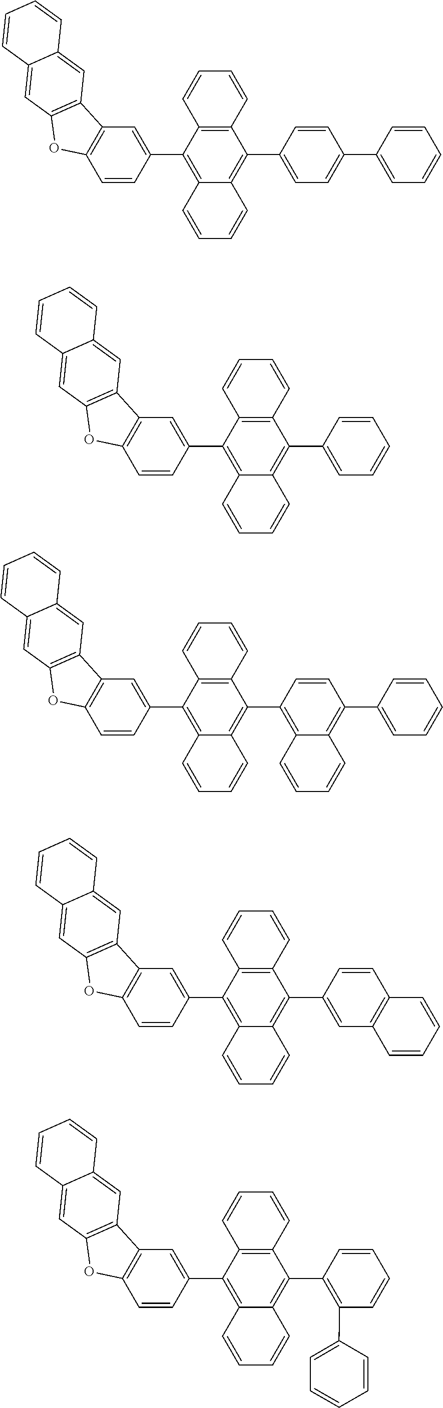 [SMILES: c1ccc(-c2ccc(-c3c4ccccc4c(-c4ccc5oc6cc7ccccc7cc6c5c4)c4ccccc34)cc2)cc1 ; c1ccc(-c2c3ccccc3c(-c3ccc4oc5cc6ccccc6cc5c4c3)c3ccccc23)cc1 ; c1ccc(-c2ccc(-c3c4ccccc4c(-c4ccc5oc6cc7ccccc7cc6c5c4)c4ccccc34)c3ccccc23)cc1 ; c1ccc2cc(-c3c4ccccc4c(-c4ccc5oc6cc7ccccc7cc6c5c4)c4ccccc34)ccc2c1 ; c1ccc(-c2ccccc2-c2c3ccccc3c(-c3ccc4oc5cc6ccccc6cc5c4c3)c3ccccc23)cc1]

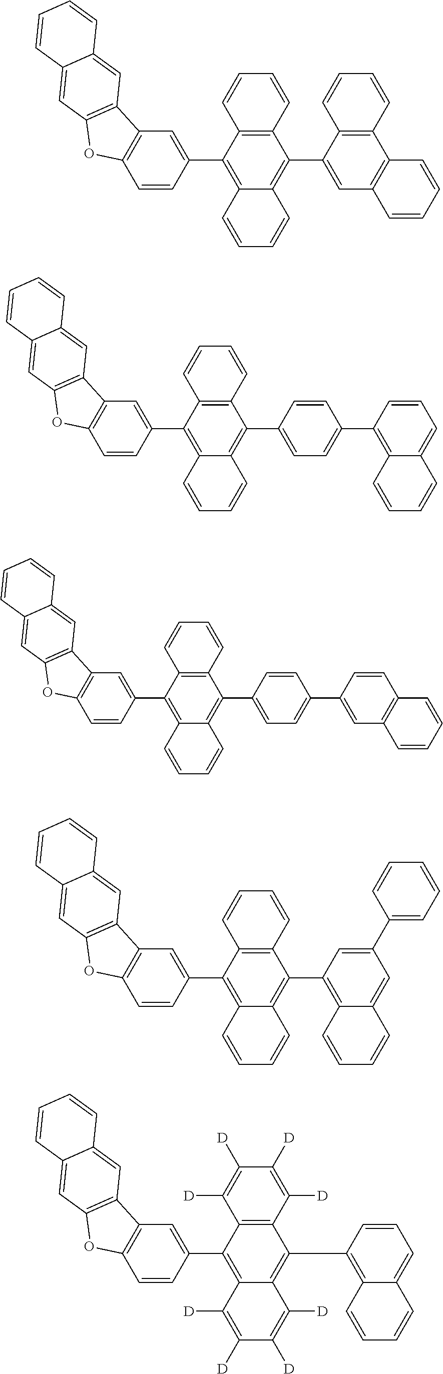 [SMILES: c1ccc2cc3c(cc2c1)oc1ccc(-c2c4ccccc4c(-c4cc5ccccc5c5ccccc45)c4ccccc24)cc13 ; c1ccc2cc3c(cc2c1)oc1ccc(-c2c4ccccc4c(-c4ccc(-c5cccc6ccccc56)cc4)c4ccccc24)cc13 ; c1ccc2cc(-c3ccc(-c4c5ccccc5c(-c5ccc6oc7cc8ccccc8cc7c6c5)c5ccccc45)cc3)ccc2c1 ; c1ccc(-c2cc(-c3c4ccccc4c(-c4ccc5oc6cc7ccccc7cc6c5c4)c4ccccc34)c3ccccc3c2)cc1 ; [2H]c1c([2H])c([2H])c2c(-c3cccc4ccccc34)c3c([2H])c([2H])c([2H])c([2H])c3c(-c3ccc4oc5cc6ccccc6cc5c4c3)c2c1[2H]]

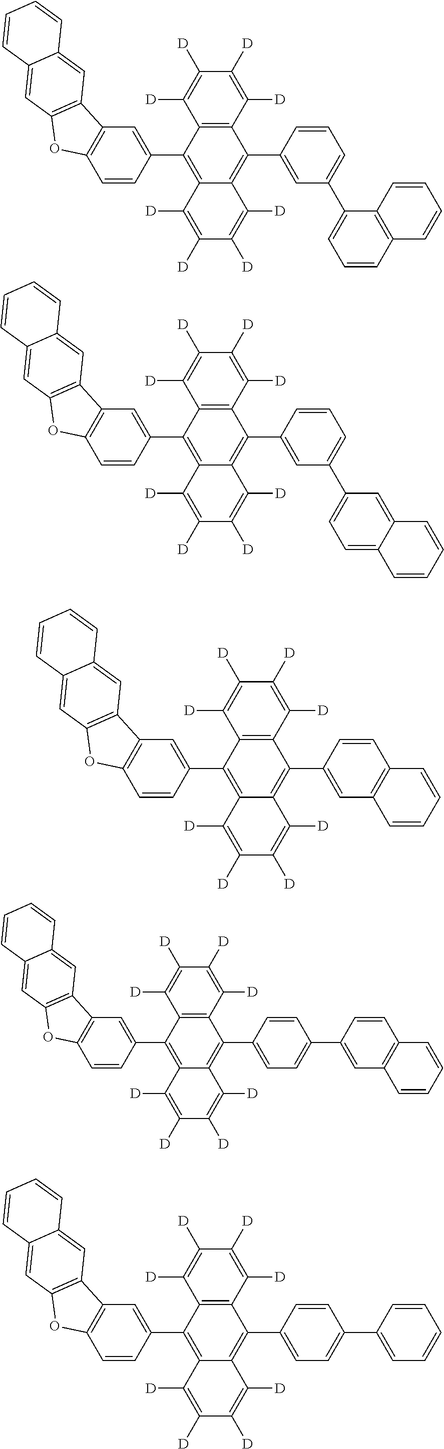

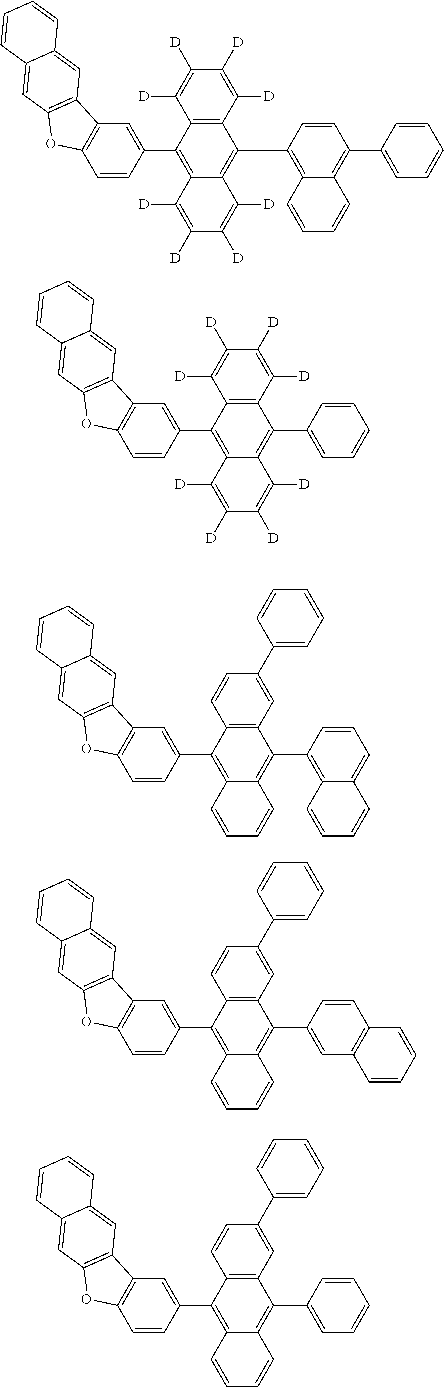

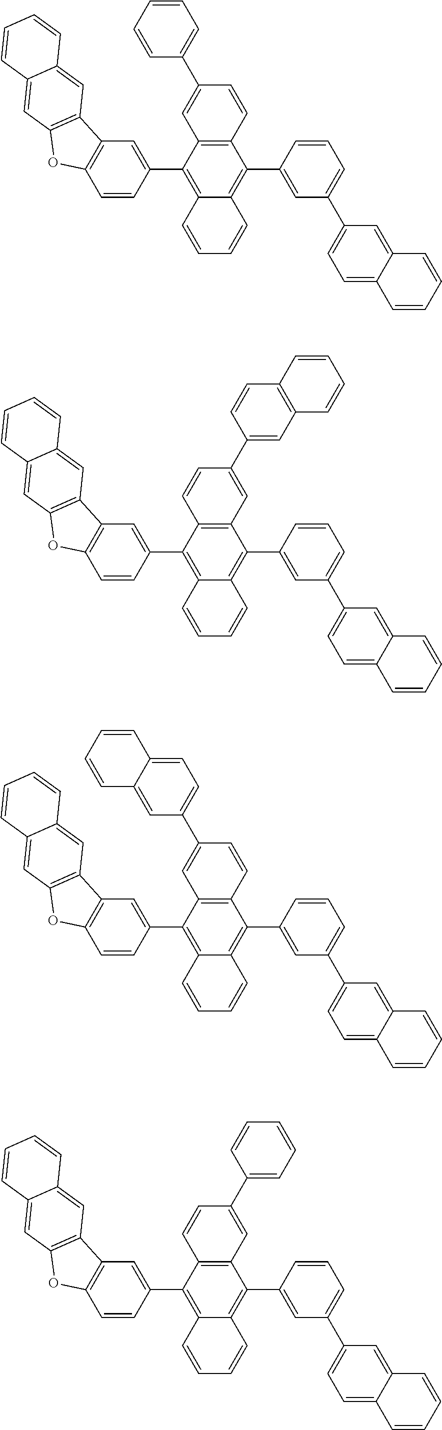 [SMILES: c1ccc(-c2ccc3c(-c4cccc(-c5ccc6ccccc6c5)c4)c4ccccc4c(-c4ccc5oc6cc7ccccc7cc6c5c4)c3c2)cc1 ; c1cc(-c2ccc3ccccc3c2)cc(-c2c3ccccc3c(-c3ccc4oc5cc6ccccc6cc5c4c3)c3ccc(-c4ccc5ccccc5c4)cc23)c1 ; c1cc(-c2ccc3ccccc3c2)cc(-c2c3ccccc3c(-c3ccc4oc5cc6ccccc6cc5c4c3)c3cc(-c4ccc5ccccc5c4)ccc23)c1 ; c1ccc(-c2ccc3c(-c4ccc5oc6cc7ccccc7cc6c5c4)c4ccccc4c(-c4cccc(-c5ccc6ccccc6c5)c4)c3c2)cc1]

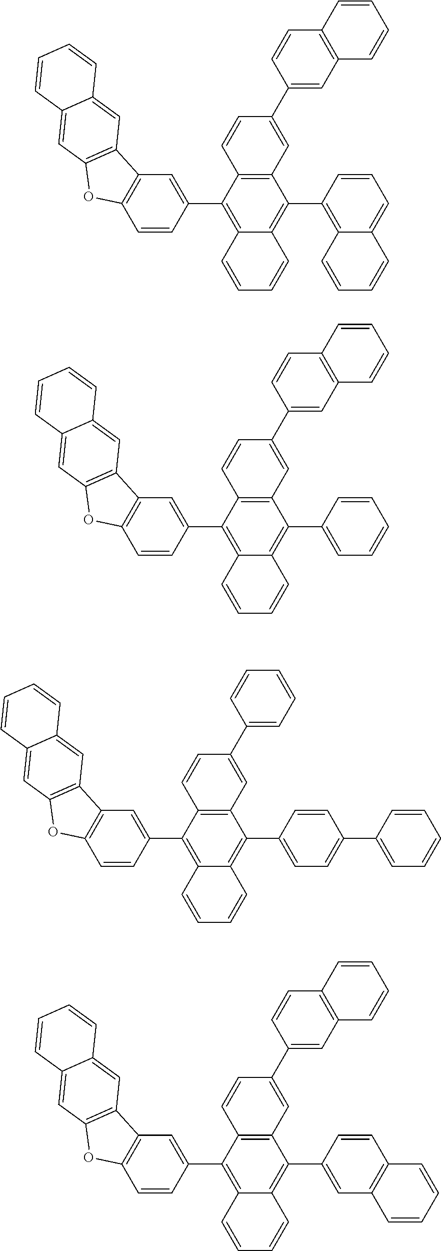

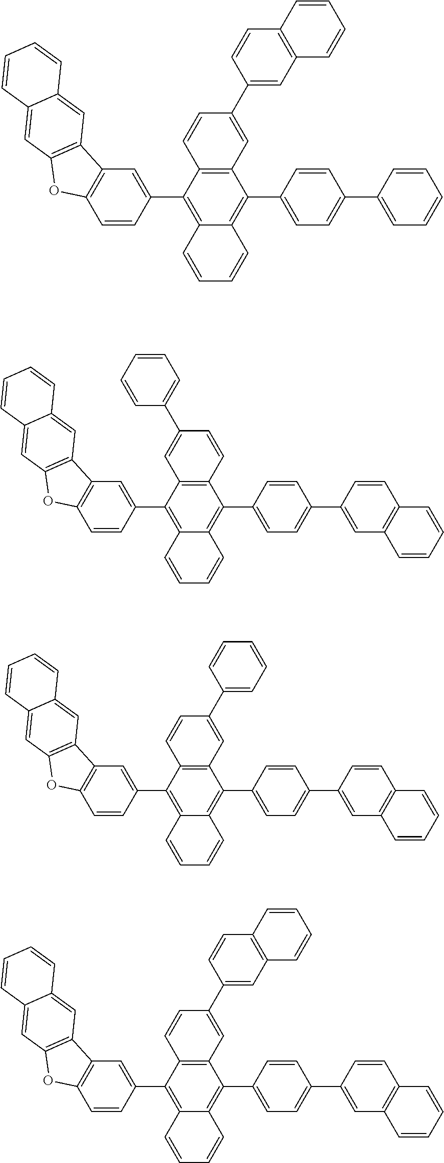 [SMILES: c1ccc(-c2ccc(-c3c4ccccc4c(-c4ccc5oc6cc7ccccc7cc6c5c4)c4ccc(-c5ccc6ccccc6c5)cc34)cc2)cc1 ; c1ccc(-c2ccc3c(-c4ccc(-c5ccc6ccccc6c5)cc4)c4ccccc4c(-c4ccc5oc6cc7ccccc7cc6c5c4)c3c2)cc1 ; c1ccc(-c2ccc3c(-c4ccc5oc6cc7ccccc7cc6c5c4)c4ccccc4c(-c4ccc(-c5ccc6ccccc6c5)cc4)c3c2)cc1 ; c1ccc2cc(-c3ccc(-c4c5ccccc5c(-c5ccc6oc7cc8ccccc8cc7c6c5)c5ccc(-c6ccc7ccccc7c6)cc45)cc3)ccc2c1]

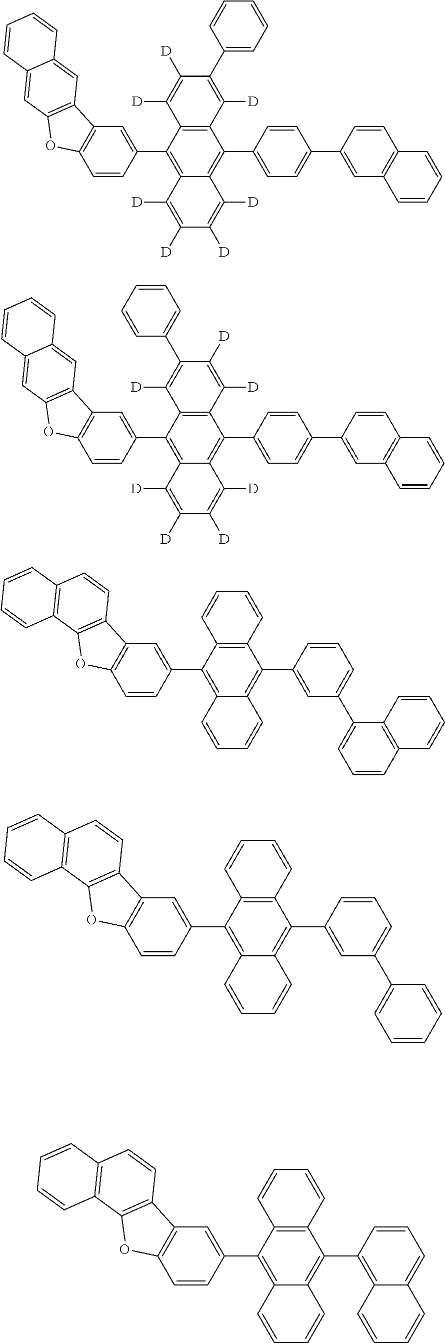

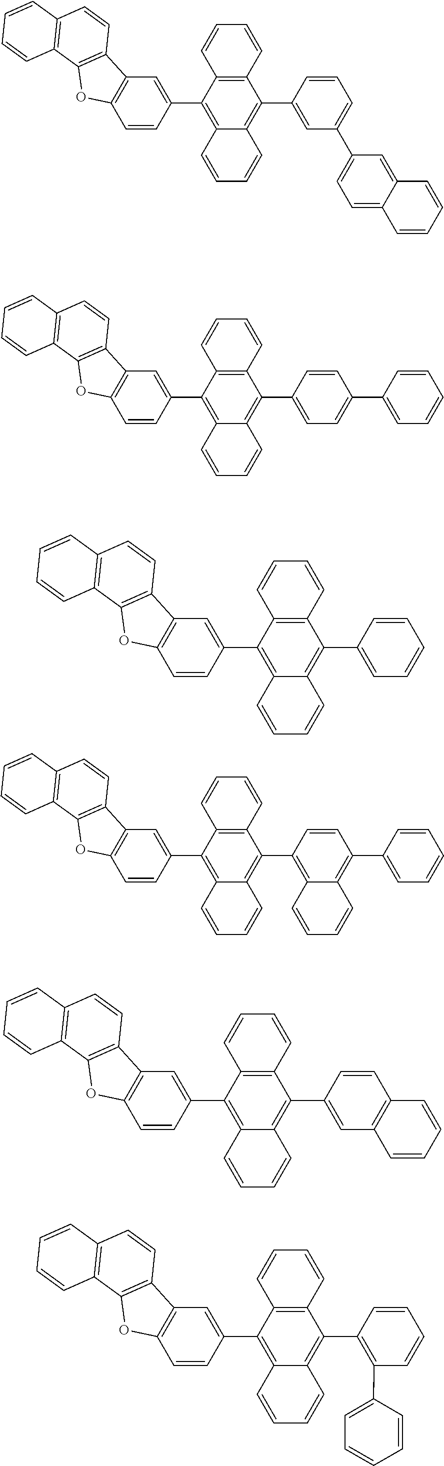 [SMILES: c1cc(-c2ccc3ccccc3c2)cc(-c2c3ccccc3c(-c3ccc4oc5c6ccccc6ccc5c4c3)c3ccccc23)c1 ; c1ccc(-c2ccc(-c3c4ccccc4c(-c4ccc5oc6c7ccccc7ccc6c5c4)c4ccccc34)cc2)cc1 ; c1ccc(-c2c3ccccc3c(-c3ccc4oc5c6ccccc6ccc5c4c3)c3ccccc23)cc1 ; c1ccc(-c2ccc(-c3c4ccccc4c(-c4ccc5oc6c7ccccc7ccc6c5c4)c4ccccc34)c3ccccc23)cc1 ; c1ccc2cc(-c3c4ccccc4c(-c4ccc5oc6c7ccccc7ccc6c5c4)c4ccccc34)ccc2c1 ; c1ccc(-c2ccccc2-c2c3ccccc3c(-c3ccc4oc5c6ccccc6ccc5c4c3)c3ccccc23)cc1]

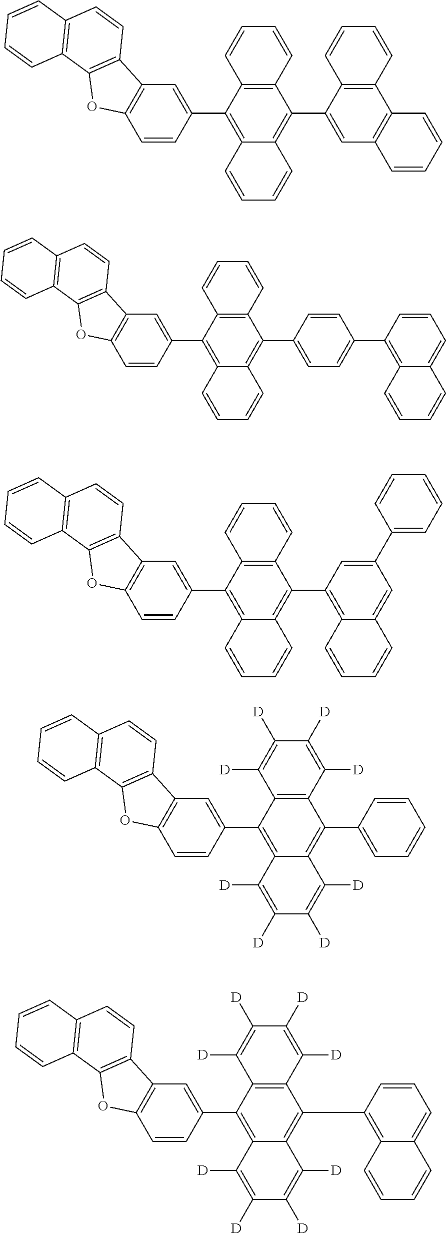

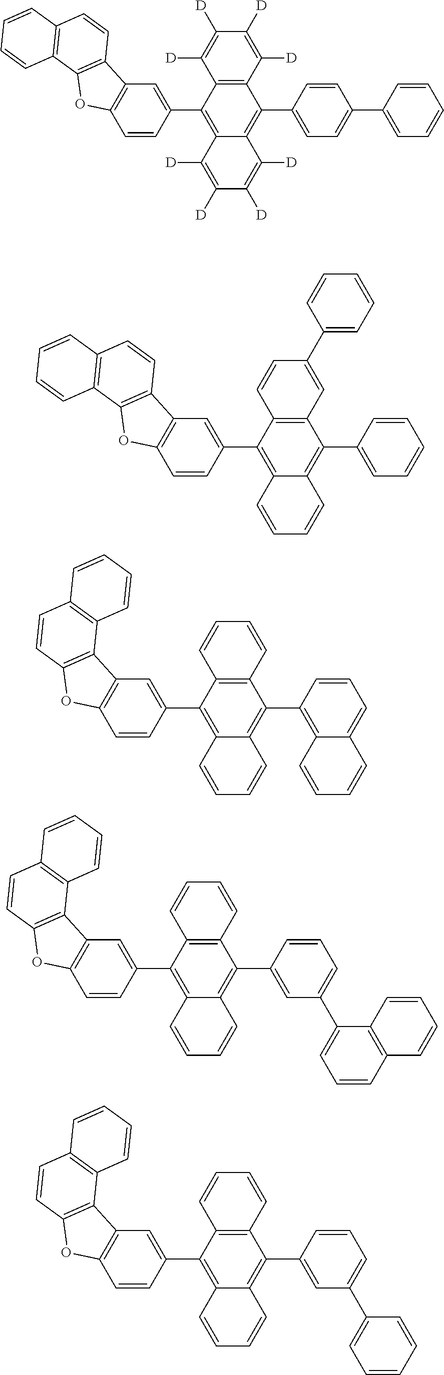 [SMILES: [2H]c1c([2H])c([2H])c2c(-c3ccc4oc5c6ccccc6ccc5c4c3)c3c([2H])c([2H])c([2H])c([2H])c3c(-c3ccc(-c4ccccc4)cc3)c2c1[2H] ; c1ccc(-c2ccc3c(-c4ccc5oc6c7ccccc7ccc6c5c4)c4ccccc4c(-c4ccccc4)c3c2)cc1 ; c1ccc2c(-c3c4ccccc4c(-c4ccc5oc6ccc7ccccc7c6c5c4)c4ccccc34)cccc2c1 ; c1cc(-c2cccc3ccccc23)cc(-c2c3ccccc3c(-c3ccc4oc5ccc6ccccc6c5c4c3)c3ccccc23)c1 ; c1ccc(-c2cccc(-c3c4ccccc4c(-c4ccc5oc6ccc7ccccc7c6c5c4)c4ccccc34)c2)cc1]

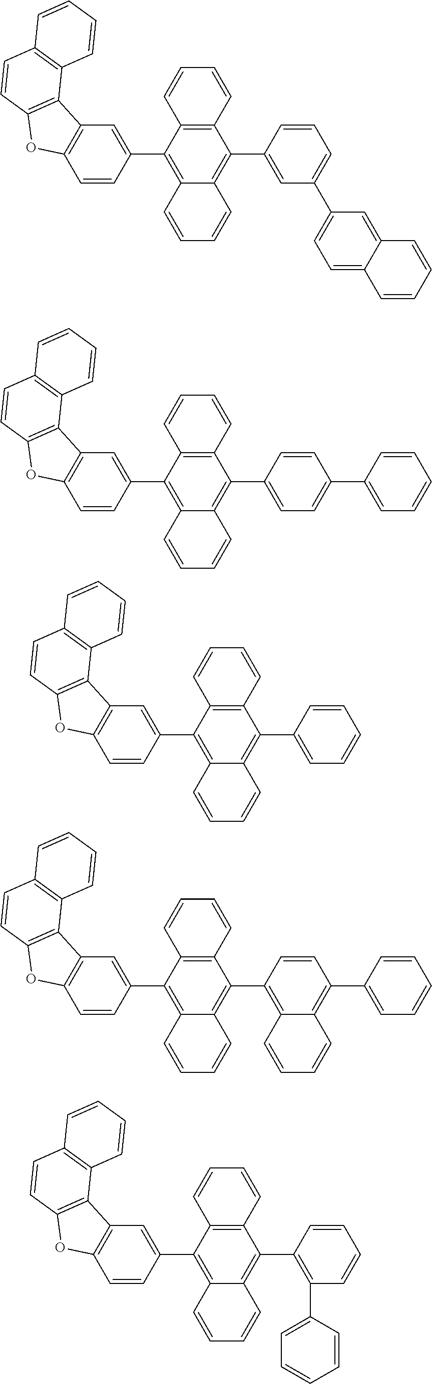 [SMILES: c1cc(-c2ccc3ccccc3c2)cc(-c2c3ccccc3c(-c3ccc4oc5ccc6ccccc6c5c4c3)c3ccccc23)c1 ; c1ccc(-c2ccc(-c3c4ccccc4c(-c4ccc5oc6ccc7ccccc7c6c5c4)c4ccccc34)cc2)cc1 ; c1ccc(-c2c3ccccc3c(-c3ccc4oc5ccc6ccccc6c5c4c3)c3ccccc23)cc1 ; c1ccc(-c2ccc(-c3c4ccccc4c(-c4ccc5oc6ccc7ccccc7c6c5c4)c4ccccc34)c3ccccc23)cc1 ; c1ccc(-c2ccccc2-c2c3ccccc3c(-c3ccc4oc5ccc6ccccc6c5c4c3)c3ccccc23)cc1]

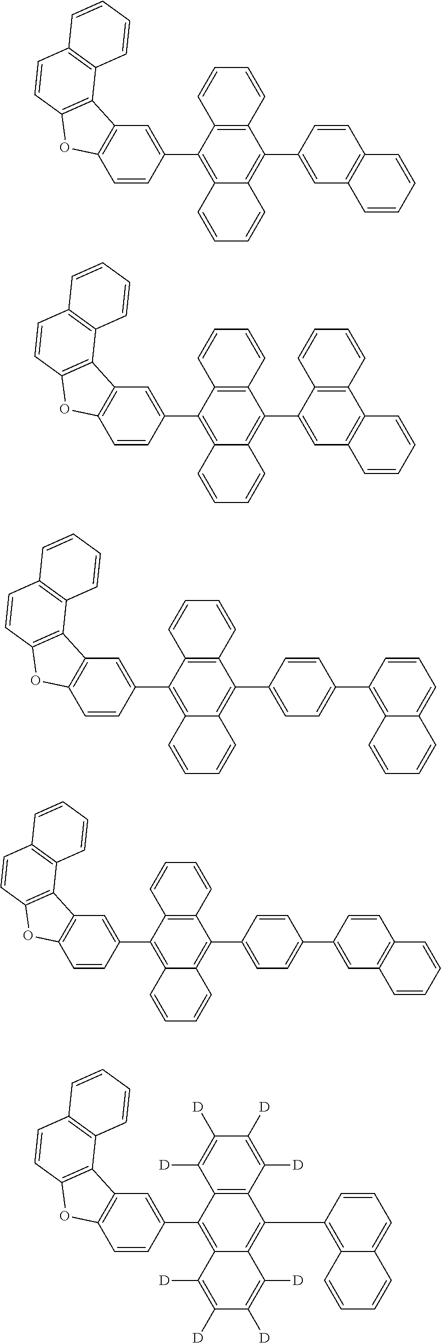 [SMILES: c1ccc2cc(-c3c4ccccc4c(-c4ccc5oc6ccc7ccccc7c6c5c4)c4ccccc34)ccc2c1 ; c1ccc2c(c1)cc(-c1c3ccccc3c(-c3ccc4oc5ccc6ccccc6c5c4c3)c3ccccc13)c1ccccc12 ; c1ccc2c(-c3ccc(-c4c5ccccc5c(-c5ccc6oc7ccc8ccccc8c7c6c5)c5ccccc45)cc3)cccc2c1 ; c1ccc2cc(-c3ccc(-c4c5ccccc5c(-c5ccc6oc7ccc8ccccc8c7c6c5)c5ccccc45)cc3)ccc2c1 ; [2H]c1c([2H])c([2H])c2c(-c3cccc4ccccc34)c3c([2H])c([2H])c([2H])c([2H])c3c(-c3ccc4oc5ccc6ccccc6c5c4c3)c2c1[2H]]

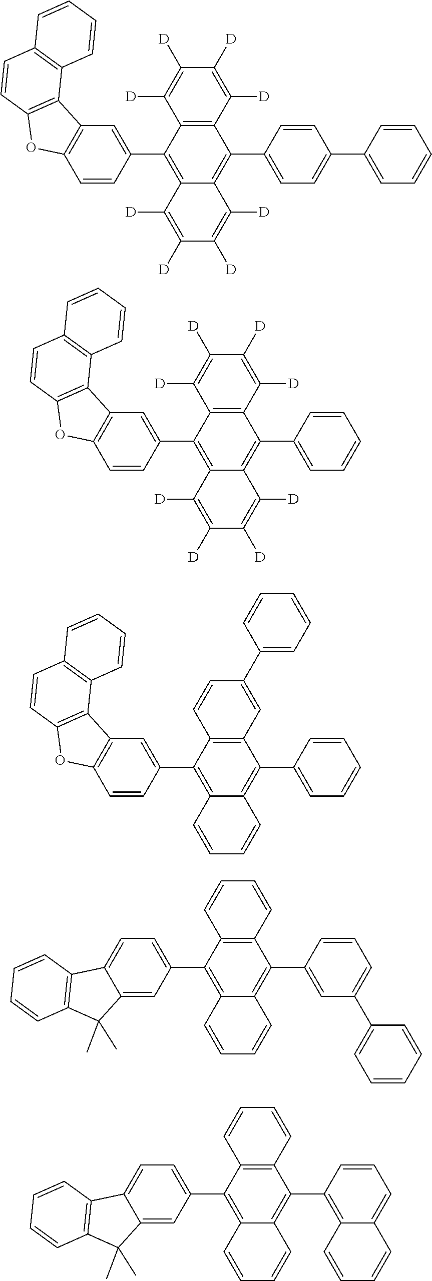

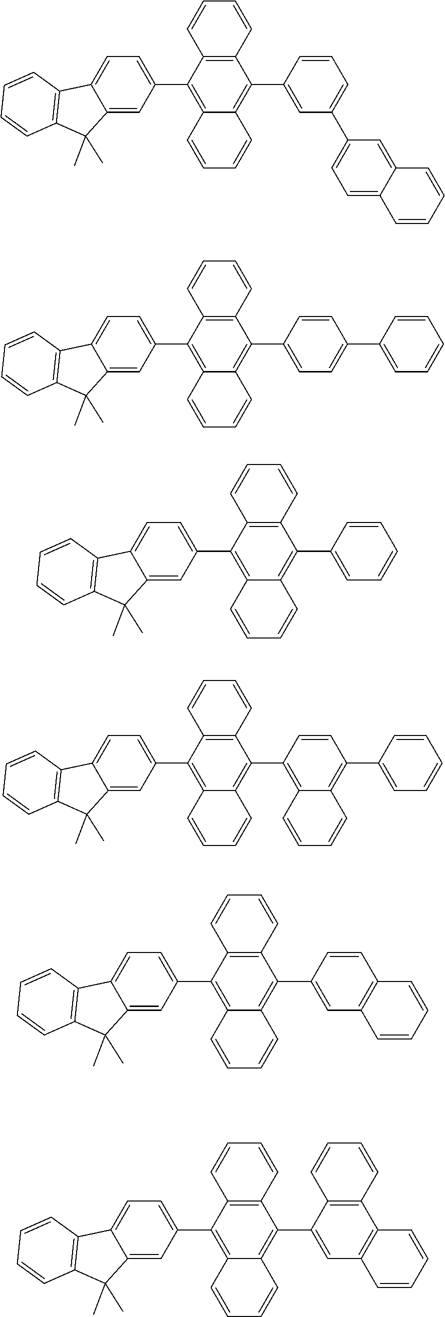

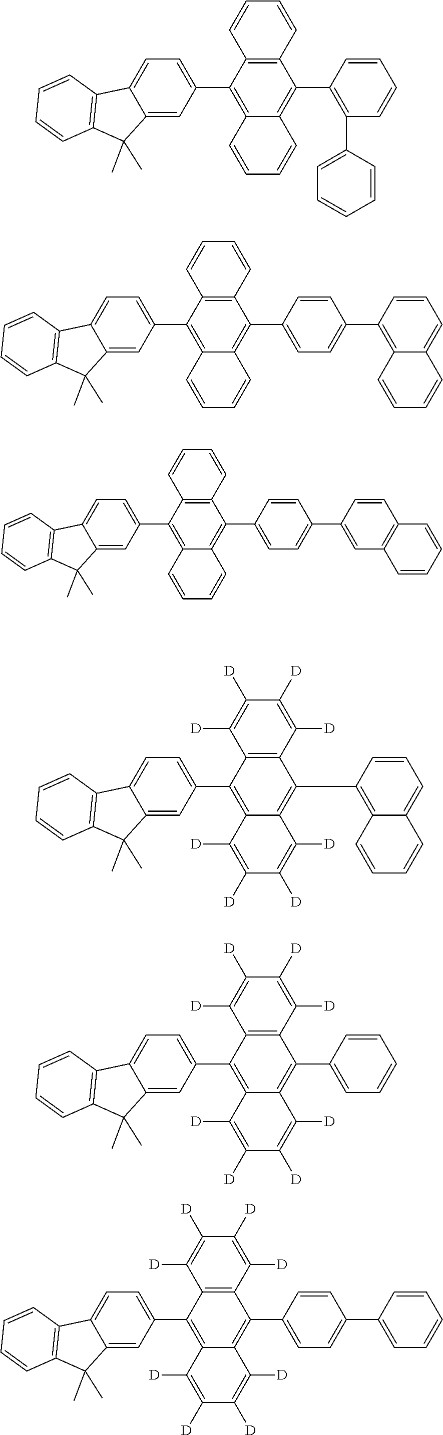

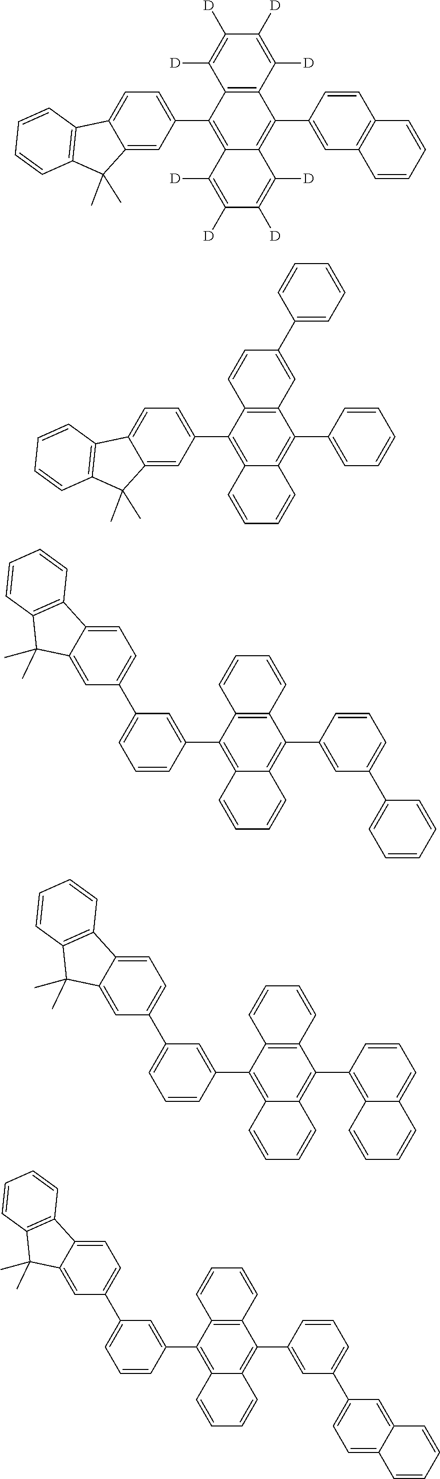

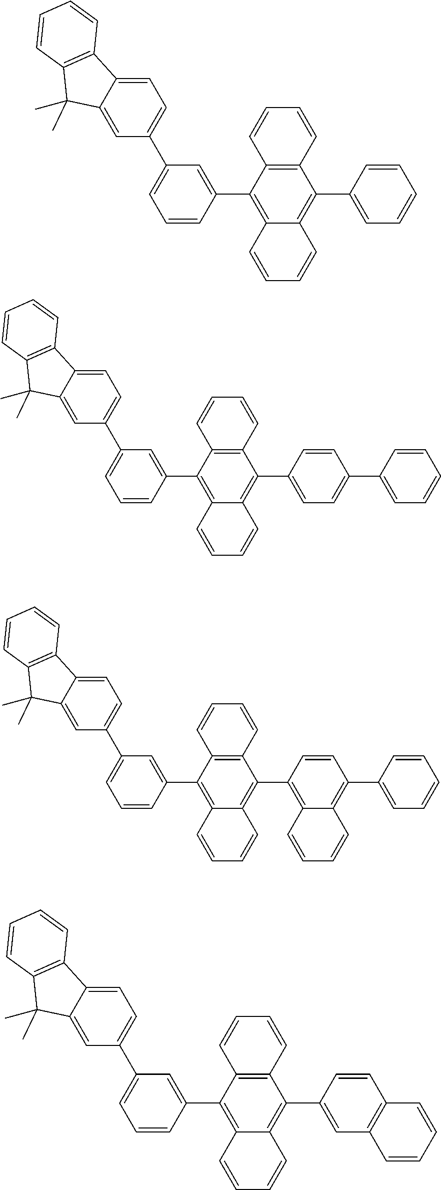

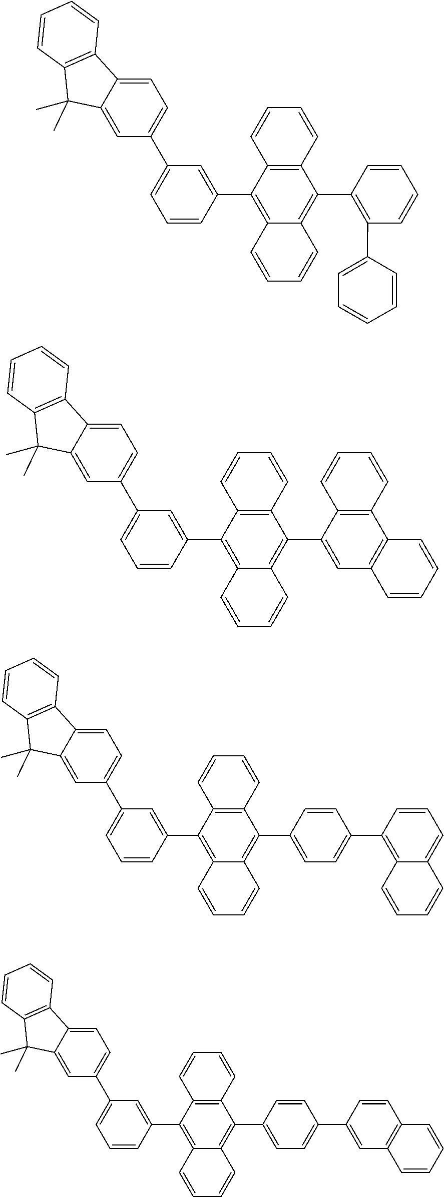

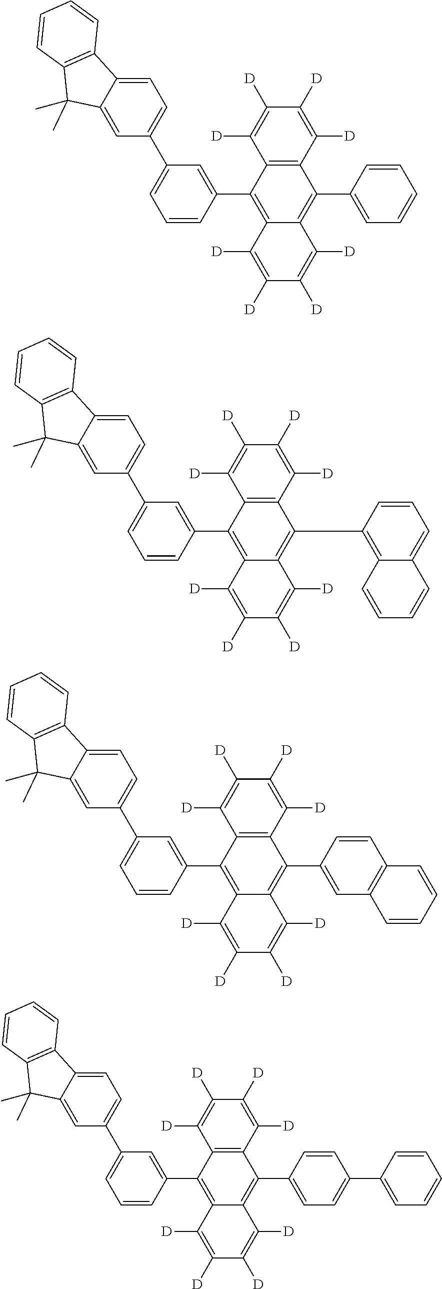 [SMILES: [2H]c1c([2H])c([2H])c2c(-c3cccc(-c4ccc5c(c4)C(C)(C)c4ccccc4-5)c3)c3c([2H])c([2H])c([2H])c([2H])c3c(-c3ccccc3)c2c1[2H] ; [2H]c1c([2H])c([2H])c2c(-c3cccc4ccccc34)c3c([2H])c([2H])c([2H])c([2H])c3c(-c3cccc(-c4ccc5c(c4)C(C)(C)c4ccccc4-5)c3)c2c1[2H] ; [2H]c1c([2H])c([2H])c2c(-c3ccc4ccccc4c3)c3c([2H])c([2H])c([2H])c([2H])c3c(-c3cccc(-c4ccc5c(c4)C(C)(C)c4ccccc4-5)c3)c2c1[2H] ; [2H]c1c([2H])c([2H])c2c(-c3cccc(-c4ccc5c(c4)C(C)(C)c4ccccc4-5)c3)c3c([2H])c([2H])c([2H])c([2H])c3c(-c3ccc(-c4ccccc4)cc3)c2c1[2H]]

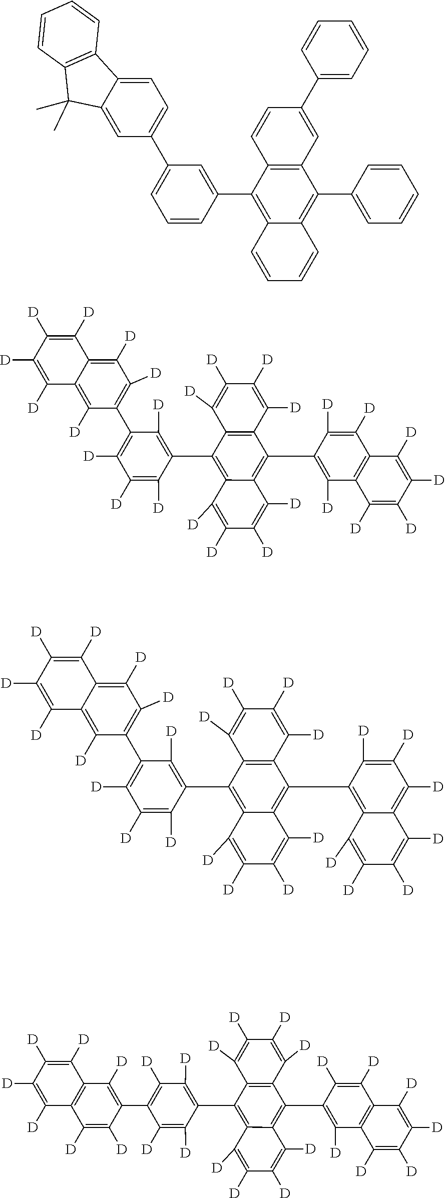

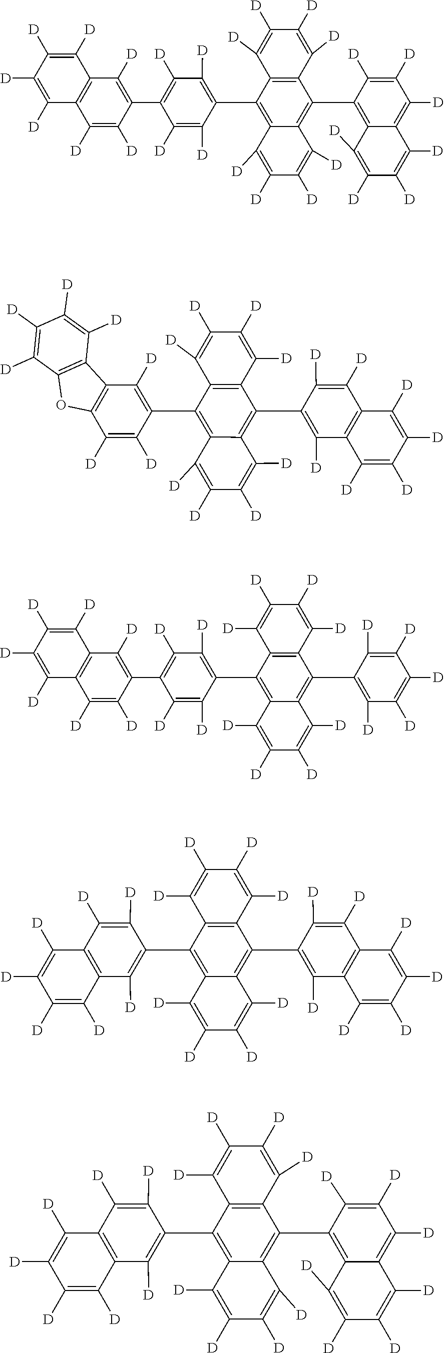

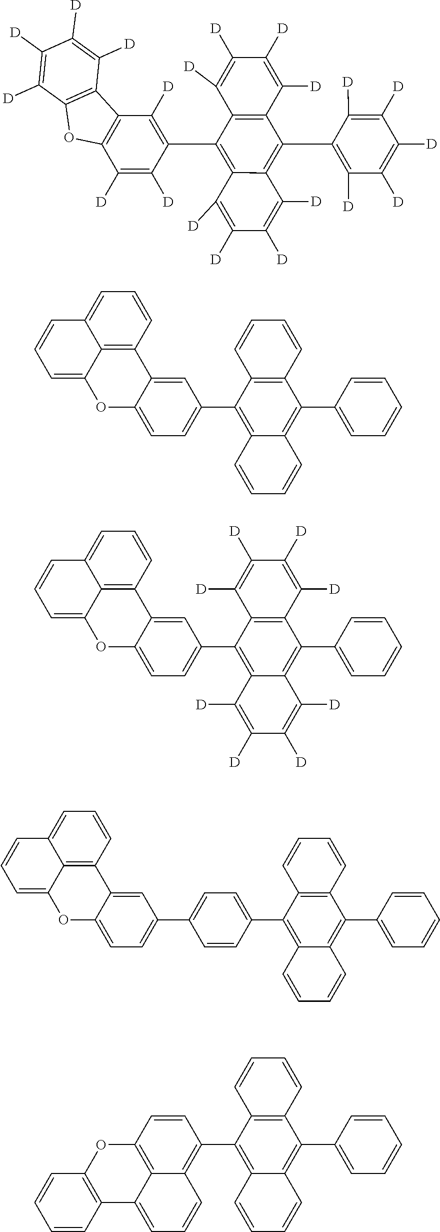

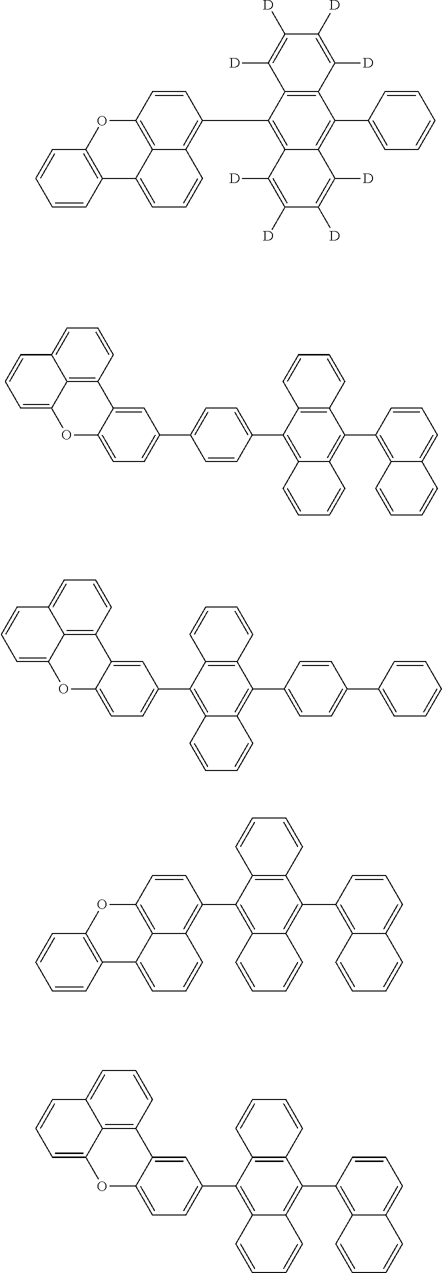 [SMILES: [2H]c1c([2H])c([2H])c2c(-c3ccc4c5c(cccc35)-c3ccccc3O4)c3c([2H])c([2H])c([2H])c([2H])c3c(-c3ccccc3)c2c1[2H] ; c1ccc2c(-c3c4ccccc4c(-c4ccc(-c5ccc6c(c5)-c5cccc7cccc(c57)O6)cc4)c4ccccc34)cccc2c1 ; c1ccc(-c2ccc(-c3c4ccccc4c(-c4ccc5c(c4)-c4cccc6cccc(c46)O5)c4ccccc34)cc2)cc1 ; c1ccc2c(c1)Oc1ccc(-c3c4ccccc4c(-c4cccc5ccccc45)c4ccccc34)c3cccc-2c13 ; c1ccc2c(-c3c4ccccc4c(-c4ccc5c(c4)-c4cccc6cccc(c46)O5)c4ccccc34)cccc2c1]

As described above, the organic EL device according to an aspect of the present invention has a cathode, an anode, and a emitting layer arranged between the cathode and the anode, wherein the emitting layer includes the compound represented by the formula (1); except that, conventionally-known materials and device configurations can be applied, as long as the effect of the present invention is not impaired.

The amount of the compound represented by the formula (1) in the emitting layer is preferably 1% by mass or more and 20% by mass or less based on the entire emitting layer.

As the representative device configuration of the organic EL device of the present invention, the following structures may be given:

(1) an anode/an emitting layer/a cathode,
(2) an anode/a hole-injecting layer/an emitting layer/a cathode,
(3) an anode/an emitting layer/an electron-injecting-transporting layer/a cathode,
(4) an anode/a hole-injecting layer/an emitting layer/an electron-injecting-transporting layer/a cathode,
(5) an anode/an organic semiconductor layer/an emitting layer/a cathode,
(6) an anode/an organic semiconductor layer/an electron-barrier layer/an emitting layer/a cathode,
(7) an anode/an organic semiconductor layer/an emitting layer/an adhesion improving layer/a cathode,
(8) an anode/a hole-injecting-transporting layer/an emitting layer/an electron-injecting-transporting layer/a cathode,
(9) an anode/an insulating layer/an emitting layer/an insulating layer/a cathode,
(10) an anode/an inorganic semiconductor layer/an insulating layer/an emitting layer/an insulating layer/a cathode,
(11) an anode/an organic semiconductor layer/an insulating layer/an emitting layer/an insulating layer/a cathode,
(12) an anode/an insulating layer/a hole-injecting-transporting layer/an emitting layer/an insulating layer/a cathode, and
(13) an anode/an insulating layer/a hole-injecting-transporting layer/an emitting layer/an electron-injecting-transporting layer/a cathode.

Among the above-described structures, the configuration of (8) is preferably used, but the device configuration of the organic EL device is not limited thereto.

In the present specification, the term “hole-injecting-transporting layer” means “at least one of the hole-injecting layer and the hole-transporting layer”, and the term “electron-injecting-transporting layer” means “at least one of the electron-injecting layer and the electron-transporting layer”.

Members which can be used in the organic EL device according to an aspect of the present invention, materials for forming each layer, other than the above-mentioned compounds, and the like, will be described below.

### (Substrate)

The substrate is used as a support of an emitting device. As the substrate, glass, quartz, plastic or the like can be used, for example. Further, a flexible substrate may be used. The term “flexible substrate” means a bendable (flexible) substrate, and specific examples thereof include a plastic substrate formed of polycarbonate, polyvinyl chloride or the like.

### (Anode)

For the anode formed on the substrate, metals, alloys, electrically conductive compounds, mixtures thereof, and the like, which have large work function (specifically 4.0 eV or more) are preferably used. Specific examples thereof include indium oxide-tin oxide (ITO: Indium Tin Oxide), indium oxide-tin oxide containing silicon or silicon oxide, indium oxide-zinc oxide, tungsten oxide, indium oxide containing zinc oxide, graphene, and the like. In addition thereto, specific examples thereof include gold (Au), platinum (Pt), a nitride of a metallic material (for example, titanium nitride), or the like.

### (Hole-Injecting Layer)

The hole-injecting layer is a layer containing a substance having high hole-injecting property. As the substance having high hole-injecting property, molybdenum oxide, titanium oxide, vanadium oxide, rhenium oxide, ruthenium oxide, chromium oxide, zirconium oxide, hafnium oxide, tantalum oxide, silver oxide, tungsten oxide, manganese oxide, an aromatic amine compound, a polymer compound (oligomers, dendrimers, polymers, and the like), or the like can be given.

### (Hole-Transporting Layer)

The hole-transporting layer is a layer containing a substance having high hole-transporting property. For the hole-transporting layer, an aromatic amine compound, a carbazole derivative, an anthracene derivative, or the like can be used. A polymer compound such as poly(N-vinylcarbazole) (abbreviation: PVK) and poly(4-vinyltriphenylamine) (abbreviation: PVTPA) can also be used. Provided that a substance other than the above-described substances may be used as long as the substance has higher hole-transporting property than electron-transporting property. The layer containing the substance having high hole-transporting property may be not only a single layer, but also layers in which two or more layers formed of the above-described substances are stacked.

### (Guest (Dopant) Material of Emitting Layer)

The emitting layer is a layer containing a substance having high luminous property, and various materials can be used in addition to the material (the compound represented by the formula (1)) used in the present invention described above. For example, as the substance having high emitting property, a fluorescent compound which emits fluorescence or a phosphorescent compound which emits phosphorescence can be used. The fluorescent compound is a compound which can emit from a singlet excited state, and the phosphorescent compound is a compound which can emit from a triplet excited state.

As a blue fluorescent emitting material which can be used for the emitting layer, pyrene derivatives, styrylamine derivatives, chrysene derivatives, fluoranthene derivatives, fluorene derivatives, diamine derivatives, triarylamine derivatives, and the like can be used. As a green fluorescent emitting material which can be used for the emitting layer, aromatic amine derivatives and the like can be used. As a red fluorescent emitting material which can be used for the emitting layer, tetracene derivatives, diamine derivatives and the like can be used.

As a blue phosphorescent emitting material which can be used for the emitting layer, metal complexes such as iridium complexes, osmium complexes and platinum complexes are used. As a green phosphorescent emitting material which can be used for the emitting layer, iridium complexes and the like are used. As a red phosphorescent emitting material which can be used for the emitting layer, metal complexes such as iridium complexes, platinum complexes, terbium complexes and europium complexes are used.

### (Host Material for Emitting Layer)

The emitting layer may have a constitution in which the substance having high emitting property (guest material) is dispersed in another substance (host material). As a substance for dispersing the substance having high emitting property, a variety of substances can be used in addition to the material (the compound represented by the formula (10)) used in the present invention described above, and it is preferable to use a substance having a higher lowest unoccupied molecular orbital level (LUMO level) and a lower highest occupied molecular orbital level (HOMO level) than a substance having high emitting property.

As a substance (host material) for dispersing the substance having high emitting property, 1) a metal complex such as an aluminum complex, a beryllium complex, and a zinc complex, 2) a heterocyclic compound such as an oxadiazole derivative, a benzimidazole derivative, and a phenanthroline derivative, 3) a fused aromatic compound such as a carbazole derivative, an anthracene derivative, a phenanthrene derivative, a pyrene derivative, a naphthacene derivative, a fluoranthene derivative, a triphenylene derivative, a fluorene derivative, and a chrysene derivative, and 3) an aromatic amine compound such as a triarylamine derivative and a fused polycyclic aromatic amine derivative are used.

A compound having delayed fluorescence (thermally activated delayed fluorescence) can also be used as the host material. It is also preferable that the emitting layer includes the material used in the present invention described above and the host compound having delayed fluorescence.

### (Electron-Transporting Layer)

The electron-transporting layer is a layer containing a substance having high electron-transporting property. For the electron-transporting layer, 1) a metal complex such as an aluminum complex, a beryllium complex, and a zinc complex; 2) a heteroaromatic complex such as an imidazole derivative, a benzimidazole derivative, an azine derivative, carbazole derivative, and a phenanthroline derivative; and 3) a polymer compound can be used.

### (Electron-Injecting Layer)

The electron-injecting layer is a layer containing a substance having high electron-injecting property. For the electron-injecting layer, lithium (Li), ytterbium (Yb), lithium fluoride (LiF), cesium fluoride (CsF), calcium fluoride (CaF₂), a metal complex compound such as 8-hydroxyquinolinolato-lithium (Liq), an alkali metal such as lithium oxide (LiOₓ), an alkaline earth metal, or a compound thereof can be used.

### (Cathode)

For the cathode, metals, alloys, electrically conductive compounds, mixtures thereof, and the like, which have small work function (specifically 3.8 eV or less) are preferably used. Specific examples of such a cathode material include an element belonging to Group 1 or Group 2 of the Periodic Table of the Elements, i.e., an alkali metal such as lithium (Li) and cesium (Cs), an alkaline earth metal such as magnesium (Mg), calcium (Ca) and strontium (Sr), and an alloy containing these (e.g., MgAg and AILi); a rare earth metal such as europium (Eu) and ytterbium (Yb), and an alloy containing these.

In the organic EL device according to an aspect of the present invention, the method for forming each layer is not particularly limited. A conventionally-known method for forming each layer such as a vacuum deposition process and a spin coating process can be used. Each layer such as the emitting layer can be formed by a known method such as a vacuum deposition process, a molecular beam deposition process (MBE process), or an application process such as a dipping process, a spin coating process, a casting process, a bar coating process and a roll coating process, using a solution prepared by dissolving the material in a solvent.

In the organic EL device according to an aspect of the present invention, the thickness of each layer is not particularly limited, but is normally preferable several nm to 1 μm generally in order to suppress defects such as pinholes, to suppress applied voltages to be low, and to improve luminous efficiency.

### [Electronic Apparatus]

An electronic apparatus according to an aspect of the present invention is characterized by including the organic EL device according to an aspect of the present invention.

Specific examples of the electronic apparatus include display components such as an organic EL panel module; display devices for a television, a cellular phone and a personal computer; and emitting devices such as a light and a vehicular lamp; and the like.

### EXAMPLES

Hereinafter, Examples according to the present invention will be described. The present invention is not limited to these Examples.

### <Compound>

Compounds represented by the formula (1) used in the Examples are shown below.

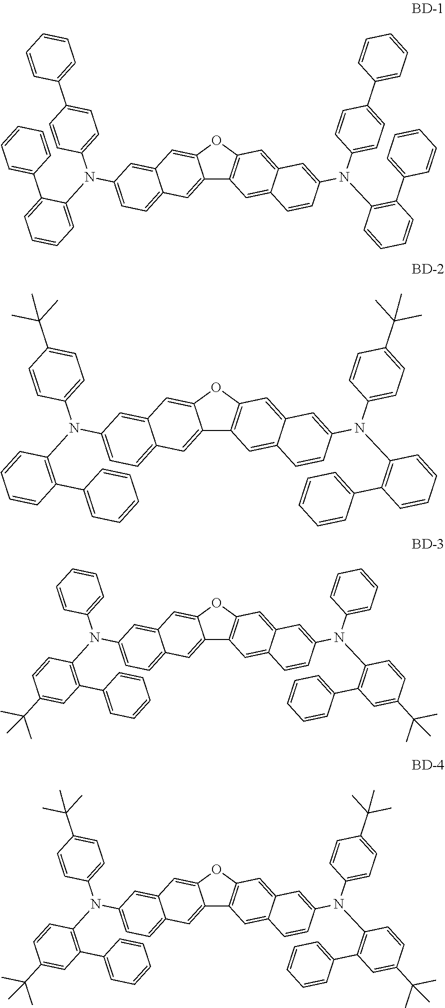

Compounds used in the Comparative Examples are shown below.

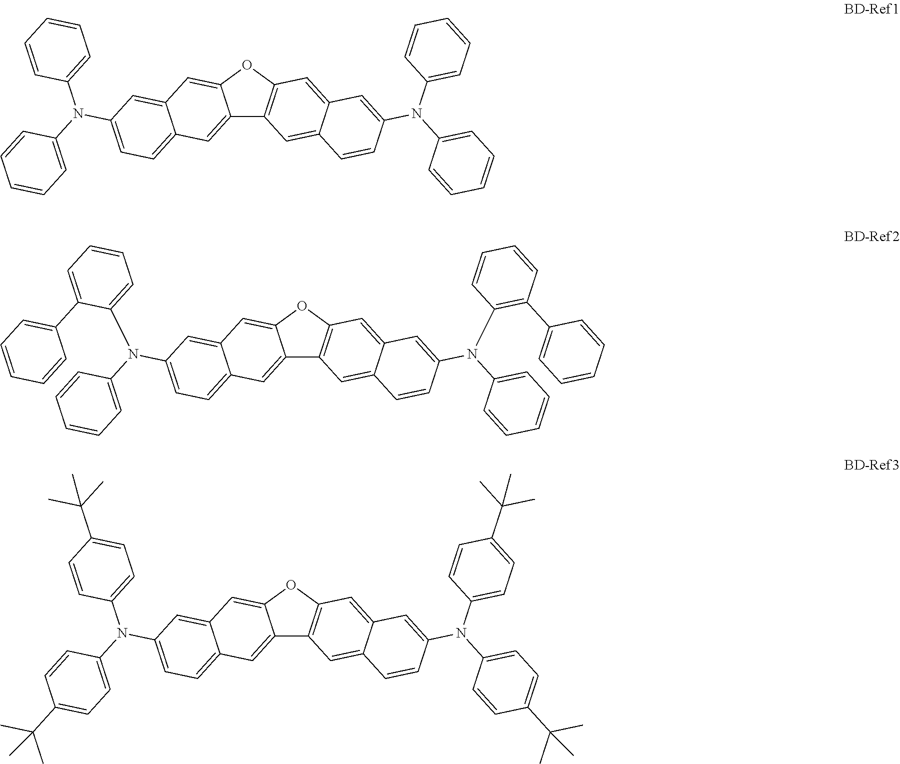

Compounds used in the Examples and the Comparative Examples are shown below.

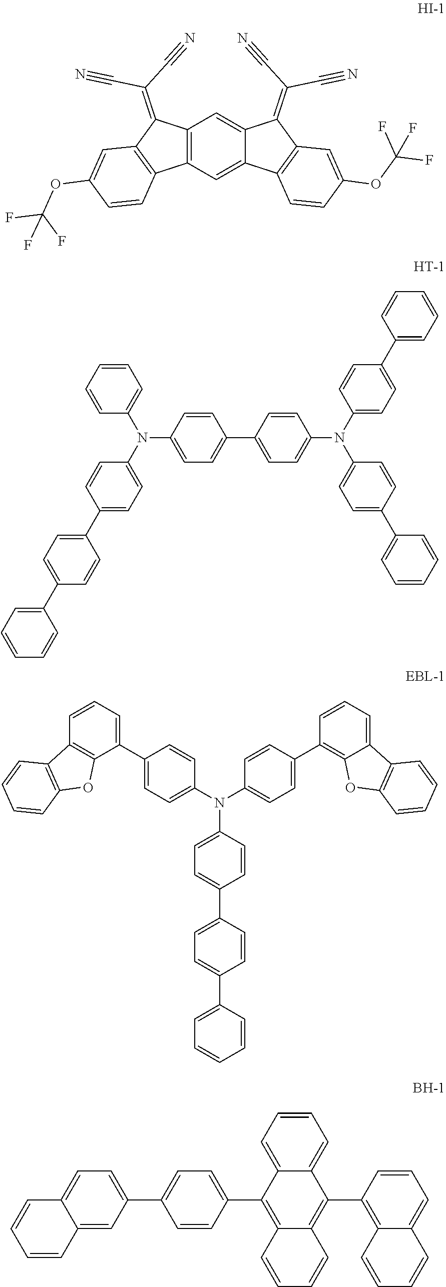

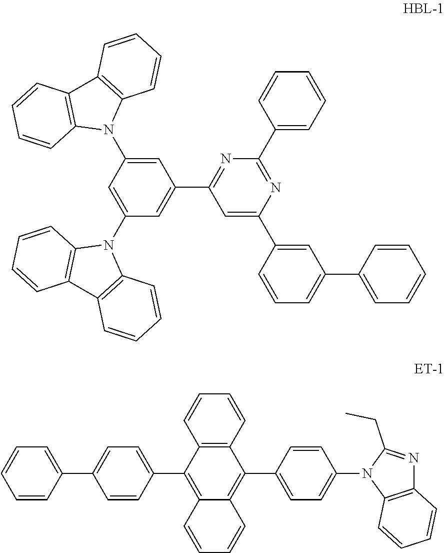 [SMILES: c1ccc(-c2cccc(-c3cc(-c4cc(-n5c6ccccc6c6ccccc65)cc(-n5c6ccccc6c6ccccc65)c4)nc(-c4ccccc4)n3)c2)cc1 ; CCc1nc2ccccc2n1-c1ccc(-c2c3ccccc3c(-c3ccc(-c4ccccc4)cc3)c3ccccc23)cc1]

### Example 1

### <Fabrication of Organic EL Device>

A 25 mm×75 mm×1.1 mm-thick glass substrate with an ITO transparent electrode (anode) (manufactured by GEOMATEC Co., Ltd.) was subjected to ultrasonic cleaning in isopropyl alcohol for 5 minutes, and then subjected to UV-ozone cleaning for 30 minutes. The ITO has the film thickness of 130 nm.

The glass substrate with the transparent electrode after being cleaned was mounted onto a substrate holder in a vacuum vapor deposition apparatus. First, a compound HI-1 was deposited on the surface on the side where the transparent electrode was formed so as to cover the transparent electrode to form a compound HI-1 film having the thickness of 5 nm. The HI-1 film functions as a hole-injecting layer.

Subsequent to the formation of the HI-1 film, a compound HT-1 was deposited thereon to form an HT-1 film having the thickness of 80 nm on the HI-1 film. The HT-1 film functions as a first hole-transporting layer.

Following the formation of the HT-1 film, a compound EBL-1 was deposited thereon to form an EBL-1 film having the thickness of 10 nm on the HT-1 film. The EBL-1 film functions as a second hole-transporting layer.

BH-1 (host material) and BD-1 (dopant material) were co-deposited on the EBL-1 film to be 2% in a proportion (weight ratio) of the compound BD-1 to form an emitting layer having the thickness of 25 nm.

A compound HBL-1 was deposited on the emitting layer to form an electron-transporting layer having the thickness of 10 nm. A compound ET-1 being an electron-injecting material was deposited on the electron-transporting layer to form an electron-injecting layer having the thickness of 15 nm. LiF was deposited on the electron-injecting layer to form a LiF film having the thickness of 1 nm. Metal Al was deposited on the LiF film to form a metal cathode having the thickness of 80 nm.

The device configuration of the organic EL device of Example 1 is schematically shown as follows.
ITO(130)/HI-1(5)/HT-1(80)/EBL-1(10)/BH-1:BD-1(25:2%)/HBL-1(10)/ET-1(15)/LiF(1)/Al(80)

The numerical values in parentheses indicate the film thickness (unit: nm).

### <Evaluation of Organic EL Device>

### (Device Lifetime)

Regarding the obtained organic EL device, a voltage was applied to the obtained organic EL device at room temperature so that the current density became 50 mA/cm², and the time until the luminance became 95% of the initial luminance (LT95 (unit: hours)) was measured. The numerical values in the table are relative values when Comparative Example 1 described later is 100%.

### (External Quantum Efficiency)

A voltage was applied to the organic EL device so that the current density became 10 mA/cm² and the EL emission spectrum was measured by using Spectroradiometer CS-2000 (manufactured by KONICA MINOLTA, INC.). External quantum efficiency (EQE) (%) was calculated from the obtained spectral emission luminance spectrum. The results are shown in Table 1. The numerical values in the table are relative values when Comparative Example 1 described later is 100%.

### Examples 2 to 4 and Comparative Examples 1 to 3

Organic EL devices were prepared and evaluated in the same manner as in Example 1, except that compounds shown in Table 1 were used as the dopant material of the emitting layer. The results are shown in Table 1.

TABLE 1

| | | External quantum | LT95 |
|---|---|---|---|
| | Dopant | efficiency (%) | (%) |
| Example 1 | BD-1 | 105 | 189 |
| Example 2 | BD-2 | 110 | 210 |
| Example 3 | BD-3 | 111 | 220 |
| Example 4 | BD-4 | 108 | 271 |
| Comparative | BD-Ref1 | 100 | 100 |
| Example 1 | | | |
| Comparative | BD-Ref2 | 100 | 140 |
| Example 2 | | | |
| Comparative | BD-Ref3 | 108 | 114 |
| Example 3 | | | |

### <Synthesis of Compound>

### Synthesis of BD-1

The compound BD-1 was synthesized through the synthetic route described below.

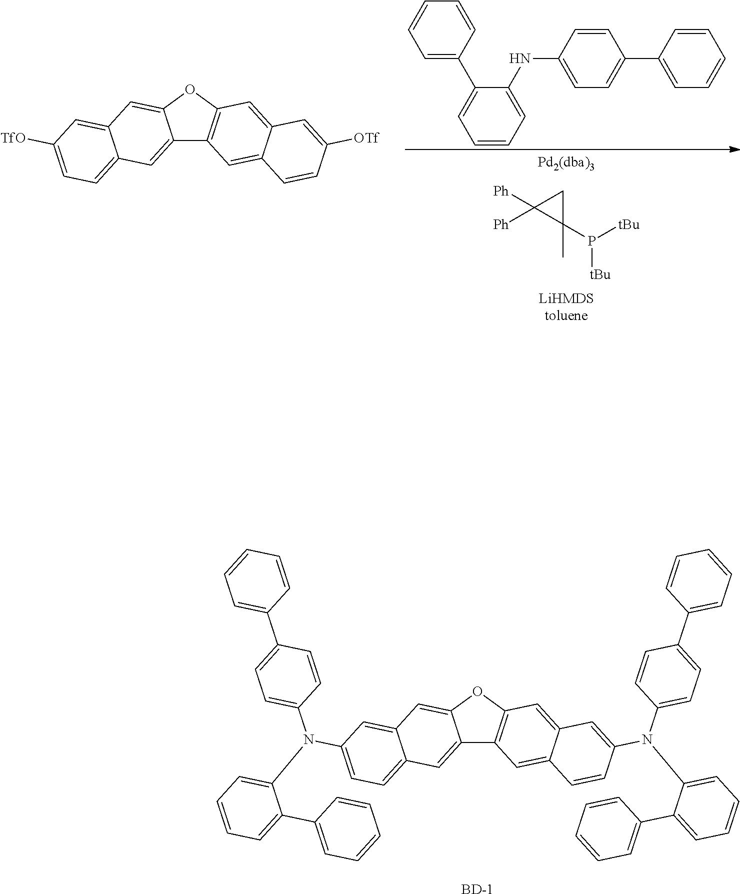

1,1′-dinaphtho[2,3-b:2′,3′-d]furan-3,9-bistrifluoromethanesulfonate (synthesized according to Example 1 of WO2018/235953) (4.0 g), N-(4-biphenylyl)-2-biphenylamine (manufactured by Tokyo Chemical Industry Co., Ltd.) (5.0 g), Tris(dibenzylideneacetone)diparazium (0) (Pd₂(dba)₃) (0.13 g), and di-tert-butyl (1-methyl-2,2-diphenylcyclopropyl)phosphine (0.20 g) were added to a three-necked eggplant flask, and dehydrated toluene (150 ml) was added thereto. The solution was heated to 70° C. under an argon atmosphere and stirred for 30 minutes, and 20 mL of a toluene solution of lithium (bistrimethylsilyl) amide (LiHMDS) (1 mol/L) was added dropwise into the system, followed by stirring for 6 hours. The solution was allowed to cool to room temperature, and then subjected to silica gel column chromatography to obtain BD-1.

The yield was 1.1 g (17% yield). The molecular weight of BD-1 was 907, and the mass spectrum of the obtained compound was analyzed as m/z (ratio of mass to charge)=907, thereby identified as BD-1.

### Synthesis of BD-2

### (1) Synthesis of Intermediate A

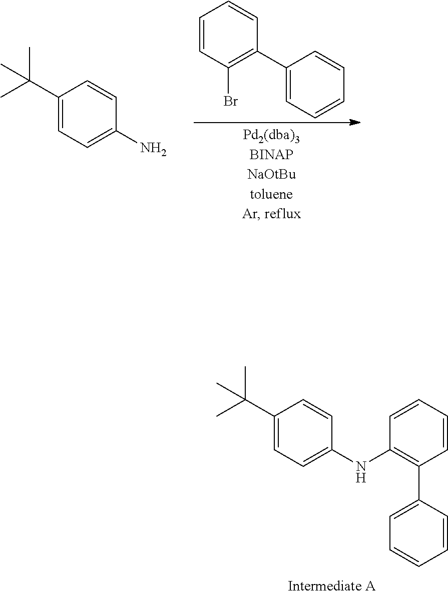 [SMILES: CC(C)(C)c1ccc(N)cc1 ; Brc1ccccc1-c1ccccc1 ; CC(C)(C)c1ccc(Nc2ccccc2-c2ccccc2)cc1]

4-tert-butyl-aniline (manufactured by Tokyo Chemical Industry Co., Ltd.) (5.4 g), 2′-bromobiphenyl (manufactured by Tokyo Chemical Industry Co., Ltd.) (5.0 μg), Tris(dibenzylideneacetone) diparazium (0) (manufactured by Sigma-Aldrich Co. LLC) (0.25 g), 2,2′-bis(diphenylphosphino)-1,1′-binaphthyl (BINAP, manufactured by FUJIFILM Wako Pure Chemical Corporation) (0.35 g), sodium butoxide (3.1 g) and toluene (150 mL) were added to a 300 mL three-necked eggplant flask, and they were refluxed for five hours under an argon atmosphere. They were cooled to room temperature, and the reaction solution was subjected to silica gel column chromatography to obtain a colorless oil (5.5 g, 55% yield). The molecular weight of Intermediate A was 301, and the mass spectrum of the obtained compound was analyzed as m/z (ratio of mass to charge)=301, thereby the present compound was identified as Intermediate A.

### (2) Synthesis of BD-2

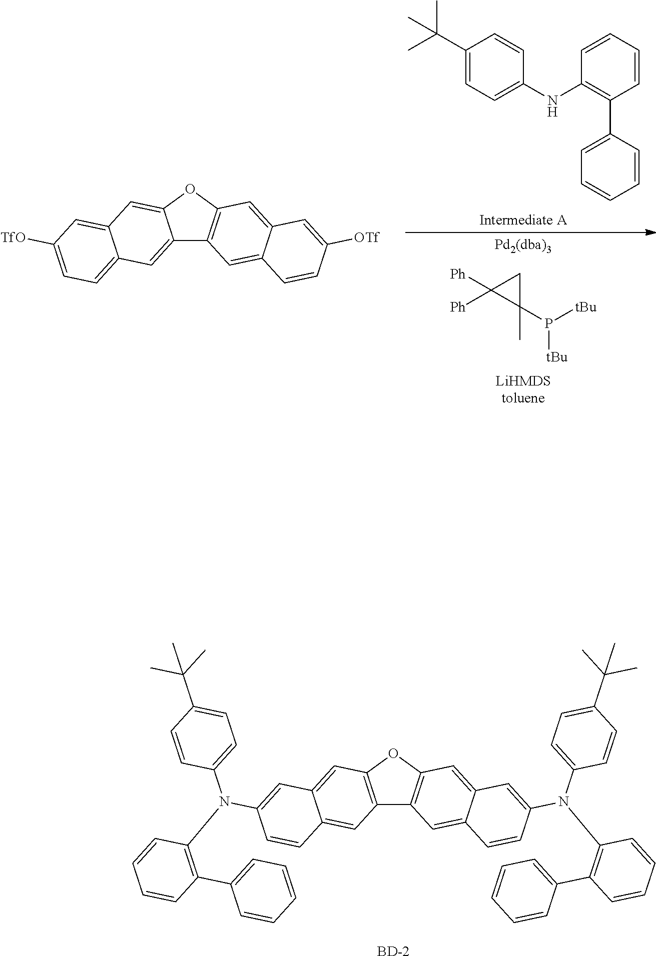 [SMILES: O=S(=O)(Oc1ccc2cc3c(cc2c1)oc1cc2cc(OS(=O)(=O)C(F)(F)F)ccc2cc13)C(F)(F)F ; CC(C)(C)c1ccc(Nc2ccccc2-c2ccccc2)cc1 ; CC(C)(C)P(C(C)(C)C)C1(C)CC1(c1ccccc1)c1ccccc1 ; CC(C)(C)c1ccc(N(c2ccc3cc4c(cc3c2)oc2cc3cc(N(c5ccc(C(C)(C)C)cc5)c5ccccc5-c5ccccc5)ccc3cc24)c2ccccc2-c2ccccc2)cc1 ; "BD-2"]

1,1′-dinaphtho[2,3-b:2′,3′-d]furan-3,9-bistrifluoromethanesulfonate (0.85 g), Intermediate A (1.0 g), Pd₂(dba)₃ (50 mg) and di-tert-butyl(1-methyl-2,2-diphenylcyclopropyl)phosphine (80 mg) were added to a three-necked eggplant flask, dehydrated toluene (30 mL) was added thereto. The solution was heated to 70° C. under an argon atmosphere and stirred for 30 minutes, and 4 mL of a toluene solution of LiHMDS (1 mol/L) was added dropwise into the system, followed by stirring for 6 hours. The solution was allowed to cool to room temperature, and then subjected to silica gel column chromatography to obtain a yellow solid. The yield was 0.85 g (66% yield). The molecular weight of BD-2 was 867, and the mass spectrum of the obtained compound was analyzed as m/z (ratio of mass to charge)=867, thereby the present compound was identified as BD-2.

### Synthesis of BD-3

### (1) Synthesis of Intermediate B

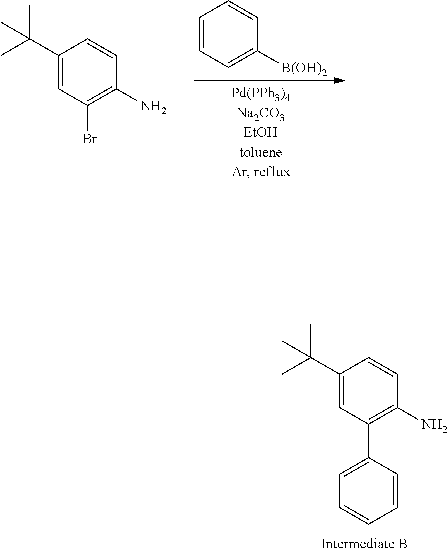 [SMILES: CC(C)(C)c1ccc(N)c(Br)c1 ; OB(O)c1ccccc1 ; CC(C)(C)c1ccc(N)c(-c2ccccc2)c1]

2-bromo-4-(tert-butyl)aniline (manufactured by Tokyo Chemical Industry Co., Ltd.) (5.3 g), phenylboronic acid (manufactured by FUJIFILM Wako Pure Chemical Corporation) (4.0 g), tetrakis (triphenylphosphine) palladium (manufactured by Sigma-Aldrich Co. LLC) (0.25 g), sodium carbonate (5.8 g), water (20 mL), ethanol (20 mL) and toluene (20 mL) were added to a three-necked eggplant flask (200 mL), heated to 80° C. under an argon atmosphere, and stirred for 6 hours. The reaction solution was cooled to room temperature, purified by silica gel column chromatography, and then recrystallized by hexane to obtain a colorless solid. The yield was 3.2 g (65% yield). The molecular weight of Intermediate B was 225, and the mass spectrum of the obtained compound was analyzed as m/z (ratio of mass to charge)=225, thereby identified as Intermediate B.

### (2) Synthesis of Intermediate C

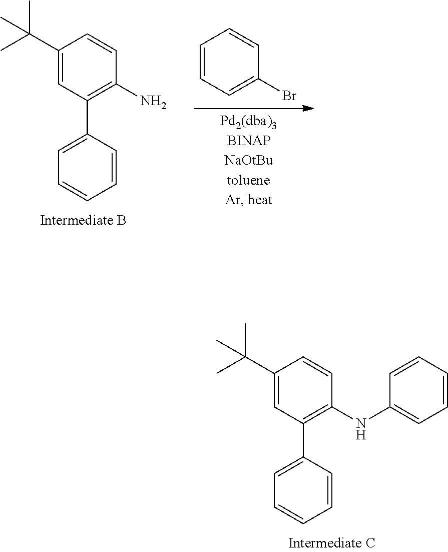 [SMILES: CC(C)(C)c1ccc(N)c(-c2ccccc2)c1 ; Brc1ccccc1 ; CC(C)(C)c1ccc(Nc2ccccc2)c(-c2ccccc2)c1]

Intermediate B (3.2 g), bromobenzene (manufactured by Sigma-Aldrich Co. LLC) (1.6 mL), BINAP (manufactured by FUJIFILM Wako Pure Chemical Corporation) (45 mg), tris (dibenzylideneacetone) dipalladium (0) (manufactured by Sigma-Aldrich Co. LLC) (22 mg), sodium butoxide (1.3 g) and toluene (30 mL) were added to a 100 mL three-necked eggplant flask, and stirred at 90° C. for 3 hours under an argon atmosphere. The reaction solution was cooled to room temperature, and purified by silica gel column chromatography to obtain 4.3 g of a pale yellow oil (100% yield). The molecular weight of Intermediate C was 301, and the mass spectrum of the obtained compound was analyzed as m/z (ratio of mass to charge)=301, thereby identified as Intermediate C.

### (3) Synthesis of BD-3

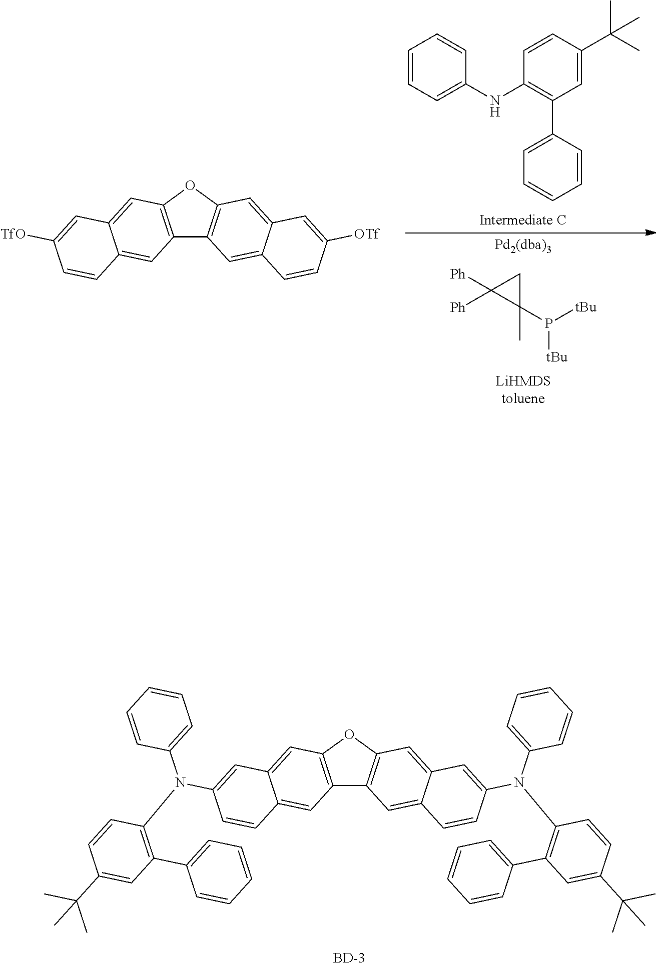 [SMILES: O=S(=O)(Oc1ccc2cc3c(cc2c1)oc1cc2cc(OS(=O)(=O)C(F)(F)F)ccc2cc13)C(F)(F)F ; CC(C)(C)c1ccc(Nc2ccccc2)c(-c2ccccc2)c1 ; CC(C)(C)P(C(C)(C)C)C1(C)CC1(c1ccccc1)c1ccccc1 ; CC(C)(C)c1ccc(N(c2ccccc2)c2ccc3cc4c(cc3c2)oc2cc3cc(N(c5ccccc5)c5ccc(C(C)(C)C)cc5-c5ccccc5)ccc3cc24)c(-c2ccccc2)c1 ; "BD-3"]

1,1′-dinaphtho[2,3-b:2′,3′-d]furan-3,9-bistrifluoromethanesulfonate (1.0 g), Intermediate C (1.2 g), Pd₂(dba)₃ (32 mg), and di-tert-butyl(1-methyl-2,2-diphenylcyclopropyl) phosphine (50 mg) were added to a three-necked eggplant flask, and dehydrated toluene (150 mL) was added thereto. The solution was heated to 70° C. under an argon atmosphere and stirred for 30 minutes, and 5 mL of a toluene solution of LiHMDS (1 mol/L) was added dropwise into the system, followed by stirring for 6 hours. The solution was allowed to cool to room temperature, and then subjected to silica gel column chromatography to obtain BD-3. The yield was 0.94 g (61% yield). The molecular weight of BD-3 was 867, and the mass spectrum of the obtained compound was analyzed as m/z (ratio of mass to charge)=867, thereby identified as BD-3.

### Synthesis of BD-4

### (1) Synthesis of Intermediate D

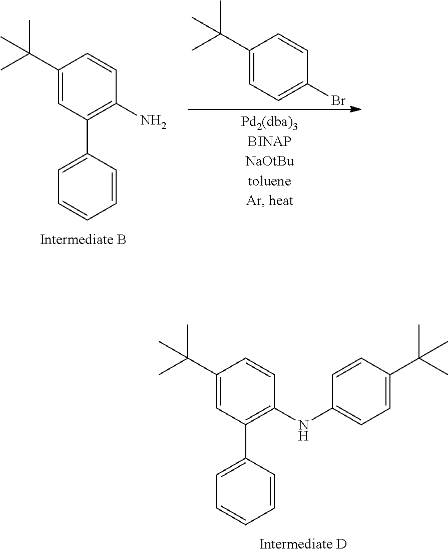 [SMILES: CC(C)(C)c1ccc(N)c(-c2ccccc2)c1 ; CC(C)(C)c1ccc(Br)cc1 ; CC(C)(C)c1ccc(Nc2ccc(C(C)(C)C)cc2-c2ccccc2)cc1]

Intermediate B (3.0 g), 1-bromo-4-(tert-butyl)benzene (3.4 g, manufactured by Tokyo Chemical Industry Co., Ltd.), BINAP (0.17 g, manufactured by FUJIFILM Wako Pure Chemical Corporation), Tris (dibenzylideneacetone) dipalladium (0) (85 mg, manufactured by Sigma-Aldrich Co., Ltd.), sodium butoxide (1.9 g) and toluene (40 mL) were added to a 100 mL three-necked eggplant flask, and stirred at 90° C. for 6 hours under an argon atmosphere. The reaction solution was cooled to room temperature, and purified by silica gel column chromatography to obtain a pale yellow oil. The yield was 2.8 g (59% yield). The molecular weight of Intermediate D was 357, and the mass spectrum of the obtained compound was analyzed as m/z (ratio of mass to charge)=357, thereby identified as Intermediate D.

### (2) Synthesis of BD-4

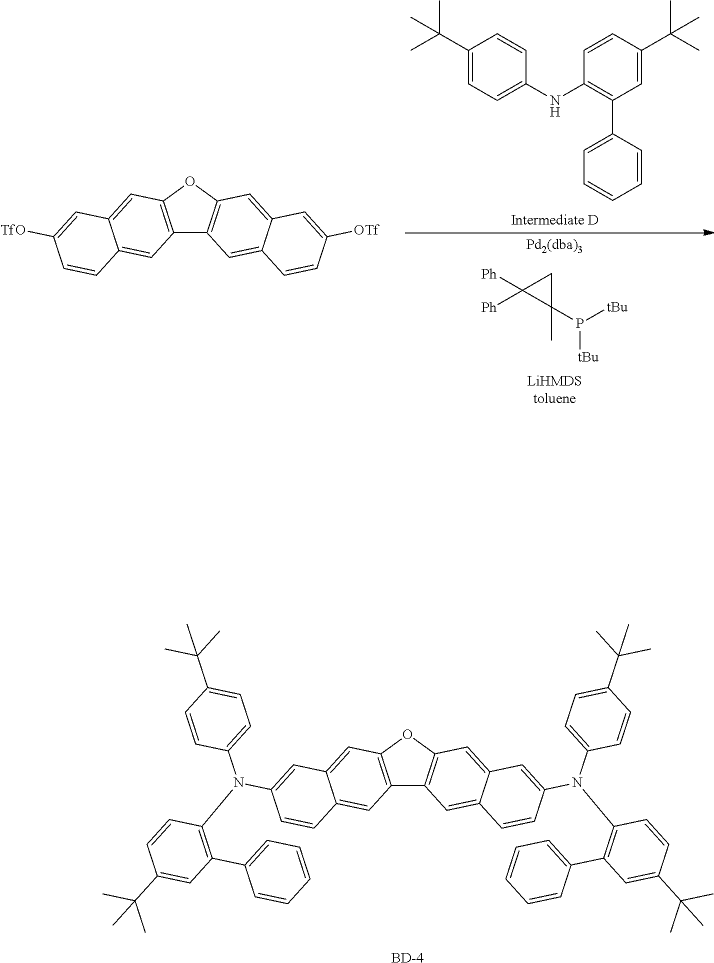 [SMILES: O=S(=O)(Oc1ccc2cc3c(cc2c1)oc1cc2cc(OS(=O)(=O)C(F)(F)F)ccc2cc13)C(F)(F)F ; CC(C)(C)c1ccc(Nc2ccc(C(C)(C)C)cc2-c2ccccc2)cc1 ; CC(C)(C)P(C(C)(C)C)C1(C)CC1(c1ccccc1)c1ccccc1 ; CC(C)(C)c1ccc(N(c2ccc3cc4c(cc3c2)oc2cc3cc(N(c5ccc(C(C)(C)C)cc5)c5ccc(C(C)(C)C)cc5-c5ccccc5)ccc3cc24)c2ccc(C(C)(C)C)cc2-c2ccccc2)cc1 ; "BD-4"]

1,1′-dinaphtho[2,3-b:2′,3′-d]furan-3,9-bistrifluoromethanesulfonate (1.5 g), Intermediate D (2.1 g), Pd₂(dba)₃ (50 mg) and di-tert-butyl(1-methyl-2,2-diphenylcyclopropyl)phosphine (75 mg) were added to a three-necked eggplant flask, and dehydrated toluene (150 mL) was added thereto. The solution was heated to 70° C. under an argon atmosphere and stirred for 30 minutes, and 7.6 mL of a toluene solution of LiHMDS (1 mol/L) was added dropwise into the system, followed by stirring for 6 hours. The solution was allowed to cool to room temperature, and then subjected to silica gel column chromatography to obtain a yellow solid. The yield was 2.0 g (77% yield). The molecular weight of BD-4 was 979, and the mass spectrum of the obtained compound was analyzed as m/z (ratio of mass to charge)=979, thereby identified as BD-4.

Although only some exemplary embodiments and/or examples of this invention have been described in detail above, those skilled in the art will readily appreciate that many modifications are possible in the exemplary embodiments and/or examples without materially departing from the novel teachings and advantages of this invention. Accordingly, all such modifications are intended to be included within the scope of this invention.

The documents described in the specification and the specification of Japanese application(s) on the basis of which the present application claims Paris convention priority are incorporated herein by reference in its entirety.

1. A compound represented by the following formula (1): 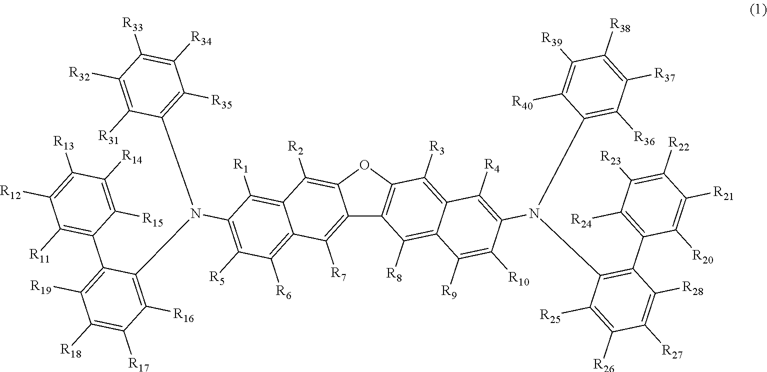
wherein in the formula (1),
R₁ to R₁₀ are independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a substituted or unsubstituted alkoxy group having 1 to 50 carbon atoms, a substituted or unsubstituted alkylthio group having 1 to 50 carbon atoms, a substituted or unsubstituted aryloxy group having 6 to 50 ring carbon atoms, a substituted or unsubstituted arylthio group having 6 to 50 ring carbon atoms, a substituted or unsubstituted aralkyl group having 7 to 50 carbon atoms, —Si(R₉₁)(R₉₂)(R₉₃), —C(═O)R₉₄, —COOR₉₅, —N(R₉₆)(R₉₇), a halogen atom, a cyano group, a nitro group, a substituted or unsubstituted aryl group having 6 to 50 ring carbon atoms, or a substituted or unsubstituted monovalent heterocyclic group having 5 to 50 ring atoms;
R₉₁ to R₉₇ are independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted monovalent heterocyclic group having 5 to 50 ring atoms;
when a plurality of each of R₉₁ to R₉₇ are present, the plurality of each of R₉₁ to R₉₇ may be the same as or different from each other;
R₁₁ to R₂₈ are independently a hydrogen atom, a substituted or unsubstituted alkyl group having 1 to 20 carbon atoms, a substituted or unsubstituted cycloalkyl group having 3 to 20 ring carbon atoms, a silyl group substituted with an alkyl group having 1 to 20 carbon atoms, or a cyano group;
R₃₁ to R₄₀ are independently a hydrogen atom, a substituted or unsubstituted alkyl group having 1 to 20 carbon atoms, a substituted or unsubstituted cycloalkyl group having 3 to 20 ring carbon atoms, a substituted or unsubstituted aryl group having 6 to 20 ring carbon atoms, a silyl group substituted with an alkyl group having 1 to 20 carbon atoms, or a cyano group;
at least one of R₁₁ to R₂₈ and R₃₁ to R₄₀ is not a hydrogen atom.

2. The compound according to claim 1, which is represented by the following formula (1-1): 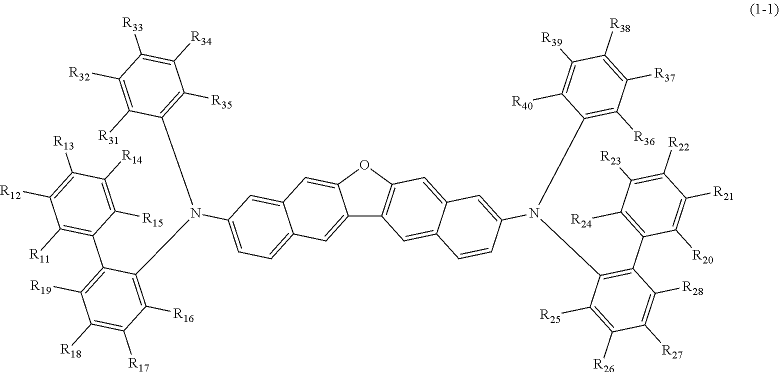
wherein in the formula (1-1), R₁₁ to R₂₈ and R₃₁ to R₄₀ are the same as defined in the formula (1).

3. The compound according to claim 1, which is represented by the following formula (1-2): 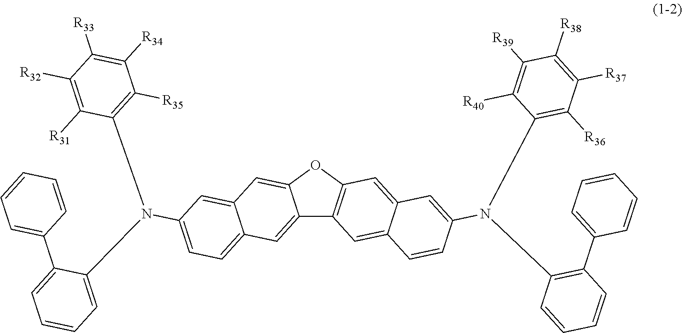
wherein in the formula (1-2), R₃₁ to R₄₀ are the same as defined in the formula (1); least one of R₃₁ to R₄₀ is not a hydrogen atom.

4. The compound according to claim 1, wherein at least one of R₃₁ to R₄₀ is a substituted or unsubstituted alkyl group having 1 to 20 carbon atoms, or a substituted or unsubstituted aryl group having 6 to 20 ring carbon atoms.

5. The compound according to claim 1, wherein at least one of R₃₁ to R₃₅ is a substituted or unsubstituted alkyl group having 1 to 20 carbon atoms, or a substituted or unsubstituted aryl group having 6 to 20 ring carbon atoms, and
at least one of R₃₆ to R₄₀ is a substituted or unsubstituted alkyl group having 1 to 20 carbon atoms, or a substituted or unsubstituted aryl group having 6 to 20 ring carbon atoms.

6. The compound according to claim 1, which is represented by the following formula (1-3): 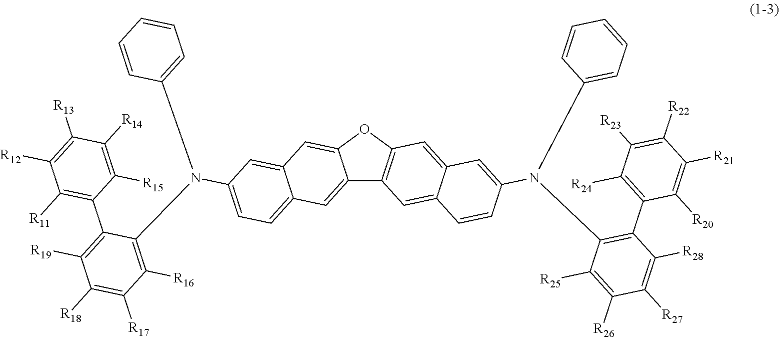
wherein in the formula (1-3), R₁₁ to R₂₈ are the same as defined in the formula (1); at least one of R₁₁ to R₂₈ is not a hydrogen atom.

7. The compound according to claim 1, wherein a substituent in the case of “substituted or unsubstituted” is selected from the group consisting of an alkyl group having 1 to 50 carbon atoms, a haloalkyl group having 1 to 50 carbon atoms, an alkenyl group having 2 to 50 carbon atoms, an alkynyl group having 2 to 50 carbon atoms, a cycloalkyl group having 3 to 50 ring carbon atoms, an alkoxy group having 1 to 50 carbon atoms, an alkylthio group having 1 to 50 carbon atoms, an aryloxy group having 6 to 50 ring carbon atoms, an arylthio group having 6 to 50 ring carbon atoms, an aralkyl group having 7 to 50 carbon atoms, —Si(R₄₁)(R₄₂)(R₄₃), —C(═O)R₄₄, —COOR₄₅, —S(═O)₂R₄₆, —P(═O)(R₄₇)(R₄₈), —Ge(R₄₉)(R₅₀)(R₅₁), —N(R₅₂)(R₅₃) (wherein, R₄₁ to R₅₃ are independently a hydrogen atom, an alkyl group having 1 to 50 carbon atoms, an aryl group having 6 to 50 ring carbon atoms, or a monovalent heterocyclic group having 5 to 50 ring atoms; when two or more of each of R₄₁ to R₅₃ are present, the two or more of each of R₄₁ to R₅₃ may be the same as or different from each other), a hydroxy group, a halogen atom, a cyano group, a nitro group, an aryl group having 6 to 50 ring carbon atoms, and a monovalent heterocyclic group having 5 to 50 ring atoms.

8. A material for an organic electroluminescence device comprising the compound according to claim 1.

9. An organic electroluminescence device comprising
a cathode;
an anode; and
at least one organic layer arranged between the cathode and the anode,
wherein at least one layer of the at least one organic layer comprises the compound according to claim 1.

10. The organic electroluminescence device according to claim 9, wherein the at least one layer of the at least one organic layer comprises a second compound that is not the same as the first compound according to claim 1.

11. The organic electroluminescence device according to claim 10, wherein the second compound is a heterocyclic compound or a fused aromatic compound.

12. The organic electroluminescence device according to claim 10, wherein the second compound is an anthracene derivative.

13. The organic electroluminescence device according to claim 12, wherein the second compound is a compound represented by the following formula (20): 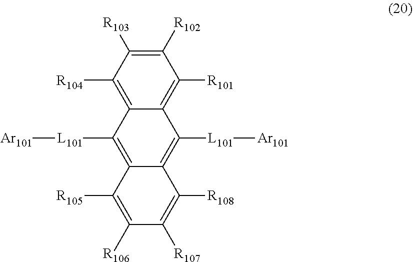
wherein one or more sets of the adjacent two or more of R₁₀₁ to R₁₀₈ form a substituted or unsubstituted, saturated or unsaturated ring, or do not form the substituted or unsubstituted, saturated or unsaturated ring;
R₁₀₁ to R₁₀₈ which do not form the substituted or unsubstituted, saturated or unsaturated ring are independently
a hydrogen atom, or
a group represented by substituent R;
L₁₀₁'s are
a single bond,
a substituted or unsubstituted arylene group having 6 to 50 ring carbon atoms, or
a substituted or unsubstituted divalent heterocyclic group having 5 to 50 ring atoms;
Ar₁₀₁'s are
a substituted or unsubstituted aryl group having 6 to 50 ring carbon atoms, or
a substituted or unsubstituted monovalent heterocyclic group having 5 to 50 ring atoms;
the substituent R is
a substituted or unsubstituted alkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
—Si(R₉₀₁)(R₉₀₂)(R₉₀₃),
—O—(R₉₀₄),
—S—(R₉₀₅),
—N(R₉₀₆)(R₉₀₇),
a halogen atom, a cyano group, a nitro group,
a substituted or unsubstituted aryl group having 6 to 50 ring carbon atoms, or
a substituted or unsubstituted monovalent heterocyclic group having 5 to 50 ring atoms;
the two Ar₁₀₁'s may be the same as or different from each other;
the two L₁₀₁'s may be the same as or different from each other;
when two or more substituents R are present, the two or more substituents R may be the same as or different from each other;
R₉₀₁ to R₉₀₇ are independently
a hydrogen atom,
a substituted or unsubstituted alkyl group having 1 to 50 carbon atoms,
a substituted or unsubstituted cycloalkyl group having 3 to 50 ring carbon atoms,
a substituted or unsubstituted aryl group having 6 to 50 ring carbon atoms, or
a substituted or unsubstituted monovalent heterocyclic group having 5 to 50 ring atoms;
when two or more of each of R₉₀₁ to R₉₀₇ are present, the two or more of each of R₉₀₁ to R₉₀₇ may be the same as or different from each other.

14. The organic electroluminescence device according to claim 9, wherein the at least one layer of the at least one organic layer is an emitting layer.

15. The organic electroluminescence device according to claim 14, wherein a hole-transporting layer is provided between the anode and the emitting layer.

16. The organic electroluminescence device according to claim 14, wherein an electron-transporting layer is provided between the cathode and the emitting layer.

17. The organic electroluminescence device according to claim 14, wherein the emitting layer includes a compound represented by the formula (20).

18. The organic electroluminescence device according to claim 14, wherein the emitting layer further comprises a host compound having delayed fluorescence.

19. An electronic apparatus, comprising the organic electroluminescence device according to claim 9.
